SEQUENCE LISTING (120 sequences)

SEQ ID NO: 1 - protein, 32 aa, Artificial Sequence
  PEST sequence
KENSISSMAPPASPPASPKTPIEKKHADEIDK

SEQ ID NO: 2 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 3 - protein, 441 aa, Artificial Sequence
  N-terminal LLO protein fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 4 - protein, 416 aa, Artificial Sequence
  LLO signal peptide
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 14 aa, Artificial Sequence
  PEST sequence
KTEEQPSEVNTGPR

SEQ ID NO: 6 - protein, 28 aa, Artificial Sequence
  PEST sequence
KASVTDTSEGDLDSSMQSADESTPQPLK

SEQ ID NO: 7 - protein, 20 aa, Artificial Sequence
  PEST sequence
KNEEVNASDFPPPPTDEELR

SEQ ID NO: 8 - protein, 33 aa, Artificial Sequence
  PEST sequence
RGGIPTSEEFSSLNSGDFTDDENSETTEEEIDR

SEQ ID NO: 9 - protein, 17 aa, Artificial Sequence
  PEST sequence
KQNTASTETTTTNEQPK

SEQ ID NO: 10 - protein, 17 aa, Artificial Sequence
  PEST-like sequence
KQNTANTETTTTNEQPK

SEQ ID NO: 11 - protein, 639 aa, Artificial Sequence
  ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISDRALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 12 - protein, 390 aa, Artificial Sequence
  Truncated ActA protein
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 13 - protein, 100 aa, Artificial Sequence
  Truncated ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 14 - protein, 93 aa, Artificial Sequence
  PEST sequence
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 15 - protein, 200 aa, Artificial Sequence
  PEST sequence
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 16 - protein, 303 aa, Artificial Sequence
  PEST sequence
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 17 - protein, 370 aa, Artificial Sequence
  PEST sequence
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 18 - DNA, 1170 bp, Artificial Sequence
  Recombinant nucleotide encoding anActA protein
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 19 - protein, 14 aa, Artificial Sequence
  PEST sequence
KTEEQPSEVNTGPR

SEQ ID NO: 20 - protein, 28 aa, Artificial Sequence
  PEST sequence
KASVTDTSEGDLDSSMQSADESTPQPLK

SEQ ID NO: 21 - protein, 20 aa, Artificial Sequence
  PEST sequence
KNEEVNASDFPPPPTDEELR

SEQ ID NO: 22 - protein, 33 aa, Artificial Sequence; identical to SEQ ID NO: 8
  PEST sequence

SEQ ID NO: 23 - protein, 17 aa, Artificial Sequence
  PEST-like sequence
KQNTASTETTTTNEQPK

SEQ ID NO: 24 - protein, 17 aa, Artificial Sequence
  PEST-like sequence
KQNTANTETTTTNEQPK

SEQ ID NO: 25 - protein, 261 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 26 - protein, 237 aa, Artificial Sequence
  KLK3 protein
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANP

SEQ ID NO: 27 - protein, 237 aa, Artificial Sequence
  KLK3 protein
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 28 - DNA, 5873 bp, Artificial Sequence
  Nucleotide molecule encoding the KLK3 protein
ggtgtcttaggcacactggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagtcccctccatattgcaacagtcctcactcccacaccaggtccccgctccctcccacttaccccagaactttcttcccatttgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttctttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgagtccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctgggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggagctgagggaggagaattgcattgaacctggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggcaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgcccttttaccctcttccttttcccttggttctctcagttctgtatctgcccttcaccctctcacactgctgtttcccaactcgttgtctgtattttggcctgaactgtgtcttcccaaccctgtgttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgagggaaaggtgagtgggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgcgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaacaaaggacccctcctgcagggcctcacctgggccacaggaggacactgcttttcctctgaggagtcaggaactgtggatggtgctggacagaagcaggacagggcctggctcaggtgtccagaggctgcgctggcctcctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttcctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaagtccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagttaggatggggtgtctgtgttatttgtgggatacagagatgaaagaggggtgggatcc

SEQ ID NO: 29 - protein, 238 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 30 - DNA, 1906 bp, Artificial Sequence
  Nucleotide molecule encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 31 - protein, 69 aa, Artificial Sequence
  KLK3 protein
MWVPVVFLTLSVTWIGAAPLILSRIVGGWECEKHSQPWQVLVASRGRAVCGGVLVHPQWVLTAAHCIRK

SEQ ID NO: 32 - DNA, 554 bp, Artificial Sequence
  Nucleotide molecule encoding the KLK3 protein
agccccaagcttaccacctgcacccggagagctgtgtcaccatgtgggtcccggttgtcttcctcacccttccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgttcctctatgtctctttgtgtcgctttcattatgtctcttggtaactggcttcggttgtgtctctccgtgtgactattttgttctctctctccctctcttctctgtcttcagt

SEQ ID NO: 33 - protein, 220 aa, Artificial Sequence
  KLK3 protein that is the source of the KLK3 peptide
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 34 - DNA, 1341 bp, Artificial Sequence
  Nucleotide molecule encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 35 - protein, 218 aa, Artificial Sequence
  KLK3 protein
MWVPVVFLTLSVTWIGAAPLILSRIVGGWECEKHSQPWQVLVASRGRAVCGGVLVHPQWVLTAAHCIRKPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 36 - DNA, 1325 bp, Artificial Sequence
  Nucleotide molecule encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattacccaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 37 - protein, 261 aa, Artificial Sequence; identical to SEQ ID NO: 25
  KLK3 protein

SEQ ID NO: 38 - DNA, 1464 bp, Artificial Sequence
  Nucleotide molecule encoding  the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 39 - protein, 261 aa, Artificial Sequence; identical to SEQ ID NO: 25
  KLK3 protein

SEQ ID NO: 40 - DNA, 1495 bp, Artificial Sequence
  Nucleotide molecule encoding the KLK3 protein
gggggagccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtagggagagggactggaccccctggaagctgattcactatggggggaggtgtattgaagtcctccagacaaccctcagatttgatgatttcctagtagaactcacagaaataaagagctgttatactgcgaaaaaaaaaaaaaaaaaaaaaaaaaa

SEQ ID NO: 41 - protein, 218 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVA

SEQ ID NO: 42 - protein, 227 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSVSHPYSQDLEGKGEWGP

SEQ ID NO: 43 - protein, 104 aa, Artificial Sequence
  KLK3 protein
MWVPVVFLTLSVTWIGERGHGWGDAGEGASPDCQAEALSPPTQHPSPDRELGSFLSLPAPLQAHTPSPSILQQSSLPHQVPAPSHLPQNFLPIAQPAPCSQLLY

SEQ ID NO: 44 - protein, 261 aa, Artificial Sequence; identical to SEQ ID NO: 25
  KLK3 protein

SEQ ID NO: 45 - DNA, 1729 bp, Artificial Sequence
  Nucleotide molecule encoding the KLK3 protein
aagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtagcctctcgtggcagggcagtctgcggcggtgttctggtgcacccccagtgggtcctcacagctacccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgaaccaaggagggagggtcttcctttggcatgggatggggatgaagtaaggagagggactgaccccctggaagctgattcactatggggggaggtgtattgaagtcctccagacaaccctcagatttgatgatttcctagtagaactcacagaaataaagagctgttatactgtgaa

SEQ ID NO: 46 - protein, 69 aa, Artificial Sequence; identical to SEQ ID NO: 31
  KLK3 protein

SEQ ID NO: 47 - protein, 220 aa, Artificial Sequence; identical to SEQ ID NO: 33
  KLK3 protein that is the source of the KLK3 peptide

SEQ ID NO: 48 - protein, 218 aa, Artificial Sequence; identical to SEQ ID NO: 35
  KLK3 protein

SEQ ID NO: 49 - protein, 24 aa, Artificial Sequence
  Sequence lacking when KLK3 protein is encoded
MWVPVVFLTLSVTWIGAAPLILSR

SEQ ID NO: 50 - DNA, 2040 bp, Artificial Sequence
  Nucleotide molecule encoding a LLO fused to a PSA protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 51 - protein, 680 aa, Artificial Sequence [sequence omitted]

SEQ ID NO: 52 - protein, 98 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 53 - protein, 105 aa, Artificial Sequence
  E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 54 - protein, 97 aa, Artificial Sequence
  E7 protein
HGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 55 - protein, 540 aa, Artificial Sequence
  Truncated LLO fused to an E7 protein
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 56 - DNA, 1263 bp, Artificial Sequence
  Nucleotide molecule encoding the Her-2 chimeric protein
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 57 - protein, 419 aa, Artificial Sequence
  Her-2 chimeric protein
THLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 58 - DNA, 2586 bp, Artificial Sequence
  Open reading frame encoding tLLO fused to Cher2
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 59 - protein, 861 aa, Artificial Sequence
  tLLO fused to a Cher2
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLL

SEQ ID NO: 60 - DNA, 3798 bp, Artificial Sequence
  Human-Her2/neu gene
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctga

SEQ ID NO: 61 - DNA, 393 bp, Artificial Sequence
  Nucleotide molecule encoding the human her2/neu EC1 fragment implemented into the chimera
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaag

SEQ ID NO: 62 - DNA, 921 bp, Artificial Sequence
  EC1 human her2/neu fragment
gccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggat

SEQ ID NO: 63 - DNA, 477 bp, Artificial Sequence
  Nucleotide molecule encoding the human her2/neu EC2 fragment implemented into the chimera
aatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgaggg

SEQ ID NO: 64 - DNA, 597 bp, Artificial Sequence
  EC2 human her2/neu fragment
tacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagag

SEQ ID NO: 65 - DNA, 391 bp, Artificial Sequence
  Nucleotide molecule encoding the human her2/neu IC1 fragment implemented into the chimera
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagact

SEQ ID NO: 66 - DNA, 1209 bp, Artificial Sequence
  Nucleotide molecule encoding  the complete human her2/neu IC1 fragment
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggct

SEQ ID NO: 67 - DNA, 6523 bp, Artificial Sequence
  Plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 68 - DNA, 36 bp, Artificial Sequence
  Adv271-actAF1
cggaattcggatccgcgccaaatcattggttgattg

SEQ ID NO: 69 - DNA, 37 bp, Artificial Sequence
  Adv272-actAR1
gcgagtcgacgtcggggttaatcgtaatgcaattggc

SEQ ID NO: 70 - DNA, 35 bp, Artificial Sequence
  Adv273-actAF2
gcgagtcgacccatacgacgttaattcttgcaatg

SEQ ID NO: 71 - DNA, 39 bp, Artificial Sequence
  Adv274-actAR2
gatactgcagggatccttcccttctcggtaatcagtcac

SEQ ID NO: 72 - DNA, 19 bp, Artificial Sequence
  Primer 3
tgggatggccaagaaattc

SEQ ID NO: 73 - DNA, 22 bp, Artificial Sequence
  Primer 4
ctaccatgtcttccgttgcttg

SEQ ID NO: 74 - DNA, 28 bp, Artificial Sequence
  Her-2-Chimera (F)
tgatctcgagacccacctggacatgctc

SEQ ID NO: 75 - DNA, 49 bp, Artificial Sequence
  HerEC1-EC2F (Junction)
ctaccaggacacgattttgtggaagaatatccaggagtttgctggctgc

SEQ ID NO: 76 - DNA, 49 bp, Artificial Sequence
  HerEC1-EC2R (Junction)
gcagccagcaaactcctggatattcttccacaaaatcgtgtcctggtag

SEQ ID NO: 77 - DNA, 50 bp, Artificial Sequence
  HerEC2-ICIF (Junction)
ctgccaccagctgtgcgcccgagggcagcagaagatccggaagtacacga

SEQ ID NO: 78 - DNA, 50 bp, Artificial Sequence
  HerEC2-ICIR (Junction)
tcgtgtacttccggatcttctgctgccctcgggcgcacagctggtggcag

SEQ ID NO: 79 - DNA, 39 bp, Artificial Sequence
  Her-2-Chimera (R)
gtggcccgggtctagattagtctaagaggcagccatagg

SEQ ID NO: 80 - DNA, 28 bp, Artificial Sequence
  Her-2-EC1(F)
ccgcctcgaggccgcgagcacccaagtg

SEQ ID NO: 81 - DNA, 31 bp, Artificial Sequence
  Her-2-EC1(R)
cgcgactagtttaatcctctgctgtcacctc

SEQ ID NO: 82 - DNA, 28 bp, Artificial Sequence
  Her-2-EC2(F)
ccgcctcgagtacctttctacggacgtg

SEQ ID NO: 83 - DNA, 30 bp, Artificial Sequence
  Her- 2- EC2(R)
cgcgactagtttactctggccggttggcag

SEQ ID NO: 84 - DNA, 31 bp, Artificial Sequence
  Her-2-Her-2-IC1(F)
ccgcctcgagcagcagaagatccggaagtac

SEQ ID NO: 85 - DNA, 30 bp, Artificial Sequence
  Her-2-IC1(R)
cgcgactagtttaagccccttcggagggtg

SEQ ID NO: 86 - DNA, 7075 bp, Artificial Sequence
  pAdv164 sequence
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 87 - protein, 9 aa, Artificial Sequence
  Extracellular domain of the Her2/neu
HLYQGCQVV

SEQ ID NO: 88 - protein, 9 aa, Artificial Sequence
  Extracellular domain of the Her2/neu
KIFGSLAFL

SEQ ID NO: 89 - protein, 9 aa, Artificial Sequence
  Intracellular domain of the Her2/neu
RLLQETELV

SEQ ID NO: 90 - DNA, 55 bp, Artificial Sequence
  Synthetic
ggtcacagctgaggacggaacacagcgttgtgagaaatgcagcaagccctgtgct

SEQ ID NO: 91 - DNA, 60 bp, Artificial Sequence
  Synthetic
cgagtgtgctatggtctgggcatggagcaccttcgaggggcgagggccatcaccagtgac

SEQ ID NO: 92 - DNA, 60 bp, Artificial Sequence
  Synthetic
aatgtccaggagtttgatggctgcaagaagatctttgggagcctggcatttttgccggag

SEQ ID NO: 93 - DNA, 60 bp, Artificial Sequence
  Synthetic
agctttgatggggacccctcctccggcattgctccgctgaggcctgagcagctccaagtg

SEQ ID NO: 94 - DNA, 60 bp, Artificial Sequence
  Synthetic
ttcgaaaccctggaggagatcacaggttacctgtacatctcagcatggccagacagtctc

SEQ ID NO: 95 - DNA, 60 bp, Artificial Sequence
  Synthetic
cgtgacctcagtgtcttccagaaccttcgaatcattcggggacggattctccacgatggc

SEQ ID NO: 96 - DNA, 60 bp, Artificial Sequence
  Synthetic
gcgtactcattgacactgcaaggcctggggatccactcgctggggctgcgctcactgcgg

SEQ ID NO: 97 - DNA, 60 bp, Artificial Sequence
  Synthetic
gagctgggcagtggattggctctgattcaccgcaacgcccatctctgctttgtacacact

SEQ ID NO: 98 - DNA, 60 bp, Artificial Sequence
  Synthetic
gtaccttgggaccagctcttccggaacccacatcaggccctgctccacagtgggaaccgg

SEQ ID NO: 99 - DNA, 60 bp, Artificial Sequence
  Synthetic
ccggaagaggattgtggtctcgagggcttggtctgtaactcactgtgtgcccacgggcac

SEQ ID NO: 100 - DNA, 59 bp, Artificial Sequence
  Synthetic
tgctgggggccagggcccacccagtgtgtcaactgcagtcatttccttcggggccagga

SEQ ID NO: 101 - DNA, 57 bp, Artificial Sequence
  Synthetic
cgcccagcggagcaatgcccaaccaggctcagatgcggatcctaaaagagacggagc

SEQ ID NO: 102 - DNA, 60 bp, Artificial Sequence
  Synthetic
taaggaaggtgaaggtgcttggatcaggagcttttggcactgtctacaagggcatctgga

SEQ ID NO: 103 - DNA, 60 bp, Artificial Sequence
  Synthetic
tcccagatggggagaatgtgaaaatccccgtggctatcaaggtgttgagagaaaacacat

SEQ ID NO: 104 - DNA, 60 bp, Artificial Sequence
  Synthetic
ctcctaaagccaacaaagaaattctagatgaagcgtatgtgatggctggtgtgggttctc

SEQ ID NO: 105 - DNA, 60 bp, Artificial Sequence
  Synthetic
cgtatgtgtcccgcctcctgggcatctgcctgacatccacagtacagctggtgacacagc

SEQ ID NO: 106 - DNA, 60 bp, Artificial Sequence
  Synthetic
ttatgccctacggctgccttctggaccatgtccgagaacaccgaggtcgcctaggctccc

SEQ ID NO: 107 - DNA, 60 bp, Artificial Sequence
  Synthetic
aggacctgctcaactggtgtgttcagattgccaaggggatgagctacctggaggacgtgc

SEQ ID NO: 108 - DNA, 60 bp, Artificial Sequence
  Synthetic
ggcttgtacacagggacctggctgcccggaatgtgctagtcaagagtcccaaccacgtca

SEQ ID NO: 109 - DNA, 60 bp, Artificial Sequence
  Synthetic
agattacagatttcgggctggctcggctgctggacattgatgagacagagtaccatgcag

SEQ ID NO: 110 - DNA, 60 bp, Artificial Sequence
  Synthetic
atgggggcaaggtgcccatcaaatggatggcattggaatctattctcagacgccggttca

SEQ ID NO: 111 - DNA, 60 bp, Artificial Sequence
  Synthetic
cccatcagagtgatgtgtggagctatggagtgactgtgtgggagctgatgacttttgggg

SEQ ID NO: 112 - DNA, 59 bp, Artificial Sequence
  Synthetic
ccaaaccttacgatggaatcccagcccgggagatccctgatttgctggagaagggagaa

SEQ ID NO: 113 - DNA, 58 bp, Artificial Sequence
  Synthetic
cgcctacctcagcctccaatctgcaccattgatgtctacatgattatggtcaaatgtt

SEQ ID NO: 114 - DNA, 54 bp, Artificial Sequence
  Synthetic
ggatgattgactctgaatgtcgcccgagattccgggagttggtgtcagaatttt

SEQ ID NO: 115 - DNA, 52 bp, Artificial Sequence
  Synthetic
cacgtatggcgagggacccccagcgttttgtggtcatccagaacgaggactt

SEQ ID NO: 116 - DNA, 60 bp, Artificial Sequence
  Synthetic
cccaggcagaaccccagaggggctgcgggagctgcagcttcgaagtctcacagagatcct

SEQ ID NO: 117 - DNA, 60 bp, Artificial Sequence
  Synthetic
gaagggaggagttttgatccgtgggaaccctcagctctgctaccaggacatggttttgtg

SEQ ID NO: 118 - DNA, 60 bp, Artificial Sequence
  Synthetic
ccgggcctgtccaccttgtgcccccgcctgcaaagacaatcactgttggggtgagagtcc

SEQ ID NO: 119 - DNA, 60 bp, Artificial Sequence
  Synthetic
ggaagactgtcagatcttgactggcaccatctgtaccagtggttgtgcccggtgcaaggg

SEQ ID NO: 120 - DNA, 60 bp, Artificial Sequence
  Synthetic
ccggctgcccactgactgctgccatgagcagtgtgccgcaggctgcacgggccccaagca

(19) United States
(12) Patent Application Publication
(10) Pub. No.: US 2018/0153974 A1
(43) Pub. Date: Jun. 7, 2018

(54) Combination Therapies With Recombinant Listeria Strains

(71) Applicants: THE TRUSTEES OF THE UNIVERSITY OF PENNSYLVANIA, Philadelphia, PA (US); ADVAXIS, INC., Princeton, NJ (US)

(72) Inventors: Robert Petit, Newton, PA (US); Anu Wallecha, Yardley, PA (US); Yvonne Patterson, Philadelphia, PA (US); Reshma Singh, Brookline, MA (US)

(21) Appl. No.: 15/534,910

(22) PCT Filed: Dec. 18, 2015
(86) PCT No.: PCT/US2015/066885

§ 371 Date: Jun. 9, 2017

Related U.S. Application Data
(60) Provisional application No. 62/094,349, filed on Dec. 19, 2014.
(60) Provisional application No. 62/094,472, filed on Dec. 19, 2014.
(60) Provisional application No. 62/147,463, filed on Apr. 14, 2015.
(60) Provisional application No. 62/262,896, filed on Dec. 3, 2015.

(51) Int. Cl. A61K 39/00; C07K 14/025; C07K 14/195; C07K 16/28

(52) U.S. Cl. CPC A61K 39/0011; C07K 14/025; C07K 14/195; C07K 16/2818; C07K 2319/95; A61K 2039/522; A61K 2039/572; C07K 2319/40; A61K 2039/505

(57) ABSTRACT

The disclosure is directed to compositions comprising an oncolytic virus, chimeric antigen receptor T cells (CAR T cells), a therapeutic or immunomodulating monoclonal antibody, a targeting thymidine kinase inhibitor (TKI), or an adoptively transferred cells incorporating engineered T cell receptors, and a live attenuated recombinant Listeria strain comprising a fusion protein of a Truncated LLO, a truncated ActA or a PEST-sequence peptide fused to a tumor-associated antigen. The disclosure is further directed to methods of treating, protecting against, and inducing an immune response against a tumor, comprising the step of administering the same, with or without an additional radiation therapy treatment.

### FIELD OF INTEREST

The disclosure is directed to combination therapies comprising use of compositions comprising a live attenuated recombinant Listeria strain comprising a fusion protein of a Truncated LLO, a truncated ActA or a PEST-sequence peptide fused to a tumor-associated antigen, wherein the compositions further comprise or are co-administered with an additional active agent. The disclosure is further directed to combination therapies comprising use of these compositions comprising live attenuated recombiant Listeria strains, in conjuction with a targeted radiation therapy for treating, protecting against, and/or inducing an immune response against a tumor.

### BACKGROUND

Listeria monocytogenes (Lm) is a Gram-positive facultative intracellular pathogen that causes listeriolysis. Once invading a host cell, Lm can escape from the phagolysosome through production of a pore-forming protein listeriolysin O (LLO) to lyse the vascular membrane, allowing it to enter the cytoplasm, where it replicates and spreads to adjacent cells based on the mobility of actin-polymerizing protein (ActA). In the cytoplasm, Lm-secreting proteins are degraded by the proteasome and processed into peptides that associate with MHC class I molecules in the endoplasmic reticulum. This unique characteristic makes it a very attractive cancer immunotherapeutic vector in that tumor antigen can be presented with MHC class I molecules to activate tumor-specific cytotoxic T lymphocytes (CTLs).

In addition, once phagocytized, Lm may then be processed in the phagolysosomal compartment and peptides presented on MHC Class II for activation of Lm-specific CD4-T cell responses. Alternatively, Lm can escape the phagosome and enter the cytosol where recognition of peptidoglycan by nuclear oligomerization domain-like receptors and Lm DNA by DNA sensor, AIM2, activate inflammatory cascades. This combination of inflammatory responses and efficient delivery of antigens to the MHC I and MHC II pathways makes Lm a powerful immunotherapeutic vector in treating, protecting against, and inducing an immune response against a tumor.

However, tumor cells often induce an immunosuppressive microenvironment, which favors the development of immunosuppressive populations of immune cells, such as myeloid-derived suppressor cells and regulatory T cells. Understanding the complexity of immunomodulation by tumors is important for the development of immunotherapy. Various strategies are being developed to enhance anti-tumor immune responses and to overcome ‘immune checkpoints’. In addition, administration of combination immunotherapies may provide a more efficacious and enduring response.

For example, one of several mechanisms of tumor-mediated immune suppression is the expression of T-cell co-inhibitory molecules by tumor. Upon engagement to their ligands these molecules can suppress effector lymphocytes in the periphery and in the tumor microenvironment.

Presently, there remains a need to provide effective combination therapies for tumor targeting methods that can eliminate tumor growth and cancer. The disclosure addresses this need by providing a combination of a Listeria-based immunotherapy with various therapies including addition of active agents such as oncolytic viruses, chimeric antigen receptor engineered cells (CAR T cells), therapeutic or immunomodulating monoclonal antibodies, a targeting thymidine kinase inhibitor, and/or adoptively transferred cells that may incorporate engineered T cell receptors, which may further be used in combination with additional therapies such as targeted radiation therapy.

Oncolytic viruses (OVs) are self-amplifying biotherapeutics that have been selected or engineered to preferentially infect and kill cancer cells in vivo. Generated from a multitude of viral species including adenoviruses, reoviruses, alphaviruses, Herpes Simplex virus, Newcastle disease virus, Coxsackie B virus, Coxsackie A21 virus, Sindbis virus, measles virus, poliovirus, vesicular stomatitis virus, myxoma virus, vaccinia virus and other poxviruses, Sendai virus, and influenza virus. OVs exploit cancer-associated cellular defects arising from genetic perturbations including mutations and epigenetic reprograming. Among others, these cellular defects lead to dysfunctional anti-viral responses and immune evasion, increased cell proliferation and metabolism, and leaky tumor vasculature. These characteristics in turn provide a fertile ground for viral replication and subsequent lysis of tumor cells and permit the growth of genetically attenuated OVs that are otherwise harmless to normal cells.

In addition to the direct killing of cancer cells, OVs can also trigger a potent anti-tumor immune response. Infected tumor cells induce the release of pro-inflammatory cytokines and expose both viral and tumor-associated antigens to patrolling immune cells, promoting the differentiation of antigen-presenting cells and T-cell activation. How much tumor infection and lysis are necessary to trigger these responses remains a topic of debate; however, it is clear that the combination of direct oncolysis and activation of anti-tumor immunity can lead to durable cures in pre-clinical mouse models of cancer.

Another immune therapy targeted approach involves engineering patients' own immune cells to recognize and attack their tumors. This approach is often known as adoptive cell transfer. For example, using recombinant technology known in the art the receptors present on T cells may have both polypeptide chains engineered to have a selected specificity (Receptor engineered T cells). In certain instances, T cells are engineered to have a chimeric antigen receptor, wherein one of the polypeptide chains is from the T cell receptor and that other polypeptide chain is from an antibody. These cells are known as chimeric antigen receptor T cells (CAR T cells). Adoptive cell transfer involves administration of T cells comprising engineer receptors, wherein the cells may be Receptor engineered T cell or CAR T cells, each engineered to produce special receptors on their surface, either engineer T cell receptors or chimeric T cell receptors called chimeric antigen receptors (CARs). CARs are proteins that allow the T cells to recognize a specific protein antigen on a tumor cell. CAR T cells are then administered to patient, wherein these engineered T cells can recognize and kill cancer cells that harbor the specific antigen on their surfaces. A combination therapy administering CAR T cells and a Listeria-based immunotherapy may provide another therapy to eliminate tumor growth and cancer. There remains a need to optimize the dosage and schedule for administrating these two treatments. The disclosure further addresses this need by providing a combination of a Listeria-based immunotherapy with targeted CAR T cell administration.

Another immune therapy targeted approach involves the use of monoclonal antibodies developed to specifically target antigens expressed on the surface of cancerous cells. Due to immunotolerance, a person's immune system does not always recognize cancer cells as foreign targets. A monoclonal antibody can be directed to attach to antigens on the surface of a cancer cell. In this way, the antibody marks the cancer cell and makes it easier for the immune system to find. Alternatively, antibodies targeting growth signals may help prevent a tumor from developing a blood supply so that the tumor fails to growth or remains small. In the case of a tumor with an already-established network of blood vessels, blocking the growth signals could cause the blood vessels to die and the tumor to shrink. A combination therapy administering a therapeutic and/or immunomodulatory antibody and a Listeria-based immunotherapy may provide another therapy to eliminate tumor growth and cancer. There remains a need to optimize the dosage and schedule for administrating these two treatments. The disclosure further addresses this need by providing a combination of a Listeria-based immunotherapy with therapeutic and/or immunomodulatory antibody administration.

Another immune therapy targeted approach is the administration of Tyrosine Kinase Inhibitor (TKI) anticancer treatments. TKI are chemical compounds that inhibit the activity of tyrosine kinase enzyme inside the body. Often, tyrosine kinases provide an activity that aids in the growth and metastasis of tumors. Therefore, incorporation of a TKI may prevent growth and spreading of a cancer. A combination therapy administering a TKI and a Listeria may provide another therapy to eliminate tumor growth and cancer. There remains a need to optimize the dosage and schedule for administrating these two treatments. The disclosure further addresses this need by providing a combination of a Listeria-based immunotherapy with TKI administration.

Evidence has recently emerged revealing the capacity of targeted radiation therapy (RT) to induce antitumor responses, suggesting a possible combination therapy combining RT with Listeria based immunotherapy to promote tumor-specific immunity. Since RT and Lm vaccine therapy each induce a different aspect of antitumor immunity, a combination of these therapies may result in an overall increase in intratumoral numbers of activated T cells, antigen specific CD8+ T cells, natural killer cells and levels of effector molecules, such as interferon-γ (IFN-γ) and granzyme B. There remains a need to optimize the dosage and schedule for administrating these two treatments. The disclosure further addresses this need by providing a combination of a Listeria-based immunotherapy with targeted radiation therapy regimes.

Given the complex nature of certain diseases, including cancer, a need exists for a combined approach in treating the same. As seen in the Detailed Description below, these combination therapies may improve the overall anti-tumor efficacy of immunotherapy.

### SUMMARY OF THE DISCLOSURE

In one aspect, the disclosure relates to an immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, said composition further comprising an additional active agent.

In a related aspect, the disclosure relates to a method of inhibiting tumor-mediated immunosuppression in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, wherein:
(a) said composition further comprises an additional active agent;
(b) said method further comprises a step of administering an effective amount of a composition comprising an additional active agent to said subject; or
(c) said method further comprises a step of administering a targeted radiation therapy to said subject; or
any combination thereof of (a)-(c).

In another related aspect, the disclosure relates to a method of eliciting an enhanced anti-tumor T cell response in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, wherein:

(a) said composition further comprises an additional active agent;
(b) said method further comprises a step of administering an effective amount of a composition comprising an additional active agent to said subject; or
(c) said method further comprises a step of administering a targeted radiation therapy to said subject; or any combination thereof of (a)-(c).

In another related aspect, the disclosure relates to a method of treating a tumor or cancer in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, wherein:
(a) said composition further comprises an additional active agent;
(b) said method further comprises a step of administering an effective amount of a composition comprising an additional active agent to said subject; or
(c) said method further comprises a step of administering a targeted radiation therapy to said subject; or
any combination thereof of (a)-(c).

In another related aspect, an additional active agent disclosed herein comprises an oncolytic virus, a T cell receptor engineered T cell (Receptor engineered T cells), a chimeric antigen receptor engineered T cell (CAR T cells), a therapeutic or immunomodulating monoclonal antibody, a targeting thymidine kinase inhibitor (TKI), or an adoptively transferred cell incorporating engineered T cell receptors, or any combination thereof.

### BRIEF DESCRIPTION OF THE DRAWINGS

The subject matter regarded as the disclosure is particularly pointed out and distinctly claimed in the concluding portion of the specification. The disclosure, however, both as to organization and method of operation, together with objects, features, and advantages thereof, may best be understood by reference to the following detailed description when read with the accompanying drawings in which:

FIG. 1. (FIG. 1A) Schematic representation of the chromosomal region of the Lmdd-143 and LmddA-143 after klk3 integration and actA deletion; (Figure B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIG. 2. (FIG. 2A) Map of the pADV134 plasmid. (FIG. 2B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (FIG. 2C) Map of the pADV142 plasmid. (FIG. 2D) Western blot showed the expression of LLO-PSA fusion protein using anti-PSA and anti-LLO antibody.

FIG. 3. (FIG. 3A) Plasmid stability in vitro of LmddA-LLO-PSA if cultured with and without selection pressure (D-alanine). Strain and culture conditions are listed first and plates used for CFU determination are listed after. (FIG. 3B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIG. 4. (FIG. 4A) In vivo clearance of the strain LmddA-LLO-PSA after administration of 10⁸ CFU in C57BL/6 mice. The number of CFU were determined by plating on BHI/str plates. The limit of detection of this method was 100 CFU. (FIG. 4B) Cell infection assay of J774 cells with 10403S, LmddA-LLO-PSA and XFL7 strains.

FIG. 5. (FIG. 5A) PSA tetramer-specific cells in the splenocytes of naïve and LmddA-LLO-PSA immunized mice on day 6 after the booster dose. (FIG. 5B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (FIG. 5C) and a europium based assay (FIG. 5D). Number of IFNγ spots in naïve and immunized splenocytes obtained after stimulation for 24 h in the presence of PSA peptide or no peptide (FIG. 5E).

FIG. 6. Immunization with LmddA-142 induces regression of Tramp-C1-PSA (TPSA) tumors. Mice were left untreated (n=8) (FIG. 6A) or immunized i.p. with LmddA-142 (1×10⁸ CFU/mouse) (n=8) (FIG. 6B) or Lm-LLO-PSA (n=8) (FIG. 6C) on days 7, 14 and 21. Tumor sizes were measured for each individual tumor and the values expressed as the mean diameter in millimeters. Each line represents an individual mouse.

FIG. 7. (FIG. 7A) Analysis of PSA-tetramer⁺CD8⁺ T cells in the spleens and infiltrating T-PSA-23 tumors of untreated mice and mice immunized with either an Lm control strain or Lm-ddA-LLO-PSA (LmddA-142). (FIG. 7B) Analysis of CD4⁺ regulatory T cells, which were defined as CD25⁺FoxP3⁺, in the spleens and infiltrating T-PSA-23 tumors of untreated mice and mice immunized with either an Lm control strain or Lm-ddA-LLO-PSA.

FIG. 8. (FIG. 8A) Schematic representation of the chromosomal region of the Lmdd-143 and LmddA-143 after klk3 integration and actA deletion; (FIG. 8B) The klk3 gene is integrated into the Lmdd and LmddA chromosome. PCR from chromosomal DNA preparation from each construct using klk3 specific primers amplifies a band of 760 bp corresponding to the klk3 gene.

FIG. 9. (FIG. 9A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (FIG. 9B) LLO produced by Lmdd-143 and LmddA-143 retains hemolytic activity. Sheep red blood cells were incubated with serial dilutions of bacterial culture supernatants and hemolytic activity measured by absorbance at 590 nm; (FIG. 9C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 10. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIGS. 11A-B show a decrease in MDSCs and Tregs in tumors. The number of MDSCs (right-hand panel) and Tregs (left-hand panel) following Lm vaccination (LmddAPSA and LmddAE7).

FIG. 12. Figures show suppressor assay data demonstrating that monocytic MDSCs from TPSA23 tumors (PSA expressing tumor) are less suppressive after Listeria vaccination. This change in the suppressive ability of the MDSCs is not antigen specific as the same decrease in suppression is seen with PSA-antigen specific T cells and also with non-specifically stimulated T cells. In FIGS. 12A and 12B Phorbol-Myristate-Acetate and lonomycin (PMA/I) represents non-specific stimulation. In FIGS. 12C and 12D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 12A and 12C show individual cell division cycles for each group. FIGS. 12B and 12D show pooled division cycles.

FIG. 13 show suppressor assay data demonstrating that Listeria has no effect on splenic monocytic MDSCs and they are only suppressive in an antigen-specific manner. In FIGS. 13A and 13B PMA/I represents non-specific stimulation. In FIGS. 13C and 13D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 13A and 13C show individual cell division cycles for each group. FIGS. 13B and 13D show pooled division cycles.

FIG. 14 show suppressor assay data demonstrating that granulocytic MDSCs from tumors have a reduced ability to suppress T cells after Listeria vaccination. This change in the suppressive ability of the MDSCs is not antigen specific as the same decrease in suppression is seen with PSA-antigen specific T cells and also with non-specifically stimulated T cells. In FIGS. 14A and 14B PMA/I represents non-specific stimulation. In FIGS. 14C and 14D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 14A and 14C show individual cell division cycles for each group. FIGS. 14B and 14D show pooled percentage division.

FIG. 15 show suppressor assay data demonstrating that Listeria has no effect on splenic granulocytic MDSCs and they are only suppressive in an antigen-specific manner. In FIGS. 15A and 15B PMA/I represents non-specific stimulation. In FIGS. 15C and 15D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 15A and 15C show individual cell division cycles for each group. FIGS. 15B and 15D show pooled percentage division.

FIG. 16 show suppressor assay data demonstrating that Tregs from tumors are still suppressive. There is a slight decrease in the suppressive ability of Tregs in a non-antigen specific manner, in this tumor model. In FIGS. 16A and 16B PMA/I represents non-specific stimulation. In FIGS. 16C and 16D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 16A and 16C show individual cell division cycles for each group. FIGS. 16B and 16D show pooled percentage division.

FIG. 17 shows suppressor assay data demonstrating that splenic Tregs are still suppressive. In FIGS. 17A and 17B PMA/I represents non-specific stimulation. In FIGS. 17C and 17D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 17A and 17C show individual cell division cycles for each group. FIGS. 17B and 17D show pooled percentage division.

FIG. 18 show suppressor assay data demonstrating that conventional CD4+ T cells have no effect on cell division regardless whether they are found in the tumors or spleens of mice. In FIGS. 18A and 18B PMA/I represents non-specific stimulation. In FIGS. 18C and 18D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 18C-18D show data from pooled percentage division.

FIG. 19 show suppressor assay data demonstrating that monocytic MDSCs from 4T1 tumors (Her2 expressing tumors) have decreased suppressive ability after Listeria vaccination. This change in the suppressive ability of the MDSCs is not antigen specific as the same decrease in suppression is seen with Her2/neu-antigen specific T cells and also with non-specifically stimulated T cells. In FIGS. 19A and 19B PMA/I represents non-specific stimulation. In FIGS. 19C and 19D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 19A and 19C show individual cell division cycles for each group. FIGS. 19B and 19D show pooled percentage division.

FIG. 20 show suppressor assay data demonstrating that there is no Listeria-specific effect on splenic monocytic MDSCs. In FIGS. 20A and 20B PMA/I represents non-specific stimulation. In FIGS. 20C and 20D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSC. FIGS. 20A and 20C show individual cell division cycles for each group. FIGS. 20B and 20D show pooled percentage division.

FIG. 21 show suppressor assay data demonstrating that granulocytic MDSCs from 4T1 tumors (Her2 expressing tumors) have decreased suppressive ability after Listeria vaccination. This change in the suppressive ability of the MDSCs is not antigen specific as the same decrease in suppression is seen with Her2/neu-antigen specific T cells and also with non-specifically stimulated T cells. In FIGS. 21A and 21B PMA/I represents non-specific stimulation. In FIGS. 21C and 21D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 21A and 21C show individual cell division cycles for each group. FIGS. 21B and 21D shows pooled percentage division.

FIG. 22 present suppressor assay data demonstrating that there is no Listeria-specific effect on splenic granulocytic MDSCs. In FIGS. 22A and 22B PMA/I represents non-specific stimulation. In FIGS. 22C and 22D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 22A and 22C show individual cell division cycles for each group. FIGS. 22B and 22D show pooled percentage division.

FIG. 23 present suppressor assay data demonstrating that decrease in the suppressive ability of Tregs from 4T1 tumors (Her2 expressing tumors) after Listeria vaccination. In FIGS. 23A and 23B PMA/I represents non-specific stimulation. In FIGS. 23C and 23D the term “peptide” represents specific antigen stimulation. Percent (%) CD8+ represents % effector (responder) T cells. This decrease is not antigen specific, as the change in Treg suppressive ability is seen with both Her2/neu-specific and non-specific responder T cells. FIGS. 23A and 23C show individual cell division cycles for each group. FIGS. 23B and 23D show pooled percentage division.

FIG. 24 show suppressor assay data demonstrating that there is no Listeria-specific effect on splenic Tregs. The responder T cells are all capable of dividing regardless of whether or not they are antigen specific. In FIGS. 24A and 24B PMA/I represents non-specific stimulation. In FIGS. 24C and 24D the term “peptide” represents specific antigen stimulation. Percent (%) CD8+ represents % effector (responder) T cells. FIGS. 24A and 24C show individual cell division cycles for each group. FIGS. 24B and 24D show pooled percentage division.

FIG. 25 show suppressor assay data demonstrating that suppressive ability of the granulocytic MDSC is due to the overexpression of tLLO and is independent of the partnering fusion antigen. Left-hand panels (FIGS. 25A and 25C) show individual cell division cycles for each group. Right-hand panels (FIGS. 25B and 25D) show pooled percentage division.

FIG. 26 show suppressor assay data also demonstrating that suppressive ability of the monocytic MDSC is due to the overexpression of tLLO and is independent of the partnering fusion antigen. Left-hand panels (FIGS. 26A and 26C) show individual cell division cycles for each group. Right-hand panels (FIGS. 26B and 26D) show pooled percentage division.

FIG. 27 show suppressor assay data demonstrating that granulocytic MDSC purified from the spleen retain their ability to suppress the division of the antigen-specific responder T cells after Lm vaccination (FIGS. 27A and 27B). However, after non-specific stimulation, activated T cells (with PMA/ionomycin) are still capable of dividing (FIGS. 27C and 27D). Left-hand panels show individual cell division cycles for each group. Right-hand panels show pooled percentage division.

FIG. 28 show suppressor assay data demonstrating that monocytic MDSC purified from the spleen retain their ability to suppress the division of the antigen-specific responder T cells after Lm vaccination (FIGS. 28A and 28B). However, after non-specific activation (stimulated by PMA/ionomycin), T cells are still capable of dividing (FIGS. 28C and 28D). Left-hand panels show individual cell division cycles for each group. Right-hand panels show pooled percentage division.

FIG. 29 show suppressor assay data demonstrating that Tregs purified from the tumors of any of the Lm-treated groups have a slightly diminished ability to suppress the division of the responder T cells, regardless of whether the responder cells are antigen specific (FIGS. 29A and 29B) or non-specifically (FIGS. 29C and 29D) activated. Left-hand panels show individual cell division cycles for each group. Right-hand panels show pooled percentage division.

FIG. 30 show suppressor assay data demonstrating that Tregs purified from the spleen are still capable of suppressing the division of both antigen specific (FIGS. 30A-30B) and non-specifically (FIGS. 30C and 30D) activated responder T cells.

FIG. 31 show suppressor assay data demonstrating that tumor Tcon cells are not capable of suppressing the division of T cells regardless of whether the responder cells are antigens specific (FIGS. 31A and 31B) or non-specifically activated (FIGS. 31C and 31D).

FIG. 32 show suppressor assay data demonstrating that spleen Tcon cells are not capable of suppressing the division of T cells regardless of whether the responder cells are antigens specific (FIGS. 32A and 32B) or non-specifically activated (FIGS. 32C and 32D).

FIG. 33. Construction of ADXS31-164. (FIG. 33A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (FIG. 33B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIG. 34. Immunogenic properties of ADXS31-164 (FIG. 34A) Cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (FIG. 34B) IFN-γ secreted by the splenocytes from immunized FVB/N mice into the cell culture medium, measured by ELISA, after 24 hours of in vitro stimulation with mitomycin C treated NT-2 cells. (FIG. 34C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. * P value<0.001.

FIG. 35. Tumor Prevention Studies for Listeria-ChHer2/neu Vaccines Her2/neu transgenic mice were injected six times with each recombinant Listeria-ChHer2 or a control Listeria strain. Immunizations started at 6 weeks of age and continued every three weeks until week 21. Appearance of tumors was monitored on a weekly basis and expressed as percentage of tumor free mice. *p<0.05, N=9 per group.

FIG. 36. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. dot-plots of the Tregs from a representative experiment showing the frequency of CD25⁺/FoxP3⁺ T cells, expressed as percentages of the total CD3⁺ or CD3+CD4⁺ T cells across the different treatment groups.

FIG. 37. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (FIG. 37A). dot-plots of the Tregs from a representative experiment. (FIG. 37B). Frequency of CD25⁺/FoxP3⁺ T cells, expressed as percentages of the total CD3⁺ or CD3+CD4⁺ T cells (left panel) and intratumoral CD8/Tregs ratio (right panel) across the different treatment groups. Data is shown as mean±SEM obtained from 2 independent experiments.

FIG. 38. Vaccination with ADXS31-164 can delay the growth of a breast cancer cell line in the brain. Balb/c mice were immunized thrice with ADXS31-164 or a control Listeria strain. EMT6-Luc cells (5,000) were injected intracranially in anesthetized mice. (FIG. 38A) Ex vivo imaging of the mice was performed on the indicated days using a Xenogen X-100 CCD camera. (FIG. 38B) Pixel intensity was graphed as number of photons per second per cm2 of surface area; this is shown as average radiance. (FIG. 38C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the disclosure. However, it will be understood by those skilled in the art that the disclosure disclosure may be practiced without these specific details. In other instances, well-known methods, procedures, and components have not been described in detail so as not to obscure disclosure the disclosure.

This disclosure provides in one embodiment, an immunogenic composition comprising a recombinant Listeria strain comprising a nucleic acid molecule, the nucleic acid molecule comprising a first open reading frame encoding fusion polypeptide, wherein said fusion polypeptide comprises a Truncated LLO, a truncated ActA or a PEST-sequence peptide fused to a heterologous antigen or fragment thereof and the composition further comprises an additional active agent. In another embodiment, an additional active agent comprise in an immunogenic composition disclosed herein comprises an attenuated oncolytic virus, a T cell receptor engineered T cell (Receptor engineered T cells), a chimeric antigen receptor engineered T cell (CAR T cells), a therapeutic or immunomodulating monoclonal antibody, or a targeting thymidine kinase inhibitor (TKI), or any combination thereof.

In another embodiment, disclosed herein is an immunogenic composition comprising an oncolytic virus, and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In one embodiment, the oncolytic virus is attenuated to eliminate viral functions that are expendable in tumor cells, but not in normal cells, thus making the virus safer and more tumor-specific. Hence, in another embodiment, disclosed herein is an immunogenic composition comprising an attenuated oncolytic virus, and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In another embodiment, disclosed herein is an immunogenic composition comprising chimeric antigen receptor-engineered T cells (CAR T cells), and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In another embodiment, disclosed herein is an immunogenic composition comprising a therapeutic or immunmodulating antibody, and a recombinant isteria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof. In another embodiment, the immunomodulating antibody is a monoclonal antibody. In another embodiment, the monoclonal antibody recognizes an epitope of said heterologous antigen on a cancer cell.

In another embodiment, disclosed herein is an immunogenic composition comprising targeting thymidine kinase (TKI), and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In another embodiment, disclosed herein is an immunogenic composition comprising a T cell receptor engineered T cell (Receptor engineered T cells), and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In one embodiment, this disclosure provides a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding a fusion polypeptide, wherein the fusion polypeptide comprises a Truncated LLO, a truncated ActA or a PEST-sequence peptide fused to a heterologous antigen or fragment thereof, wherein: (a) the composition further comprises an additional active agent; (b) the method further comprises a step of administering an effective amount of a composition comprising an additional active agent to the subject; or (c) the method further comprises a step of administering a targeted radiation therapy to the subject; or any combination thereof of (a)-(c).

In another embodiment, a method disclosed herein is for inhibiting tumor-mediated immunosuppression in a subject, the method comprising the step of administering to the subject an effective amount of an immunogenic composition comprising a recombinant Listeria strain comprising a nucleic acid molecule, the nucleic acid molecule comprising a first open reading frame encoding a fusion polypeptide, wherein the fusion polypeptide comprises a Truncated LLO, a truncated ActA or a PEST-sequence peptide fused to a heterologous antigen or fragment thereof, wherein: (a) the composition further comprises an additional active agent; (b) the method further comprises a step of administering an effective amount of a composition comprising an additional active agent to the subject; or (c) the method further comprises a step of administering a targeted radiation therapy to the subject; or any combination thereof of (a)-(c).

In another embodiment, a method disclosed herein is for a method for increasing the ratio of T effector cells to regulatory T cells (Tregs) in the spleen and tumor of a subject, the method comprising the step of administering to the subject an immunogenic composition comprising a recombinant Listeria strain comprising a nucleic acid molecule, the nucleic acid molecule comprising a first open reading frame encoding a fusion polypeptide, wherein the fusion polypeptide comprises a Truncated LLO, a truncated ActA or a PEST-sequence peptide fused to a heterologous antigen or fragment thereof, wherein: (a) the composition further comprises an additional active agent; (b) the method further comprises a step of administering an effective amount of a composition comprising an additional active agent to the subject; or (c) the method further comprises a step of administering a targeted radiation therapy; or any combination thereof of (a)-(c).

### Recombinant Listeria Strains

In one embodiment, a recombinant Listeria strain disclosed herein comprises a nucleic acid molecule, the nucleic acid molecule comprising a first open reading frame encoding a fusion polypeptide, wherein the fusion polypeptide comprises a Truncated LLO, a truncated ActA or a PEST-sequence peptide fused to a heterologous antigen or fragment thereof. In one embodiment, the recombinant Listeria strain is attenuated.

In one embodiment a Truncated LLO, a truncated ActA or a PEST-sequence peptide Truncated LLO, a truncated ActA or a PEST-sequence peptide comprises a truncated listeriolysin O (LLO) protein or a truncated actA protein. In one embodiment, a Truncated LLO, a truncated ActA or a PEST-sequence peptide is a truncated LLO protein. In another embodiment, a Truncated LLO, a truncated ActA or a PEST-sequence peptide is a truncated actA protein. In one embodiment, a Truncated LLO, a truncated ActA or a PEST-sequence peptide is a full-length LLO protein. In another embodiment, a Truncated LLO, a truncated ActA or a PEST-sequence peptide is a full-length ActA protein.

In one embodiment, a PEST amino acid (AA) sequence comprises a truncated LLO sequence. In another embodiment, the PEST amino acid sequence is KENSISSMAPPASPPASPKTPIEKKHADEIDK (SEQ ID NO: 1). In another embodiment, fusion of an antigen to other LM PEST AA sequences from Listeria will also enhance immunogenicity of the antigen.

The N-terminal LLO protein fragment of methods and compositions of the disclosure comprises, in another embodiment, SEQ ID No: 3. In another embodiment, the fragment comprises an LLO signal peptide. In another embodiment, the fragment comprises SEQ ID No: 4. In another embodiment, the fragment consists approximately of SEQ ID No: 4. In another embodiment, the fragment consists essentially of SEQ ID No: 4. In another embodiment, the fragment corresponds to SEQ ID No: 4. In another embodiment, the fragment is homologous to SEQ ID No: 4. In another embodiment, the fragment is homologous to a fragment of SEQ ID No: 4. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the disclosure. In another embodiment, fusion of a heterologous antigen to any ΔLLO, including the PEST AA sequence, SEQ ID NO: 1, enhances cell mediated and anti-tumor immunity of the antigen.

It will be appreciated by the skilled artisan that the term “PEST sequence-peptide” or “PEST sequence-containing protein” may encompass a truncated LLO protein, which in one embodiment is a N-terminal LLO, and a truncated ActA protein which in one embodiment is an N-terminal LLO, or fragments thereof. It will also be appreciated by the skilled artisan that the term “PEST-sequence peptide” may encompass a PEST sequence peptide or peptide fragments of an LLO protein or an ActA protein thereof. PEST sequence peptides are known in the art and are described in U.S. Pat. No. 7,635,479, and in US Patent Publication Serial No. 2014/0186387, both of which are hereby incorporated in their entirety herein.

In another embodiment, the a PEST sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected to include, but are not limited to, other Listeria species. For example, the L. monocytogenes protein ActA contains four such sequences. These are KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: 7), and RGGIPTSEEFSSLNSGDFTDDENSETTEEEIDR (SEQ ID NO: 8). Also Streptolysin O from Streptococcus sp. contain a PEST sequence. For example, Streptococcus pyogenes Streptolysin O comprises the PEST sequence KQNTASTETTTTNEQPK (SEQ ID NO: 9) at amino acids 35-51 and Streptococcus equisimilis Streptolysin O comprises the PEST-like sequence KQNTANTETTTTNEQPK (SEQ ID NO: 10) at amino acids 38-54. Further, it is believed that the PEST sequence can be embedded within the antigenic protein. Thus, for purposes of the disclosure herein, by “fusion” when in relation to PEST sequence fusions, it is meant that the antigenic protein comprises both the antigen and the PEST amino acid sequence either linked at one end of the antigen or embedded within the antigen.

In another embodiment, the construct or nucleic acid molecule is expressed from an episomal or plasmid vector, with a nucleic acid sequence encoding a truncated LLO, a truncated ActA or a PEST-sequence peptide. In another embodiment, the plasmid is stably maintained in the recombinant Listeria strain in the absence of antibiotic selection. In another embodiment, the plasmid does not confer antibiotic resistance upon the recombinant Listeria. In another embodiment, the fragment is a functional fragment. In another embodiment, the fragment is an immunogenic fragment.

The LLO protein utilized to construct vaccines of the disclosure has, in another embodiment, the sequence:

MKKIMLVFITLILVSLPIAQQTEAKDASAFNKENSISSMAPPASPPASPKTPIEK KHADEIDKYIQGLDYNKNNVLVYHGDAVTNVPPRKGYKDGNEYIVVEKKKKSINQN NADIQVVNAISSLTYPGALVKANSELVENQPDVLPVKRDSLTLSIDLPGMTNQDNKIV VKNATKSNVNNAVNTLVERWNEKYAQAYPNVSAKIDYDDEMAYSESQLIAKFGTAF KAVNNSLNVNFGAISEGKMQEEVISFKQIYYNVNVNEPTRPSRFFGKAVTKEQLQALG VNAENPPAYISSVAYGRQVYLKLSTNSHSTKVKAAFDAAVSGKSVSGDVELTNIIKNS SFKAVIYGGSAKDEVQIIDGNLGDLRDILKKGATFNRETPGVPIAYTTNFLKDNELAVI KNNSEYIETTSKAYTDGKINIDHSGGYVAQFNISWDEVNYDPEGNEIVQHKNWSENN KSKLAHFTSSIYLPGNARNINVYAKECTGLAWEWWRTVIDDRNLPLVKNRNISIWGTT LYPKYSNKVDNPIE (GenBank Accession No. P13128; SEQ ID NO: 2; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a immunotherapy of the disclosure. In another embodiment, the N-terminal fragment of an LLO protein utilized in compositions and methods of the disclosure has the sequence:

(SEQ ID NO: 3)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINTIDHSGGYVAQFNISWDEVNYD.

In one embodiment, the term “vaccine” and “immunotherapy” or their plural form, have the same meanings and qualifications for the purposes of the disclosure and are used interchangeably herein.

In another embodiment, the LLO fragment corresponds to about AA 20-442 of an LLO protein utilized herein.

In another embodiment, the LLO fragment has the sequence:

(SEQ ID NO: 4)
MKKIMLVFITLILVSLPIAQQTEAKDASAFNKENSISSVAPPASPPASP
KTPIEKKHADEIDKYIQGLDYNKNNVLVYHGDAVTNVPPRKGYKDGNEY
IVVEKKKKSINTQNNADIQVVNAISSLTYPGALVKANSELVENQPDVLP
VKRDSLTLSIDLPGMTNQDNKIVVKNATKSNVNNAVNTLVERWNEKYAQ
AYSNVSAKIDYDDEMAYSESQLIAKFGTAFKAVNNSLNVNFGAISEGKM
QEEVISFKQIYYNVNVNEPTRPSRFFGKAVTKEQLQALGVNAENPPAYI
SSVAYGRQVYLKLSTNSHSTKVKAAFDAAVSGKSVSGDVELTNIIKNSS
FKAVIYGGSAKDEVQIIDGNLGDLRDILKKGATFNRETPGVPIAYTTNF
LKDNELAVIKNNSEYIETTSKAYTD.

In another embodiment, “truncated LLO” or “ΔLLO” refers to a fragment of LLO that comprises the PEST-like domain. In another embodiment, the terms refer to an LLO fragment that comprises a PEST sequence.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location. In another embodiment, the LLO is rendered non-hemolytic by a deletion or mutation of the cholesterol binding domain (CBD) as detailed in U.S. Pat. No. 8,771,702, which is incorporated by reference herein.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the disclosure.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

In another embodiment, a homologous LLO refers to identity to an LLO sequence disclosed herein of greater than 70%. In another embodiment, a homologous LLO refers to identity to an LLO sequence disclosed herein of greater than 72%. In another embodiment, a homologous refers to identity to an LLO sequence disclosed herein of greater than 75%. In another embodiment, a homologous refers to identity to an LLO sequence disclosed herein of greater than 78%. In another embodiment, a homologous refers to identity to an LLO sequence disclosed herein of greater than 80%. In another embodiment, a homologous refers to identity to an LLO sequence disclosed herein of greater than 82%. In another embodiment, a homologous refers to identity to an LLO sequence disclosed herein of greater than 83%. In another embodiment, a homologous refers to identity to an LLO sequence disclosed herein of greater than 85%. In another embodiment, a homologous refers to identity to an LLO sequence disclosed herein of greater than 87%. In another embodiment, a homologous refers to identity to an LLO sequence disclosed herein of greater than 88%. In another embodiment, a homologous refers to identity to an LLO sequence disclosed herein of greater than 90%. In another embodiment, a homologous refers to identity to an LLO sequence disclosed herein of greater than 92%. In another embodiment, a homologous refers to identity to an LLO sequence disclosed herein of greater than 93%. In another embodiment, a homologous refers to identity to an LLO sequence disclosed herein of greater than 95%. In another embodiment, a homologous refers to identity to an LLO sequence disclosed herein of greater than 96%. In another embodiment, a homologous refers to identity to an LLO sequence disclosed herein of greater than 97%. In another embodiment, a homologous refers to identity to an LLO sequence disclosed herein of greater than 98%. In another embodiment, a homologous refers to identity to an LLO sequence disclosed herein of greater than 99%. In another embodiment, a homologous refers to identity to an LLO sequence disclosed herein of 100%. Each possibility represents a separate embodiment of the disclosure.

In one embodiment, an ActA protein comprises the sequence set forth in SEQ ID NO: 11:

MGLNRFMRAMMVVFITANCITINPDIIFAATDSEDSSLNTDEWEEEKTEEQPSE VNTGPRYETAREVSSRDIKELEKSNKVRNTNKADLIAMLKEKAEKGPNINNNNSEQTE NAAINEEASGADRPAIQVERRHPGLPSDSAAEIKKRRKAIASSDSELESLTYPDKPTKV NKKKVAKESVADASESDLDSSMQSADESSPQPLKANQQPFFPKVFKKIKDAGKWVRD KIDENPEVKKAIVDKSAGLIDQLLTKKKSEEVNASDFPPPPTDEELRLALPETPMLLGF NAPATSEPSSFEFPPPPTDEELRLALPETPMLLGFNAPATSEPSSFEFPPPPTEDELEIIRET ASSLDSSFTRGDLASLRNAINRHSQNFSDFPPIPTEEELNGRGGRPTSEEFSSLNSGDFTD DENSETTEEEIDRLADLRDRGTGKHSRNAGFLPLNPFASSPVPSLSPKVSKISDRALISDI TKKTPFKNPSQPLNVFNKKTTTKTVTKKPTPVKTAPKLAELPATKPQETVLRENKTPFI EKQAETNKQSINMPSLPVIQKEATESDKEEMKPQTEEKMVEESESANNANGKNRSAGI EEGKLIAKSAEDEKAKEEPGNHTTLILAMLAIGVFSLGAFIKIIQLRKNN. The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 11 above. In another embodiment, an ActA polypeptide or peptide does not include the signal sequence, AA 1-29 of SEQ ID NO: 11 above.

In one embodiment, a truncated ActA protein comprises an N-terminal fragment of an ActA protein. In another embodiment, a truncated ActA protein is an N-terminal fragment of an Acta protein. In one embodiment, a truncated ActA protein comprises the sequence set forth in SEQ ID NO: 12:

MRAMMVVFITANCITINPDHFAATDSEDSSLNTDEWEEEKTEEQPSEVN
TGPRYETAREVSSRDIKELEKSNKVRNTNKADLIAMLKEKAEKGPNINN
NNSEQTENAMNEEASGADRPAIQVERRHPGLPSDSAAEIKKRRKAIASS
DSELESLTYPDKPTKVNKKKVAKESVADASESDLDSSMQSADESSPQPL
KANQQPFFPKVFKKIKDAGKWVRDKIDENPEVKKAIVDKSAGLIDQLLT
KKKSEEVNASDFPPPPTDEELRLALPETPMLLGFNAPATSEPSSFEFPP
PPTDEELRLALPETPMLLGFNAPATSEPSSFEFPPPPTEDELEIIRETA
SSLDSSFTRGDLASLRNAINRHSQNFSDFPPIPTEEELNGRGGRP.
In another embodiment, the ActA fragment
comprises the sequence set forth in SEQ ID NO: 12.

In another embodiment, a truncated ActA protein comprises the sequence set forth in SEQ ID NO: 13: MGLNRFMRAMMVVFITANCITINPDIIFAATDSEDSSLNTDEWEE EKTEEQPSEVNTGPRYETAREVSSRDIKELEKSNKVRNTNKADLIAMLKEKAEKG.

In one embodiment, a truncated ActA protein comprises an N-terminal fragment of an ActA protein additionally lacking all or a portion of the ActA signal sequence, AA 1-29 of SEQ ID NO: 11 above. In another embodiment, a truncated ActA protein is an N-terminal fragment of an ActA protein additionally lacking all or a portion of the ActA signal sequence, AA 1-29 of SEQ ID NO: 11 above. In another embodiment a truncated ActA protein lacks AA 1-29, which is the ActA signal sequence of SEQ ID NO: 11 above. In another embodiment, a truncated ActA protein comprises at least one PEST sequence.

In one embodiment, the full length ActA protein comprises a PEST region, the sequence of which is set forth in SEQ ID NO: 14. In one embodiment, the fusion protein disclosed herein comprises SEQ ID NO: 14. In one embodiment, a truncated ActA protein comprises the sequence set forth in SEQ ID NO: 14:

(SEQ ID NO: 14)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G.
In one embodiment, a truncated ActA protein is
the sequence set forth in SEQ ID NO: 14.

In one embodiment, a truncated ActA protein comprises one to four PEST sequences. In one embodiment, the full length ActA protein comprises a PEST region comprising one to four PEST sequences, the sequence of which is set forth in SEQ ID NO: 15. In one embodiment, the fusion protein disclosed herein comprises SEQ ID NO: 15. In one embodiment, a truncated ActA protein comprises the sequence set forth in SEQ ID NO: 15:

(SEQ ID NO: 15)
A T D S E D S S L N T D E W E E E K T E E Q P S
E V N T G P R Y E T A R E V S S R D I E E L E K
S N K V K N T N K A D L I A M L K A K A E K G P
N N N N N N G E Q T G N V A I N E E A S G V D R
P T L Q V E R R H P G L S S D S A A E I K K R R
K A I A S S D S E L E S L T Y P D K P T K A N K
R K V A K E S V V D A S E S D L D S S M Q S A D
E S T P Q P L K A N Q K P F F P K V F K K I K D
A G K W V R D K.
In one embodiment, a truncated ActA protein is
the sequence set forth in SEQ ID NO: 15.

In one embodiment, a truncated ActA protein comprises one to four PEST sequences. In one embodiment, the full length ActA protein comprises a PEST region comprising one to four PEST sequences, the sequence of which is set forth in SEQ ID NO: 16. In one embodiment, the fusion protein disclosed herein comprises SEQ ID NO: 16. In one embodiment, a truncated ActA protein comprises the sequence set forth in SEQ ID NO: 16:

(SEQ ID NO: 16)
A T D S E D S S L N T D E W E E E K T E E Q P S
E V N T G P R Y E T A R E V S S R D I E E L E K
S N K V K N T N K A D L I A M L K A K A E K G P
N N N N N N G E Q T G N V A I N E E A S G V D R
P T L Q V E R R H P G L S S D S A A E I K K R R
K A I A S S D S E L E S L T Y P D K P T K A N K
R K V A K E S V V D A S E S D L D S S M Q S A D
E S T P Q P L K A N Q K P F F P K V F K K I K D
A G K W V R D K I D E N P E V K K A I V D K S A
G L I D Q L L T K K K S E E V N A S D F P P P P
T D E E L R L A L P E T P M L L G F N A P T P S
E P S S F E F P P P P T D E E L R L A L P E T P
M L L G F N A P A T S E P S S.
In one embodiment, a truncated ActA protein
is the sequence set forth in SEQ ID NO: 16.

In one embodiment, a truncated ActA protein comprises one to four PEST sequences. In one embodiment, the full length ActA protein comprises a PEST region comprising one to four PEST sequences, the sequence of which is set forth in SEQ ID NO: 17. In one embodiment, the fusion protein disclosed herein comprises SEQ ID NO: 17. In one embodiment, a truncated ActA protein comprises the sequence set forth in SEQ ID NO: 17:

(SEQ ID NO: 17)
A T D S E D S S L N T D E W E E E K T E E Q P S
E V N T G P R Y E T A R E V S S R D I E E L E K
S N K V K N T N K A D L I A M L K A K A E K G P
N N N N N N G E Q T G N V A I N E E A S G V D R
P T L Q V E R R H P G L S S D S A A E I K K R R
K A I A S S D S E L E S L T Y P D K P T K A N K
R K V A K E S V V D A S E S D L D S S M Q S A D
E S T P Q P L K A N Q K P F F P K V F K K I K D
A G K W V R D K I D E N P E V K K A I V D K S A
G L I D Q L L T K K K S E E V N A S D F P P P P
T D E E L R L A L P E T P M L L G F N A P T P S
E P S S F E F P P P P T D E E L R L A L P E T P
M L L G F N A P A T S E P S S F E F P P P P T E
D E L E I M R E T A P S L D S S F T S G D L A S
L R S A I N R H S E N F S D F P L I P T E E E L
N G R G G R P T S E.
In one embodiment, a truncated ActA protein is
the sequence set forth in SEQ ID NO: 17.

In another embodiment, the ActA fragment is any other ActA fragment known in the art. Each possibility represents a separate embodiment of the disclosure.

In another embodiment, the recombinant nucleotide encoding anActA protein comprises the sequence set forth in SEQ ID NO: 18:

Atgcgtgcgatgatggtggttttcattactgccaattgcattacgattaaccccgacataatatttgcagcgacagatagcgaa gattctagtctaaacacagatgaatgggaagaagaaaaaacagaagagcaaccaagcgaggtaaatacgggaccaagatacgaaact gcacgtgaagtaagttcacgtgatattaaagaactagaaaaatcgaataaagtgagaaatacgaacaaagcagacctaatagcaatgttga aagaaaaagcagaaaaaggtccaaatatcaataataacaacagtgaacaaactgagaatgcggctataaatgaagaggcttcaggagcc gaccgaccagctatacaagtggagcgtcgtcatccaggattgccatcggatagcgcagcggaaattaaaaaaagaaggaaagccatag catcatcggatagtgagcttgaaagccttacttatccggataaaccaacaaaagtaaataagaaaaaagtggcgaaagagtcagttgcgg atgcttctgaaagtgacttagattctagcatgcagtcagcagatgagtcttcaccacaacctttaaaagcaaaccaacaaccatttttccctaa agtatttaaaaaaataaaagatgcggggaaatgggtacgtgataaaatcgacgaaaatcctgaagtaaagaaagcgattgttgataaaagt gcagggttaattgaccaattattaaccaaaaagaaaagtgaagaggtaaatgcttcggacttcccgccaccacctacggatgaagagttaa gacttgctttgccagagacaccaatgcttcttggttttaatgctcctgctacatcagaaccgagctcattcgaatttccaccaccacctacgga tgaagagttaagacttgctttgccagagacgccaatgcttcttggttttaatgctcctgctacatcggaaccgagctcgttcgaatttccaccg cctccaacagaagatgaactagaaatcatccgggaaacagcatcctcgctagattctagttttacaagaggggatttagctagtttgagaaa tgctattaatcgccatagtcaaaatttctctgatttcccaccaatcccaacagaagaagagttgaacgggagaggcggtagacca. In another embodiment, the recombinant nucleotide has the sequence set forth in SEQ ID NO: 18. In another embodiment, the recombinant nucleotide comprises any other sequence that encodes a fragment of an ActA protein.

In another embodiment, “truncated ActA” or “AActA” refers to a fragment of ActA that comprises the PEST-like domain. In another embodiment, the terms refer to an ActA fragment that comprises a PEST sequence.

In one embodiment LM PEST sequences and PEST sequences derived from other prokaryotic organisms will enhance immunogenicity of the antigen. In one embodiment, the PEST sequence is a PEST sequence from the LM ActA protein. In another embodiment, the terms “PEST-sequence peptide,” and “PEST sequence” are used interchangeably herein. In another embodiment, the PEST sequence is KTEEQPSEVNTGPR (SEQ ID NO: 19), KASVTDTSEGDLDSSMQSADESTPQPLK (SEQ ID NO: 20), KNEEVNASDFPPPPTDEELR (SEQ ID NO: 21), or RGGIPTSEEFSSLNSGDFTDDENSETTEEEIDR (SEQ ID NO: 22). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 23) at AA 35-51. In another embodiment, the PEST-like sequence is from Streptococcus equisimilis Streptolysin O, e.g. KQNTANTETTTTNEQPK (SEQ ID NO: 24) at AA 38-54. In another embodiment, the PEST-like sequence is another PEST AA sequence derived from a prokaryotic organism. In another embodiment, the PEST sequence is any other PEST sequence known in the art.

In another embodiment, the ActA fragment consists of about the first 100 AA of the ActA protein.

In another embodiment, the ActA fragment consists of about residues 1-25. In another embodiment, the ActA fragment consists of about residues 1-50. In another embodiment, the ActA fragment consists of about residues 1-75. In another embodiment, the ActA fragment consists of about residues 1-100. In another embodiment, the ActA fragment consists of about residues 1-125. In another embodiment, the ActA fragment consists of about residues 1-150. In another embodiment, the ActA fragment consists of about residues 1-175. In another embodiment, the ActA fragment consists of about residues 1-200. In another embodiment, the ActA fragment consists of about residues 1-225. In another embodiment, the ActA fragment consists of about residues 1-250. In another embodiment, the ActA fragment consists of about residues 1-275. In another embodiment, the ActA fragment consists of about residues 1-300. In another embodiment, the ActA fragment consists of about residues 1-325. In another embodiment, the ActA fragment consists of about residues 1-338. In another embodiment, the ActA fragment consists of about residues 1-350. In another embodiment, the ActA fragment consists of about residues 1-375. In another embodiment, the ActA fragment consists of about residues 1-400. In another embodiment, the ActA fragment consists of about residues 1-450. In another embodiment, the ActA fragment consists of about residues 1-500. In another embodiment, the ActA fragment consists of about residues 1-550. In another embodiment, the ActA fragment consists of about residues 1-600. In another embodiment, the ActA fragment consists of about residues 1-639. In another embodiment, the ActA fragment consists of about residues 30-100. In another embodiment, the ActA fragment consists of about residues 30-125. In another embodiment, the ActA fragment consists of about residues 30-150. In another embodiment, the ActA fragment consists of about residues 30-175. In another embodiment, the ActA fragment consists of about residues 30-200. In another embodiment, the ActA fragment consists of about residues 30-225. In another embodiment, the ActA fragment consists of about residues 30-250. In another embodiment, the ActA fragment consists of about residues 30-275. In another embodiment, the ActA fragment consists of about residues 30-300. In another embodiment, the ActA fragment consists of about residues 30-325. In another embodiment, the ActA fragment consists of about residues 30-338. In another embodiment, the ActA fragment consists of about residues 30-350. In another embodiment, the ActA fragment consists of about residues 30-375. In another embodiment, the ActA fragment consists of about residues 30-400. In another embodiment, the ActA fragment consists of about residues 30-450. In another embodiment, the ActA fragment consists of about residues 30-500. In another embodiment, the ActA fragment consists of about residues 30-550. In another embodiment, the ActA fragment consists of about residues 1-600. In another embodiment, the ActA fragment consists of about residues 30-604.

In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly. In another embodiment, the ActA fragment is any other ActA fragment known in the art.

In another embodiment, a homologous ActA refers to identity to an ActA sequence disclosed herein of greater than 70%. In another embodiment, a homologous ActA refers to identity to an ActA sequence of greater than 72%. In another embodiment, a homologous refers to identity to an ActA sequence of greater than 75%. In another embodiment, a homologous refers to identity to an ActA sequence disclosed herein of greater than 78%. In another embodiment, a homologous refers to identity to an ActA sequence disclosed herein of greater than 80%. In another embodiment, a homologous refers to identity to an ActA sequence disclosed herein of greater than 82%. In another embodiment, a homologous refers to identity to an ActA sequence disclosed herein of greater than 83%. In another embodiment, a homologous refers to identity to an ActA sequence disclosed herein of greater than 85%. In another embodiment, a homologous refers to identity to an ActA sequence disclosed herein of greater than 87%. In another embodiment, a homologous refers to identity to an ActA sequence disclosed herein of greater than 88%. In another embodiment, a homologous refers to identity to an ActA sequence disclosed herein greater than 90%. In another embodiment, a homologous refers to identity to one of SEQ ID No: 11 of greater than 92%. In another embodiment, a homologous refers to identity to an ActA sequence disclosed herein of greater than 93%. In another embodiment, a homologous refers to identity to an ActA sequence disclosed herein of greater than 95%. In another embodiment, a homologous refers to identity to an ActA sequence disclosed herein of greater than 96%. In another embodiment, a homologous refers to identity to an ActA sequence disclosed herein of greater than 97%. In another embodiment, a homologous refers to identity to an ActA sequence disclosed herein of greater than 98%. In another embodiment, a homologous refers to identity to one of SEQ ID No: 11 of greater than 99%. In another embodiment, a homologous refers to identity to identity to an ActA sequence of 100%.

As used herein, the term “homology,” when in reference to any nucleic acid sequence disclosed herein refers in one embodiment to a percentage of nucleotides in a candidate sequence that is identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), LALIGN, GENPEPT and TREMBL packages.

In another embodiment, “homology” refers to identity to a sequence selected from the sequences disclosed herein of greater than 68%. In another embodiment, “homology” refers to identity to a sequence selected from the sequences disclosed herein of greater than 70%. In another embodiment, “homology” refers to identity to a sequence selected from the sequences disclosed herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5×Denhardt's solution, 10% dextran sulfate, and 20 μg/ml denatured, sheared salmon sperm DNA.

In one embodiment, the recombinant Listeria strain disclosed herein lacks antibiotic resistance genes. In another embodiment, the recombinant Listeria strain disclosed herein comprises a plasmid comprising a nucleic acid encoding an antibiotic resistance gene.

In one embodiment, the recombinant Listeria disclosed herein is capable of escaping the phagolysosome.

In one embodiment, the Listeria genome comprises a deletion of the endogenous ActA gene, which in one embodiment is a virulence factor. In one embodiment, the heterologous antigen or antigenic polypeptide is integrated in frame with LLO in the Listeria chromosome. In another embodiment, the integrated nucleic acid molecule is integrated in frame with ActA into the ActA locus. In another embodiment, the chromosomal nucleic acid encoding ActA is replaced by a nucleic acid molecule encoding an antigen.

In one embodiment, a heterologous antigen is a tumor-associated antigen. In another embodiment, the tumor-associated antigen is a naturally occurring tumor-associated antigen. In another embodiment, the tumor-associated antigen is a synthetic tumor-associated antigen. In yet another embodiment, the tumor-associated antigen is a chimeric tumor-associated antigen.

In one embodiment, the nucleic acid molecule disclosed herein comprises a first open reading frame encoding recombinant polypeptide comprising a heterologous antigen or fragment thereof. In another embodiment, the recombinant polypeptide further comprises a truncated LLO protein, a truncated ActA protein or PEST sequence peptide fused to the heterologous antigen. In another embodiment, the truncated LLO protein is a N-terminal LLO or fragment thereof. In another embodiment, the truncated ActA protein is a N-terminal ActA protein or fragment thereof.

In one embodiment, “antigenic polypeptide” is used herein to refer to a polypeptide, peptide or recombinant peptide as described herein that is processed and presented on MHC class I and/or class II molecules present in a subject's cells leading to the mounting of an immune response when present in, or in another embodiment, detected by, the host. In one embodiment, the antigen may be foreign to the host. In another embodiment, the antigen might be present in the host but the host does not elicit an immune response against it because of immunologic tolerance.

In one embodiment, the nucleic acid molecule disclosed herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains disclosed herein comprise a mutation in the endogenous dal/dat genes. In another embodiment, the Listeria lacks the dal/dat genes.

In another embodiment, a nucleic acid molecule of the methods and compositions of the disclosure is operably linked to a promoter/regulatory sequence. In another embodiment, the first open reading frame of methods and compositions of the disclosure is operably linked to a promoter/regulatory sequence. In another embodiment, the second open reading frame of methods and compositions of the disclosure is operably linked to a promoter/regulatory sequence. In another embodiment, each of the open reading frames are operably linked to a promoter/regulatory sequence.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

In another embodiment, the recombinant Listeria is an attenuated auxotrophic strain. In another embodiment, the recombinant Listeria is an Lm-LLO-E7 strain described in U.S. Pat. No. 8,114,414, which is incorporated by reference herein in its entirety.

In one embodiment the attenuated strain is Lm dal(−)dat(−) (Lmdd). In another embodiment, the attenuated strains is Lm dal(−)dat(−)AactA (LmddA). LmddA is based on a Listeria vector which is attenuated due to the deletion of virulence gene actA and retains the plasmid for a desired heterologous antigen or truncated LLO expression in vivo and in vitro by complementation of dal gene.

In another embodiment the attenuated strain is LmddA. In another embodiment, the attenuated strain is LmΔactA. In another embodiment, the attenuated strain is LmΔPrfA. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain used in the disclosure is a Listeria strain that expresses a non-hemolytic LLO.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as disclosed herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemase enzyme. In another embodiment, the metabolic enzyme is a D-amino acid transferase enzyme.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a vector harbored by the recombinant Listeria strain. In another embodiment, the episomal expression vector lacks an antibiotic resistance marker. In one embodiment, an antigen of the methods and compositions as disclosed herein is fused to a polypeptide comprising a PEST sequence. In another embodiment, the polypeptide comprising a PEST sequence is a truncated LLO. In another embodiment, the polypeptide comprising a PEST sequence is ActA.

In another embodiment, the Listeria strain is deficient in an amino acid (AA) metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is flil. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, disclosed herein is a nucleic acid molecule that is used to transform the Listeria in order to arrive at a recombinant Listeria. In another embodiment, the nucleic acid disclosed herein used to transform a Listeria strain lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence in the recombinant Listeria.

In yet another embodiment the Listeria strain is an inlA mutant, an inlB mutant, an inlC mutant, an inlJ mutant, prfA mutant, ActA mutant, a dal/dat mutant, a prfA mutant, a plcB deletion mutant, or a double mutant lacking both plcA and plcB. In another embodiment, the Listeria comprise a deletion or mutation of these genes individually or in combination. In another embodiment, the Listeria disclosed herein lack each ne of genes. In another embodiment, the Listeria disclosed herein lack at least one and up to ten of any gene disclosed herein, including the actA, prfA, and dal/dat genes. In another embodiment, the prfA mutant is a D133V prfA mutant.

In one embodiment, the live attenuated Listeria is a recombinant Listeria. In another embodiment, the recombinant Listeria comprises a mutation or a deletion of a genomic internalin C (inlC) gene. In another embodiment, the recombinant Listeria comprises a mutation or a deletion of a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a vaccine backbone.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In one embodiment, the recombinant Listeria strain disclosed herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain disclosed herein comprise an inactivating mutation of the endogenous actA gene. In another embodiment, the recombinant Listeria strain disclosed herein comprise an inactivating mutation of the endogenous inlB gene. In another embodiment, the recombinant Listeria strain disclosed herein comprise an inactivating mutation of the endogenous inlC gene. In another embodiment, the recombinant Listeria strain disclosed herein comprise an inactivating mutation of the endogenous actA and inlB genes. In another embodiment, the recombinant Listeria strain disclosed herein comprise an inactivating mutation of the endogenous actA and inlC genes. In another embodiment, the recombinant Listeria strain disclosed herein comprise an inactivating mutation of the endogenous actA, inlB, and inlC genes. In another embodiment, the recombinant Listeria strain disclosed herein comprise an inactivating mutation of the endogenous actA, inlB, and inlC genes. In another embodiment, the recombinant Listeria strain disclosed herein comprise an inactivating mutation of the endogenous actA, inlB, and inlC genes. In another embodiment, the recombinant Listeria strain disclosed herein comprise an inactivating mutation in any single gene or combination of the following genes: actA, dal, dat, inlB, inlC, prfA, plcA, plcB.

It will be appreciated by the skilled artisan that the term “mutation” and grammatical equivalents thereof, include any type of mutation or modification to the sequence (nucleic acid or amino acid sequence), and includes a deletion mutation, a truncation, an inactivation, a disruption, a replacement mutation, or a translocation. These types of mutations are readily known in the art.

In one embodiment, in order to select for an auxotrophic bacteria comprising a plasmid encoding a metabolic enzyme or a complementing gene disclosed herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the disclosure if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.).

In another embodiment, once the auxotrophic bacteria comprising the plasmid of the disclosure have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vector by adjusting the volume of the media in which the auxotrophic bacteria comprising the plasmid are growing.

The skilled artisan will appreciate that, in another embodiment, other auxotroph strains and complementation systems are adopted for the use with this disclosure.

In one embodiment, the N-terminal LLO protein fragment and heterologous antigen are fused directly to one another. In another embodiment, the genes encoding the N-terminal LLO protein fragment and heterologous antigen are fused directly to one another. In another embodiment, the N-terminal LLO protein fragment and heterologous antigen are operably attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is expressed and used alone, i.e., in unfused form. In another embodiment, the N-terminal LLO protein fragment is the N-terminal-most portion of the fusion protein. In another embodiment, a truncated LLO is truncated at the C-terminal to arrive at an N-terminal LLO.

In one embodiment, the N-terminal ActA protein fragment and heterologous antigen are fused directly to one another. In another embodiment, the genes encoding the N-terminal ActA protein fragment and heterologous antigen are fused directly to one another. In another embodiment, the N-terminal ActA protein fragment and heterologous antigen are operably attached via a linker peptide. In another embodiment, the N-terminal ActA protein fragment and heterologous antigen are attached via a heterologous peptide. In another embodiment, the N-terminal ActA protein fragment is N-terminal to the heterologous antigen. In another embodiment, the N-terminal ActA protein fragment is expressed and used alone, i.e., in unfused form. In another embodiment, the N-terminal ActA protein fragment is the N-terminal-most portion of the fusion protein. In another embodiment, a truncated ActA is truncated at the C-terminal to arrive at an N-terminal ActA.

As disclosed herein, there was an unexpected change in the suppressive ability of the granulocytic MDSC and this is due to the overexpression of tLLO and is independent of the partnering fusion antigen (see Example 13).

In one embodiment, the recombinant Listeria strain disclosed herein expresses the recombinant polypeptide. In another embodiment, the recombinant Listeria strain comprises a plasmid that encodes the recombinant polypeptide. In another embodiment, a recombinant nucleic acid disclosed herein is in a plasmid in the recombinant Listeria strain disclosed herein. In another embodiment, the plasmid is an episomal plasmid that does not integrate into the recombinant Listeria strain's chromosome. In another embodiment, the plasmid is an integrative plasmid that integrates into the Listeria strain's chromosome. In another embodiment, the plasmid is a multicopy plasmid.

In one embodiment, the heterologous antigen is a tumor-associated antigen. In one embodiment, the recombinant Listeria strain of the compositions and methods as disclosed herein express a heterologous antigenic polypeptide that is expressed by a tumor cell. In one embodiment, a tumor-associated antigen is a prostate specific antigen (PSA). In another embodiment, a tumor-associated antigen is a human papilloma virus (HPV) antigen. In yet another embodiment, a tumor-associated antigen is a Her2/neu chimeric antigen as described in US Patent Pub. No. US2011/014279, which is incorporated by reference herein in its entirety. In still another embodiment, a tumor-associated antigen is an angiogenic antigen.

In one embodiment, the recombinant Listeria strain of the compositions and methods as disclosed herein comprise a first or second nucleic acid molecule that encodes a Prostate Specific Antigen (PSA), which in one embodiment, is a marker for prostate cancer that is highly expressed by prostate tumors. In one embodiment, PSA is a kallikrein serine protease (KLK3) secreted by prostatic epithelial cells, which in one embodiment, is widely used as a marker for prostate cancer. As used herein, the terms PSA and KLK3 are interchangeable having all the same meanings and qualities.

In one embodiment, the recombinant Listeria strain as disclosed herein comprises a nucleic acid molecule encoding a tumor associated antigen. In one embodiment, a tumor associated antigen comprises an KLK3 polypeptide or a fragment thereof. In one embodiment, the recombinant Listeria strain as disclosed herein comprises a nucleic acid molecule encoding KLK3 protein.

In another embodiment, the KLK3 protein has the sequence:

MWVPVVFLTLSVTWIGAAPLILSRIVGGWECEKHSQPWQVLVASRGRAVCG GVLVHPQWVLTAAHCIRNKSVILLGRHSLFHPEDTGQVFQVSHSFPHPLYDMSLLKNR FLRPGDDSSHDLMLLRLSEPAELTDAVKVMDLPTQEPALGTTCYASGWGSIEPEEFLT PKKLQCVDLHVISNDVCAQVHPQKVTKFMLCAGRWTGGKSTCSGDSGGPLVCNGVL QGITSWGSEPCALPERPSLYTKVVHYRKWIKDTIVANP (SEQ ID No: 25; GenBank Accession No. CAA32915). In another embodiment, the KLK3 protein is a homologue of SEQ ID No: 25. In another embodiment, the KLK3 protein is a variant of SEQ ID No: 25. In another embodiment, the KLK3 protein is an isomer of SEQ ID No: 25. In another embodiment, the KLK3 protein is a fragment of SEQ ID No: 25.

In another embodiment, the KLK3 protein has the sequence:

IVGGWECEKHSQPWQVLVASRGRAVCGGVLVHPQWVLTAAHCIRNKSVILL GRHSLFHPEDTGQVFQVSHSFPHPLYDMSLLKNRFLRPGDDSSHDLMLLRLSEPAELT DAVKVMDLPTQEPALGTTCYASGWGSIEPEEFLTPKKLQCVDLHVISNDVCAQVHPQ KVTKFMLCAGRWTGGKSTCSGDSGGPLVCYGVLQGITSWGSEPCALPERPSLYTKVV HYRKWIKDTIVANP (SEQ ID No: 26). In another embodiment, the KLK3 protein is a homologue of SEQ ID No: 26. In another embodiment, the KLK3 protein is a variant of SEQ ID No: 26. In another embodiment, the KLK3 protein is an isomer of SEQ ID No: 26. In another embodiment, the KLK3 protein is a fragment of SEQ ID No: 26. Each possibility represents a separate embodiment of the methods and compositions as disclosed herein.

In another embodiment, the KLK3 protein has the sequence: IVGGWECEKHSQPWQVLVASRGRAVCGGVLVHPQWVLTAAHCIRNKSVILLGRHSL FHPEDTGQVFQVSHSFPHPLYDMSLLKNRFLRPGDDSSHDLMLLRLSEPAELTDAVKV MDLPTQEPALGTTCYASGWGSIEPEEFLTPKKLQCVDLHVISNDVCAQVHPQKVTKF MLCAGRWTGGKSTCSGDSGGPLVCNGVLQGITSWGSEPCALPERPSLYTKVVHYRK WIKDTIVANP (SEQ ID No: 27; GenBank Accession No. AAA59995.1). In another embodiment, the KLK3 protein is a homologue of SEQ ID No: 27. In another embodiment, the KLK3 protein is a variant of SEQ ID No: 27. In another embodiment, the KLK3 protein is an isomer of SEQ ID No: 27. In another embodiment, the KLK3 protein is a fragment of SEQ ID No: 27.

In another embodiment, the KLK3 protein is encoded by a nucleotide molecule having the sequence: ggtgtcttaggcacactggtcttggagtgcaaaggatctaggcacgtgaggctttgtatgaagaatcggggatcgtacccaccccctgtttc tgtttcatcctgggcatgtctcctctgcctttgtcccctagatgaagtctccatgagctacaagggcctggtgcatccagggtgatctagtaatt gcagaacagcaagtgctagctctccctccccttccacagctctgggtgtgggagggggttgtccagcctccagcagcatggggagggc cttggtcagcctctgggtgccagcagggcaggggcggagtcctggggaatgaaggttttatagggctcctgggggaggctccccagcc ccaagcttaccacctgcacccggagagctgtgtcaccatgtgggtcccggttgtcttcctcaccctgtccgtgacgtggattggtgagagg ggccatggttggggggatgcaggagagggagccagccctgactgtcaagctgaggctctttcccccccaacccagcaccccagccca gacagggagctgggctcttttctgtctctcccagccccacttcaagcccatacccccagtcccctccatattgcaacagtcctcactcccac accaggtccccgctccctcccacttaccccagaactttcttcccatttgcccagccagctccctgctcccagctgctttactaaaggggaagt tcctgggcatctccgtgtttctctttgtggggctcaaaacctccaaggacctctctcaatgccattggttccttggaccgtatcactggtccatc tcctgagcccctcaatcctatcacagtctactgacttttcccattcagctgtgagtgtccaaccctatcccagagaccttgatgcttggcctccc aatcttgccctaggatacccagatgccaaccagacacctccttctttcctagccaggctatctggcctgagacaacaaatgggtccctcagt ctggcaatgggactctgagaactcctcattccctgactcttagccccagactcttcattcagtggcccacattttccttaggaaaaacatgagc atccccagccacaactgccagctctctgagtccccaaatctgcatccttttcaaaacctaaaaacaaaaagaaaaacaaataaaacaaaac caactcagaccagaactgttttctcaacctgggacttcctaaactttccaaaaccttcctcttccagcaactgaacctcgccataaggcactta tccctggttcctagcaccccttatcccctcagaatccacaacttgtaccaagtttcccttctcccagtccaagaccccaaatcaccacaaagg acccaatccccagactcaagatatggtctgggcgctgtcttgtgtctcctaccctgatccctgggttcaactctgctcccagagcatgaagc ctctccaccagcaccagccaccaacctgcaaacctagggaagattgacagaattcccagcctttcccagctccccctgcccatgtcccag gactcccagccttggttctctgcccccgtgtcttttcaaacccacatcctaaatccatctcctatccgagtcccccagttccccctgtcaaccct gattcccctgatctagcaccccctctgcaggcgctgcgcccctcatcctgtctcggattgtgggaggctgggagtgcgagaagcattccc aaccctggcaggtgcttgtggcctctcgtggcagggcagtctgcggcggtgttctggtgcacccccagtgggtcctcacagctgcccact gcatcaggaagtgagtaggggcctggggtctggggagcaggtgtctgtgtcccagaggaataacagctgggcattttccccaggataac ctctaaggccagccttgggactgggggagagagggaaagttctggttcaggtcacatggggaggcagggttggggctggaccaccctc cccatggctgcctgggtctccatctgtgtccctctatgtctctttgtgtcgctttcattatgtctcttggtaactggcttcggttgtgtctctccgtgt cctgtatctctctgccaggctctgtctctcggtctctgtctcacctgtgccttctccctactgaacacacgcacgggatgggcctgggggacc ctgagaaaaggaagggctttggctgggcgcggtggctcacacctgtaatcccagcactttgggaggccaaggcaggtagatcacctga ggtcaggagttcgagaccagcctggccaactggtgaaaccccatctctactaaaaatacaaaaaattagccaggcgtggtggcgcatgc ctgtagtcccagctactcaggagctgagggaggagaattgcattgaacctggaggttgaggttgcagtgagccgagaccgtgccactgc actccagcctgggtgacagagtgagactccgcctcaaaaaaaaaaaaaaaaaaaaaaaaaaaaaagaaaagaaaagaaaagaaaag gaagtgttttatccctgatgtgtgtgggtatgagggtatgagagggcccctctcactccattccttctccaggacatccctccactcttgggag acacagagaagggctggttccagctggagctgggaggggcaattgagggaggaggaaggagaagggggaaggaaaacagggtat gggggaaaggaccctggggagcgaagtggaggatacaaccttgggcctgcaggcaggctacctacccacttggaaacccacgccaa agccgcatctacagctgagccactctgaggcctcccctccccggcggtccccactcagctccaaagtctctctcccttttctctcccacactt gccctttcattctctctgcccttttaccctcttccttttcccttggttctctcagttctgtatctgcccttcaccctctcacactgctgtttcccaactcg ttgtctgtattttggcctgaactgtgtcttcccaaccctgtgttttctcactgtttctttttctcttttggagcctcctccttgctcctctgtcccttctctc tttccttatcatcctcgctcctcattcctgcgtctgcttcctccccagcaaaagcgtgatcttgctgggtcggcacagcctgtttcatcctgaag acacaggccaggtatttcaggtcagccacagcttcccacacccgctctacgatatgagcctcctgaagaatcgattcctcaggccaggtg atgactccagccacgacctcatgctgctccgcctgtcagagcctgccgagctcacggatgctgtgaaggtcatggacctgcccacccag gagccagcactggggaccacctgctacgcctcaggctggggcagcattgaaccagaggagtgtacgcctgggccagatggtgcagcc gggagcccagatgcctgggtctgagggaggaggggacaggactcctgggtctgagggaggagggccaaggaaccaggtggggtcc agcccacaacagtgtttttgcctggcccgtagtcttgaccccaaagaaacttcagtgtgtggacctccatgttatttccaatgacgtgtgtgcg caagttcaccctcagaaggtgaccaagttcatgctgtgtgctggacgctggacagggggcaaaagcacctgctcggtgagtcatccctac tcccaagatcttgagggaaaggtgagtgggaccttaattctgggctggggtctagaagccaacaaggcgtctgcctcccctgctccccag ctgtagccatgccacctccccgtgtctcatctcattccctccttccctcttctttgactccctcaaggcaataggttattcttacagcacaactcat ctgttcctgcgttcagcacacggttactaggcacctgctatgcacccagcactgccctagagcctgggacatagcagtgaacagacagag agcagcccctcccttctgtagcccccaagccagtgaggggcacaggcaggaacagggaccacaacacagaaaagctggagggtgtc aggaggtgatcaggctctcggggagggagaaggggtggggagtgtgactgggaggagacatcctgcagaaggtgggagtgagcaa acacctgcgcaggggaggggagggcctgcggcacctgggggagcagagggaacagcatctggccaggcctgggaggaggggcct agagggcgtcaggagcagagaggaggttgcctggctggagtgaaggatcggggcagggtgcgagagggaacaaaggacccctcct gcagggcctcacctgggccacaggaggacactgcttttcctctgaggagtcaggaactgtggatggtgctggacagaagcaggacagg gcctggctcaggtgtccagaggctgcgctggcctcctatgggatcagactgcagggagggagggcagcagggatgtggagggagtg atgatggggctgacctgggggtggctccaggcattgtccccacctgggcccttacccagcctccctcacaggctcctggccctcagtctct cccctccactccattctccacctacccacagtgggtcattctgatcaccgaactgaccatgccagccctgccgatggtcctccatggctccc tagtgccctggagaggaggtgtctagtcagagagtagtcctggaaggtggcctctgtgaggagccacggggacagcatcctgcagatg gtcctggcccttgtcccaccgacctgtctacaaggactgtcctcgtggaccctcccctctgcacaggagctggaccctgaagtcccttccta ccggccaggactggagcccctacccctctgttggaatccctgcccaccttcttctggaagtcggctctggagacatttctctcttcttccaaa gctgggaactgctatctgttatctgcctgtccaggtctgaaagataggattgcccaggcagaaactgggactgacctatctcactctctccct gcttttacccttagggtgattctgggggcccacttgtctgtaatggtgtgcttcaaggtatcacgtcatggggcagtgaaccatgtgccctgc ccgaaaggccttccctgtacaccaaggtggtgcattaccggaagtggatcaaggacaccatcgtggccaacccctgagcacccctatca agtccctattgtagtaaacttggaaccttggaaatgaccaggccaagactcaagcctccccagttctactgacctttgtccttaggtgtgagg tccagggttgctaggaaaagaaatcagcagacacaggtgtagaccagagtgtttcttaaatggtgtaattttgtcctctctgtgtcctgggga atactggccatgcctggagacatatcactcaatttctctgaggacacagttaggatggggtgtctgtgttatttgtgggatacagagatgaaa gaggggtgggatcc (SEQ ID No: 28; GenBank Accession No. X14810). In another embodiment, the KLK3 protein is encoded by residues 401 . . . 446, 1688 . . . 1847, 3477 . . . 3763, 3907 . . . 4043, and 5413 . . . 5568 of SEQ ID No: 28. In another embodiment, the KLK3 protein is encoded by a homologue of SEQ ID No: 28. In another embodiment, the KLK3 protein is encoded by a variant of SEQ ID No: 28. In another embodiment, the KLK3 protein is encoded by an isomer of SEQ ID No: 28. In another embodiment, the KLK3 protein is encoded by a fragment of SEQ ID No: 28.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKLQ CVDLHVISNDVCAQVHPQKVTKFMLCAGRWTGGKSTCSWVILITELTMPALPMVLH GSLVPWRGGV (SEQ ID No: 29; GenBank Accession No. NP_001025218) In another embodiment, the KLK3 protein is a homologue of SEQ ID No: 29. In another embodiment, the KLK3 protein is a variant of SEQ ID No: 29. In another embodiment, the KLK3 protein is an isomer of SEQ ID No: 29. In another embodiment, the KLK3 protein is a fragment of SEQ ID No: 29.

In another embodiment, the KLK3 protein is encoded by a nucleotide molecule having the sequence: agccccaagcttaccacctgcacccggagagctgtgtcaccatgtgggtcccggttgtcttcctcaccctgtccgtgacgtggattggtgct gcacccctcatcctgtctcggattgtgggaggctgggagtgcgagaagcattcccaaccctggcaggtgcttgtggcctctcgtggcagg gcagtctgcggcggtgttctggtgcacccccagtgggtcctcacagctgcccactgcatcaggaacaaaagcgtgatcttgctgggtcgg cacagcctgtttcatcctgaagacacaggccaggtatttcaggtcagccacagcttcccacacccgctctacgatatgagcctcctgaaga atcgattcctcaggccaggtgatgactccagccacgacctcatgctgctccgcctgtcagagcctgccgagctcacggatgctgtgaagg tcatggacctgcccacccaggagccagcactggggaccacctgctacgcctcaggctggggcagcattgaaccagaggagttcttgac cccaaagaaacttcagtgtgtggacctccatgttatttccaatgacgtgtgtgcgcaagttcaccctcagaaggtgaccaagttcatgctgtg tgctggacgctggacagggggcaaaagcacctgctcgtgggtcattctgatcaccgaactgaccatgccagccctgccgatggtcctcc atggctccctagtgccctggagaggaggtgtctagtcagagagtagtcctggaaggtggcctctgtgaggagccacggggacagcatc ctgcagatggtcctggcccttgtcccaccgacctgtctacaaggactgtcctcgtggaccctcccctctgcacaggagctggaccctgaa gtcccttccccaccggccaggactggagcccctacccctctgttggaatccctgcccaccttcttctggaagtcggctctggagacatttct ctcttcttccaaagctgggaactgctatctgttatctgcctgtccaggtctgaaagataggattgcccaggcagaaactgggactgacctatc tcactctctccctgcttttacccttagggtgattctgggggcccacttgtctgtaatggtgtgcttcaaggtatcacgtcatggggcagtgaac catgtgccctgcccgaaaggccttccctgtacaccaaggtggtgcattaccggaagtggatcaaggacaccatcgtggccaacccctga gcacccctatcaaccccctattgtagtaaacttggaaccttggaaatgaccaggccaagactcaagcctccccagttctactgacctttgtcc ttaggtgtgaggtccagggttgctaggaaaagaaatcagcagacacaggtgtagaccagagtgtttcttaaatggtgtaattttgtcctctct gtgtcctggggaatactggccatgcctggagacatatcactcaatttctctgaggacacagataggatggggtgtctgtgttatttgtggggt acagagatgaaagaggggtgggatccacactgagagagtggagagtgacatgtgctggacactgtccatgaagcactgagcagaagc tggaggcacaacgcaccagacactcacagcaaggatggagctgaaaacataacccactctgtcctggaggcactgggaagcctagag aaggctgtgagccaaggagggagggtcttcctttggcatgggatggggatgaagtaaggagagggactggaccccctggaagctgatt cactatggggggaggtgtattgaagtcctccagacaaccctcagatttgatgatttcctagtagaactcacagaaataaagagctgttatact gtg (SEQ ID No: 30; GenBank Accession No. NM_001030047). In another embodiment, the KLK3 protein is encoded by residues 42-758 of SEQ ID No: 30. In another embodiment, the KLK3 protein is encoded by a homologue of SEQ ID No: 30. In another embodiment, the KLK3 protein is encoded by a variant of SEQ ID No: 30. In another embodiment, the KLK3 protein is encoded by an isomer of SEQ ID No: 30. In another embodiment, the KLK3 protein is encoded by a fragment of SEQ ID No: 30.

In another embodiment, the KLK3 protein has the sequence: MWVPVVFLTLSVTWIGAAPLILSRIVGGWECEKHSQPWQVLVASRGRAVCGGVLVH PQWVLTAAHCIRK (SEQ ID No: 31; GenBank Accession No. NP_001025221). In another embodiment, the KLK3 protein is a homologue of SEQ ID No: 31. In another embodiment, the KLK3 protein is a variant of SEQ ID No: 31. In another embodiment, the sequence of the KLK3 protein comprises SEQ ID No: 31. In another embodiment, the KLK3 protein is an isomer of SEQ ID No: 31. In another embodiment, the KLK3 protein is a fragment of SEQ ID No: 31.

In another embodiment, the KLK3 protein is encoded by a nucleotide molecule having the sequence: agccccaagcttaccacctgcacccggagagctgtgtcaccatgtgggtcccggttgtcttcctcacccttccgtgacgtggattggtgctg cacccctcatcctgtctcggattgtgggaggctgggagtgcgagaagcattcccaaccctggcaggtgcttgtggcctctcgtggcaggg cagtctgcggcggtgttctggtgcacccccagtgggtcctcacagctgcccactgcatcaggaagtgagtaggggcctggggtctgggg agcaggtgtctgtgtcccagaggaataacagctgggcattttccccaggataacctctaaggccagccttgggactgggggagagaggg aaagttctggttcaggtcacatggggaggcagggttggggctggaccaccctccccatggctgcctgggtctccatctgtgttcctctatgt ctctttgtgtcgctttcattatgtctcttggtaactggcttcggttgtgtctctccgtgtgactattttgttctctctctccctctcttctctgtcttcagt (SEQ ID No: 32). In another embodiment, the KLK3 protein is encoded by residues 42-758 of SEQ ID No: 32. In another embodiment, the KLK3 protein is encoded by a homologue of SEQ ID No: 32. In another embodiment, the KLK3 protein is encoded by a variant of SEQ ID No: 32. In another embodiment, the KLK3 protein is encoded by an isomer of SEQ ID No: 32. In another embodiment, the KLK3 protein is encoded by a fragment of SEQ ID No: 32.

In another embodiment, the KLK3 protein that is the source of the KLK3 peptide has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSIEPEEFLTPKKLQCVDLHVISNDVCAQVHPQKVTKFMLCAGRWTGGKSTCSGD SGGPLVCNGVLQGITSWGSEPCALPERPSLYTKVVHYRKWIKDTIVANP (SEQ ID No: 33). In another embodiment, the KLK3 protein is a homologue of SEQ ID No: 33. In another embodiment, the KLK3 protein is a variant of SEQ ID No: 33. In another embodiment, the KLK3 protein is an isomer of SEQ ID No: 33. In another embodiment, the KLK3 protein is a fragment of SEQ ID No: 33.

In another embodiment, the KLK3 protein is encoded by a nucleotide molecule having the sequence: agccccaagcttaccacctgcacccggagagctgtgtcaccatgtgggtcccggttgtcttcctcaccctgtccgtgacgtggattggtgct gcacccctcatcctgtctcggattgtgggaggctgggagtgcgagaagcattcccaaccctggcaggtgcttgtggcctctcgtggcagg gcagtctgcggcggtgttctggtgcacccccagtgggtcctcacagctgcccactgcatcaggaacaaaagcgtgatcttgctgggtcgg cacagcctgtttcatcctgaagacacaggccaggtatttcaggtcagccacagcttcccacacccgctctacgatatgagcctcctgaaga atcgattcctcaggccaggtgatgactccagcattgaaccagaggagttcttgaccccaaagaaacttcagtgtgtggacctccatgttattt ccaatgacgtgtgtgcgcaagttcaccctcagaaggtgaccaagttcatgctgtgtgctggacgctggacagggggcaaaagcacctgc tcgggtgattctgggggcccacttgtctgtaatggtgtgcttcaaggtatcacgtcatggggcagtgaaccatgtgccctgcccgaaaggc cttccctgtacaccaaggtggtgcattaccggaagtggatcaaggacaccatcgtggccaacccctgagcacccctatcaaccccctattg tagtaaacttggaaccttggaaatgaccaggccaagactcaagcctccccagttctactgacctttgtccttaggtgtgaggtccagggttg ctaggaaaagaaatcagcagacacaggtgtagaccagagtgtttcttaaatggtgtaattttgtcctctctgtgtcctggggaatactggcca tgcctggagacatatcactcaatttctctgaggacacagataggatggggtgtctgtgttatttgtggggtacagagatgaaagaggggtgg gatccacactgagagagtggagagtgacatgtgctggacactgtccatgaagcactgagcagaagctggaggcacaacgcaccagac actcacagcaaggatggagctgaaaacataacccactctgtcctggaggcactgggaagcctagagaaggctgtgagccaaggaggg agggtcttcctttggcatgggatggggatgaagtaaggagagggactggaccccctggaagctgattcactatggggggaggtgtattga agtcctccagacaaccctcagatttgatgatttcctagtagaactcacagaaataaagagctgttatactgtg (SEQ ID No: 34). In another embodiment, the KLK3 protein is encoded by residues 42-758 of SEQ ID No: 34. In another embodiment, the KLK3 protein is encoded by a homologue of SEQ ID No: 34. In another embodiment, the KLK3 protein is encoded by a variant of SEQ ID No: 34. In another embodiment, the KLK3 protein is encoded by an isomer of SEQ ID No: 34. In another embodiment, the KLK3 protein is encoded by a fragment of SEQ ID No: 34.

In another embodiment, the KLK3 protein has the sequence: MWVPVVFLTLSVTWIGAAPLILSRIVGGWECEKHSQPWQVLVASRGRAVCGGVLVH PQWVLTAAHCIRKPGDDSSHDLMLLRLSEPAELTDAVKVMDLPTQEPALGTTCYASG WGSIEPEEFLTPKKLQCVDLHVISNDVCAQVHPQKVTKFMLCAGRWTGGKSTCSGDS GGPLVCNGVLQGITSWGSEPCALPERPSLYTKVVHYRKWIKDTIVANP (SEQ ID No: 35; GenBank Accession No. NP_001025219). In another embodiment, the KLK3 protein is a homologue of SEQ ID No: 35. In another embodiment, the KLK3 protein is a variant of SEQ ID No: 35. In another embodiment, the KLK3 protein is an isomer of SEQ ID No: 35. In another embodiment, the KLK3 protein is a fragment of SEQ ID No: 35.

In another embodiment, the KLK3 protein is encoded by a nucleotide molecule having the sequence: agccccaagcttaccacctgcacccggagagctgtgtcaccatgtgggtcccggttgtcttcctcaccctgtccgtgacgtggattggtgct gcacccctcatcctgtctcggattgtgggaggctgggagtgcgagaagcattcccaaccctggcaggtgcttgtggcctctcgtggcagg gcagtctgcggcggtgttctggtgcacccccagtgggtcctcacagctgcccactgcatcaggaagccaggtgatgactccagccacga cctcatgctgctccgcctgtcagagcctgccgagctcacggatgctgtgaaggtcatggacctgcccacccaggagccagcactgggg accacctgctacgcctcaggctggggcagcattgaaccagaggagttcttgaccccaaagaaacttcagtgtgtggacctccatgttatttc caatgacgtgtgtgcgcaagttcaccctcagaaggtgaccaagttcatgctgtgtgctggacgctggacagggggcaaaagcacctgct cgggtgattctgggggcccacttgtctgtaatggtgtgcttcaaggtatcacgtcatggggcagtgaaccatgtgccctgcccgaaaggcc ttccctgtacaccaaggtggtgcattacccaaggacaccatcgtggccaacccctgagcacccctatcaaccccctattgtagtaaacttgg aaccttggaaatgaccaggccaagactcaagcctccccagttctactgacctttgtccttaggtgtgaggtccagggttgctaggaaaaga aatcagcagacacaggtgtagaccagagtgtttcttaaatggtgtaattttgtcctctctgtgtcctggggaatactggccatgcctggagac atatcactcaatttctctgaggacacagataggatggggtgtctgtgttatttgtggggtacagagatgaaagaggggtgggatccacactg agagagtggagagtgacatgtgctggacactgtccatgaagcactgagcagaagctggaggcacaacgcaccagacactcacagcaa ggatggagctgaaaacataacccactctgtcctggaggcactgggaagcctagagaaggctgtgagccaaggagggagggtcttccttt ggcatgggatggggatgaagtaaggagagggactggaccccctggaagctgattcactatggggggaggtgtattgaagtcctccaga caaccctcagatttgatgatttcctagtagaactcacagaaataaagagctgttatactgtg (SEQ ID No: 36; GenBank Accession No. NM_001030048). In another embodiment, the KLK3 protein is encoded by residues 42-758 of SEQ ID No: 36. In another embodiment, the KLK3 protein is encoded by a homologue of SEQ ID No: 36. In another embodiment, the KLK3 protein is encoded by a variant of SEQ ID No: 36. In another embodiment, the KLK3 protein is encoded by an isomer of SEQ ID No: 36. In another embodiment, the KLK3 protein is encoded by a fragment of SEQ ID No: 36.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKLQ CVDLHVISNDVCAQVHPQKVTKFMLCAGRWTGGKSTCSGDSGGPLVCNGVLQGITS WGSEPCALPERPSLYTKVVHYRKWIKDTIVANP (SEQ ID No: 37; GenBank Accession No. NP_001639). In another embodiment, the KLK3 protein is a homologue of SEQ ID No: 37. In another embodiment, the KLK3 protein is a variant of SEQ ID No: 37. In another embodiment, the KLK3 protein is an isomer of SEQ ID No: 37. In another embodiment, the KLK3 protein is a fragment of SEQ ID No: 37.

In another embodiment, the KLK3 protein is encoded by a nucleotide molecule having the sequence: agccccaagcttaccacctgcacccggagagctgtgtcaccatgtgggtcccggttgtcttcctcaccctgtccgtgacgtggattggtgct gcacccctcatcctgtctcggattgtgggaggctgggagtgcgagaagcattcccaaccctggcaggtgcttgtggcctctcgtggcagg gcagtctgcggcggtgttctggtgcacccccagtgggtcctcacagctgcccactgcatcaggaacaaaagcgtgatcttgctgggtcgg cacagcctgtttcatcctgaagacacaggccaggtatttcaggtcagccacagcttcccacacccgctctacgatatgagcctcctgaaga atcgattcctcaggccaggtgatgactccagccacgacctcatgctgctccgcctgtcagagcctgccgagctcacggatgctgtgaagg tcatggacctgcccacccaggagccagcactggggaccacctgctacgcctcaggctggggcagcattgaaccagaggagttcttgac cccaaagaaacttcagtgtgtggacctccatgttatttccaatgacgtgtgtgcgcaagttcaccctcagaaggtgaccaagttcatgctgtg tgctggacgctggacagggggcaaaagcacctgctcgggtgattctgggggcccacttgtctgtaatggtgtgcttcaaggtatcacgtca tggggcagtgaaccatgtgccctgcccgaaaggccttccctgtacaccaaggtggtgcattaccggaagtggatcaaggacaccatcgt ggccaacccctgagcacccctatcaaccccctattgtagtaaacttggaaccttggaaatgaccaggccaagactcaagcctccccagttc tactgacctttgtccttaggtgtgaggtccagggttgctaggaaaagaaatcagcagacacaggtgtagaccagagtgtttcttaaatggtgt aattttgtcctctctgtgtcctggggaatactggccatgcctggagacatatcactcaatttctctgaggacacagataggatggggtgtctgt gttatttgtggggtacagagatgaaagaggggtgggatccacactgagagagtggagagtgacatgtgctggacactgtccatgaagca ctgagcagaagctggaggcacaacgcaccagacactcacagcaaggatggagctgaaaacataacccactctgtcctggaggcactg ggaagcctagagaaggctgtgagccaaggagggagggtcttcctttggcatgggatggggatgaagtaaggagagggactggacccc ctggaagctgattcactatggggggaggtgtattgaagtcctccagacaaccctcagatttgatgatttcctagtagaactcacagaaataaa gagctgttatactgtg (SEQ ID No: 38; GenBank Accession No. NM_001648). In another embodiment, the KLK3 protein is encoded by residues 42-827 of SEQ ID No: 38. In another embodiment, the KLK3 protein is encoded by a homologue of SEQ ID No: 38. In another embodiment, the KLK3 protein is encoded by a variant of SEQ ID No: 38. In another embodiment, the KLK3 protein is encoded by an isomer of SEQ ID No: 38. In another embodiment, the KLK3 protein is encoded by a fragment of SEQ ID No: 38.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKLQ CVDLHVISNDVCAQVHPQKVTKFMLCAGRWTGGKSTCSGDSGGPLVCNGVLQGITS WGSEPCALPERPSLYTKVVHYRKWIKDTIVANP (SEQ ID No: 39 GenBank Accession No. AAX29407.1). In another embodiment, the KLK3 protein is a homologue of SEQ ID No: 39. In another embodiment, the KLK3 protein is a variant of SEQ ID No: 39. In another embodiment, the KLK3 protein is an isomer of SEQ ID No: 39. In another embodiment, the sequence of the KLK3 protein comprises SEQ ID No: 39. In another embodiment, the KLK3 protein is a fragment of SEQ ID No: 39.

In another embodiment, the KLK3 protein is encoded by a nucleotide molecule having the sequence: gggggagccccaagcttaccacctgcacccggagagctgtgtcaccatgtgggtcccggttgtcttcctcaccctgtccgtgacgtggatt ggtgctgcacccctcatcctgtctcggattgtgggaggctgggagtgcgagaagcattcccaaccctggcaggtgcttgtggcctctcgtg gcagggcagtctgcggcggtgttctggtgcacccccagtgggtcctcacagctgcccactgcatcaggaacaaaagcgtgatcttgctg ggtcggcacagcctgtttcatcctgaagacacaggccaggtatttcaggtcagccacagcttcccacacccgctctacgatatgagcctcc tgaagaatcgattcctcaggccaggtgatgactccagccacgacctcatgctgctccgcctgtcagagcctgccgagctcacggatgctg tgaaggtcatggacctgcccacccaggagccagcactggggaccacctgctacgcctcaggctggggcagcattgaaccagaggagtt cttgaccccaaagaaacttcagtgtgtggacctccatgttatttccaatgacgtgtgtgcgcaagttcaccctcagaaggtgaccaagttcat gctgtgtgctggacgctggacagggggcaaaagcacctgctcgggtgattctgggggcccacttgtctgtaatggtgtgcttcaaggtatc acgtcatggggcagtgaaccatgtgccctgcccgaaaggccttccctgtacaccaaggtggtgcattaccggaagtggatcaaggacac catcgtggccaacccctgagcacccctatcaactccctattgtagtaaacttggaaccttggaaatgaccaggccaagactcaggcctccc cagttctactgacctttgtccttaggtgtgaggtccagggttgctaggaaaagaaatcagcagacacaggtgtagaccagagtgtttcttaaa tggtgtaattttgtcctctctgtgtcctggggaatactggccatgcctggagacatatcactcaatttctctgaggacacagataggatggggt gtctgtgttatttgtggggtacagagatgaaagaggggtgggatccacactgagagagtggagagtgacatgtgctggacactgtccatg aagcactgagcagaagctggaggcacaacgcaccagacactcacagcaaggatggagctgaaaacataacccactctgtcctggagg cactgggaagcctagagaaggctgtgagccaaggagggagggtcttcctttggcatgggatggggatgaagtagggagagggactgg accccctggaagctgattcactatggggggaggtgtattgaagtcctccagacaaccctcagatttgatgatttcctagtagaactcacaga aataaagagctgttatactgcgaaaaaaaaaaaaaaaaaaaaaaaaaa (SEQ ID No: 40; GenBank Accession No. BC056665). In another embodiment, the KLK3 protein is encoded by residues 47-832 of SEQ ID No: 40. In another embodiment, the KLK3 protein is encoded by a homologue of SEQ ID No: 40. In another embodiment, the KLK3 protein is encoded by a variant of SEQ ID No: 40. In another embodiment, the KLK3 protein is encoded by an isomer of SEQ ID No: 40. In another embodiment, the KLK3 protein is encoded by a fragment of SEQ ID No: 40.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSIEPEEFLTPKKLQCVDLHVISNDVCAQVHPQKVTKFMLCAGRWTGGKSTCSGD SGGPLVCNGVLQGITSWGSEPCALPERPSLYTKVVHYRKWIKDTIVA (SEQ ID No: 41; GenBank Accession No. AJ459782). In another embodiment, the KLK3 protein is a homologue of SEQ ID No: 41. In another embodiment, the KLK3 protein is a variant of SEQ ID No: 41. In another embodiment, the KLK3 protein is an isomer of SEQ ID No: 41. In another embodiment, the KLK3 protein is a fragment of SEQ ID No: 41.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKLQ CVDLHVISNDVCAQVHPQKVTKFMLCAGRWTGGKSTCSVSHPYSQDLEGKGEWGP (SEQ ID No: 42, GenBank Accession No. AJ512346). In another embodiment, the KLK3 protein is a homologue of SEQ ID No: 42. In another embodiment, the KLK3 protein is a variant of SEQ ID No: 42. In another embodiment, the KLK3 protein is an isomer of SEQ ID No: 42. In another embodiment, the sequence of the KLK3 protein comprises SEQ ID No: 42. In another embodiment, the KLK3 protein is a fragment of SEQ ID No: 42.

In another embodiment, the KLK3 protein has the sequence: MWVPVVFLTLSVTWIGERGHGWGDAGEGASPDCQAEALSPPTQHPSPDRELGSFLSL PAPLQAHTPSPSILQQSSLPHQVPAPSHLPQNFLPIAQPAPCSQLLY (SEQ ID No: 43; GenBank Accession No. AJ459784). In another embodiment, the KLK3 protein is a homologue of SEQ ID No: 43. In another embodiment, the KLK3 protein is a variant of SEQ ID No: 43. In another embodiment, the sequence of the KLK3 protein comprises SEQ ID No: 43. In another embodiment, the KLK3 protein is an isomer of SEQ ID No: 43. In another embodiment, the KLK3 protein is a fragment of SEQ ID No: 43.

In another embodiment, the KLK3 protein has the sequence: MWVPVVFLTLSVTWIGAAPLILSRIVGGWECEKHSQPWQVLVASRGRAVCGGVLVH PQWVLTAAHCIRNKSVILLGRHSLFHPEDTGQVFQVSHSFPHPLYDMSLLKNRFLRPG DDSSHDLMLLRLSEPAELTDAVKVMDLPTQEPALGTTCYASGWGSIEPEEFLTPKKLQ CVDLHVISNDVCAQVHPQKVTKFMLCAGRWTGGKSTCSGDSGGPLVCNGVLQGITS WGSEPCALPERPSLYTKVVHYRKWIKDTIVANP (SEQ ID NO: 44 GenBank Accession No. AJ459783). In another embodiment, the KLK3 protein is a homologue of SEQ ID No: 44. In another embodiment, the KLK3 protein is a variant of SEQ ID No: 44. In another embodiment, the KLK3 protein is an isomer of SEQ ID No: 44. In another embodiment, the KLK3 protein is a fragment of SEQ ID No: 44.

In another embodiment, the KLK3 protein is encoded by a nucleotide molecule having the sequence: aagtttcccttctcccagtccaagaccccaaatcaccacaaaggacccaatccccagactcaagatatggtctgggcgctgtcttgtgtctc ctaccctgatccctgggttcaactctgctcccagagcatgaagcctctccaccagcaccagccaccaacctgcaaacctagggaagattg acagaattcccagcctttcccagctccccctgcccatgtcccaggactcccagccttggttctctgcccccgtgtcttttcaaacccacatcct aaatccatctcctatccgagtcccccagttcctcctgtcaaccctgattcccctgatctagcaccccctctgcaggtgctgcacccctcatcct gtctcggattgtgggaggctgggagtgcgagaagcattcccaaccctggcaggtgcttgtagcctctcgtggcagggcagtctgcggcg gtgttctggtgcacccccagtgggtcctcacagctacccactgcatcaggaacaaaagcgtgatcttgctgggtcggcacagcctgtttca tcctgaagacacaggccaggtatttcaggtcagccacagcttcccacacccgctctacgatatgagcctcctgaagaatcgattcctcagg ccaggtgatgactccagccacgacctcatgctgctccgcctgtcagagcctgccgagctcacggatgctatgaaggtcatggacctgccc acccaggagccagcactggggaccacctgctacgcctcaggctggggcagcattgaaccagaggagttcttgaccccaaagaaacttc agtgtgtggacctccatgttatttccaatgacgtgtgtgcgcaagttcaccctcagaaggtgaccaagttcatgctgtgtgctggacgctgga cagggggcaaaagcacctgctcgggtgattctgggggcccacttgtctgtaatggtgtgcttcaaggtatcacgtcatggggcagtgaac catgtgccctgcccgaaaggccttccctgtacaccaaggtggtgcattaccggaagtggatcaaggacaccatcgtggccaacccctga gcacccctatcaactccctattgtagtaaacttggaaccttggaaatgaccaggccaagactcaggcctccccagttctactgacctttgtcc ttaggtgtgaggtccagggttgctaggaaaagaaatcagcagacacaggtgtagaccagagtgtttcttaaatggtgtaattttgtcctctct gtgtcctggggaatactggccatgcctggagacatatcactcaatttctctgaggacacagataggatggggtgtctgtgttatttgtggggt acagagatgaaagaggggtgggatccacactgagagagtggagagtgacatgtgctggacactgtccatgaagcactgagcagaagc tggaggcacaacgcaccagacactcacagcaaggatggagctgaaaacataacccactctgtcctggaggcactgggaagcctagag aaggctgtgaaccaaggagggagggtcttcctttggcatgggatggggatgaagtaaggagagggactgaccccctggaagctgattc actatggggggaggtgtattgaagtcctccagacaaccctcagatttgatgatttcctagtagaactcacagaaataaagagctgttatactg tgaa (SEQ ID No: 45; GenBank Accession No. X07730). In another embodiment, the KLK3 protein is encoded by residues 67-1088 of SEQ ID No: 45. In another embodiment, the KLK3 protein is encoded by a homologue of SEQ ID No: 45. In another embodiment, the KLK3 protein is encoded by a variant of SEQ ID No: 45. In another embodiment, the KLK3 protein is encoded by an isomer of SEQ ID No: 45. In another embodiment, the KLK3 protein is encoded by a fragment of SEQ ID No: 45.

In another embodiment, the KLK3 protein has the sequence:

MWVPVVFLTLSVTWIGAAPLILSRIVGGWECEKHSQPWQVLVASRGRAVCG GVLVHPQWVLTAAHCIRK (SEQ ID No: 46; GenBank Accession No. NM_001030050). In another embodiment, the KLK3 protein is a homologue of SEQ ID No: 46. In another embodiment, the KLK3 protein is a variant of SEQ ID No: 46. In another embodiment, the sequence of the KLK3 protein comprises SEQ ID No: 46. In another embodiment, the KLK3 protein is an isomer of SEQ ID No: 46. In another embodiment, the KLK3 protein is a fragment of SEQ ID No: 46.

In another embodiment, the KLK3 protein that is the source of the KLK3 peptide has the sequence:

MWVPVVFLTLSVTWIGAAPLILSRIVGGWECEKHSQPWQVLVASRGRAVCG GVLVHPQWVLTAAHCIRNKSVILLGRHSLFHPEDTGQVFQVSHSFPHPLYDMSLLKNR FLRPGDDSSIEPEEFLTPKKLQCVDLHVISNDVCAQVHPQKVTKFMLCAGRWTGGKST CSGDSGGPLVCNGVLQGITSWGSEPCALPERPSLYTKVVHYRKWIKDTIVANP (SEQ ID No: 47; GenBank Accession No. NM_001064049). In another embodiment, the KLK3 protein is a homologue of SEQ ID No: 47. In another embodiment, the KLK3 protein is a variant of SEQ ID No: 47. In another embodiment, the KLK3 protein is an isomer of SEQ ID No: 47. In another embodiment, the KLK3 protein is a fragment of SEQ ID No: 47.

In another embodiment, the KLK3 protein has the sequence:

MWVPVVFLTLSVTWIGAAPLILSRIVGGWECEKHSQPWQVLVASRGRAVCG GVLVHPQWVLTAAHCIRKPGDDSSHDLMLLRLSEPAELTDAVKVMDLPTQEPALGTT CYASGWGSIEPEEFLTPKKLQCVDLHVISNDVCAQVHPQKVTKFMLCAGRWTGGKST CSGDSGGPLVCNGVLQGITSWGSEPCALPERPSLYTKVVHYRKWIKDTIVANP (SEQ ID No: 48; GenBank Accession No. NM_001030048). In another embodiment, the KLK3 protein is a homologue of SEQ ID No: 48. In another embodiment, the KLK3 protein is a variant of SEQ ID No: 48. In another embodiment, the KLK3 protein is an isomer of SEQ ID No: 48. In another embodiment, the KLK3 protein is a fragment of SEQ ID No: 48.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers.

In another embodiment, the KLK3 protein is encoded by a sequence set forth in one of the following GenBank Accession Numbers: NM_001030050, NM_001030049, NM_001030048, NM_001030047, NM_001648, AJ459782, AJ512346, or AJ459784. Each possibility represents a separate embodiment of the methods and compositions as disclosed herein. In one embodiment, the KLK3 protein is encoded by a variation of any of the sequences described herein wherein the sequence lacks MWVPVVFLTLSVTWIGAAPLILSR (SEQ ID NO: 49).

In another embodiment, the KLK3 protein has the sequence that comprises a sequence set forth in one of the following GenBank Accession Numbers: X13943, X13942, X13940, X13941, and X13944.

In another embodiment, the KLK3 protein is any other KLK3 protein known in the art.

In another embodiment, the KLK3 peptide is any other KLK3 peptide known in the art. In another embodiment, the KLK3 peptide is a fragment of any other KLK3 peptide known in the art.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions as disclosed herein does not contain any signal sequence.

In another embodiment, the kallikrein-related peptidase 3 (KLK3 protein) that is the source of a KLK3 peptide for use in the methods and compositions as disclosed herein is a PSA protein. In another embodiment, the KLK3 protein is a P-30 antigen protein. In another embodiment, the KLK3 protein is a gamma-seminoprotein protein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art.

In another embodiment, the KLK3 protein is a splice variant 1 KLK3 protein. In another embodiment, the KLK3 protein is a splice variant 2 KLK3 protein. In another embodiment, the KLK3 protein is a splice variant 3 KLK3 protein. In another embodiment, the KLK3 protein is a transcript variant 1 KLK3 protein. In another embodiment, the KLK3 protein is a transcript variant 2 KLK3 protein. In another embodiment, the KLK3 protein is a transcript variant 3 KLK3 protein. In another embodiment, the KLK3 protein is a transcript variant 4 KLK3 protein. In another embodiment, the KLK3 protein is a transcript variant 5 KLK3 protein. In another embodiment, the KLK3 protein is a transcript variant 6 KLK3 protein. In another embodiment, the KLK3 protein is a splice variant RP5 KLK3 protein. In another embodiment, the KLK3 protein is any other splice variant KLK3 protein known in the art. In another embodiment, the KLK3 protein is any other transcript variant KLK3 protein known in the art.

In another embodiment, the KLK3 protein is a mature KLK3 protein. In another embodiment, the KLK3 protein is a pro-KLK3 protein. In another embodiment, the leader sequence has been removed from a mature KLK3 protein of methods and compositions as disclosed herein.

In another embodiment, the KLK3 protein that is the source of a KLK3 peptide of methods and compositions as disclosed herein is a human KLK3 protein. In another embodiment, the KLK3 protein is a primate KLK3 protein. In another embodiment, the KLK3 protein is a KLK3 protein of any other species known in the art. In another embodiment, one of the above KLK3 proteins is referred to in the art as a “KLK3 protein.”

In one embodiment, a recombinant polypeptide disclosed herein comprising a truncated LLO fused to a PSA protein disclosed herein is encoded by a sequence comprising:

ATGAAAAAAATAATGCTAGTTTTTATTACACTTATATTAGTTAGTCTACCA ATTGCGCAACAAACTGAAGCAAAGGATGCATCTGCATTCAATAAAGAAAATTCAA TTTCATCCATGGCACCACCAGCATCTCCGCCTGCAAGTCCTAAGACGCCAATCGAA AAGAAACACGCGGATGAAATCGATAAGTATATACAAGGATTGGATTACAATAAAA ACAATGTATTAGTATACCACGGAGATGCAGTGACAAATGTGCCGCCAAGAAAAGG TTACAAAGATGGAAATGAATATATTGTTGTGGAGAAAAAGAAGAAATCCATCAAT CAAAATAATGCAGACATTCAAGTTGTGAATGCAATTTCGAGCCTAACCTATCCAGG TGCTCTCGTAAAAGCGAATTCGGAATTAGTAGAAAATCAACCAGATGTTCTCCCTG TAAAACGTGATTCATTAACACTCAGCATTGATTTGCCAGGTATGACTAATCAAGAC AATAAAATAGTTGTAAAAAATGCCACTAAATCAAACGTTAACAACGCAGTAAATA CATTAGTGGAAAGATGGAATGAAAAATATGCTCAAGCTTATCCAAATGTAAGTGC AAAAATTGATTATGATGACGAAATGGCTTACAGTGAATCACAATTAATTGCGAAAT TTGGTACAGCATTTAAAGCTGTAAATAATAGCTTGAATGTAAACTTCGGCGCAATC AGTGAAGGGAAAATGCAAGAAGAAGTCATTAGTTTTAAACAAATTTACTATAACG TGAATGTTAATGAACCTACAAGACCTTCCAGATTTTTCGGCAAAGCTGTTACTAAA GAGCAGTTGCAAGCGCTTGGAGTGAATGCAGAAAATCCTCCTGCATATATCTCAAG TGTGGCGTATGGCCGTCAAGTTTATTTGAAATTATCAACTAATTCCCATAGTACTAA AGTAAAAGCTGCTTTTGATGCTGCCGTAAGCGGAAAATCTGTCTCAGGTGATGTAG AACTAACAAATATCATCAAAAATTCTTCCTTCAAAGCCGTAATTTACGGAGGTTCC GCAAAAGATGAAGTTCAAATCATCGACGGCAACCTCGGAGACTTACGCGATATTTT GAAAAAAGGCGCTACTTTTAATCGAGAAACACCAGGAGTTCCCATTGCTTATACAA CAAACTTCCTAAAAGACAATGAATTAGCTGTTATTAAAAACAACTCAGAATATATT GAAACAACTTCAAAAGCTTATACAGATGGAAAAATTAACATCGATCACTCTGGAG GATACGTTGCTCAATTCAACATTTCTTGGGATGAAGTAAATTATGATCTCGAGattgtg ggaggctgggagtgcgagaagcattcccaaccctggcaggtgcttgtggcctctcgtggcagggcagtctgcggcggtgttctggtgc acccccagtgggtcctcacagctgcccactgcatcaggaacaaaagcgtgatcttgctgggtcggcacagcctgtttcatcctgaagac acaggccaggtatttcaggtcagccacagcttcccacacccgctctacgatatgagcctcctgaagaatcgattcctcaggccaggtga tgactccagccacgacctcatgctgctccgcctgtcagagcctgccgagctcacggatgctgtgaaggtcatggacctgcccacccag gagccagcactggggaccacctgctacgcctcaggctggggcagcattgaaccagaggagttcttgaccccaaagaaacttcagtgt gtggacctccatgttatttccaatgacgtgtgtgcgcaagttcaccctcagaaggtgaccaagttcatgctgtgtgctggacgctggaca gggggcaaaagcacctgctcgggtgattctgggggcccacttgtctgttatggtgtgcttcaaggtatcacgtcatggggcagtgaacc atgtgccctgcccgaaaggccttccctgtacaccaaggtggtgcattaccggaagtggatcaaggacaccatcgtggccaacccc (SEQ ID NO: 50). In another embodiment, the fusion protein is encoded by a homologue of SEQ ID No: 50. In another embodiment, the fusion protein is encoded by a variant of SEQ ID No: 50. In another embodiment, the fusion protein is encoded by an isomer of SEQ ID No: 50. In one embodiment, the “ctcgag” sequence within the fusion protein represents a Xho I restriction site used to ligate the tumor antigen to truncated LLO in the plasmid.

In another embodiment, a recombinant polypeptide disclosed herein comprising a truncated LLO fused to a PSA protein disclosed herein comprises the following sequence:

MKKIMLVFITLILVSLPIAQQTEAKDASAFNKENSISSMAPPASPPASPKTPIEK KHADEIDKYIQGLDYNKNNVLVYHGDAVTNVPPRKGYKDGNEYIVVEKKKKSINQNN ADIQVVNAISSLTYPGALVKANSELVENQPDVLPVKRDSLTLSIDLPGMTNQDNKIVVK NATKSNVNNAVNTLVERWNEKYAQAYPNVSAKIDYDDEMAYSESQLIAKFGTAFKA VNNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSFK AVIYGGSAKDEVQIIDGNLGDLRDILKKGATFNRETPGVPIAYTTNFLKDNELAVIKNN SEYIETTSKAYTDGKINIDHSGGYVAQFNISWDEVNYDLE

(SEQ ID NO: 51)
IVGGWECEKHSQPWQVLVASRGRAVCGGVLVHPQWVLTAAHCIRNKSVI
LLGRHSLFHPEDTGQVFQVSHSFPHPLYDMSLLKNRFLRPGDDSSHDLM
LLRLSEPAELTDAVKVMDLPTQEPALGTTCYASGWGSIEPEEFLTPKK
LQCVDLHVISNDVCAQVHPQKVTKFMLCAGRWTGGKSTCSGDSGG
PLVCYGVLQGITSWGSEPCALPERPSLYTKVVHYRKWIKDTIVANP.
(PSA sequence is underlined)

In another embodiment, the tLLO-PSA fusion protein is a homologue of SEQ ID NO: 51. In another embodiment, the tLLO-PSA fusion protein is a variant of SEQ ID NO: 51. In another embodiment, the tLLO-PSA fusion protein is an isomer of SEQ ID NO: 51. In another embodiment, the tLLO-PSA fusion protein is a fragment of SEQ ID NO: 51.

In one embodiment, the recombinant Listeria strain as disclosed herein comprises a nucleic acid molecule encoding a tumor associated antigen, wherein the antigen comprises an HPV-E7 protein. In one embodiment, the recombinant Listeria strain as disclosed herein comprises a nucleic acid molecule encoding HPV-E7 protein.

In one embodiment, either a whole E7 protein or a fragment thereof is fused to a LLO protein or truncation or peptide thereof, an ActA protein or truncation or peptide thereof, or a PEST-like sequence-containing peptide to generate a recombinant polypeptide or peptide of the composition and methods of the disclosure. The E7 protein that is utilized (either whole or as the source of the fragments) has, in another embodiment, the sequence

MHGDTPTLHEYMLDLQPETTDLYCYEQLNDSSEEEDEIDGPAGQAEPDRAHY NIVTFCCKCDSTLRLCVQSTHVDIRTLEDLLMGTLGIVCPICSQKP (SEQ ID No: 52). In another embodiment, the E7 protein is a homologue of SEQ ID No: 52. In another embodiment, the E7 protein is a variant of SEQ ID No: 52. In another embodiment, the E7 protein is an isomer of SEQ ID No: 52. In another embodiment, the E7 protein is a fragment of SEQ ID No: 52. In another embodiment, the E7 protein is a fragment of a homologue of SEQ ID No: 52. In another embodiment, the E7 protein is a fragment of a variant of SEQ ID No: 52. In another embodiment, the E7 protein is a fragment of an isomer of SEQ ID No: 52.

In another embodiment, the sequence of the E7 protein is:

MHGPKATLQDIVLHLEPQNEIPVDLLCHEQLSDSEEENDEIDGVNHQHLPARR AEPQRHTMLCMCCKCEARIELVVESSADDLRAFQQLFLNTLSFVCPWCASQQ (SEQ ID No: 53). In another embodiment, the E6 protein is a homologue of SEQ ID No: 53. In another embodiment, the E6 protein is a variant of SEQ ID No: 53. In another embodiment, the E6 protein is an isomer of SEQ ID No: 53. In another embodiment, the E6 protein is a fragment of SEQ ID No: 53. In another embodiment, the E6 protein is a fragment of a homologue of SEQ ID No: 53. In another embodiment, the E6 protein is a fragment of a variant of SEQ ID No: 53. In another embodiment, the E6 protein is a fragment of an isomer of SEQ ID No: 53.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries.

In one embodiment the HPV antigen is an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In one embodiment, the HPV E6 is from HPV-16. In another embodiment, the HPV E7 is from HPV-16. In another embodiment, the HPV-E6 is from HPV-18. In another embodiment, the HPV-E7 is from HPV-18. In another embodiment, an HPV E6 antigen is utilized instead of or in addition to an E7 antigen in a composition or method of the disclosure for treating or ameliorating an HPV-mediated disease, disorder, or symptom. In another embodiment, an HPV-16 E6 and E7 is utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria disclosed herein. In another embodiment, the HPV-16 E6 and E7 antigens and the HPV-18 E6 and E7 antigens are expressed from the chromosome of a recombinant Listeria disclosed herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In another embodiment, either a whole E7 protein or a fragment thereof is fused to a LLO protein, ActA protein, or PEST amino acid sequence-containing peptide to generate a recombinant polypeptide disclosed herein. In one embodiment, the E7 protein that is utilized (either whole or as the source of the fragments) comprises the amino acid sequence set forth in SEQ ID NO: 54

(SEQ ID NO: 54)
H G D T P T L H E Y M L D L Q P E T T D L Y C Y E
Q L N D S S E E E D E I D G P A G Q A E P D R A H
Y N I V T F C C K C D S T L R L C V Q S T H V D I
R T L E D L L M G T L G I V C P I C S Q K P.

In another embodiment, the E7 protein is a homologue of SEQ ID No: 117. In another embodiment, the E7 protein is a variant of SEQ ID No: 54. In another embodiment, the E7 protein is an isomer of SEQ ID No: 54. In another embodiment, the E7 protein is a fragment of SEQ ID No: 54. In another embodiment, the E7 protein is a fragment of a homologue of SEQ ID No: 54. In another embodiment, the E7 protein is a fragment of a variant of SEQ ID No: 54. In another embodiment, the E7 protein is a fragment of an isomer of SEQ ID No: 54.

In another embodiment, the amino acid sequence of a truncated LLO fused to an E7 protein comprises the following amino acid sequence:

(SEQ ID NO: 55)
MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYPNV
SASAKIDYDDEMAYSESQLIAKFGTAFKAVNNSLNVNFGAISEGKMQEEV
ISFKQIYYNVNVNEPTRPRSRFFGKAVTKEQLQALGVNAENPPAYISSVA
YGRQVYLKLSTNSHSTKVKAAFDAAVSGKSVSGDVELTNIIKNSSFKAVI
YGGSAKDEVQIIDGNLGDLRDILKKGARFNRETPGVPIAYTTNFLKDNEL
AVIKNNSEYIETTSKAYTDGKINIDHSGGYVAQFNISWDEVNYDELHGDT
PTLHEYMLDLQPETTDLYCYEQLNDSSEEEDEIDGPAQQAEPDRAHYNIV
TFCCKCDSTLRLCVQSTHVDIRTLEDLLMGTLGIVCPICSQKP.

In another embodiment, the fusion protein of tLLO-E7 is a homologue of SEQ ID No: 55. In another embodiment, the fusion protein is a variant of SEQ ID No: 55. In another embodiment, the tLLO-E7 fusion protein is an isomer of SEQ ID No: 55. In another embodiment, the tLLO-E7 fusion protein is a fragment of SEQ ID No: 55. In another embodiment, the tLLO-E7 fusion protein is a fragment of a homologue of SEQ ID No: 55. In another embodiment, the tLLO-E7 fusion protein is a fragment of a variant of SEQ ID No: 55. In another embodiment, the tLLO-E7 fusion protein is a fragment of an isomer of SEQ ID No: 55.

In one embodiment, the recombinant Listeria strain as disclosed herein comprises a nucleic acid molecule encoding a tumor associated antigen, wherein the tumor associated antigen comprises an Her-2/neu peptide. In one embodiment, a tumor associated antigen comprises an Her-2/neu antigen. In one embodiment, the Her-2/neu peptide comprises a chimeric Her-2/neu antigen (cHer-2).

In one embodiment, the attenuated auxotrophic Listeria strain is based on a Listeria vector which is attenuated due to the deletion of virulence gene actA and retains the plasmid for Her2/neu expression in vivo and in vitro by complementation of dal gene. In one embodiment, the Listeria strain expresses and secretes a chimeric Her2/neu protein fused to the first 441 amino acids of listeriolysin O (LLO). In another embodiment, the Listeria is a dal/dat/actA Listeria having a mutation in the dal, dat and actA endogenous genes. In another embodiment, the mutation is a deletion, a truncation or an inactivation of the mutated genes. In another embodiment, Listeria strain exerts strong and antigen specific anti-tumor responses with ability to break tolerance toward HER2/neu in transgenic animals. In another embodiment, the dal/dat/actA strain is highly attenuated and has a better safety profile than previous Listeria generation, as it is more rapidly cleared from the spleens of the immunized mice. In another embodiment, the Listeria strain results in a longer delay of tumor onset in transgenic animals than Lm-LLO-ChHer2, the antibiotic resistant and more virulent version of this vaccine (see US Publication No. 2011/0142791, which is incorporated by reference herein in its entirety). In another embodiment, the Listeria strain causes a significant decrease in intra-tumoral T regulatory cells (Tregs). In another embodiment, the lower frequency of Tregs in tumors treated with LmddA vaccines result in an increased intratumoral CD8/Tregs ratio, suggesting that a more favorable tumor microenvironment can be obtained after immunization with LmddA vaccines. In one embodiment, the disclosure provides a recombinant polypeptide comprising an N-terminal fragment of an LLO protein fused to a Her-2 chimeric protein or fused to a fragment thereof. In one embodiment, the disclosure provides a recombinant polypeptide consisting of an N-terminal fragment of an LLO protein fused to a Her-2 chimeric protein or fused to a fragment thereof. In the embodiment, the heterologous antigen is a Her-2 chimeric protein or fragment thereof.

In another embodiment, the Her-2 chimeric protein of the methods and compositions of the disclosure is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of human or any other animal species or combinations thereof known in the art. Each possibility represents a separate embodiment of the disclosure.

In another embodiment, a Her-2 protein is a protein referred to as “HER-2/neu,” “Erbb2,” “v-erb-b2,” “c-erb-b2,” “neu,” or “cNeu.”

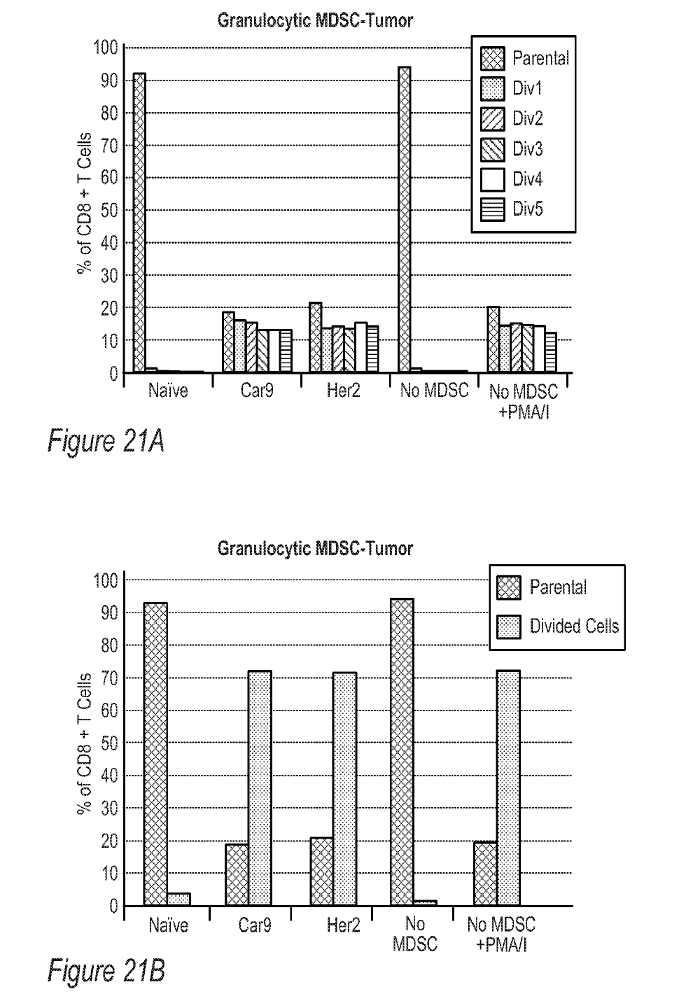

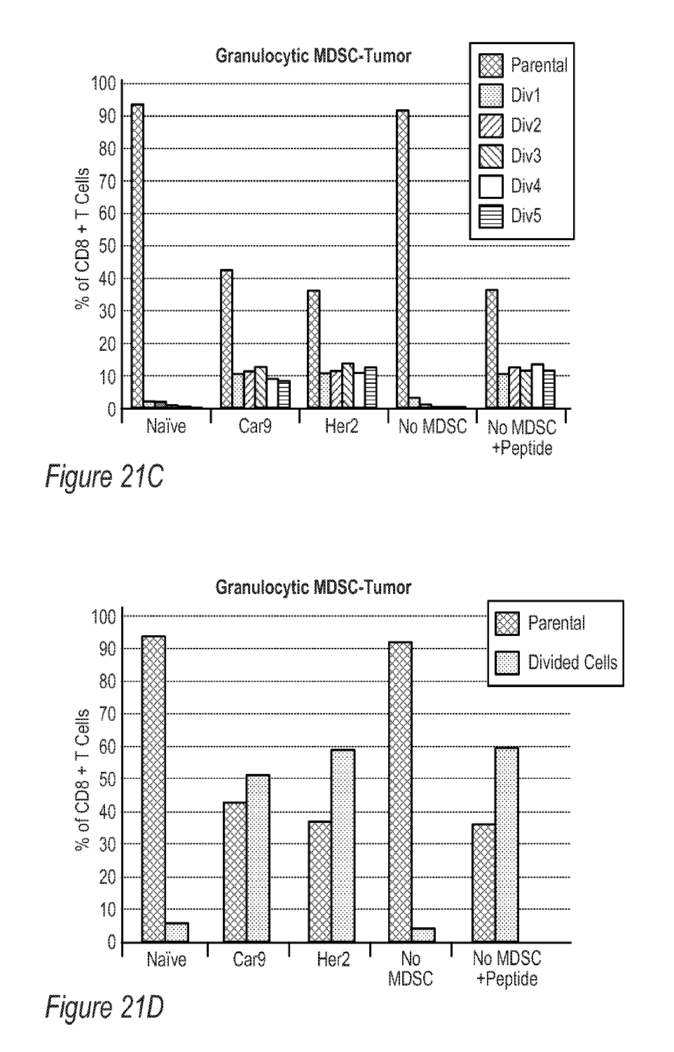

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen (fragments EC1, EC2, and IC1) (See FIG. 21). In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2). In another embodiment, the Her2-neu chimeric protein is fused to a non-hemolytic listeriolysin O (LLO). In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the expression and secretion of the fusion protein tLLO-ChHer2 from the attenuated auxotrophic strain disclosed herein that expresses a chimeric Her2/neu antigen/LLO fusion protein is comparable to that of the Lm-LLO-ChHer2 in TCA precipitated cell culture supernatants after 8 hours of in vitro growth (See FIG. 21B).

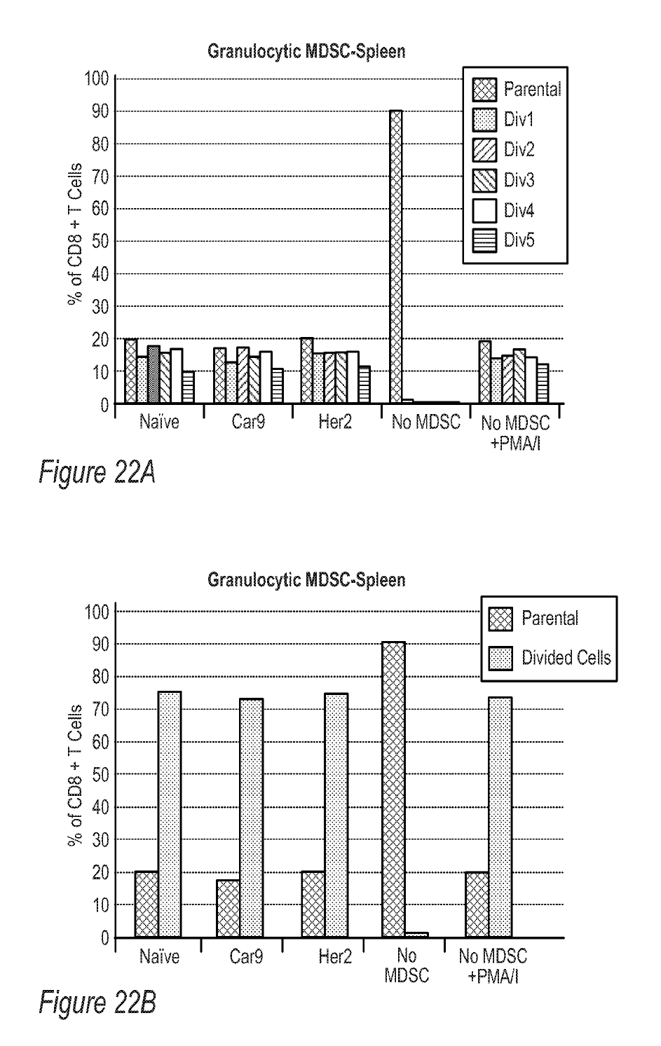

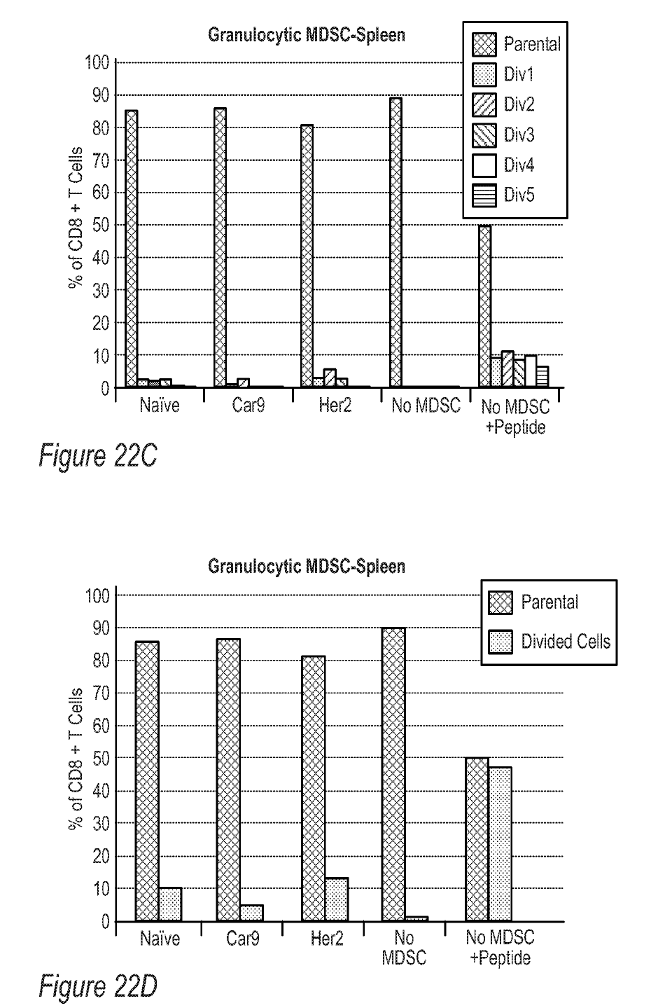

In one embodiment, no CTL activity is detected in naïve animals or mice injected with an irrelevant Listeria (See FIG. 22A). While in another embodiment, the attenuated auxotrophic strain disclosed herein is able to stimulate the secretion of IFN-γ by the splenocytes from wild type FVB/N mice (FIG. 22B).

In one embodiment, the antigen is a chimeric Her2 antigen described in US patent application publication US2011/0142791, which is hereby incorporated by reference herein in its entirety.

In another embodiment, the Her-2 chimeric protein is encoded by the following nucleic acid sequence set forth in SEQ ID NO:56

gagacccacctggacatgctccgccacctctaccagggctgccaggtggtgcagggaaacctggaactcacctacctgcc caccaatgccagcctgtccttcctgcaggatatccaggaggtgcagggctacgtgctcatcgctcacaaccaagtgaggcaggtcccact gcagaggctgcggattgtgcgaggcacccagctctttgaggacaactatgccctggccgtgctagacaatggagacccgctgaacaata ccacccctgtcacaggggcctccccaggaggcctgcgggagctgcagcttcgaagcctcacagagatcttgaaaggaggggtcttgat ccagcggaacccccagctctgctaccaggacacgattttgtggaagaatatccaggagtttgctggctgcaagaagatctttgggagcct ggcatttctgccggagagctttgatggggacccagcctccaacactgccccgctccagccagagcagctccaagtgtttgagactctgga agagatcacaggttacctatacatctcagcatggccggacagcctgcctgacctcagcgtcttccagaacctgcaagtaatccggggacg aattctgcacaatggcgcctactcgctgaccctgcaagggctgggcatcagctggctggggctgcgctcactgagggaactgggcagtg gactggccctcatccaccataacacccacctctgcttcgtgcacacggtgccctgggaccagctctttcggaacccgcaccaagctctgct ccacactgccaaccggccagaggacgagtgtgtgggcgagggcctggcctgccaccagctgtgcgcccgagggcagcagaagatcc ggaagtacacgatgcggagactgctgcaggaaacggagctggtggagccgctgacacctagcggagcgatgcccaaccaggcgca gatgcggatcctgaaagagacggagctgaggaaggtgaaggtgcttggatctggcgcttttggcacagtctacaagggcatctggatcc ctgatggggagaatgtgaaaattccagtggccatcaaagtgttgagggaaaacacatcccccaaagccaacaaagaaatcttagacgaa gcatacgtgatggctggtgtgggctccccatatgtctcccgccttctgggcatctgcctgacatccacggtgcagctggtgacacagcttat gccctatggctgcctcttagactaa (SEQ ID NO: 56).

In another embodiment, the Her-2 chimeric protein has the sequence:

(SEQ ID NO: 57)
T H L D M L R H L Y Q G C Q V V Q G N L E L T Y L
P T N A S L S F L Q D I Q E V Q G Y V L I A H N Q
V R Q V P L Q R L R I V R G T Q L F E D N Y A L A
V L D N G D P L N N T T P V T G A S P G G L R E L
Q L R S L T E I L K G G V L I Q R N P Q L C Y Q D
T I L W K N I Q E F A G C K K I F G S L A F L P E
S F D G D P A S N T A P L Q P E Q L Q V F E T L E
E I T G Y L Y I S A W P D S L P D L S V F Q N L Q
V I R G R I L H N G A Y S L T L Q G L G I S W L G
L R S L R E L G S G L A L I H H N T H L C F V H T
V P W D Q L F R N P H Q A L L H T A N R P E D E C
V G E G L A C H Q L C A R G Q Q K I R K Y T M R R
L L Q E T E L V E P L T P S G A M P N Q A Q M R I
L K E T E L R K V K V L G S G A F G T V K Y G I W
I P D G E N V K I P V A I K V L R E N T S P K A N
K E I L D E A Y V M A G V G S P Y V S R L L G I C
L T S T V Q L V T Q L M P Y G C L L D.

In one embodiment, the Her2 chimeric protein or fragment thereof of the methods and compositions disclosed herein does not include a signal sequence thereof. In another embodiment, omission of the signal sequence enables the Her2 fragment to be successfully expressed in Listeria, due the high hydrophobicity of the signal sequence. Each possibility represents a separate embodiment of the disclosure.

In another embodiment, the fragment of a Her2 chimeric protein of methods and compositions of the disclosure does not include a transmembrane domain (TM) thereof. In one embodiment, omission of the TM enables the Her-2 fragment to be successfully expressed in Listeria, due the high hydrophobicity of the TM. In one embodiment, LmddA164 comprises a nucleic acid sequence comprising an open reading frame encoding tLLO fused to cHER2, wherein said nucleic acid sequence comprises SEQ ID NO: 58: atgaaaaaaataatgctagtttttattacacttatattagttagtctaccaattgcgcaacaaactgaagcaaaggatgcatctgcattcaata aagaaaattcaatttcatccatggcaccaccagcatctccgcctgcaagtcctaagacgccaatcgaaaagaaacacgcggatgaaat cgataagtatatacaaggattggattacaataaaaacaatgtattagtataccacggagatgcagtgacaaatgtgccgccaagaaaag gttacaaagatggaaatgaatatattgttgtggagaaaaagaagaaatccatcaatcaaaataatgcagacattcaagttgtgaatgcaat ttcgagcctaacctatccaggtgctctcgtaaaagcgaattcggaattagtagaaaatcaaccagatgttctccctgtaaaacgtgattcat taacactcagcattgatttgccaggtatgactaatcaagacaataaaatagttgtaaaaaatgccactaaatcaaacgttaacaacgcagt aaatacattagtggaaagatggaatgaaaaatatgctcaagcttatccaaatgtaagtgcaaaaattgattatgatgacgaaatggcttac agtgaatcacaattaattgcgaaatttggtacagcatttaaagctgtaaataatagcttgaatgtaaacttcggcgcaatcagtgaaggga aaatgcaagaagaagtcattagttttaaacaaatttactataacgtgaatgttaatgaacctacaagaccttccagatttttcggcaaagctg ttactaaagagcagttgcaagcgcttggagtgaatgcagaaaatcctcctgcatatatctcaagtgtggcgtatggccgtcaagtttatttg aaattatcaactaattcccatagtactaaagtaaaagctgcttttgatgctgccgtaagcggaaaatctgtctcaggtgatgtagaactaac aaatatcatcaaaaattcttccttcaaagccgtaatttacggaggttccgcaaaagatgaagttcaaatcatcgacggcaacctcggaga cttacgcgatattttgaaaaaaggcgctacttttaatcgagaaacaccaggagttcccattgcttatacaacaaacttcctaaaagacaatg aattagctgttattaaaaacaactcagaatatattgaaacaacttcaaaagcttatacagatggaaaaattaacatcgatcactctggagga tacgttgctcaattcaacatttcttgggatgaagtaaattatgatctcgagACCCACCTGGACATGCTCCGCCACC TCTACCAGGGCTGCCAGGTGGTGCAGGGAAACCTGGAACTCACCTACCTGCCCAC CAATGCCAGCCTGTCCTTCCTGCAGGATATCCAGGAGGTGCAGGGCTACGTGCTC ATCGCTCACAACCAAGTGAGGCAGGTCCCACTGCAGAGGCTGCGGATTGTGCGA GGCACCCAGCTCTTTGAGGACAACTATGCCCTGGCCGTGCTAGACAATGGAGACC CGCTGAACAATACCACCCCTGTCACAGGGGCCTCCCCAGGAGGCCTGCGGGAGCT GCAGCTTCGAAGCCTCACAGAGATCTTGAAAGGAGGGGTCTTGATCCAGCGGAA CCCCCAGCTCTGCTACCAGGACACGATTTTGTGGAAGAATATCCAGGAGTTTGCT GGCTGCAAGAAGATCTTTGGGAGCCTGGCATTTCTGCCGGAGAGCTTTGATGGGG ACCCAGCCTCCAACACTGCCCCGCTCCAGCCAGAGCAGCTCCAAGTGTTTGAGAC TCTGGAAGAGATCACAGGTTACCTATACATCTCAGCATGGCCGGACAGCCTGCCT GACCTCAGCGTCTTCCAGAACCTGCAAGTAATCCGGGGACGAATTCTGCACAATG GCGCCTACTCGCTGACCCTGCAAGGGCTGGGCATCAGCTGGCTGGGGCTGCGCTC ACTGAGGGAACTGGGCAGTGGACTGGCCCTCATCCACCATAACACCCACCTCTGC TTCGTGCACACGGTGCCCTGGGACCAGCTCTTTCGGAACCCGCACCAAGCTCTGC TCCACACTGCCAACCGGCCAGAGGACGAGTGTGTGGGCGAGGGCCTGGCCTGCC ACCAGCTGTGCGCCCGAGGGCAGCAGAAGATCCGGAAGTACACGATGCGGAGAC TGCTGCAGGAAACGGAGCTGGTGGAGCCGCTGACACCTAGCGGAGCGATGCCCA ACCAGGCGCAGATGCGGATCCTGAAAGAGACGGAGCTGAGGAAGGTGAAGGTGC TTGGATCTGGCGCTTTTGGCACAGTCTACAAGGGCATCTGGATCCCTGATGGGGA GAATGTGAAAATTCCAGTGGCCATCAAAGTGTTGAGGGAAAACACATCCCCCAA AGCCAACAAAGAAATCTTAGACGAAGCATACGTGATGGCTGGTGTGGGCTCCCC ATATGTCTCCCGCCTTCTGGGCATCTGCCTGACATCCACGGTGCAGCTGGTGACA CAGCTTATGCCCTATGGCTGCCTCTTAGAC (SEQ ID NO: 58), wherein the UPPERCASE sequences encode cHER2, the lowercase sequences encode tLLO and the underlined “ctcgag” sequence represents the Xho I restriction site used to ligate the tumor antigen to truncated LLO in the plasmid. In another embodiment, plasmid pAdv168 comprises SEQ ID NO: 58. In one embodiment, the truncated LLO-cHER2 fusion is a homolog of SEQ ID NO: 58. In another embodiment, the truncated LLO-cHER2 fusion is a variant of SEQ ID NO: 58. In another embodiment, the truncated LLO-cHER2 fusion is an isomer of SEQ ID NO: 58.

In one embodiment, an amino acid sequence of a recombinant protein comprising tLLO fused to a cHER2 comprises SEQ ID NO: 59: MKKIMLVFITLILVSLPIAQQTEAKDASAFNKENSISSMAPPASPPASPKTPIEKKHADE IDKYIQGLDYNKNNVLVYHGDAVTNVPPRKGYKDGNEYIVVEKKKKSINQNNADIQ VVNAISSLTYPGALVKANSELVENQPDVLPVKRDSLTLSIDLPGMTNQDNKIVVKNA TKSNVNNAVNTLVERWNEKYAQAYPNVSAKIDYDDEMAYSESQLIAKFGTAFKAV NNSLNVNFGAISEGKMQEEVISFKQIYYNVNVNEPTRPSRFFGKAVTKEQLQALGVN AENPPAYISSVAYGRQVYLKLSTNSHISTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTDGKINIDHSGGYVAQFNISWDEVNYDLETHLDMLRHLYQGCQV VQGNLELTYLPTNASLSFLQDIQEVQGYVLIAHNQVRQVPLQRLRIVRGTQLFEDNY ALAVLDNGDPLNNTTPVTGASPGGLRELQLRSLTEILKGGVLIQRNPQLCYQDTILWK NIQEFAGCKKIFGSLAFLPESFDGDPASNTAPLQPEQLQVFETLEEITGYLYISAWPDSL PDLSVFQNLQVIRGRILHNGAYSLTLQGLGISWLGLRSLRELGSGLALIHHNTHLCFV HTVPWDQLFRNPHQALLHITANRPEDECVGEGLACHQLCARGQQKIRKYTMRRLLQ TELVEPLTPSGAMPNQAQMRILKETELRKVKVLGSGAFGTVYKGIWIPDGENVKIPV AIKVLRENTSPKANKEILDEAYVMAGVGSPYVSRLLGICLTSTVQLVTQLMPYGCLL D (SEQ ID NO: 59). In one embodiment, the truncated LLO-cHER2 fusion is a homolog of SEQ ID NO: 59. In another embodiment, the truncated LLO-cHER2 fusion is a variant of SEQ ID NO: 59. In another embodiment, the truncated LLO-cHER2 fusion is an isomer of SEQ ID NO: 59.

In one embodiment, the nucleic acid sequence of human-Her2/neu gene is:

(SEQ ID NO: 60)
ATGGAGCTGGCGGCCTTGTGCCGCTGGGGGCTCCTCCTCGCCCTCTTGCC
CCCCGGAGCCGCGAGCACCCAAGTGTGCACCGGCACAGACATGAAGCTGC
GGCTCCCTGCCAGTCCCGAGACCCACCTGGACATGCTCCGCCACCTCTAC
CAGGGCTGCCAGGTGGTGCAGGGAAACCTGGAACTCACCTACCTGCCCAC
CAATGCCAGCCTGTCCTTCCTGCAGGATATCCAGGAGGTGCAGGGCTACG
TGCTCATCGCTCACAACCAAGTGAGGCAGGTCCCACTGCAGAGGCTGCGG
ATTGTGCGAGGCACCCAGCTCTTTGAGGACAACTATGCCCTGGCCGTGCT
AGACAATGGAGACCCGCTGAACAATACCACCCCTGTCACAGGGGCCTCCC
CAGGAGGCCTGCGGGAGCTGCAGCTTCGAAGCCTCACAGAGATCTTGAAA
GGAGGGGTCTTGATCCAGCGGAACCCCCAGCTCTGCTACCAGGACACGAT
TTTGTGGAAGGACATCTTCCACAAGAACAACCAGCTGGCTCTCACACTGA
TAGACACCAACCGCTCTCGGGCCTGCCACCCCTGTTCTCCGATGTGTAAG
GGCTCCCGCTGCTGGGGAGAGAGTTCTGAGGATTGTCAGAGCCTGACGCG
CACTGTCTGTGCCGGTGGCTGTGCCCGCTGCAAGGGGCCACTGCCCACTG
ACTGCTGCCATGAGCAGTGTGCTGCCGGCTGCACGGGCCCCAAGCACTCT
GACTGCCTGGCCTGCCTCCACTTCAACCACAGTGGCATCTGTGAGCTGCA
CTGCCCAGCCCTGGTCACCTACAACACAGACACGTTTGAGTCCATGCCCA
ATCCCGAGGGCCGGTATACATTCGGCGCCAGCTGTGTGACTGCCTGTCCC
TACAACTACCTTTCTACGGACGTGGGATCCTGCACCCTCGTCTGCCCCCT
GCACAACCAAGAGGTGACAGCAGAGGATGGAACACAGCGGTGTGAGAAGT
GCAGCAAGCCCTGTGCCCGAGTGTGCTATGGTCTGGGCATGGAGCACTTG
CGAGAGGTGAGGGCAGTTACCAGTGCCAATATCCAGGAGTTTGCTGGCTG
CAAGAAGATCTTTGGGAGCCTGGCATTTCTGCCGGAGAGCTTTGATGGGG
ACCCAGCCTCCAACACTGCCCCGCTCCAGCCAGAGCAGCTCCAAGTGTTT
GAGACTCTGGAAGAGATCACAGGTTACCTATACATCTCAGCATGGCCGGA
CAGCCTGCCTGACCTCAGCGTCTTCCAGAACCTGCAAGTAATCCGGGGAC
GAATTCTGCACAATGGCGCCTACTCGCTGACCCTGCAAGGGCTGGGCATC
AGCTGGCTGGGGCTGCGCTCACTGAGGGAACTGGGCAGTGGACTGGCCCT
CATCCACCATAACACCCACCTCTGCTTCGTGCACACGGTGCCCTGGGACC
AGCTCTTTCGGAACCCGCACCAAGCTCTGCTCCACACTGCCAACCGGCCA
GAGGACGAGTGTGTGGGCGAGGGCCTGGCCTGCCACCAGCTGTGCGCCCG
AGGGCACTGCTGGGGTCCAGGGCCCACCCAGTGTGTCAACTGCAGCCAGT
TCCTTCGGGGCCAGGAGTGCGTGGAGGAATGCCGAGTACTGCAGGGGCTC
CCCAGGGAGTATGTGAATGCCAGGCACTGTTTGCCGTGCCACCCTGAGTG
TCAGCCCCAGAATGGCTCAGTGACCTGTTTTGGACCGGAGGCTGACCAGT
GTGTGGCCTGTGCCCACTATAAGGACCCTCCCTTCTGCGTGGCCCGCTGC
CCCAGCGGTGTGAAACCTGACCTCTCCTACATGCCCATCTGGAAGTTTCC
AGATGAGGAGGGCGCATGCCAGCCTTGCCCCATCAACTGCACCCACTCCT
GTGTGGACCTGGATGACAAGGGCTGCCCCGCCGAGCAGAGAGCCAGCCCT
CTGACGTCCATCGTCTCTGCGGTGGTTGGCATTCTGCTGGTCGTGGTCTT
GGGGGTGGTCTTTGGGATCCTCATCAAGCGACGGCAGCAGAAGATCCGGA
AGTACACGATGCGGAGACTGCTGCAGGAAACGGAGCTGGTGGAGCCGCTG
ACACCTAGCGGAGCGATGCCCAACCAGGCGCAGATGCGGATCCTGAAAGA
GACGGAGCTGAGGAAGGTGAAGGTGCTTGGATCTGGCGCTTTTGGCACAG
TCTACAAGGGCATCTGGATCCCTGATGGGGAGAATGTGAAAATTCCAGTG
GCCATCAAAGTGTTGAGGGAAAACACATCCCCCAAAGCCAACAAAGAAAT
CTTAGACGAAGCATACGTGATGGCTGGTGTGGGCTCCCCATATGTCTCCC
GCCTTCTGGGCATCTGCCTGACATCCACGGTGCAGCTGGTGACACAGCTT
ATGCCCTATGGCTGCCTCTTAGACCATGTCCGGGAAAACCGCGGACGCCT
GGGCTCCCAGGACCTGCTGAACTGGTGTATGCAGATTGCCAAGGGGATGA
GCTACCTGGAGGATGTGCGGCTCGTACACAGGGACTTGGCCGCTCGGAAC
GTGCTGGTCAAGAGTCCCAACCATGTCAAAATTACAGACTTCGGGCTGGC
TCGGCTGCTGGACATTGACGAGACAGAGTACCATGCAGATGGGGGCAAGG
TGCCCATCAAGTGGATGGCGCTGGAGTCCATTCTCCGCCGGCGGTTCACC
CACCAGAGTGATGTGTGGAGTTATGGTGTGACTGTGTGGGAGCTGATGAC
TTTTGGGGCCAAACCTTACGATGGGATCCCAGCCCGGGAGATCCCTGACC
TGCTGGAAAAGGGGGAGCGGCTGCCCCAGCCCCCCATCTGCACCATTGAT
GTCTACATGATCATGGTCAAATGTTGGATGATTGACTCTGAATGTCGGCC
AAGATTCCGGGAGTTGGTGTCTGAATTCTCCCGCATGGCCAGGGACCCCC
AGCGCTTTGTGGTCATCCAGAATGAGGACTTGGGCCCAGCCAGTCCCTTG
GACAGCACCTTCTACCGCTCACTGCTGGAGGACGATGACATGGGGGACCT
GGTGGATGCTGAGGAGTATCTGGTACCCCAGCAGGGCTTCTTCTGTCCAG
ACCCTGCCCCGGGCGCTGGGGGCATGGTCCACCACAGGCACCGCAGCTCA
TCTACCAGGAGTGGCGGTGGGGACCTGACACTAGGGCTGGAGCCCTCTGA
AGAGGAGGCCCCCAGGTCTCCACTGGCACCCTCCGAAGGGGCTGGCTCCG
ATGTATTTGATGGTGACCTGGGAATGGGGGCAGCCAAGGGGCTGCAAAGC
CTCCCCACACATGACCCCAGCCCTCTACAGCGGTACAGTGAGGACCCCAC
AGTACCCCTGCCCTCTGAGACTGATGGCTACGTTGCCCCCCTGACCTGCA
GCCCCCAGCCTGAATATGTGAACCAGCCAGATGTTCGGCCCCAGCCCCCT
TCGCCCCGAGAGGGCCCTCTGCCTGCTGCCCGACCTGCTGGTGCCACTCT
GGAAAGGGCCAAGACTCTCTCCCCAGGGAAGAATGGGGTCGTCAAAGACG
TTTTTGCCTTTGGGGGTGCCGTGGAGAACCCCGAGTACTTGACACCCCAG
GGAGGAGCTGCCCCTCAGCCCCACCCTCCTCCTGCCTTCAGCCCAGCCTT
CGACAACCTCTATTACTGGGACCAGGACCCACCAGAGCGGGGGGCTCCAC
CCAGCACCTTCAAAGGGACACCTACGGCAGAGAACCCAGAGTACCTGGGT
CTGGACGTGCCAGTGTGAACCAGAAGGCCAAGTCCGCAGAAGCCCTGA.

In another embodiment, the nucleic acid sequence encoding the human her2/neu EC1 fragment implemented into the chimera spans from 120-510 bp of the human EC1 region and is set forth in (SEQ ID NO: 61).

(SEQ ID NO: 61)
GAGACCCACCTGGACATGCTCCGCCACCTCTACCAGGGCTGCCAGGTGGT
GCAGGGAAACCTGGAACTCACCTACCTGCCCACCAATGCCAGCCTGTCCT
TCCTGCAGGATATCCAGGAGGTGCAGGGCTACGTGCTCATCGCTCACAAC
CAAGTGAGGCAGGTCCCACTGCAGAGGCTGCGGATTGTGCGAGGCACCCA
GCTCTTTGAGGACAACTATGCCCTGGCCGTGCTAGACAATGGAGACCCGC
TGAACAATACCACCCCTGTCACAGGGGCCTCCCCAGGAGGCCTGCGGGAG
CTGCAGCTTCGAAGCCTCACAGAGATCTTGAAAGGAGGGGTCTTGATCCA
GCGGAACCCCCAGCTCTGCTACCAGGACACGATTTTGTGGAAG.

In one embodiment, the complete EC1 human her2/neu fragment spans from (58-979 bp of the human her2/neu gene and is set forth in (SEQ ID NO: 62).

(SEQ ID NO: 62)
GCCGCGAGCACCCAAGTGTGCACCGGCACAGACATGAAGCTGCGGCTCCC
TGCCAGTCCCGAGACCCACCTGGACATGCTCCGCCACCTCTACCAGGGCT
GCCAGGTGGTGCAGGGAAACCTGGAACTCACCTACCTGCCCACCAATGCC
AGCCTGTCCTTCCTGCAGGATATCCAGGAGGTGCAGGGCTACGTGCTCAT
CGCTCACAACCAAGTGAGGCAGGTCCCACTGCAGAGGCTGCGGATTGTGC
GAGGCACCCAGCTCTTTGAGGACAACTATGCCCTGGCCGTGCTAGACAAT
GGAGACCCGCTGAACAATACCACCCCTGTCACAGGGGCCTCCCCAGGAGG
CCTGCGGGAGCTGCAGCTTCGAAGCCTCACAGAGATCTTGAAAGGAGGGG
TCTTGATCCAGCGGAACCCCCAGCTCTGCTACCAGGACACGATTTTGTGG
AAGGACATCTTCCACAAGAACAACCAGCTGGCTCTCACACTGATAGACAC
CAACCGCTCTCGGGCCTGCCACFCCCTGTTCTCCGATGTGTAAGGGCTCC
CGCTGCTGGGGAGAGAGTTCTGAGGATTGTCAGAGCCTGACGCGCACTGT
CTGTGCCGGTGGCTGTGCCCGCTGCAAGGGGCCACTGCCCACTGACTGCT
GCCATGAGCAGTGTGCTGCCGGCTGCACGGGCCCCAAGCACTCTGACTGC
CTGGCCTGCCTCCACTTCAACCACAGTGGCATCTGTGAGCTGCACTGCCC
AGCCCTGGTCACCTACAACACAGACACGTTTGAGTCCATGCCCAATCCCG
AGGGCCGGTATACATTCGGCGCCAGCTGTGTGACTGCCTGTCCCTACAAC
TACCTTTCTACGGACGTGGGATCCTGCACCCTCGTCTGCCCCCTGCACAA
CCAAGAGGTGACAGCAGAGGAT.

In another embodiment, the nucleic acid sequence encoding the human her2/neu EC2 fragment implemented into the chimera spans from 1077-1554 bp of the human her2/neu EC2 fragment and includes a 50 bp extension, and is set forth in (SEQ ID NO: 63).

(SEQ ID NO: 63)
AATATCCAGGAGTTTGCTGGCTGCAAGAAGATCTTTGGGAGCCTGGCATT
TCTGCCGGAGAGCTTTGATGGGGACCCAGCCTCCAACACTGCCCCGCTCC
AGCCAGAGCAGCTCCAAGTGTTTGAGACTCTGGAAGAGATCACAGGTTAC
CTATACATCTCAGCATGGCCGGACAGCCTGCCTGACCTCAGCGTCTTCCA
GAACCTGCAAGTAATCCGGGGACGAATTCTGCACAATGGCGCCTACTCGC
TGACCCTGCAAGGGCTGGGCATCAGCTGGCTGGGGCTGCGCTCACTGAGG
GAACTGGGCAGTGGACTGGCCCTCATCCACCATAACACCCACCTCTGCTT
CGTGCACACGGTGCCCTGGGACCAGCTCTTTCGGAACCCGCACCAAGCTC
TGCTCCACACTGCCAACCGGCCAGAGGACGAGTGTGTGGGCGAGGGCCTG
GCCTGCCACCAGCTGTGCGCCCGAGGG.

In one embodiment, complete EC2 human her2/neu fragment spans from 907-1504 bp of the human her2/neu gene and is set forth in (SEQ ID NO: 64).

(SEQ ID NO: 64)
TACCTTTCTACGGACGTGGGATCCTGCACCCTCGTCTGCCCCCTGCACAA
CCAAGAGGTGACAGCAGAGGATGGAACACAGCGGTGTGAGAAGTGCAGCA
AGCCCTGTGCCCGAGTGTGCTATGGTCTGGGCATGGAGCACTTGCGAGAG
GTGAGGGCAGTTACCAGTGCCAATATCCAGGAGTTTGCTGGCTGCAAGAA
GATCTTTGGGAGCCTGGCATTTCTGCCGGAGAGCTTTGATGGGGACCCAG
CCTCCAACACTGCCCCGCTCCAGCCAGAGCAGCTCCAAGTGTTTGAGACT
CTGGAAGAGATCACAGGTTACCTATACATCTCAGCATGGCCGGACAGCCT
GCCTGACCTCAGCGTCTTCCAGAACCTGCAAGTAATCCGGGGACGAATTC
TGCACAATGGCGCCTACTCGCTGACCCTGCAAGGGCTGGGCATCAGCTGG
CTGGGGCTGCGCTCACTGAGGGAACTGGGCAGTGGACTGGCCCTCATCCA
CCATAACACCCACCTCTGCTTCGTGCACACGGTGCCCTGGGACCAGCTCT
TTCGGAACCCGCACCAAGCTCTGCTCCACACTGCCAACCGGCCAGAG.

In another embodiment, the nucleic acid sequence encoding the human her2/neu IC1 fragment implemented into the chimera is set forth in (SEQ ID NO: 65).

(SEQ ID NO: 65)
CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACT.

In another embodiment, the nucleic acid sequence encoding the complete human her2/neu IC1 fragment spans from 2034-3243 of the human her2/neu gene and is set forth in (SEQ ID NO: 66).

(SEQ ID NO: 66)
CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACCATGTCCGGG
AAAACCGCGGACGCCTGGGCTCCCAGGACCTGCTGAACTGGTGTATGCAG
ATTGCCAAGGGGATGAGCTACCTGGAGGATGTGCGGCTCGTACACAGGGA
CTTGGCCGCTCGGAACGTGCTGGTCAAGAGTCCCAACCATGTCAAAATTA
CAGACTTCGGGCTGGCTCGGCTGCTGGACATTGACGAGACAGAGTACCAT
GCAGATGGGGGCAAGGTGCCCATCAAGTGGATGGCGCTGGAGTCCATTCT
CCGCCGGCGGTTCACCCACCAGAGTGATGTGTGGAGTTATGGTGTGACTG
TGTGGGAGCTGATGACTTTTGGGGCCAAACCTTACGATGGGATCCCAGCC
CGGGAGATCCCTGACCTGCTGGAAAAGGGGGAGCGGCTGCCCCAGCCCCC
CATCTGCACCATTGATGTCTACATGATCATGGTCAAATGTTGGATGATTG
ACTCTGAATGTCGGCCAAGATTCCGGGAGTTGGTGTCTGAATTCTCCCGC
ATGGCCAGGGACCCCCAGCGCTTTGTGGTCATCCAGAATGAGGACTTGGG
CCCAGCCAGTCCCTTGGACAGCACCTTCTACCGCTCACTGCTGGAGGACG
ATGACATGGGGGACCTGGTGGATGCTGAGGAGTATCTGGTACCCCAGCAG
GGCTTCTTCTGTCCAGACCCTGCCCCGGGCGCTGGGGGCATGGTCCACCA
CAGGCACCGCAGCTCATCTACCAGGAGTGGCGGTGGGGACCTGACACTAG
GGCTGGAGCCCTCTGAAGAGGAGGCCCCCAGGTCTCCACTGGCACCCTCC
GAAGGGGCT.

Point mutations or amino-acid deletions in the oncogenic protein Her2/neu, have been reported to mediate treatment of resistant tumor cells, when these tumors have been targeted by small fragment Listeria-based vaccines or trastuzumab (a monoclonal antibody against an epitope located at the extracellular domain of the Her2/neu antigen). Described herein is a chimeric Her2/neu based composition which harbors two of the extracellular and one intracellular fragments of Her2/neu antigen showing clusters of MHC-class I epitopes of the oncogene. This chimeric protein, which harbors 3 H2Dq and at least 17 of the mapped human MHC-class I epitopes of the Her2/neu antigen was fused to the first 441 amino acids of the Listeria-monocytogenes listeriolysin O protein and expressed and secreted by the Listeria monocytogenes attenuated strain LmddA.

In another embodiment, the antigen of interest is a KLK9 polypeptide.

In another embodiment, the tumor-associated antigen is HPV-E7. In another embodiment, the antigen is HPV-E6. In another embodiment, the antigen is Her-2. In another embodiment, the antigen is NY-ESO-1. In another embodiment, the antigen is telomerase. In another embodiment, the antigen is SCCE. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from E7, E6, Her-2, NY-ESO-1, telomerase, SCCE, WT-1, HIV-1 Gag, Proteinase 3, Tyrosinase related protein 2, PSA (prostate-specific antigen). In another embodiment, the antigen is a tumor-associated antigen. In another embodiment, the antigen is an infectious disease antigen.

In another embodiment, the tumor-associated antigen is an angiogenic antigen. In another embodiment, the angiogenic antigen is expressed on both activated pericytes and pericytes in tumor angiogeneic vasculature, which in another embodiment, is associated with neovascularization in vivo. In another embodiment, the angiogenic antigen is HMW-MAA. In another embodiment, the angiogenic antigen is one known in the art and are provided in WO2010/102140, which is incorporated by reference herein.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, mesothelin, EGFRVIII or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment, the heterologous antigen disclosed herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods as disclosed herein are melanoma-associated antigens, which in one embodiment are TRP-2, MAGE-1, MAGE-3, gp-100, tyrosinase, HSP-70, beta-HCG, or a combination thereof. In another embodiment, the tumor associated antigen is an angiogenic antigen.

In another embodiment, the heterologous antigen is an infectious disease antigen. In one embodiment, the antigen is an auto antigen or a self-antigen.

In another embodiment, the heterologous antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In another embodiments, the heterologous antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment of the methods and compositions as disclosed herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

In one embodiment, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example.

In another embodiment, the construct or nucleic acid molecule disclosed herein is integrated into the Listerial chromosome using homologous recombination. Techniques for homologous recombination are well known in the art, and are described, for example, in Baloglu S, Boyle S M, et al. (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the disclosure further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current disclosure enables the establishment of a phage based chromosomal integration system that does not require selection with antibiotics. Instead, an auxotrophic host strain can be complemented.

In one embodiment of the methods and compositions as disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions as disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the disclosure provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” as used herein describes an item that has developed or originated within the reference organism or arisen from causes within the reference organism. In another embodiment, endogenous refers to native.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the disclosure.

In another embodiment, a recombinant Listeria strain of the methods and compositions as disclosed herein comprise a nucleic acid molecule operably integrated into the Listeria genome as an open reading frame with an endogenous ActA sequence. In another embodiment, a recombinant Listeria strain of the methods and compositions as disclosed herein comprise an episomal expression vector comprising a nucleic acid molecule encoding fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387.

In another embodiment, a “functional fragment” is an immunogenic fragment and elicits an immune response when administered to a subject alone or in a vaccine composition disclosed herein. In another embodiment, a functional fragment has biological activity as will be understood by a skilled artisan and as further disclosed herein.

The recombinant Listeria strain of methods and compositions of the disclosure is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, a recombinant Listeria strain of the disclosure has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art.

In another embodiment, a recombinant nucleic acid of the disclosure is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter.

In another embodiment, inducible and tissue specific expression of the nucleic acid encoding a peptide of the disclosure is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the disclosure includes the use of any promoter/regulatory sequence, which is either known or unknown, and which is capable of driving expression of the desired protein operably linked thereto. It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein”, “heterologous protein antigen”, “protein antigen”, “antigen”, and the like.

It will be appreciated by the skilled artisan that the term “episomal expression vector” encompasses a nucleic acid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P. B. G. M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as disclosed herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule.

In another embodiment, conjugation is used to introduce genetic material and/or plasmids into bacteria. Methods for conjugation are well known in the art, and are described, for example, in Nikodinovic J. et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9).

In one embodiment, the term “attenuation,” refers to a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the disclosure are therefore environmentally safe in that they are incapable of uncontrolled replication.

### Compositions

In one embodiment, compositions of the disclosure are immunogenic compositions. In one embodiment, compositions of the disclosure induce a strong innate stimulation of interferon-gamma, which in one embodiment, has anti-angiogenic properties. In one embodiment, a Listeria of the disclosure induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J. Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, administration of compositions of the disclosure induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the disclosure is involved in the release of anti-angiogenic factors, and, therefore, in one embodiment, has a therapeutic role in addition to its role as a vector for introducing an antigen to a subject. The immune response induced by methods and compositions as disclosed herein is, in another embodiment, a T cell response. In another embodiment, the immune response comprises a T cell response. In another embodiment, the response is a CD8+ T cell response. In another embodiment, the response comprises a CD8⁺ T cell response.

As used throughout, the terms “composition” and “immunogenic composition” are interchangeable having all the same meanings and qualities. The term “pharmaceutical composition” refers, in some embodiments, to a composition suitable for pharmaceutical use, for example, to administer to a subject in need.

Compositions disclosed herein may be used in methods disclosed herein in order to elicit an enhanced anti-tumor T cell response in a subject, in order to inhibit tumor-medicated immunosuppression in a subject, or for increasing the ratio or T effector cells to regulatory T cells (Tregs) in the spleen and tumor of a subject, or any combination thereof.

In another embodiment, a composition comprising a Listeria strain of the disclosure further comprises an adjuvant. In one embodiment, a composition of the disclosure further comprises an adjuvant. The adjuvant utilized in methods and compositions of the disclosure is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In one embodiment, an immunogenic composition disclosed herein comprises a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof. In one embodiment, an immunogenic composition disclosed herein comprises a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, said composition further comprising an additional active agent. In one embodiment said additional active agent comprises an oncolytic virus. In another embodiment, the additional active agent comprises a T cell receptor engineered T cell (Receptor engineered T cells). In another embodiment, the additional active agent comprises a chimeric antigen receptor engineered cells (CAR T cells). In another embodiment, the additional active agent comprises a therapeutic or immunomodulating monoclonal antibody. In another embodiment, the additional active agent comprises a targeting thymidine kinase inhibitor (TKI). In another embodiment, the additional active agent comprises an adoptively transferred cell incorporating engineered T cell receptors. In another embodiment, an additional active agent disclosed herein comprises an attenuated oncolytic virus, a T cell receptor engineered T cell (Receptor engineered T cells), a chimeric antigen receptor engineered T cell (CAR T cells), a therapeutic or immunomodulating monoclonal antibody, a targeting thymidine kinase inhibitor (TKI), or an adoptively transferred cells incorporating engineered T cell receptors, or any combination thereof.

In one embodiment, the Receptor engineered T cells comprises a receptor engineered to have a selected specificity. In another embodiment, both polypeptides of the engineered receptor have been recombinantly engineered to have a selected specificity. In one embodiment, selected specificity is to cell-surface tumor ligands.

In one embodiment, the Receptor engineered T cells are autologous. In another embodiment, the Receptor engineered T cells are allogeneic.

In one embodiment, the CAR T cell is an autologous CAR T cell. In another embodiment, the CAR T cell is allogeneic. In another embodiment, the CART T cell is a single-source CAR T cell. In another embodiment, the CAR T cell is an autologous HLA masked CAR T cell. In another embodiment, the CAR T cell is an autologous HLA deleted CAR T cell. In another embodiment, the CAR T cell is a single-source HLA masked CAR T cell. In another embodiment, the CART T cell is a single-source HLA deleted CAR T cell.

In one embodiment, an additional active agent is an oncolytic virus. The term “oncolytic virus” refers in one embodiment to a genetically engineered virus capable of selectively replicating in and slowing the growth of or inducing the death of a cancerous or hyperproliferative cell, either in vitro or in vivo, while having no or minimal effect on normal cells.

In one embodiment, the oncolytic virus is selected from the group comprising a vesicular stomatitis virus (VSV), a newcastle disease virus (NDV), a retrovirus, a reovirus, a measles virus, a sinbis virus, an influenza virus, a herpes simplex virus, a vaccinia virus, and an adenovirus. In one embodiment, the oncolytic virus infects tumor cells. In some embodiments, the oncolytic virus infects prostate tumor cells. In other embodiments, the oncolytic virus infects cervical cancer tumor cells. In one embodiment, an oncolytic virus comprises a nucleic acid sequence encoding a heterologous antigen. In one embodiment, the heterologous antigen is a tumor associated antigen or fragment thereof. In one embodiment, the heterologous antigen is a PSA antigen or fragment thereof, a HPV antigen or a fragment thereof or a chimeric Her-2/neu antigen or fragment thereof. In another embodiment, the heterologous antigen is a programmed cell death receptor (PD-1) binding agonist or antagonist.

In one embodiment, the therapeutic or immunomodulating monoclonal antibody recognizes an epitope of said heterologous antigen present on the surface of a cancer cell. In one embodiment, the heterologous antigen is a tumor associated antigen. In one embodiment, the heterologous antigen is a PSA antigen, a HPV antigen, or a chimeric Her-2/neu antigen. In one embodiment, the monoclonal antibody recognizes a PSA epitope. In one embodiment, the monoclonal antibody recognizes an HPV epitope. In one embodiment, the monoclonal antibody recognizes a Her-2/neu epitope. In another embodiment, the monoclonal antibody recognizing a Her-2/neu epitope comprises trastuzuman (trademarked as Heceptin®), panitumumab, or any other known in the art to recognize a Her-2/neu epitope. In another embodiment, the therapeutic or immunomodulating monoclonal antibody recognizes an epitope that is not present on said heterologous antigen.

In some embodiments, the term “antibody” refers to intact molecules as well as functional fragments thereof, such as Fab, F(ab′)2, and Fv that are capable of specifically interacting with a desired target as described herein, for example, binding to phagocytic cells. In some embodiments, the antibody fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

In some embodiments, the antibody fragments may be prepared by proteolytic hydrolysis of the antibody or by expression in E. coli or mammalian cells (e.g. Chinese hamster ovary cell culture or other protein expression systems) of DNA encoding the fragment.

Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In some embodiments, the antibodies or fragments described herein comprise “humanized forms” of antibodies. In some embodiments, the term “humanized forms of antibodies” refers to non-human (e.g. murine) antibodies, which are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The term “epitope” or antigenic determinant” refers to a site on an antigen to which an immunoglobulin or antibody, or fragment thereof, specifically binds. Epitopes can be formed both from contiguous amino acids or noncontiguous amino acids juxtaposed by tertiary folding of a protein. Epitopes formed from continuous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, or 15 amino acids in a unique spatial conformation. Methods of determining spatial conformation of epitopes include, for example, x-ray crystallography and 2-dimensional nuclear magnetic resonance.

In one embodiment, compositions disclosed herein comprise a therapeutic or immunomodulating monoclonal antibody. In another embodiment, a composition disclosed herein comprises an Lm strain and a therapeutic or immunomodulating monoclonal antibody. In another embodiment, a composition disclosed herein comprises a therapeutic or immunomodulating monoclonal antibody, wherein the composition does not include a Listeria strain disclosed herein.

In one embodiment, the thymidine kinase inhibitor comprises imatinib mesylate (IM), dasatinib (D), nilotinib (N) bosutinib (B) or INNO 406, or any combination thereof.

In one embodiment, compositions disclosed herein comprise a targeting thymidine kinase inhibitor (TKI). In another embodiment, a composition disclosed herein comprises an Lm strain and a targeting thymidine kinase inhibitor (TKI). In another embodiment, a composition disclosed herein comprises a targeting thymidine kinase inhibitor (TKI), wherein the composition does not include a Listeria strain disclosed herein.

In one embodiment, the disease disclosed herein is a cancer or a tumor. In one embodiment, the cancer treated by a method of the disclosure is breast cancer. In another embodiment, the cancer is a cervical cancer. In another embodiment, the cancer is an Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiform. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. In another embodiment, the cancer is mesothelioma.

In one embodiment the heterologous antigen is PD-1 or an immunogenic fragment thereof. In another embodiment, the heterologous antigen is a PD-1 antagonist. In one embodiment, the PD-1 antagonist is selected from the group comprising an antibody or fragment thereof, an PD-1 antagonist or a fragment thereof, or a PD-1 partial antagonist or fragment thereof, or any combination thereof.

In another embodiment, the antigen is HPV-E7. In another embodiment, the antigen is HPV-E6. In another embodiment, the antigen is Her-2/neu. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is p53. In another embodiment, the antigen is carboxic anhydrase IX (CAIX). In another embodiment, the antigen is PSMA. In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from HPV-E7, HPV-E6, Her-2, NY-ESO-1, telomerase (TERT), SCCE, HMW-MAA, EGFR-III, survivin, baculoviral inhibitor of apoptosis repeat-containing 5 (BIRC5), WT-1, HIV-1 Gag, CEA, LMP-1, p53, PSMA, PSCA, Proteinase 3, Tyrosinase related protein 2, Muc1, PSA (prostate-specific antigen), or a combination thereof.

In another embodiment, an “immunogenic fragment” is one that elicits an immune response when administered to a subject alone or in a vaccine composition disclosed herein. Such a fragment contains, in another embodiment, the necessary epitopes in order to elicit either a humoral immune response, and/or an adaptive immune response.

In one embodiments, the heterologous antigen expressed in an oncolytic virus comprises the same or nearly the same heterologous antigen or fragment thereof expressed in a Lm strain disclosed herein. For example, an Lm strain disclosed herein comprising a fusion polypeptide comprising a heterologous antigen or fragment thereof, may comprise the same heterologous antigen or fragment thereof as is expressed from an oncolytic virus disclosed herein. In another embodiment, while the heterologous antigen may be the same, the fragment of the heterologous antigen may be different or may include different domains of the heterologous antigen. For example, the Lm strain might express an N-terminal region of an antigen, while the oncolytic virus expresses the C-terminal region of the same antigen, or vice versa.

In one embodiment, the oncolytic virus infects a tumor cell.

In one embodiment, an oncolytic virus infects a PSA overexpressing tumor cell. In one embodiment, an oncolytic virus infects a prostate tumor cell. In another embodiment, an oncolytic virus infects a precursor of a prostate tumor cell. In another embodiment, an oncolytic virus infects an HPV overexpressing tumor cell. In another embodiment, an oncolytic virus infects a cervical cancer tumor cell. In another embodiment, an oncolytic virus infects a precursor of a cervical cancer cell. In yet another embodiment, an oncolytic virus infects an Her2/neu over-expressing tumor cell. In yet another embodiment, an oncolytic virus infects a osteosarcoma or Ewing's sarcoma (ES) cell.

In one embodiment, an oncolytic virus disclosed herein expresses a programmed cell death receptor (PD-1) binding agonist or antagonist. In one embodiment, a PD-1 antagonist is selected from the group comprising an antibody or a fragment thereof, a PD-1 antagonist, or a PD-1 partial antagonist, or any combination thereof. It will be well appreciated by the skilled artisan that the term “PD-1” is an acronym for the Programmed Cell Death 1 protein, a 50-55 kDa type I transmembrane receptor originally identified by subtractive hybridization of a mouse T cell line undergoing apoptosis (Ishida et al., 1992, Embo J. 1 1:3887-95). PD-1 is expressed on activated T, B, and myeloid lineage cells (Greenwald ef al., 2005, Annu. Rev. Immunol. 23:515-48; Sharpe et al., 2007, Nat. Immunol. 8:239-45). The amino acid sequence of human PD-1 is GenBank Accession No. NP_005009.2. The amino acid sequence of murine PD-1 is GenBank Accession No. AAI 19180.1.

In one embodiment, oncolytic viruses that target tumor cells lead to tumor cell death.

In one embodiment, compositions disclosed herein comprise an oncolytic virus. In another embodiment, a composition disclosed herein comprises an Lm strain and an oncolytic virus. In another embodiment, a composition disclosed herein comprises an oncolytic virus, wherein the composition does not include a Listeria strain disclosed herein.

In one embodiment, an additional active agent of the compositions disclosed herein is a T cell receptor engineered T cell (Receptor engineered T cells). In another embodiment, T cells are transduced to express a receptor engineered for selected specificity. The receptors of Receptor engineered T cells are molecules that exhibit a specific tumor specificity. In one embodiment, the genetically modified receptors of Receptor engineered T cells have selected specificity to an human HPV tumor ligand, a PSA tumor ligand or a Her-2/neu tumor ligand.

The Receptor engineered cells of the disclosure are genetically modified to stably express a desired genetically modified T cell receptor. In one embodiment, the T cell is genetically modified to stably express a modified receptor on its surface, conferring novel tumor specificity.

In one embodiment, Receptor engineered T cells comprise a nucleic acid that encodes a receptor that would recognize a tumor cell-surface ligand. In one embodiment, Receptor engineered T cells express a receptor that recognizes a tumor cell-surface ligand. In one embodiment, Receptor engineered T cells express a receptor that binds to a tumor cell-surface ligand. In one embodiment, a tumor cell-surface ligand comprises an HPV tumor cell-surface ligand, or a portion thereof. In another embodiment, a tumor cell-surface ligand comprises a PSA tumor cell-surface ligand, or a portion thereof. In another embodiment, a tumor cell-surface ligand comprises a Her-2/neu tumor cell-surface ligand or a portion thereof.

In one embodiment, the genetically modified receptor of a Receptor engineered T cell binds to a prostate specific antigen (PSA) cell-surface ligand domain or a fragment thereof, a human papilloma virus (HPV) cell-surface ligand domain or a fragment thereof, or a chimeric Her2/neu cell-surface ligand domain or a fragment thereof. In another embodiment, the genetically modified receptor of a Receptor engineered T cell has selective binding specificity to the same or nearly the same tumor cell-surface ligand or fragment thereof expressed in a Lm strain disclosed herein as a heterologous antigen. For example, an Lm strain disclosed herein comprising a fusion polypeptide comprising a heterologous antigen or fragment thereof, may comprise the same heterologous antigen or fragment thereof as is specifically recognized by Receptor engineered T cells disclosed herein. While the heterologous antigen of an Lm strain disclosed herein may comprise the same the antigen recognized by Receptor engineered T cells disclosed herein, the actual binding specificity recognized by the receptors of Receptor engineered T cells may not be included within the heterologous antigen expressed from the Lm strain. For example, the Lm strain might express an N-terminal region of an antigen, while the Receptor engineered T cells have selective specificity to the C-terminal region of the same antigen, or vice versa.

In one embodiment, compositions disclosed herein comprise Receptor engineered T cells. In one embodiment, a composition disclosed herein comprises an Lm strain and Receptor engineered T cells. In another embodiment, a composition disclosed herein comprises Receptor engineered T cells, wherein the composition does not include a Listeria strain as described herein.

In one embodiment, a composition disclosed herein comprises a recombinant Listeria monocytogenes (Lm) strain.

In one embodiment, an immunogenic composition comprises Receptor engineered T cells disclosed herein, and a recombinant attenuated Listeria strain disclosed herein. In another embodiment, each component of the immunogenic compositions disclosed herein is administered prior to, concurrently with, of after another component of the immunogenic compositions disclosed herein. In one embodiment, even when administered concurrently, an composition comprising Lm and a composition comprising Receptor engineered T cells may be administered as two separate compositions. In another embodiment, even when administered concurrently, an Lm composition and a Receptor engineered T cells composition may be administered as two separate compositions. In yet another embodiment, an Lm composition comprises Receptor engineered T cells.

In another embodiment, an additional active agent of the compositions disclosed herein is a chimeric antigen receptor engineered T cells (CAR T cells). In one embodiment, T cells are transduced to express a chimeric antigen receptor (CAR). CARs are molecules that combine antibody-based specificity for a desired antigen (e.g., tumor antigen) with a T cell receptor-activating intracellular domain to generate a chimeric protein that exhibits a specific anti-tumor cellular immune activity.

The CAR T cells of the disclosure are genetically modified to stably express a desired CAR. In one embodiment, the T cell is genetically modified to stably express an antibody binding domain on its surface, conferring novel antigen specificity that is MHC independent.

In one embodiment, the antigen recognized by CAR T cells is a tumor associated antigen. In one embodiment, the antigen specificity is for a PSA antigen or an immunogenic fragment thereof. In another embodiment, the antigen specificity is for a HPV antigen or an immunogenic fragment thereof. In another embodiment, the antigen specificity is for a tumor-associated antigen as disclosed herein or an immunogenic fragment thereof.

In yet another embodiment, the antigen specificity is for a chimeric Her-2/neu antigen or fragment thereof. In another embodiment, CAR T cells comprise a nucleic acid encoding a polypeptide that specifically recognizes the tumor associated antigen. In another embodiment, the polypeptide comprises an antigen binding domain. In another embodiment, the antigen binding domain comprises an antibody or an antigen binding domain thereof.

The term “antibody fragment” refers to a portion of an intact antibody that is capable of specifically binding to an antigen. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, K and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, CAR T cells comprise a nucleic acid that encodes an antigen binding region. In one embodiment, CAR T cells express an antigen binding region. In one embodiment, an antigen binding regions is an antibody or an antigen-binding domain thereof. In one embodiment, the antigen-binding domain thereof is a Fab or a scFv.

It will be appreciated by a skilled artisan that the term “specifically binds,” with respect to an antibody, encompasses an antibody which recognizes a specific antigen, but does not substantially recognize or bind other molecules in a sample. For example, an antibody that specifically binds to an antigen from one species may also bind to that antigen from one or more species, but, such cross-species reactivity does not itself alter the classification of an antibody as specific, in another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than a specific amino acid sequence. I

In one embodiment, the antigen binding domain of a CAR binds to a prostate specific antigen (PSA) domain or a fragment thereof, a human papilloma virus (HPV) antigen domain or a fragment thereof, or a chimeric Her2/neu antigen domain or a fragment thereof. In another embodiment, the antigen binding domain of a CAR specifically recognizes the same or nearly the same heterologous antigen or fragment thereof expressed in a Lm strain disclosed herein. For example, an Lm strain disclosed herein comprising a fusion polypeptide comprising a heterologous antigen or fragment thereof, may comprise the same heterologous antigen or fragment thereof as is specifically recognized by CAR T cells disclosed herein. While the heterologous antigen of an Lm strain disclosed herein may comprise the same the antigen recognized by CAR T cells disclosed herein, the actual antigen epitope recognized by the CAR T cells may not be included within the heterologous antigen expressed from the Lm strain. For example, the Lm strain might express an N-terminal region of an antigen, while the CAR T cells specifically recognize an epitope within the C-terminal region of the same antigen, or vice versa.

In one embodiment, compositions disclosed herein comprise CAR T cells. In one embodiment, a composition disclosed herein comprises an Lm strain and CAR T cells. In another embodiment, a composition disclosed herein comprises CAR T cells, wherein the composition does not include a Listeria strain as described herein.

In one embodiment, a composition disclosed herein comprises a recombinant Listeria monocytogenes (Lm) strain.

In one embodiment, an immunogenic composition comprises an oncolytic virus disclosed herein and/or a chimeric antigen receptor engineered cells (CAR T cells) disclosed herein, and a recombinant attenuated Listeria disclosed herein. In another embodiment, each component of the immunogenic compositions disclosed herein is administered prior to, concurrently with, of after another component of the immunogenic compositions disclosed herein. In one embodiment, even when administered concurrently, an Lm composition and CAR T cells may be administered as two separate compositions. In another embodiment, even when administered concurrently, an Lm composition and CAR T cells may be administered as two separate compositions. Alternately, in another embodiment, an Lm composition may comprise an CAR T cells. In yet another embodiment, an Lm composition comprises CAR T cells.

The compositions disclosed herein, in another embodiment, are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the disclosure, the active ingredient is formulated in a capsule. In accordance with this embodiment, the compositions of the disclosure comprise, in addition to the active compound and the inert carrier or diluent, a hard gelating capsule.

In another embodiment, compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In some embodiments, when the oncolytic virus is administered separately from a composition comprising a recombinant Lm strain, the oncolytic viruses may be injected intravenously, subcutaneously, or directly into the tumor or tumor bed. In one embodiment, a composition comprising an oncolytic virus is injected into the space left after a tumor has been surgically removed, e.g., the space in a prostate gland following removal of a prostate tumor.

In some embodiments, when the Receptor engineered T cells are administered separately from a composition comprising a recombinant Lm strain, the Receptor engineered T cells may be injected intravenously, subcutaneously, or directly into the tumor or tumor bed. In one embodiment, a composition comprising Receptor engineered T cells is injected into the space left after a tumor has been surgically removed, e.g., the space in a prostate gland following removal of a prostate tumor.

In some embodiments, when the CAR T cells are administered separately from a composition comprising a recombinant Lm strain, the CAR T cells may be injected intravenously, subcutaneously, or directly into the tumor or tumor bed. In one embodiment, a composition comprising CAR T cells is injected into the space left after a tumor has been surgically removed, e.g., the space in a prostate gland following removal of a prostate tumor.

In some embodiments, when the monoclonal antibodies are administered separately from a composition comprising a recombinant Lm strain, the monoclonal antibodies may be injected intravenously, subcutaneously, or directly into the tumor or tumor bed. In one embodiment, a composition comprising monoclonal antibodies is injected into the space left after a tumor has been surgically removed, e.g., the space in a prostate gland following removal of a prostate tumor.

In some embodiments, when the TKI is administered separately from a composition comprising a recombinant Lm strain, the TKI may be injected intravenously, subcutaneously, or directly into the tumor or tumor bed. In one embodiment, a composition comprising TKI is injected into the space left after a tumor has been surgically removed, e.g., the space in a prostate gland following removal of a prostate tumor.

In one embodiment, the term “immunogenic composition” encompasses the recombinant Listeria disclosed herein, and an adjuvant, an oncolytic virus, a chimeric antigen receptor engineered cells (CAR T cells), a therapeutic or immunomodulatory monoclonal antibody, a targeting thymidine kinase inhibitor (TKI), or Receptor engineered T cells, or any combination thereof. In another embodiment, an immunogenic composition comprises a recombinant Listeria disclosed herein. In another embodiment, an immunogenic composition comprises an adjuvant known in the art or as disclosed herein. It is also to be understood that administration of such compositions enhance an immune response, or increase a T effector cell to regulatory T cell ratio or elicit an anti-tumor immune response, as further disclosed herein.

In one embodiment, this disclosure provides methods of use which comprise administering a composition comprising the described Listeria strains, and further comprising additional agents such as oncolytic viruses, CAR T cells, a therapeutic or immunomodulatory monoclonal antibody, a targeting thymidine kinase inhibitor (TKI), or Receptor engineered T cells. In one embodiment, the term “pharmaceutical composition” encompasses a therapeutically effective amount of the active ingredient or ingredients including the Listeria strain, the oncolytic viruses, the CAR T cells), the therapeutic or immunomodulatory monoclonal antibody, the targeting thymidine kinase inhibitor (TKI), or Receptor engineered T cells, together with a pharmaceutically acceptable carrier or diluent. It is to be understood that the term a “therapeutically effective amount” refers to that amount which provides a therapeutic effect for a given condition and administration regimen.

It will be understood by the skilled artisan that the term “administering” encompasses bringing a subject in contact with a composition of the disclosure. In one embodiment, administration can be accomplished in vitro, i.e. in a test tube, or in vivo, i.e. in cells or tissues of living organisms, for example humans. In one embodiment, the disclosure encompasses administering the Listeria strains and compositions thereof of the disclosure to a subject.

The term “about” as used herein means in quantitative terms plus or minus 5%, or in another embodiment plus or minus 10%, or in another embodiment plus or minus 15%, or in another embodiment plus or minus 20%. It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

Following the administration of the immunogenic compositions disclosed herein, the methods disclosed herein induce the expansion of T effector cells in peripheral lymphoid organs leading to an enhanced presence of T effector cells at the tumor site. In another embodiment, the methods disclosed herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs. It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites.

### Combination Therapies and Methods of Use Thereof

In one embodiment, this disclosure provides a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof, wherein (a) said composition further comprises an additional active agent; (b) said method further comprises a step of administering an effective amount of a composition comprising an additional active agent to said subject; or (c) said method further comprises a step of administering a targeted radiation therapy to said subject; or any combination thereof of (a)-(c).

In one embodiment, any composition comprising a Listeria strain described herein may be used in the methods disclosed herein. In one embodiment, any composition comprising a Listeria strain and an additional active agent, for example an oncolytic virus, chimeric antigen receptor engineered cells (CAR T cells), a therapeutic or immunomodulatory monoclonal antibody, a targeting thymidine kinase inhibitor (TKI), or adoptively transferred cells incorporating engineered T cell receptors, or any combination thereof, described herein may be used in the methods disclosed herein. In one embodiment, any composition comprising an additional agent described herein may be used in the methods disclosed herein. Compositions comprising Listeria strains with and without additional agents have been described in detail above. Compositions with additional agents have also been described in detail above. In some embodiment, in a method disclosed herein a composition comprising an additional active agent, for example an oncolytic virus, CAR T cells, a therapeutic or immunomodulatory monoclonal antibody, a targeting thymidine kinase inhibitor (TKI), or adoptively transferred cells incorporating engineered T cell receptors may be administered prior to, concurrent with or following administration of a composition comprising a Listeria strain.

Radiation therapy (RT) is a local therapy and does not demonstrate the systemic toxicity and immunosuppressive effects of chemotherapy. There are sufficient pre-clinical evidence showing that RT induces an immunogenic cell death by increasing tumor neo-antigen presentation in MHC I on tumor cell surface, by increasing translocation of calreticulin, and by inducing the secretion of HMGB1.

In one embodiment, “radiation therapy” is a term commonly used in the art to refer to multiple types of radiation therapy including internal and external radiation therapy, radioimmunotherapy, and the use of various types of radiation including X-rays, gamma rays, alpha particles, beta particles, photons, electrons, neutrons, radioisotopes, implants of radioactive isotopes and other forms of ionizing radiation. Recent experimental therapy employs monoclonal antibodies specific to the malignant tumor to deliver radioactive isotopes directly to the site of the tumor, termed radioimmunotherapy. The most common type of radiation treatment is radiation directed to the body area containing the neoplastic tumor, which is known as regional or local radiation therapy.

In one embodiment, administration of radiation therapy includes methods well known in the art, such as internal and external radiation therapy. In another embodiment, external therapy includes the administration of radiation via high-energy external beam radiation, administered either regionally (locally) to the tumor site or whole body irradiation. In another embodiment, examples of internal radiation (brachytherapy) include the implantation of radioactive isotopes in permanent, temporary, sealed, unsealed, intracavity or interstitial implants. In one embodiment, the choice of implant is determined by the characteristics of the neoplasia, including the location and extent of the tumor. In another embodiment, the choice between external or internal radiation treatment and type of external radiation treatment is also determined by the characteristics of the neoplasia and can be determined by those skilled in the art.

In one embodiment, “radiation therapy” or “radiotherapy” refers to the medical use of ionizing radiation as part of cancer treatment to control malignant cells. Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, etc. This list is not meant to be limiting. In one embodiment, radiation therapy comprises a targeted radiation therapy wherein the procedure uses computers to create a 3-dimensional picture of the tumor in order to target the tumor as accurately as possible and give it the highest possible dose of radiation while sparing normal tissue as much as possible. It is also known as 3-D conformal (or conformational). The radiation used for cancer treatment may come from a machine outside the body, or it may come from radioactive material placed in the body near tumor cells or injected into the bloodstream. In one embodiment, targeted radiation therapy comprises a method wherein a radioactive material is targeted to a particular place in the body, for example near the tumor cells in order to target and limit the killing of cells to cancer cells, while sparing normal tissue.

In one embodiment, targeted radiation therapy is Internal Radiation Therapy, also known as brachytherapy, which is radiation delivered from radiation sources (radioactive materials) placed inside or on the body. Several brachytherapy techniques are used in cancer treatment. Interstitial brachytherapy uses a radiation source placed within tumor tissue, such as within a prostate tumor. Intracavitary brachytherapy uses a source placed within a surgical cavity or a body cavity, such as the chest cavity, near a tumor. Episcleral brachytherapy, which is used to treat melanoma inside the eye, uses a source that is attached to the eye.

In brachytherapy, radioactive isotopes are sealed in tiny pellets or “seeds.” These seeds are placed in patients using delivery devices, such as needles, catheters, or some other type of carrier. As the isotopes decay naturally, they give off radiation that damages nearby cancer cells. If left in place, after a few weeks or months, the isotopes decay completely and no longer give off radiation. The seeds will not cause harm if they are left in the body (see permanent brachytherapy, described below).

Brachytherapy may be able to deliver higher doses of radiation to some cancers than external-beam radiation therapy while causing less damage to normal tissue.

Brachytherapy can be given as a low-dose-rate or a high-dose-rate treatment:

In low-dose-rate treatment, cancer cells receive continuous low-dose radiation from the source over a period of several days. In high-dose-rate treatment, a robotic machine attached to delivery tubes placed inside the body guides one or more radioactive sources into or near a tumor, and then removes the sources at the end of each treatment session. High-dose-rate treatment can be given in one or more treatment sessions. In one embodiment, administration of a targeted radiation therapy comprises the placement of brachytherapy sources in or near a tumor. In one embodiment the placement of the source is temporary. In another embodiment, the placement of the source is permanent.

For permanent brachytherapy, the sources are surgically sealed within the body and left there, even after all of the radiation has been given off. The remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment.

In one embodiment, a patient may receive radiation therapy before, during, or after administration of a composition disclosed herein, depending on the type of cancer being treated. In one embodiment, methods disclosed herein comprise administering a composition disclosed herein comprising a recombinant Listeria and administering a targeted radiation therapy. In one embodiment, methods disclosed herein comprise administering a composition disclosed herein comprising a recombinant Listeria and an additional active agent, and administering a targeted radiation therapy. In another embodiment, methods disclosed herein comprise administering a composition disclosed herein comprising a recombinant Listeria, administering a composition comprising an additional active agent, and administering a targeted radiation therapy. In one embodiment, an additional active agent is an oncolytic virus. In another embodiment, an additional active agent is CAR T cells. In another embodiment, an additional active agent is a therapeutic or immunomodulatory monoclonal antibody. In another embodiment, an additional active agent is a targeting thymidine kinase inhibitor (TKI). In another embodiment, an additional active agent is an adoptively transferred cell incorporating engineered T cell receptors.

In one embodiment, the amount/course of physical energy administered to the individual is determined by the clinician(s) administering the therapy. In another embodiment, the amount/course of physical energy administered to the individual during radiation therapy is determined by the characteristics of the individual's disease, the method of delivery and the weight, age, general health and response of the individual. For radiation therapy in particular, the location of the tumor is a determining factor in the administration, as the radio-sensitivity of the tumor and surrounding tissue are variable according to tissue type, oxygen supply and other factors. In another embodiment, the amount of radiation administered is the dosage known in the art to be effective given the characteristics of the individual and the disease. In other embodiments, the amount of physical energy administered is about 2×, about 5×, about 10×, or about 15× less than that known in the art to be effective for the particular individual and characteristics of the disease. In another embodiment, the amount of physical energy administered is about 20×, about 50×, about 100× or about 1000× less than that known in the art to be effective for the particular individual and characteristics of the disease. In another embodiment the dosage is a sub-lethal or sub-toxic dosage.

In one embodiment, the radiation dose administered to a subject disclosed herein is from about 1.0 to 10 cGy/min. In another embodiment, the radiation dose administered to a subject disclosed herein is from about 11 to 20 cGy/min. In another embodiment, the radiation dose administered to a subject disclosed herein is from about 21 to 30 cGy/min. In another embodiment, the radiation dose administered to a subject disclosed herein is from about 31 to 40 cGy/min. In another embodiment, the radiation dose administered to a subject disclosed herein is from about 41 to 50 cGy/min. In another embodiment, the radiation dose administered to a subject disclosed herein is from about 61 to 70 cGy/min. In another embodiment, the radiation dose administered to a subject disclosed herein is from about 71 to 80 cGy/min. In another embodiment, the radiation dose administered to a subject disclosed herein is from about 81 to 90 cGy/min. In another embodiment, the radiation dose administered to a subject disclosed herein is from about 91 to 100 cGy/min. In another embodiment, the radiation dose administered to a subject disclosed herein is from about 100 to 150 cGy/min. In another embodiment, the radiation dose administered to a subject disclosed herein is from about 151 to 200 cGy/min. In another embodiment, the radiation dose administered to a subject disclosed herein is from about 200 to 500 cGy/min. In another embodiment, the radiation dose administered to a subject disclosed herein is from about 501 to 1,000 cGy/min. In another embodiment, the radiation dose administered to a subject disclosed herein is from about 1,001 to 10,000 cGy/min.

In another embodiment, the radiation dose administered to a subject disclosed herein is a total fraction ranging from about 11 to 20 cGy. In another embodiment, the radiation dose administered to a subject ranges from about 21 to 30 cGy. In another embodiment, the radiation dose administered to a subject ranges from about 31 to 40 cGy. In another embodiment, the radiation dose administered to a subject ranges from about 41 to 50 cGy. In another embodiment, the radiation dose administered to a subject ranges from about 51 to 60 cGy. In another embodiment, the radiation dose administered to a subject ranges from about 61 to 70 cGy. In another embodiment, the radiation dose administered to a subject ranges from about 71 to 80 cGy. In another embodiment, the radiation dose administered to a subject ranges from about 81 to 90 cGy. In another embodiment, the radiation dose administered to a subject ranges from about 91 to 100 cGy. In another embodiment, the radiation dose administered to a subject ranges from about 101 to 200 cGy. In another embodiment, the radiation dose administered to a subject ranges from about 201 to 500 cGy. In another embodiment, the radiation dose administered to a subject ranges from about 501 to 1,000 cGy. In another embodiment, the radiation dose administered to a subject ranges from about 1,001 to 10,000 cGy.

In one embodiment, repeat doses may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. A particular course of treatment according to the above-described methods, for example, combined Listeria and physical energy treatment, may later be followed by a course of combined chemotherapy and Listeria treatment. Assessment may be determined by any of the techniques known in the art, including diagnostic methods such as imaging techniques, analysis of serum tumor markers, biopsy, or the presence, absence or amelioration of tumor associated symptoms.

In one embodiment, radiation treatment entails the administration of a radiosensitizing agent or radioprotectant to facilitate the treatment. Recent evidence suggests that the antineoplastic agent TAXOL™ (paclitaxel) may function as a radiosensitizer. Liebmann et al., J. National Cancer Inst. 86:441, 1994. Similar evidence has been found for TAXOTERE™ (docetaxel). Creane et al., Int. J. Radiat. Biol. 75:731, 1999; Sikov et al., Front. Biosci. May 1: 221, 1997. Other radiation sensitizers include E2F-1, anti-ras single chain antibody, p53, GM-CSF, and cytosine deaminase. A tumor specific adenovirus may further comprise a radiation sensitizer, such as p53 for example, or a chemo sensitizer.

In one embodiment, combination treatment with compositions comprising a recombinant Listeria plus or minus an additional active agent and radiation therapy are used as components of a combined modality treatment, and the choice of additional active agent(s) and type and course of radiation therapy treatment is generally governed by the characteristics of the individual cancer and the response of the individual. While target cell-specific Listeria strains can be used with either radiation therapy or with additional active agents such as oncolytic viruses, CAR T cells, therapeutic or immunomodulatory monoclonal antibodies, targeting thymidine kinase inhibitors (TKI) and/or Receptor engineered T cells, as separate courses of treatment, they can also be combined with both methods of treatment in the same course of therapy. Accordingly, the disclosure encompasses combinations of the methods discussed above.

Accordingly, the disclosure includes methods for suppressing tumor growth in an individual comprising the following steps, in any order: a) administering to the individual an effective amount of a composition comprising a target cell-specific Listeria strain and optionally, at least one additional active agent; and b) administering an effective amount of an appropriate course of radiation therapy to the individual.

In one embodiment, the method may further comprise the step of: c) administering to the individual an additional dose of the Listeria and optionally a composition comprising an additional active agent, such as an oncolytic virus, CAR T cells, a therapeutic or immunomodulatory monoclonal antibody, TKI, or Receptor engineered T cells s, and the radiation therapy as necessary to treat the individual's neoplasia.

In another embodiment, the method may further comprise time delays after any one of steps a), b) and c). A time delay interval may be hours, days, weeks or months.

In one embodiment, the above-described methods include administration of the Listeria strains, and radiation therapy, and optionally additional active agent(s) such as oncolytic viruses, CAR T cells, a therapeutic or immunomodulatory monoclonal antibody, TKI, or Receptor engineered T cells, in any order and may include sequential administration or simultaneous administration of all or some of the components (i.e. simultaneous administration of physical energy and Listeria strain followed sequentially by radiation therapy, or sequential administration of Listeria strain first, radiation therapy second and thirdly, an oncolytic virus, CAR T cells, a therapeutic or immunomodulatory monoclonal antibody, TKI, and/or Receptor engineered T cells, etc.).

In one embodiment, disclosed herein are methods and compositions for preventing, treating and vaccinating against a heterologous antigen-expressing tumor and inducing an immune response against sub-dominant epitopes of the heterologous antigen, while preventing an escape mutation of the tumor.

In one embodiment, the methods and compositions for preventing, treating and vaccinating against a heterologous antigen-expressing tumor comprise the use of a Listeriolysin (LLO) adjuvant. In another embodiment, the methods and compositions disclosed herein comprise a recombinant Listeria strain overexpressing LLO. In one embodiment, the LLO is expressed from the chromosome of the Listeria strain. In another embodiment, the LLO is expressed from a plasmid within the Listeria strain.

In another embodiment, disclosed herein is a method of inhibiting tumor-mediated immunosuppression in a subject, the method comprising the step of administering to the subject an immunogenic composition comprising a programmed cell death receptor-1 (PD-1) signaling pathway inhibitor, and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In another embodiment, disclosed herein is a method of preventing or treating a tumor growth or cancer in a subject, the method comprising the step of administering to the subject an immunogenic composition comprising a programmed cell death receptor-1 (PD-1) signaling pathway inhibitor, and a recombinant Listeria strain comprising a nucleic acid molecule, the nucleic acid molecule comprising a first open reading frame encoding fusion polypeptide, wherein the fusion polypeptide comprises a Truncated LLO, a truncated ActA or a PEST-sequence peptide fused to a heterologous antigen or fragment thereof.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing.

In one embodiment, disclosed herein is a method of increasing a ratio of T effector cells to regulatory T cells (Tregs) in the spleen and tumor microenvironments of a subject, comprising administering the immunogenic composition disclosed herein. In another embodiment, increasing a ratio of T effector cells to regulatory T cells (Tregs) in the spleen and tumor microenvironments in a subject allows for a more profound anti-tumor response in the subject.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In one embodiment, the disclosure provides methods of treating, protecting against, and inducing an immune response against a tumor or a cancer, comprising the step of administering to a subject the immunogenic composition disclosed herein.

In one embodiment, the disclosure provides a method of preventing or treating a tumor or cancer in a human subject, comprising the step of administering to the subject the immunogenic composition strain disclosed herein, the recombinant Listeria strain comprising a recombinant polypeptide comprising an N-terminal fragment of an LLO protein and tumor-associated antigen, whereby the recombinant Listeria strain induces an immune response against the tumor-associated antigen, thereby treating a tumor or cancer in a human subject. In another embodiment, the immune response is an T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response. In another embodiment, the disclosure provides a method of protecting a subject against a tumor or cancer, comprising the step of administering to the subject the immunogenic composition disclosed herein. In another embodiment, the disclosure provides a method of inducing regression of a tumor in a subject, comprising the step of administering to the subject the immunogenic composition disclosed herein. In another embodiment, the disclosure provides a method of reducing the incidence or relapse of a tumor or cancer, comprising the step of administering to the subject the immunogenic composition disclosed herein. In another embodiment, the disclosure provides a method of suppressing the formation of a tumor in a subject, comprising the step of administering to the subject the immunogenic composition disclosed herein. In another embodiment, the disclosure provides a method of inducing a remission of a cancer in a subject, comprising the step of administering to the subject the immunogenic composition disclosed herein. In one embodiment, the nucleic acid molecule comprising a first open reading frame encoding a fusion polypeptide is integrated into the Listeria genome. In another embodiment, the nucleic acid is in a plasmid in the recombinant Listeria strain. In another embodiment, the nucleic acid molecule is in a bacterial artificial chromosome in the recombinant Listeria strain.

In one embodiment, the method comprises the step of co-administering the recombinant Listeria with an additional therapy. In another embodiment, the additional therapy is surgery, chemotherapy, an immunotherapy, a radiation therapy, CAR T cell therapy, oncolytic virus based therapy, a therapeutic or immunomodulatory monoclonal antibody therapy, targeting TKI therapy, or Receptor engineered T cell therapy, or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the antibody therapy is an anti-PD1, anti-CTLA4. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in U.S. Pat. No. 6,991,785, incorporated by reference herein.

In one embodiment, the methods disclosed herein further comprise the step of co-administering an immunogenic composition disclosed herein with an oncolytic virus that enhances an anti-tumor immune response in said subject.

In one embodiment, the methods disclosed herein further comprise the step of co-administering an immunogenic composition disclosed herein with a indoleamine 2,3-dioxygenase (IDO) pathway inhibitor. IDO pathway inhibitors for use in the disclosure include any IDO pathway inhibitor known in the art, including but not limited to, 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody or a small molecule IDO inhibitor. In another embodiment, the compositions and methods disclosed herein are also used in conjunction with, prior to, or following a chemotherapeutic or radiotherapeutic regiment. In another embodiment, IDO inhibition enhances the efficiency of chemotherapeutic agents.

In some embodiments, selecting a dosage regimen (also referred to herein as an administration regimen) for a combination therapy disclosed herein depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

It will be appreciated by a skilled artisan that the terms “synergy” or “synergistic” may encompass an immune response as a result of the combination of the two or more compositions disclosed herein that is more potent than the sum of each composition's individual immune response as a result of their individual administration. More specifically, in the in vitro setting one measure of synergy is known as “Bliss synergy.” Bliss synergy may encompass “excess over Bliss independence,” as determined by the Bliss value defined above. When the Bliss value is greater than zero (0), or more preferably greater than 0.2, it is considered indicative of synergy. Of course, the use of “synergy” herein also encompasses in vitro synergy as measured by additional and/or alternate methods. References herein to a combination's in vitro biological effects, including but not limited to anti-cancer effects, being greater than, or equal to, the sum of the combination's components individually, may be correlated to Bliss values. Again, the use of “synergy” herein, including whether a combination of components demonstrates activity equal to or greater than the sum of the components individually, may be measured by additional and/or alternate methods and are known, or will be apparent, to those skilled in this art.

In one embodiment, a combination therapy disclosed is used to treat a tumor that is large enough to be found by palpation or by imaging techniques well known in the art, such as MRI, ultrasound, or CAT scan. In some embodiments, a combination therapy disclosed herein is used to treat an advanced stage tumor having dimensions of at least about 200 mm³, 300 mm³, 400 mm³, 500 mm³, 750 mm³, or up to 1000 mm³.

In one embodiment, a disclosed combination therapy is administered to a patient diagnosed with a cancer that tests positive for PD-L1 expression. In some embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 antagonist or PD-L1 antagonist and the live-attenuated Listeria strains provided for herein, but it is envisioned that the physician could order the first or subsequent diagnostic tests at any time after initiation of treatment, such as for example after completion of a treatment cycle.

In one embodiment, a disclosed combination therapy is administered to a patient diagnosed with a cancer that tests positive for CTLA-4 expression. In some embodiments, CTLA-4 expression is detected using a diagnostic anti-human CTLA-4 antibody, or antigen binding fragment thereof, in an IHC assay on an FFPE or frozen tissue section of a tumor sample removed from the patient. Typically, the patient's physician would order a diagnostic test to determine CTLA-4 expression in a tumor tissue sample removed from the patient prior to initiation of treatment with the CTLA-4 antagonist and the live-attenuated Listeria strains provided for herein, but it is envisioned that the physician could order the first or subsequent diagnostic tests at any time after initiation of treatment, such as for example after completion of a treatment cycle.

In some embodiments that employ one or more disclosed immune-modulating antibody in the combination therapy, the dosing regimen will comprise administering said one or more immune-modulating antibodies at a flat dose of 100 to 500 mg or a weight-based dose of 1 to 10 mg/kg at intervals of about 14 days (±2 days) or about 21 days (±2 days) or about 30 days (±2 days) throughout the course of treatment.

In other embodiments that employ an anti-human PD-1 mAb as the PD-1 antagonist in the combination therapy, the dosing regimen will comprise administering the immune-modulating antibody at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In one embodiment, the terms “treatment regimen”, “dosing protocol” and “dosing regimen” are used interchangeably herein and encompass the dose and timing of administration of each therapeutic agent in a combination therapy disclosed herein.

In certain embodiments, a subject will be administered an intravenous (IV) infusion of a medicament comprising any of the immune-modulating antibodies described herein.

In one embodiment, an immune-modulating antibody in the combination therapy is an antagonist antibody. In another embodiment, an immune-modulating antibody in the combination therapy is an antagonist antibody.

In another embodiment, an immune-modulating antibody is administered intravenously at a dose selected from the group consisting of: 1 mg/kg, one dose every 2 weeks (Q2W), 2 mg/kg Q2W, 3 mg/kg Q2W, 5 mg/kg Q2W, 10 mg Q2W, 1 mg/kg one dose every three weeks (Q3W), 2 mg/kg Q3W, 3 mg/kg Q3W, 5 mg/kg Q3W, and 10 mg Q3W.

In another embodiment, the immune-modulating antibody in the combination therapy is administered in a liquid medicament at a dose selected from the group consisting of 200 mg Q3W, 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W or equivalents of any of these doses (e.g., a PK model of an immune-modulating antibody estimates that the fixed dose of 200 mg Q3W provides exposures that are consistent with those obtained with 2 mg/kg Q3W). In some embodiments, an immune-modulating antibody is administered as a liquid medicament which comprises 25 mg/ml the antibody, 7% (w/v) sucrose, 0.02% (w/v) polysorbate 80 in 10 mM histidine buffer pH 5.5, and the selected dose of the medicament is administered by IV infusion over a time period of 30 minutes+/−10 min.

In another embodiment, the attenuated bacterial or attenuated Listeria in the combination therapy is a live-attenuated Listeria strain disclosed herein, which is administered in a liquid medicament at a dose selected from the group consisting of 1×10⁹, 5×10⁹ and 1×10¹⁰ CFU. In some embodiments, a dose ranges from about 1×10⁹ CFU up to 3.31×10¹⁰ CFU, from about 5×10⁸ CFU up to 5×10¹⁰ CFU, from about 7×10⁸ CFU up to 5×10¹⁰ CFU, from about 1×10⁹ CFU up to 5×10¹⁰ CFU, from about 2×10⁹ CFU up to 5×10¹⁰ CFU, from about 3×10⁹ CFU up to 5×10¹⁰ CFU, from about 5×10⁹ CFU up to 5×10¹⁰ CFU, from about 7×10⁹ CFU up to 5×10¹⁰ CFU, from about 1×10¹⁰ CFU up to 5×10¹⁰ CFU, from about 1.5×10⁹ CFU up to 5×10¹⁰ CFU, from about 5×10⁸ CFU—up to 3×10¹⁰ CFU, from about 5×10⁸ CFU up to 2×10¹⁰ CFU, from about 5×10⁸ CFU up to 1.5×10⁹ CFU, from about 5×10⁸ CFU up to 1×10¹⁰ CFU, from about 5×10⁸ CFU up to 7×10⁹ CFU, from about 5×10⁸ CFU up to 5×10⁹ CFU, from about 5×10⁸ CFU up to 3×10⁹ CFU, from about 5×10⁸ CFU up to 2×10⁹ CFU, from about 1×10⁹ CFU up to 3×10¹⁰ CFU, from about 1×10⁹ CFU up to 2×10¹⁰ CFU, from about 2×10⁹ CFU up to 3×10¹⁰ CFU, from about 1×10⁹ CFU up to 1×10¹⁰ CFU, from about 2×10⁹ CFU up to 1×10¹⁰ CFU, from about 3×10⁹ CFU up to 1×10¹⁰ CFU, from about 2×10⁹ CFU up to 7×10⁹ CFU, from about 2×10⁹ CFU up to 5×10⁹ CFU.

In other embodiments, a dose of recombinant Listeria ranges from about 1×10⁷ organisms to about 1.5×10⁸ organisms. In another embodiment, a dose of recombinant Listeria ranges from about 1×10⁸ organisms to about 1.5×10⁹ organisms. In another embodiment, a dose of recombinant Listeria ranges from about 1×10⁹ organisms to about 2×10⁹ organisms. In another embodiment, a dose of recombinant Listeria ranges from about 2×10⁹ organisms to about 5×10⁹ organisms. In another embodiment, a dose of recombinant Listeria ranges from about 2×10⁹ organisms to about 1×10¹⁰ organisms. In another embodiment, a dose of recombinant Listeria ranges from about 3×10⁹ organisms to about 1×10¹⁰ organisms. In another embodiment, a dose of recombinant Listeria ranges from about 4×10⁹ organisms to about 1×10¹⁰ organisms. In another embodiment, a dose of recombinant Listeria ranges from about 5×10⁹ organisms to about 1×10¹⁰ organisms. In another embodiment, a dose of recombinant Listeria ranges from about 6×10⁹ organisms to about 1×10¹⁰ organisms. In another embodiment, a dose of recombinant Listeria ranges from about 7×10⁹ organisms to about 1×10¹⁰ organisms. In another embodiment, a dose of recombinant Listeria ranges from about 1×10⁹ organisms to about 5×10⁹ organisms. In another embodiment, a dose of recombinant Listeria ranges from about 1×10⁹ organisms to about 4×10⁹ organisms. In another embodiment, a dose of recombinant Listeria ranges from about 1×10⁹ organisms to about 3×10⁹ organisms. In another embodiment, a dose of recombinant Listeria ranges from about 5×10⁹ organisms to about 8×10⁹ organisms. In another embodiment, a dose of recombinant Listeria ranges from about 5×10⁹ organisms to about 1.5×10¹⁰ organisms. In another embodiment, a dose of recombinant Listeria ranges from about 5×10⁹ organisms to about 2×10¹⁰ organisms. In another embodiment, a dose of recombinant Listeria ranges from about 5×10⁹ organisms to about 2.5×10¹⁰ organisms. In another embodiment, a dose of recombinant Listeria ranges from about 5×10⁹ organisms to about 3×10¹⁰ organisms. In another embodiment, a dose of recombinant Listeria ranges from about 5×10⁹ organisms to about 3.5×10¹⁰ organisms. In another embodiment, a dose of recombinant Listeria ranges from about 5×10⁹ organisms to about 4×10¹⁰ organisms. In another embodiment, a dose of recombinant Listeria ranges from about 5×10⁹ organisms to about 5×10¹⁰ organisms. In another embodiment, the dose ranges from 1×10⁷ organisms-5×10¹⁰ organisms.

The optimal dose for a combination therapy comprising a disclosed immune-modulating antibody in combination with a disclosed live-attenuated Listeria strain is identified by dose escalation of one or both of these agents. In another embodiment, the optimal dose for a composition comprising either the immune-modulating antibody disclosed herein or the live-attenuated Listeria strain disclosed herein is identified by dose escalation of one or both of these agents.

In one embodiment, a patient is treated with the combination therapy disclosed herein on day 1 of weeks 1, 4 and 7 in a 12 week cycle, starting with at least one immune-modulating antibody that is administered at a starting dose of 50, 100, 150, or 200 mg, and a live-attenuated Listeria strain disclosed herein at a starting dose of ranging from about 1×10⁷ CFU to about 3.5×10¹⁰ CFU.

In an embodiment, the immune-modulating antibody infusion is administered first, followed by a NSAIDS, e.g., naproxen or ibuprofen, and oral antiemetic medication within a predetermined amount of time prior to administration of a live-attenuated Listeria strain provided herein. In another embodiment, the predetermined amount of time is 5-10 min, 11-20 min, 21-40 min, 41-60 min. In another embodiment, the predetermined amount of time is at least one hour. In another embodiment, the predetermined amount of time is 1-2 hours, 2-4 hours, 4-6 hours, 6-10 hours. In another embodiment, administrations of a NSAIDS, e.g., naproxen or ibuprofen, and oral antiemetic medication is repeated on a need basis to the subject, prior to administration of a live-attenuated Listeria strain disclosed herein.

In another embodiment, at least one immune-modulating antibody is administered at a starting dose of 50, 100, 150 or 200 mg Q3W and a live-attenuated Listeria strain disclosed herein is administered Q3W at a starting dose of between 1×10⁷ and 3.5×10¹⁰ CFU.

In one embodiment, a live-attenuated Listeria strain disclosed herein is administered in combination with at least one immune-modulating antibody. In one embodiment, a live-attenuated Listeria strain disclosed herein is administered in combination with an agonist antibody. In another embodiment, a live-attenuated Listeria strain disclosed herein is administered in combination with an immune checkpoint inhibitor antibody. In another embodiment, a live-attenuated Listeria strain disclosed herein is administered in combination with two types of immune-modulating antibodies. In another embodiment, a live-attenuated Listeria strain disclosed herein is administered in combination with an agonist and an immune checkpoint inhibitor antibody. In another embodiment, a live-attenuated Listeria strain disclosed herein is administered at a starting dose of 5×10⁹ Q3W and at least one immune-modulating antibody is administered at a starting dose of 200 mg Q3W, and if the starting dose of the combination is not tolerated by the patient, then the dose of the live-attenuated Listeria strain is reduced to 1×10⁹ cfu Q3W or the dose of least one immune-modulating antibody is reduced to 150 mg Q3W. It is to be understood by a skilled artisan that the doses of any of the components of a combination therapy provided herein may be incrementally adjusted to a lower or higher dose based on a subjects response to the combination therapy.

In some embodiments, dosage levels below the lower limit of the aforesaid range may be more than adequate, while in other cases still larger doses may be employed, as determined by those skilled in the art.

In some embodiments, a treatment cycle begins with the first day of combination treatment and lasts for at least 12 weeks, 24 weeks or 48 weeks. On any day of a treatment cycle that the drugs are co-administered, the timing between the separate IV infusions of an anti-PD1 antibody and a live-attenuated Listeria strain disclosed herein is between about 15 minutes to about 45 minutes. The disclosure contemplates that an immune-modulating antibody and a live-attenuated Listeria strain disclosed herein may be administered in either order or by simultaneous IV infusion.

In some embodiments, the combination therapy is administered for at least 2 to 4 weeks after the patient achieves a complete remission (CR).

In some embodiments, a patient selected for treatment with the combination therapy of the disclosure has been diagnosed with a metastatic cancer and the patient has progressed or become resistant to no more than 2 prior systemic treatment regimens. In some embodiments, a patient selected for treatment with the combination therapy of the disclosure has been diagnosed with a metastatic cancer and the patient has progressed or become resistant to no more than 3 prior systemic treatment regimens.

The present disclosure also provides a medicament which comprises at least one immune-modulating antibody herein and a pharmaceutically acceptable excipient. When an immune-modulating antibody disclosed herein is a biotherapeutic agent, e.g., a mAb, the antibody may be produced in a producing cell line known in the art, such as, but not limited to CHO cells using conventional cell culture and recovery/purification technologies.

In some embodiments, a medicament comprising an immune-modulating antibody disclosed herein may be provided as a liquid formulation or prepared by reconstituting a lyophilized powder with sterile water for injection prior to use. WO 2012/135408 describes the preparation of liquid and lyophilized medicaments comprising an anti-PD-1 antibody that are suitable for use in the disclosure.

The present disclosure also provides a medicament which comprises a live-attenuated Listeria strain disclosed herein and a pharmaceutically acceptable excipient.

An immune-modulating antibody medicament and a live-attenuated Listeria strain disclosed herein medicament may be provided as a kit which comprises a first container and a second container and a package insert. The first container contains at least one dose of a medicament comprising at least one immune-modulating antibody, the second container contains at least one dose of a medicament comprising a live-attenuated Listeria strain disclosed herein, and the package insert, or label, which comprises instructions for treating a patient for a cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some embodiments of a kit disclosed herein, the immune checkpoint inhibitor antibody and the instructions state that the medicaments are intended for use in treating a patient having a cancer that tests positive for expression of a target disclosed herein (e.g. CTLA-4, PD-L1) by an IHC assay.

In another embodiment, a method of disclosure further comprises the step of boosting the subject with a recombinant Listeria strain, an oncolytic virus, CAR T cells, a therapeutic or immunomodulatory monoclonal antibody, TKI, or Receptor engineered T cells, as disclosed herein. In another embodiment, the recombinant Listeria strain used in the booster inoculation is the same as the strain used in the initial “priming” inoculation. In another embodiment, the booster strain is different from the priming strain. In another embodiment, the recombinant immune checkpoint inhibitor used in the booster inoculation is the same as the inhibitor used in the initial “priming” inoculation. In another embodiment, the booster inhibitor is different from the priming inhibitor. In another embodiment, the same doses are used in the priming and boosting inoculations. In another embodiment, a larger dose is used in the booster. In another embodiment, a smaller dose is used in the booster. In another embodiment, the methods of the disclosure further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In another embodiment, a method of the disclosure further comprises boosting the subject with a immunogenic composition comprising an attenuated Listeria strain disclosed herein. In another embodiment, a method of the disclosure comprises the step of administering a booster dose of the immunogenic composition comprising the attenuated Listeria strain disclosed herein. In another embodiment, the booster dose is an alternate form of said immunogenic composition. In another embodiment, the methods of the disclosure further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria disclosed herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun. 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, a treatment protocol of the disclosure is therapeutic. In another embodiment, the protocol is prophylactic. In another embodiment, the compositions of the disclosure are used to protect people at risk for cancer such as breast cancer or other types of tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the disclosure are administered so that the CTL response to the tumor antigen of the vaccine destroys remaining metastases and prolongs remission from the cancer. In another embodiment, vaccines of the disclosure are used to effect the growth of previously established tumors and to kill existing tumor cells. Each possibility represents a separate embodiment of the disclosure.

In some embodiments, the term “comprise” or grammatical forms thereof, refers to the inclusion of the indicated active agent, such as the Lm strains disclosed herein, as well as inclusion of other active agents, such as oncolytic viruses, CAR T cells, a therapeutic or immunomodulatory monoclonal antibody, TKI, or adoptively transferred cells incorporating engineered T cell receptors, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and may be distinct from that of the indicated active ingredient, by acting via a different mechanism of action, for example. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments disclosed herein may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In the following examples, numerous specific details are set forth in order to provide a thorough understanding of the disclosure. However, it will be understood by those skilled in the art that the disclosure may be practiced without these specific details. In other instances, well-known methods, procedures, and components have not been described in detail so as not to obscure the disclosure.

### EXAMPLES

### Materials and Methods (Examples 1-8)

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA vaccine based on the pADV142 plasmid, which has no antibiotic resistance markers, and referred as LmddA-142 (Table 2). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram(−) and gram(+) cm resistance. |
| | LLO-E7 expression cassette and a copy of Lm prfA gene |
| pTV3 | Derived from pGG55 by deleting cm genes and inserting the Lm dal gene |
| pADV119 | Derived from pTV3 by deleting the prfA gene |
| pADV134 | Derived from pADV119 by replacing the Lm dal gene by the Bacillus dal |
| | gene |
| pADV142 | Derived from pADV134 by replacing HPV16 e7 with klk3 |
| pADV168 | Derived from pADV134 by replacing HPV16 e7 with hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly gene in the chromosome |
| LmddA-143 | 10403S dal⁽⁻⁾ dat⁽⁻⁾ actA⁽⁻⁾ with klk3 fused to the hly gene in the chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA- | LmddA-143 pADV134 |
| 143/134 | |
| Lmdd-143/168 | Lmdd-143 pADV168 |
| LmddA- | LmddA-143 pADV168 |
| 143/168 | |

The sequence of the plasmid pAdv142 (6523 bp) was as follows:

(SEQ ID NO: 67)
cggagtgtatactggcttactatgttggcactgatgagggtgtcagtgaagtgcttcatgtggcaggagaaaaaaggctgcaccggt
gcgtcagcagaatatgtgatacaggatatattccgcttcctcgctcactgactcgctacgctcggtcgttcgactgcggcgagcgga
aatggcttacgaacggggcggagatttcctggaagatgccaggaagatacttaacagggaagtgagagggccgcggcaaagccgttt
ttccataggctccgcccccctgacaagcatcacgaaatctgacgctcaaatcagtggtggcgaaacccgacaggactataaagatac
caggcgtttccccctggcggctccctcgtgcgctctcctgttcctgcctttcggtttaccggtgtcattccgctgttatggccgcgt
ttgtctcattccacgcctgacactcagttccgggtaggcagttcgctccaagctggactgtatgcacgaaccccccgttcagtccga
ccgctgcgccttatccggtaactatcgtcttgagtccaacccggaaagacatgcaaaagcaccactggcagcagccactggtaattg
atttagaggagttagtcttgaagtcatgcgccggttaaggctaaactgaaaggacaagttttggtgactgcgctcctccaagccagt
tacctcggttcaaagagttggtagctcagagaaccttcgaaaaaccgccctgcaaggcggttttttcgttttcagagcaagagatta
cgcgcagaccaaaacgatctcaagaagatcatcttattaatcagataaaatatttctagccctcctttgattagtatattcctatct
taaagttacttttatgtggaggcattaacatttgttaatgacgtcaaaaggatagcaagactagaataaagctataaagcaagcata
taatattgcgtttcatctttagaagcgaatttcgccaatattataattatcaaaagagaggggtggcaaacggtatttggcattatt
aggttaaaaaatgtagaaggagagtgaaacccatgaaaaaaataatgctagtttttattacacttatattagttagtctaccaattg
cgcaacaaactgaagcaaaggatgcatctgcattcaataaagaaaattcaatttcatccatggcaccaccagcatctccgcctgcaa
agtcctaagacgccaatcgaaaagaaacacgcggatgaaatcgataagtatatacaaggattggattacaataaaaacaatgtatta
gtataccacggagatgcagtgacaaatgtgccgccaagaaaaggttacaaagatggaaatgaatatattgttgtggagaaaaagaag
aaatccatcaatcaaaataatgcagacattcaagttgtgaatgcaatttcgagcctaacctatccaggtgctctcgtaaaagcgaat
tcggaattagtagaaaatcaaccagatgttctccctgtaaaacgtgattcattaacactcagcattgatttgccaggtatgactaat
caagacaataaaatagttgtaaaaaatgccactaaatcaaacgttaacaacgcagtaaatacattagtggaaagatggaatgaaaaa
tatgctcaagcttatccaaatgtaagtgcaaaaattgattatgatgacgaaatggcttacagtgaatcacaattaattgcgaaattt
ggtacagcatttaaagctgtaaataatagcttgaatgtaaacttcggcgcaatcagtgaagggaaaatgcaagaagaagtcattagt
tttaaacaaatttactataacgtgaatgttaatgaacctacaagaccttccagatttttcggcaaagctgttactaaagagcagttg
caagcgcttggagtgaatgcagaaaatcctcctgcatatatctcaagtgtggcgtatggccgtcaagtttatttgaaattatcaact
aattcccatagtactaaagtaaaagctgcttttgatgctgccgtaagcggaaaatctgtctcaggtgatgtagaactaacaaatatc
atcaaaaattcttccttcaaagccgtaatttacggaggttccgcaaaagatgaagttcaaatcatcgacggcaacctcggagactta
cgcgatattttgaaaaaaggcgctacttttaatcgagaaacaccaggagttcccattgcttatacaacaaacttcctaaaagacaat
gaattagctgttattaaaaacaactcagaatatattgaaacaacttcaaaagcttatacagatggaaaaattaacatcgatcactct
ggaggatacgttgctcaattcaacatttcttggggatgaagtaaattatgatctcgagattgtgggaggctgggagtgcgagaccct
agcattcccaaggcaggtgcttgtggcctctcgtggcagggcagtctgcggcggtgttctggtgcacccccagtgggtcctcacagc
tgcccactgcatcaggaacaaaagcgtgatcttgctgggtcggcacagcctgtttcatcctgaagacacaggccaggtatttcaggt
cagccacagcttcccacacccgctctacgatatgagcctcctgaagaatcgattcctcaggccaggtgatgactccagccacgacct
catgctgctccgcctgtcagagcctgccgagctcacggatgctgtgaaggtcatggacctgcccacccaggagccagcactggggac
cacctgctacgcctcaggctggggcagcattgaaccagaggagttcttgaccccaaagaaacttcagtgtgtggacctccatgttat
ttccaatgacgtgtgtgcgcaagttcaccctcagaaggtgaccaagttcatgctgtgtgctggacgctggacagggggcaaaagcac
ctgctcgggtgattctgggggcccacttgtctgttatggtgtgcttcaaggtatcacgtcatggggcagtgaaccatgtgccctgcc
cgaaaggccttccctgtacaccaaggtggtgcattaccggaagtggatcaaggacaccatcgtggccaaccccTAAcccgggccact
aactcaacgctagtagtggatttaatcccaaatgagccaacagaaccagaaccagaaacagaacaagtaacattggagttagaaatg
gaagaagaaaaaagcaatgatttcgtgtgaataatgcacgaaatcattgcttatttttttaaaaagcgatatactagatataacgaa
acaacgaactgaataaagaatacaaaaaaagagccacgaccagttaaagcctgagaaactttaactgcgagccttaattgattacca
ccaatcaattaaagaagtcgagacccaaatttggtaaagtatttaattactttattaatcagatacttaaatatctgtaaacccatt
atatcgggtttttgaggggatttcaagtctttaagaagataccaggcaatcaattaagaaaaacttagttgattgccttttttgttg
tgattcaactttgatcgtagcttctaactaattaattttcgtaagaaaggagaacagctgaatgaatatcccttttgttgtagaaac
tgtgcttcatgacggcttgttaaagtacaaatttaaaaatagtaaaattcgctcaatcactaccaagccaggtaaaagtaaaggggc
tatttttgcgtatcgctcaaaaaaaagcatgattggcggacgtggcgttgttctgacttccgaagaagcgattcacgaaaatcaaga
tacatttacgcattggacaccaaacgtttatcgttatggtacgtatgcagacgaaaaccgttcatacactaaaggacattctgaaaa
caatttaagacaaatcaataccttctttattgattttgatattcacacggaaaaagaaactatttcagcaagcgatattttaacaac
agctattgatttaggttttatgcctacgttaattatcaaatctgataaaggttatcaagcatattttgttttagaaacgccagtcta
tgtgacttcaaaatcagaatttaaatctgtcaaagcagccaaaataatctcgcaaaatatccgagaatattttggaaagtctttgcc
agttgatctaacgtgcaatcattttgggattgctcgtataccaagaacggacaatgtagaattttttgatcccaattaccgttattc
tttcaaagaatggcaagattggtctttcaaacaaacagataataagggctttactcgttcaagtctaacggttttaagcggtacaga
aggcaaaaaacaagtagatgaaccctggtttaatctcttattgcacgaaacgaaattttcaggagaaaagggtttagtagggcgcaa
tagcgttatgtttaccctctctttagcctactttagttcaggctattcaatcgaaacgtgcgaatataatatgtttgagtttaataa
tcgattagatcaacccttagaagaaaaagaagtaatcaaaattgttagaagtgcctattcagaaaactatcaaggggctaataggga
atacattaccattctttgcaaagcttgggtatcaagtgatttaaccagtaaagatttatttgtccgtcaagggtggtttaaattcaa
gaaaaaaagaagcgaacgtcaacgtgttcatttgtcagaatggaaagaagatttaatggcttatattagcgaaaaaagcgatgtata
caagccttatttagcgacgaccaaaaaagagattagagaagtgctaggcattcctgaacggacattagataaattgctgaaggtact
gaaggcgaatcaggaaattttctttaagattaaaccaggaagaaatggtggcattcaacttgctagtgttaaatcattgttgctatc
gatcattaaattaaaaaaagaagaacgagaaagctatataaaggcgctgacagcttcgtttaatttagaacgtacatttattcaaga
aactctaaacaaattggcagaacgccccaaaacggacccacaactcgatttgtttagctacgatacaggctgaaaataaaacccgca
ctatgccattacatttatatctatgatacgtgtttgtttttctttgctggctagcttaattgcttatatttacctgcaataaaggat
ttcttacttccattatactcccattttccaaaaacatacggggaacacgggaacttattgtacaggccacctcatagttaatggttt
cgagccttcctgcaatctcatccatggaaatatattcatccccctgccggcctattaatgtgacttttgtgcccggcggatattcct
gatccagctccaccataaattggtccatgcaaattcggccggcaattttcaggcgttttcccttcacaaggatgtcggtccctttca
attttcggagccagccgtccgcatagcctacaggcaccgtcccgatccatgtgtctttttccgctgtgtactcggctccgtagctga
cgctctcgcctttctgatcagtttgacatgtgacagtgtcgaatgcagggtaaatgccggacgcagctgaaacggtatctcgtccga
catgtcagcagacgggcgaaggccatacatgccgatgccgaatctgactgcattaaaaaagccttttttcagccggagtccagcggc
gctgttcgcgcagtggaccattagattctttaacggcagcggagcaatcagctctttaaagcgctcaaactgcattaagaaatagcc
tctttctttttcatccgctgtcgcaaaatgggtaaatacccctttgcactttaaacgagggttgcggtcaagaattgccatcacgtt
ctgaacttcttcctctgtttttacaccaagtctgttcatccccgtatcgaccttcagatgaaaatgaagagaaccttttttcgtgtg
gcgggctgcctcctgaagccattcaacagaataacctgttaaggtcacgtcatactcagcagcgattgccacatactccgggggaac
cgcgccaagcaccaatataggcgccttcaatccctttttgcgcagtgaaatcgcttcatccaaaatggccacggccaagcatgaagc
acctgcgtcaagagcagcctttgctgtttctgcatcaccatgcccgtaggcgtttgctttcacaactgccatcaagtggacatgttc
accgatatgttttttcatattgctgacattttcctttatcgcggacaagtcaatttccgcccacgtatctctgtaaaaaggttttgt
gctcatggaaaactcctctcttttttcagaaaatcccagtacgtaattaagtatttgagaattaattttatattgattaatactaag
tttacccagttttcacctaaaaaacaaatgatgagataatagctccaaaggctaaagaggactataccaactatttgttaatta.
This plasmid was sequences at Genewiz facility from the E. coli strain on Feb. 20, 2008.

### Example 1: Construction of Attenuated Listeria Strain-LmddΔactA and Insertion of the Human klk3 Gene in Frame to the Hly Gene in the Lmdd and Lmdda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat AactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 2
Sequence of primers that was used for the amplification
of DNA sequences upstream and downstream of actA

| Primer | Sequence | SEQ ID NO: |
|---|---|---|
| Adv271-actAF1 | cg GAATTCGGATCCgcgccaaatcattggttgattg | 68 |
| Adv272-actAR1 | gcgaGTCGACgtcggggttaatcgtaatgcaattggc | 69 |
| Adv273-actAF2 | gcgaGTCGACccatacgacgttaattcttgcaatg | 70 |
| Adv274-actAR2 | gataCTGCAGGGATCCttcccttctcggtaatcagtcac | 71 |

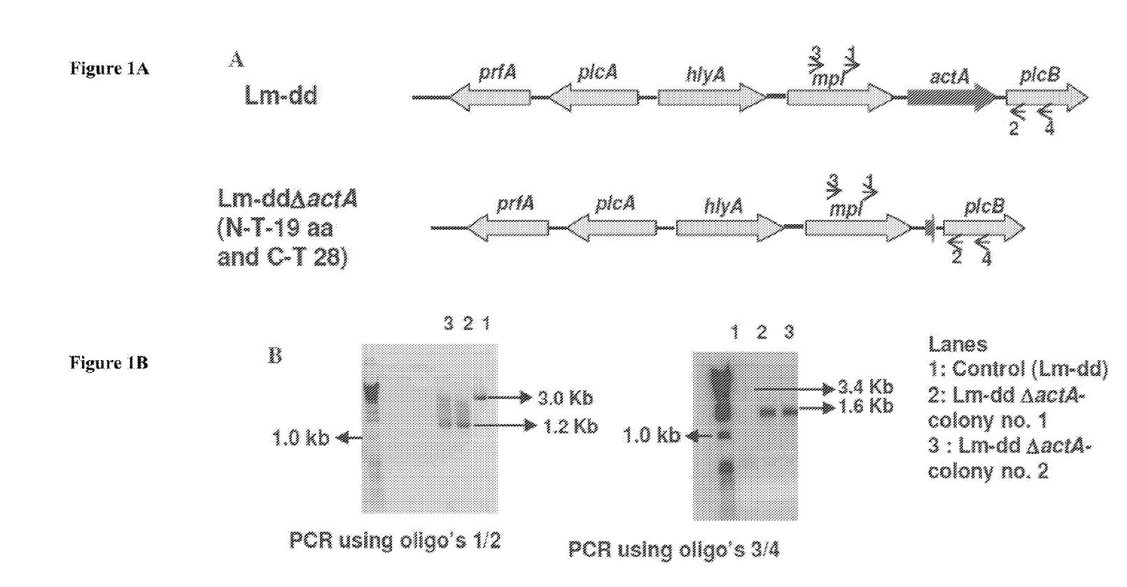

The deletion of the gene from its chromosomal location was verified using primers that bind externally to the actA deletion region, which are shown in FIG. 1 as primer 3 (Adv 305-tgggatggccaagaaattc, SEQ ID NO: 72) and primer 4 (Adv304-ctaccatgtcttccgttgcttg; SEQ ID NO: 73). The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIG. 1 confirms that the 1.8 kb region of actA was deleted in the LmddΔactA strain. DNA sequencing was also performed on PCR products to confirm the deletion of actA containing region in the strain, LmddΔactA.

### Example 2: Construction of the Antibiotic-Independent Episomal Expression System for Antigen Delivery by Lm Vectors

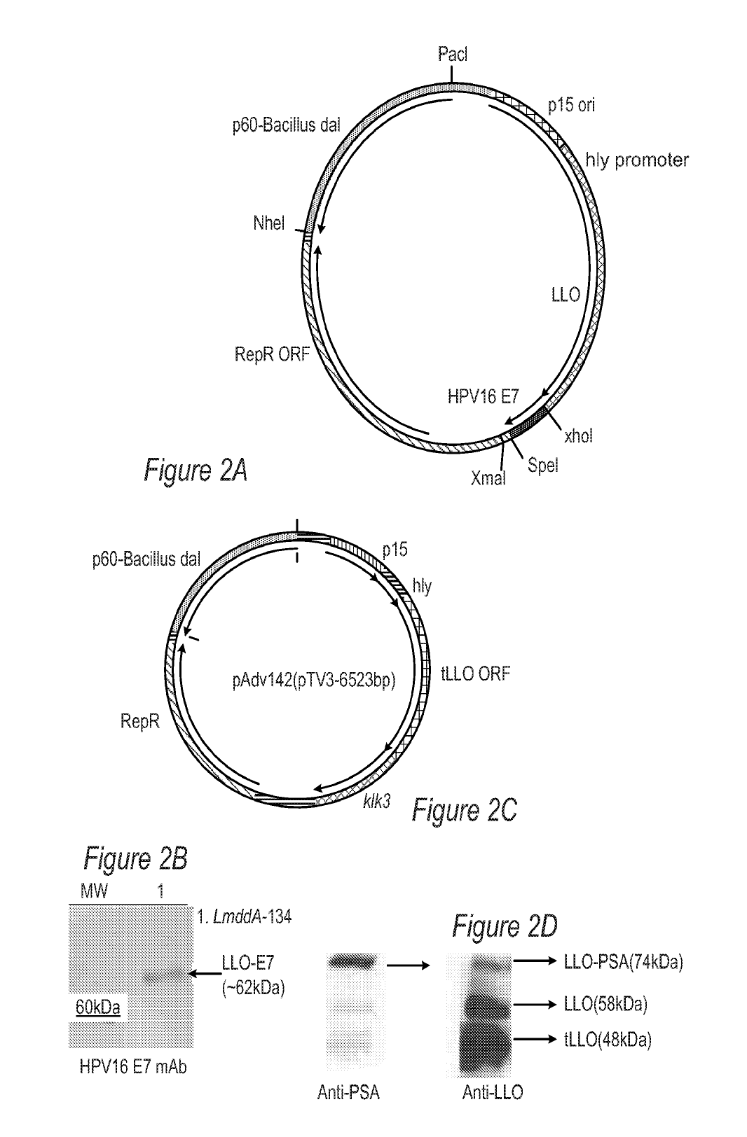

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is −30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3: In Vitro and In Vivo Stability of the Strain LmddA-LLO-PSA

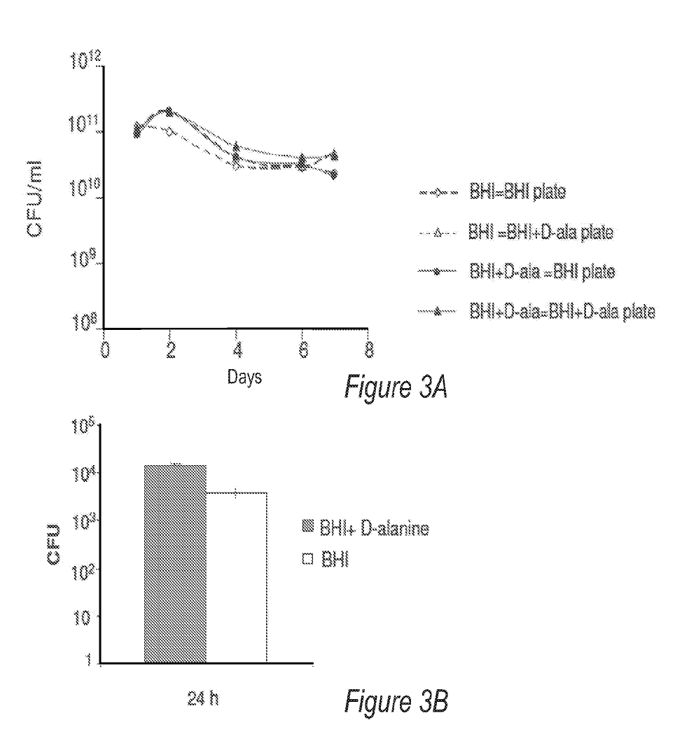

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+ 100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+ 100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

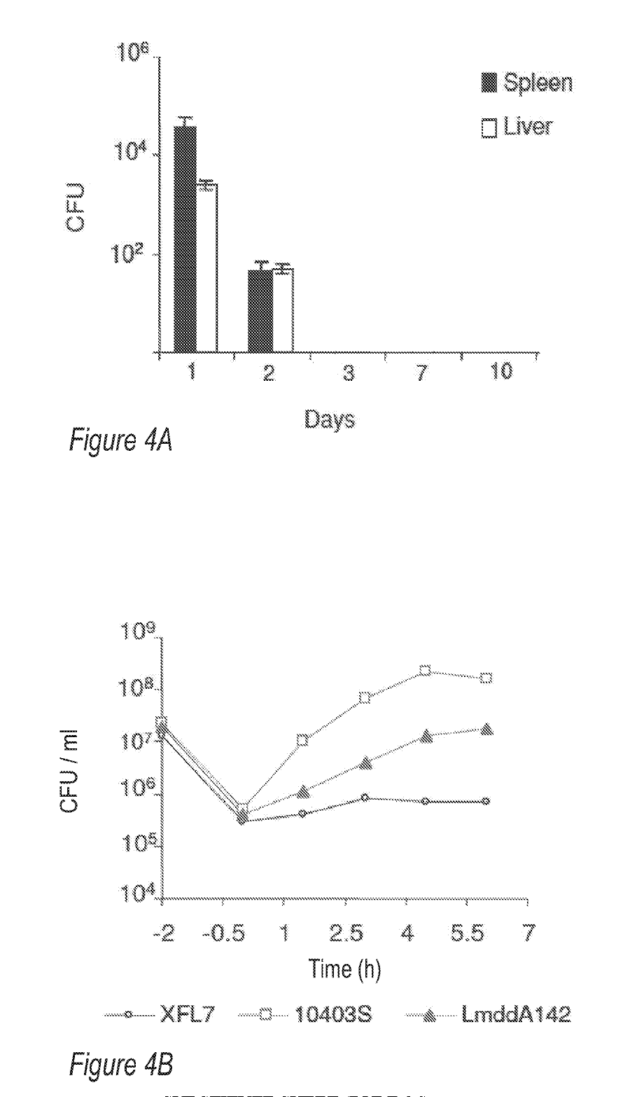

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5: Immunogenicity of the Strain-LmddA-LLO-PSA in C57BL/6 Mice

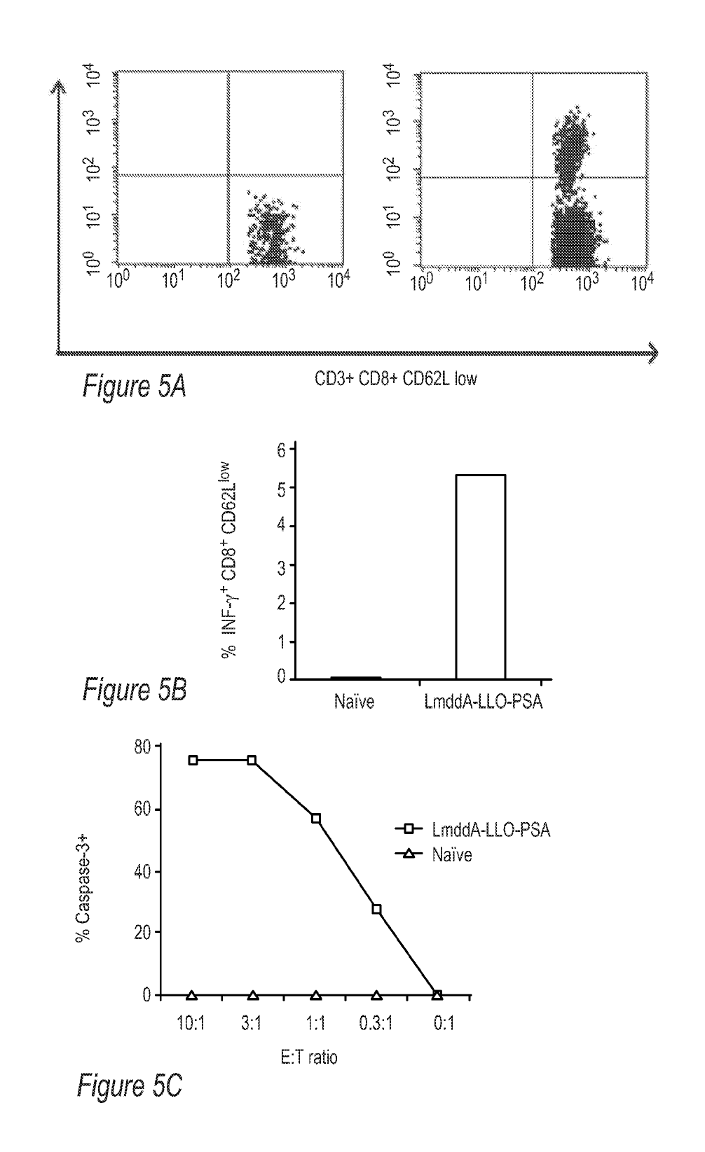

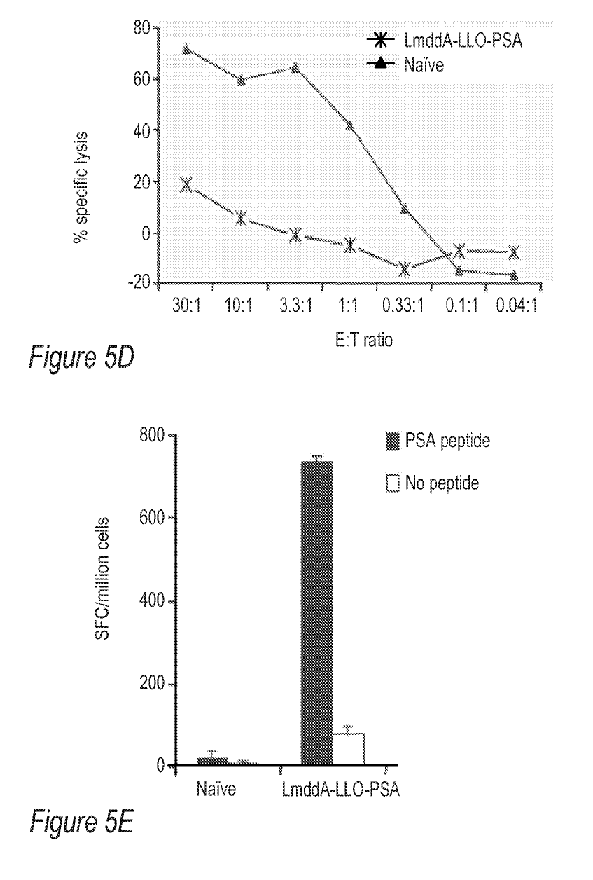

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 5A).

The functional ability of the PSA-specific T cells to secrete IFN-γ after stimulation with PSA peptide for 5 h was examined using intracellular cytokine staining. There was a 200-fold increase in the percentage of CD8+CD62L^{low} IFN-γ secreting cells stimulated with PSA peptide in the LmddA-LLO-PSA group compared to the naïve mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 5E).

### Example 6: Immunization with the LmddA-142 Strains Induces Regression of a Tumor Expressing PSA and Infiltration of the Tumor by PSA-Specific CTLs

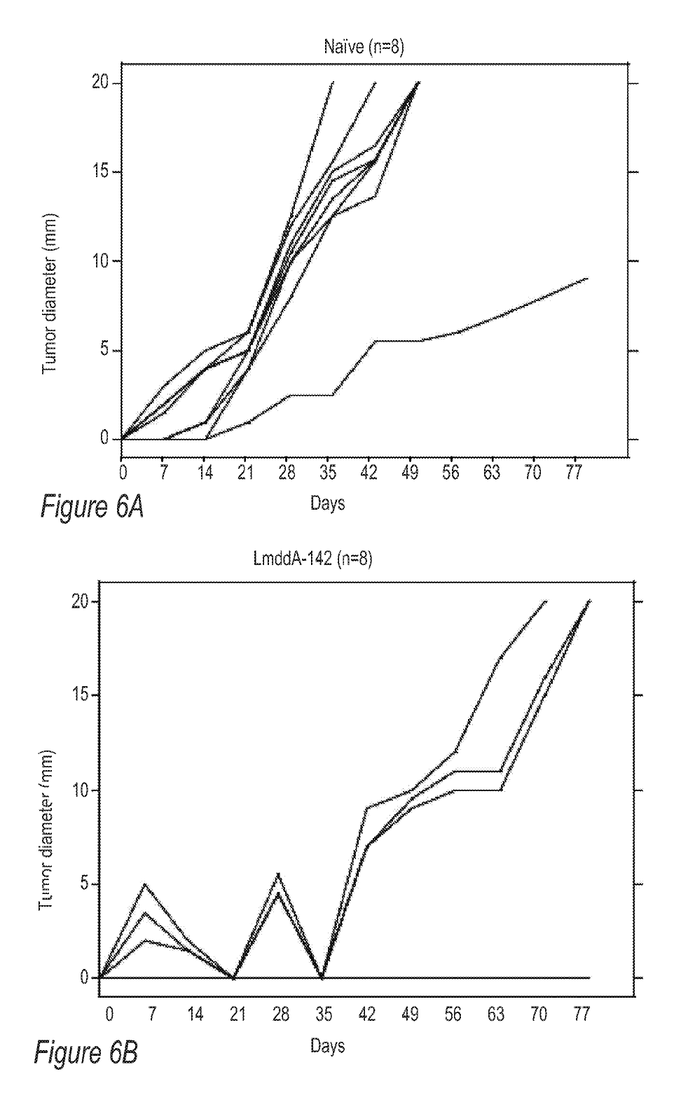

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 6A). The mice immunized with LmddA-142 were all tumor-free until day 35 and gradually 3 out of 8 mice developed tumors, which grew at a much slower rate as compared to the naïve mice (FIG. 6B). Five out of eight mice remained tumor free through day 70. As expected, Lm-LLO-PSA-vaccinated mice had fewer tumors than naïve controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C tumors that were engineered to express PSA in more than 60% of the experimental animals (FIG. 6B), compared to none in the untreated group (FIG. 6A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62L^{low} PSA^{tetramer+} and CD4⁺ CD25⁺FoxP3⁺ regulatory T cells infiltrating in the tumors.

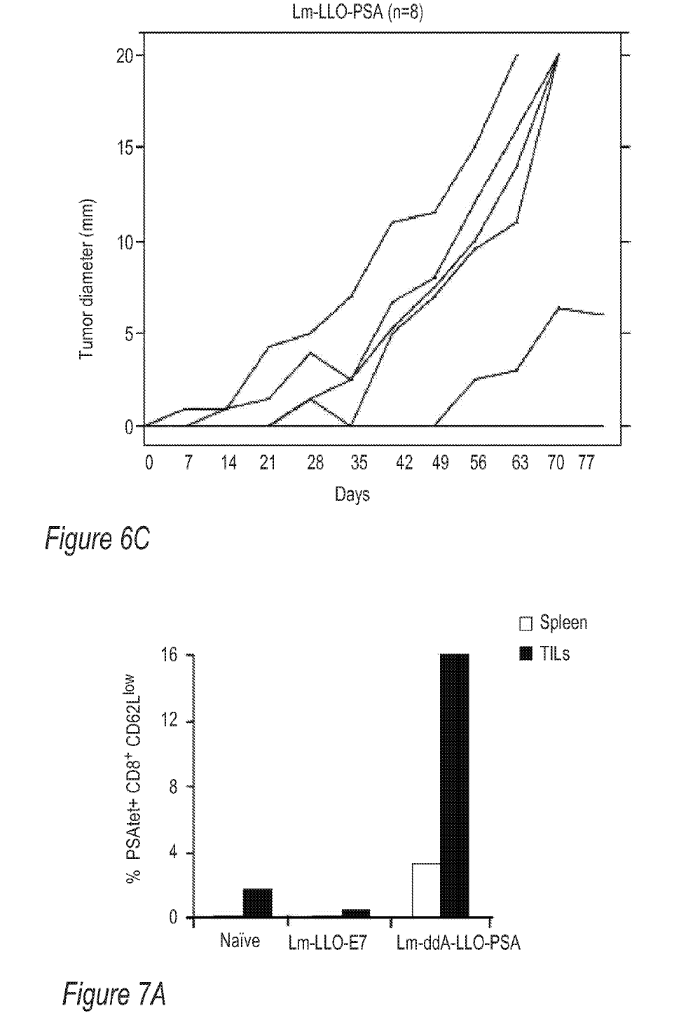

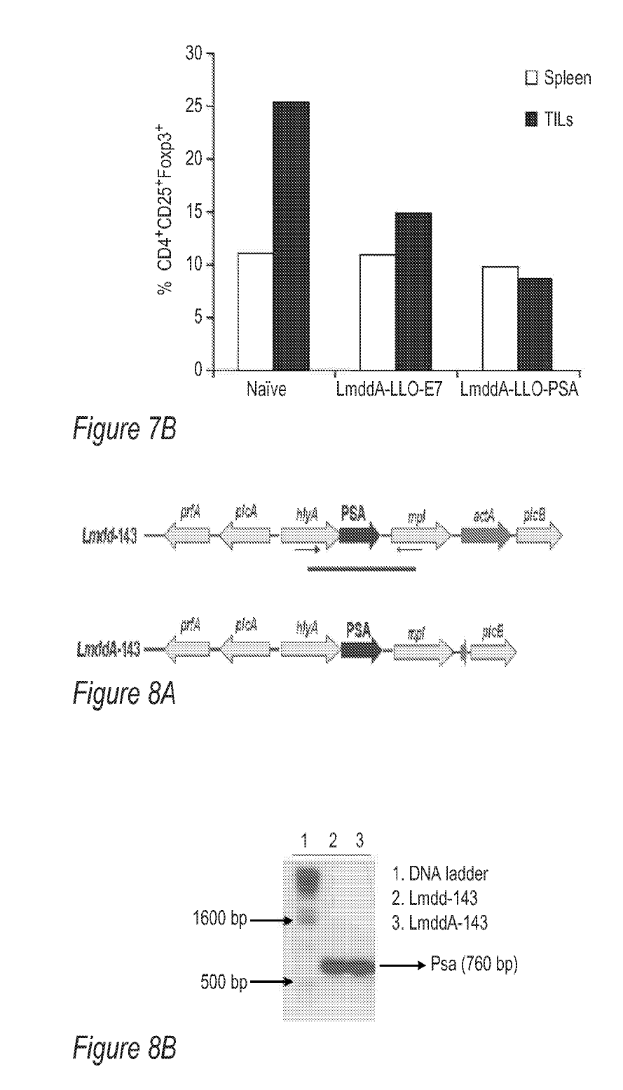

A very low number of CD8+CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8+CD62L^{low} PSA^{tetramer+} TILs in the mice immunized with LmddA-LLO-PSA (FIG. 7A). Interestingly, the population of CD8+CD62L^{low} PSA^{tetramer+} cells in spleen was 7.5 fold less than in tumor (FIG. 7A).

In addition, the presence of CD4⁺/CD25⁺/Foxp3⁺ T regulatory cells (regs) in the tumors of untreated mice and Listeria immunized mice was determined. Interestingly, immunization with Listeria resulted in a considerable decrease in the number of CD4⁺CD25⁺FoxP3⁺ T-regs in tumor but not in spleen (FIG. 7B). However, the construct LmddA-LLO-PSA had a stronger impact in decreasing the frequency of CD4⁺CD25⁺FoxP3⁺ T-regs in tumors when compared to the naïve and Lm-LLO-E7 immunized group (FIG. 7B).

Thus, the LmddA-142 vaccine can induce PSA-specific CD8⁺ T cells that are able to infiltrate the tumor site (FIG. 7A). Interestingly, immunization with LmddA-142 was associated with a decreased number of regulatory T cells in the tumor (FIG. 7B), probably creating a more favorable environment for an efficient anti-tumor CTL activity.

### Example 7: Lmdd-143 and LmddA-143 Secretes a Functional LLO Despite the PSA Fusion

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie. 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 8A). The insertion of klk3 in frame with hly into the chromosome was verified by PCR (FIG. 8B) and sequencing (data not shown) in both constructs.

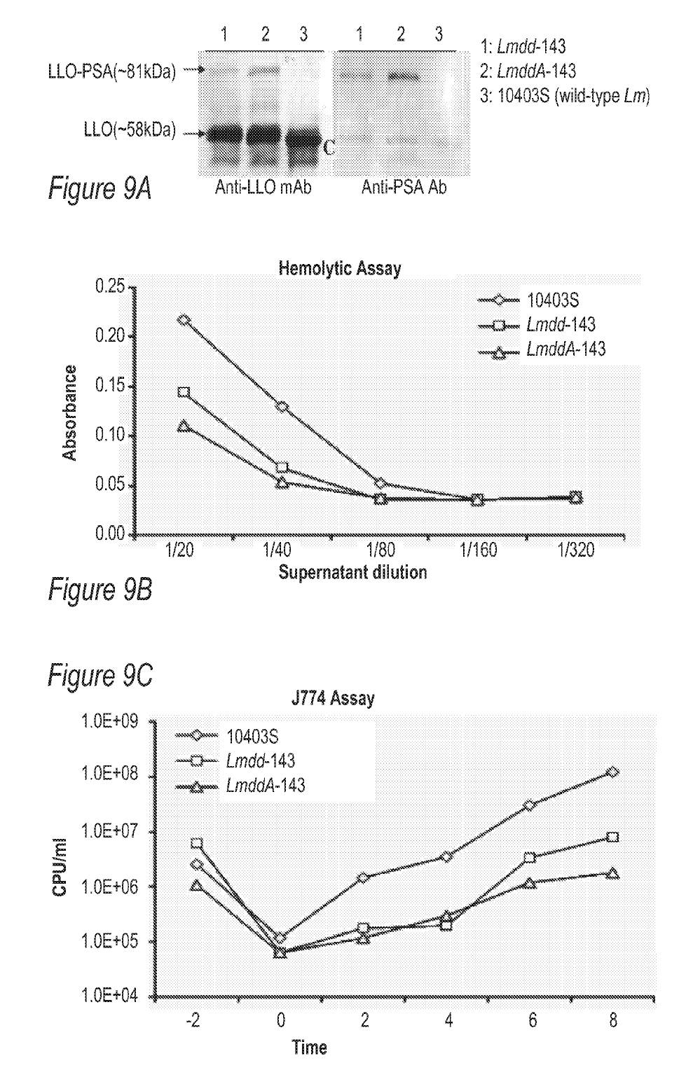

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 9A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 9B). In agreement with these results, both Lmdd-143 and LmddA-143 were able to replicate intracellularly in the macrophage-like J774 cell line (FIG. 9C).

### Example 8: Both Lmdd-143 and LmddA-143 Elicit Cell-Mediated Immune Responses Against the PSA Antigen

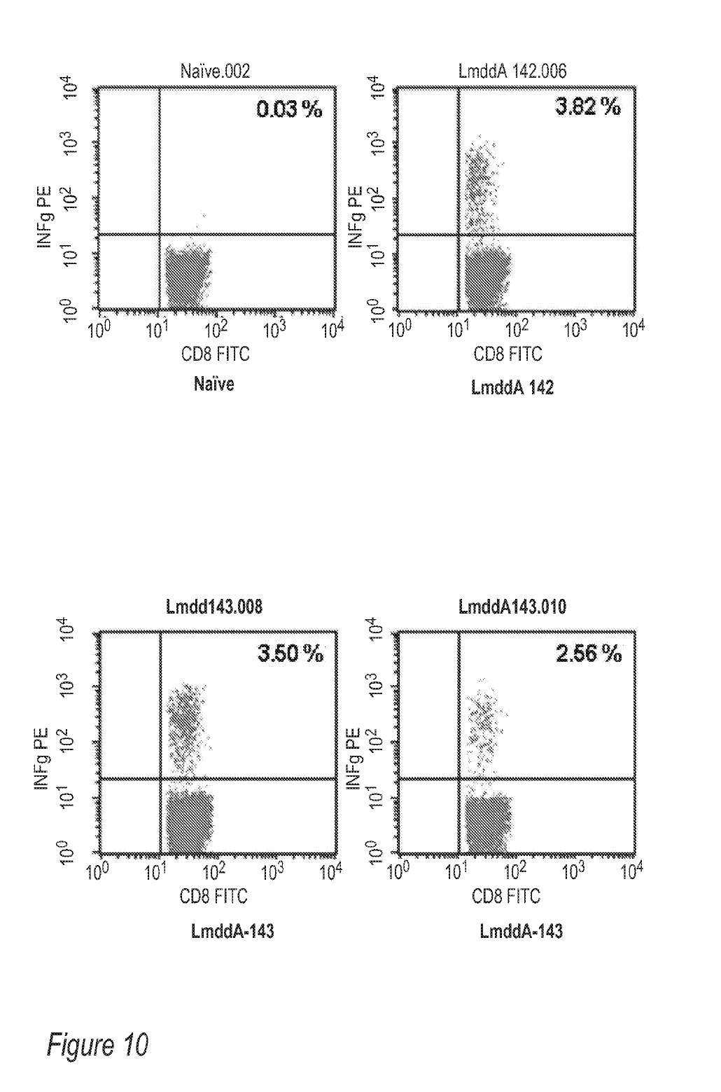

After showing that both Lmdd-143 and LmddA-143 were able to secrete PSA fused to LLO, the question of if these strains could elicit PSA-specific immune responses in vivo was investigated. C57B1/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 10, the immune response induced by the chromosomal and the plasmid-based vectors is similar.

### Materials and Methods (Examples 9-13)

### MDSC and Treg Function

Tumors were implanted in mice on the flank or a physiological site depending on the tumor model. After 7 days, mice were then vaccinated, the initial vaccination day depends on the tumor model being used. The mice were then administered a booster vaccine one week after the vaccine was given.

Mice were then sacrificed and tumors and spleen were harvested 1 week after the boost or, in the case of an aggressive tumor model, 3-4 days after the boost. Five days before harvesting the tumor, non-tumor bearing mice were vaccinated to use for responder T cells. Splenocytes were prepared using standard methodology.

Briefly, single cell suspensions of both the tumors and the spleens were prepared. Spleens were crushed manually and red blood cells were lysed. Tumors were minced and incubated with collagenase/DNase. Alternatively, the GENTLEMACS™ dissociator was used with the tumor dissociation kit.

MDSCs or Tregs were purified from tumors and spleens using a Miltenyi kit and columns or the autoMACs separator. Cells were then counted.

Single cell suspension was prepared and the red blood cells were lysed. Responder T cells were then labeled with CFSE.

Cells were plated together at a 2:1 ratio of responder T cells (from all division cycle stages) to MDSCs or Tregs at a density of 1×10⁵ T cells per well in 96 well plates. Responder T cells were then stimulated with either the appropriate peptide (PSA OR CA9) or non-specifically with PMA/ionomycin. Cells were incubated in the dark for 2 days at 37° C. with 5% CO₂. Two days later, the cells were stained for FACS and analyzed on a FACS machine.

### Analysis of T-Cell Responses

For cytokine analysis by ELISA, splenocytes were harvested and plated at 1.5 million cells per well in 48-well plates in the presence of media, SEA or conA (as a positive control). After incubation for 72 hours, supernatants were harvested and analyzed for cytokine level by ELISA (BD). For antigen-specific IFN-γ ELISpot, splenocytes were harvested and plated at 300K and 150K cells per well in IFN-γ ELISpot plates in the presence of media, specific CTL peptide, irrelevant peptide, specific helper peptide or conA (as a positive control). After incubation for 20 hours, ELISpots (BD) were performed and spots counted by the Immunospot analyzer (C.T.L.). Number of spots per million splenocytes were graphed.

Splenocytes were counted using a Coulter Counter, Z1. The frequency of IFN-γ producing CD8+ T cells after re-stimulation with gag-CTL, gag-helper, medium, an irrelevant antigen, and con A (positive control) was determined using a standard IFN-γ-based ELISPOT assay.

Briefly, IFN-γ was detected using the mAb R46-A2 at 5 mg/ml and polyclonal rabbit anti-IFN-γ used at an optimal dilution (kindly provided by Dr. Phillip Scott, University of Pennsylvania, Philadelphia, Pa.). The levels of IFN-γ were calculated by comparison with a standard curve using murine rIFN-γ (Life Technologies, Gaithersburg, Md.). Plates were developed using a peroxidase-conjugated goat anti-rabbit IgG Ab (IFN-γ). Plates were then read at 405 nm. The lower limit of detection for the assays was 30 pg/ml.

### Results

### Example 9: Suppressor Cell Function after Listeria Treatment

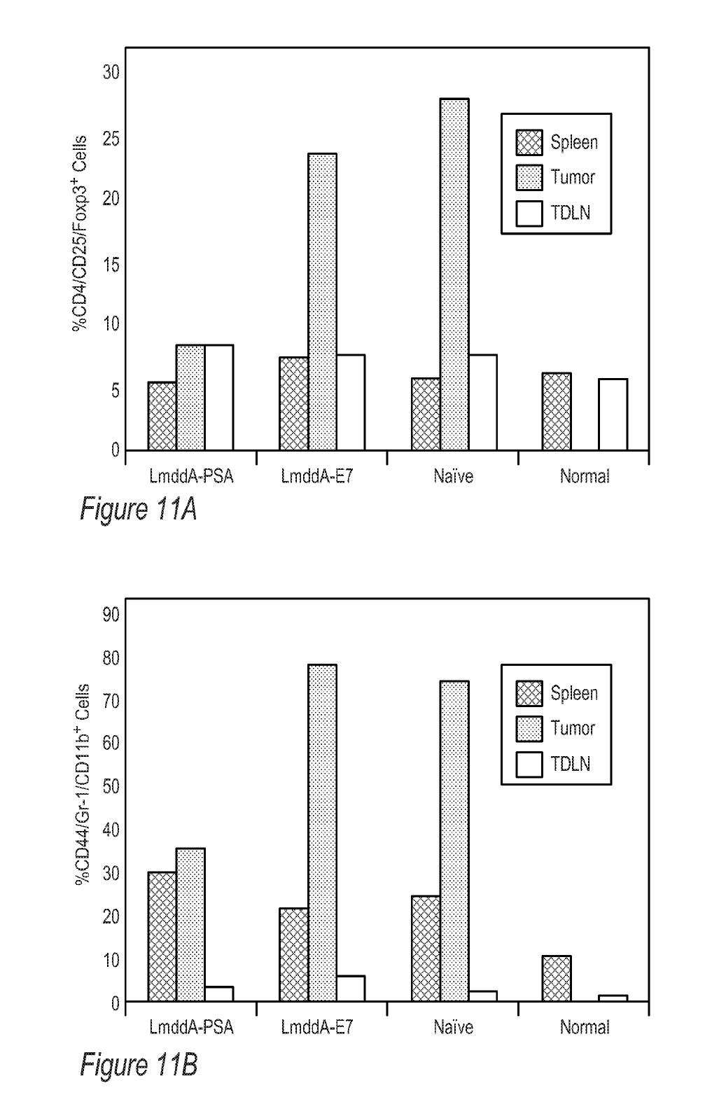

At day 0 tumors were implanted in mice. At day 7 mice were vaccinated with Lmdda-E7 or LmddA-PSA. At day 14 tumors were harvested and the number and percentages of infiltrating MDSCs and Treg were measured for vaccinated and naïve groups. It was found that there is a decrease in the percentages of both MDSC and Tregs in the tumors of Listeria-treated mice, and the absolute number of MDSC, whereas the same effect is not observed in the spleens or the draining lymph nodes (TLDN) (FIG. 11).

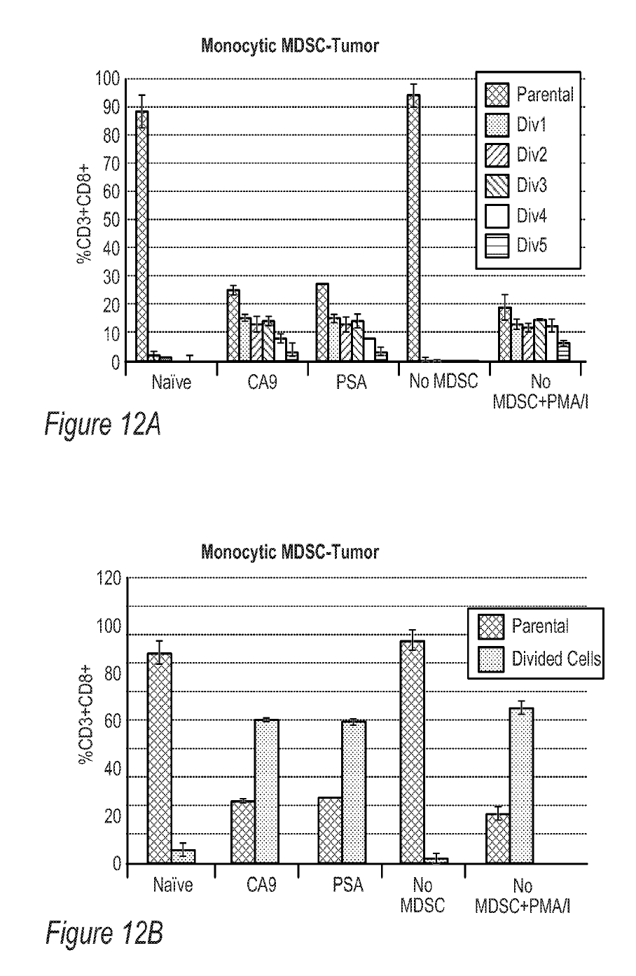

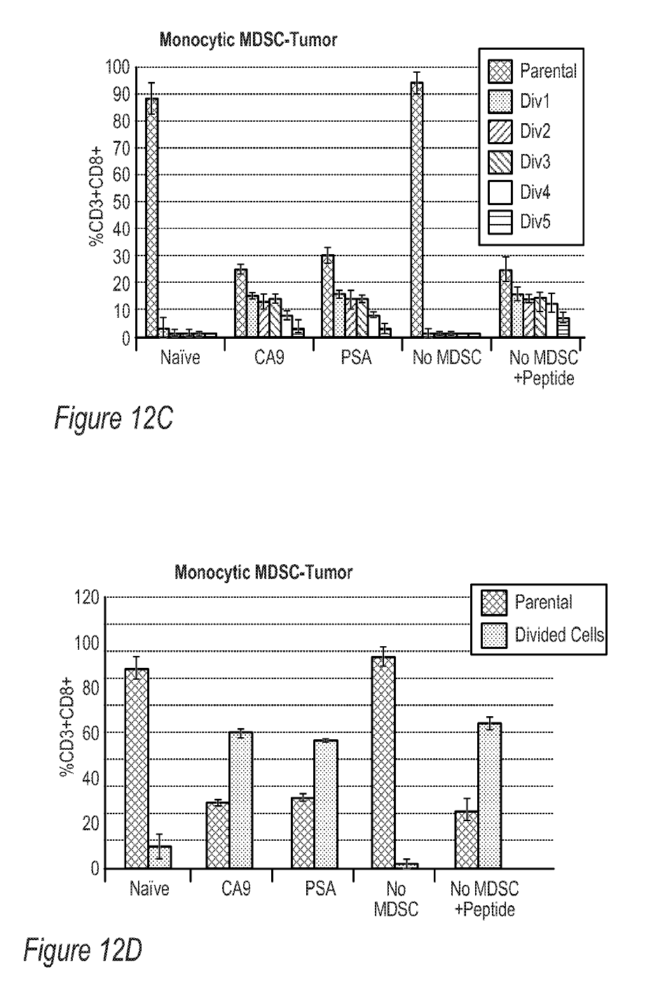

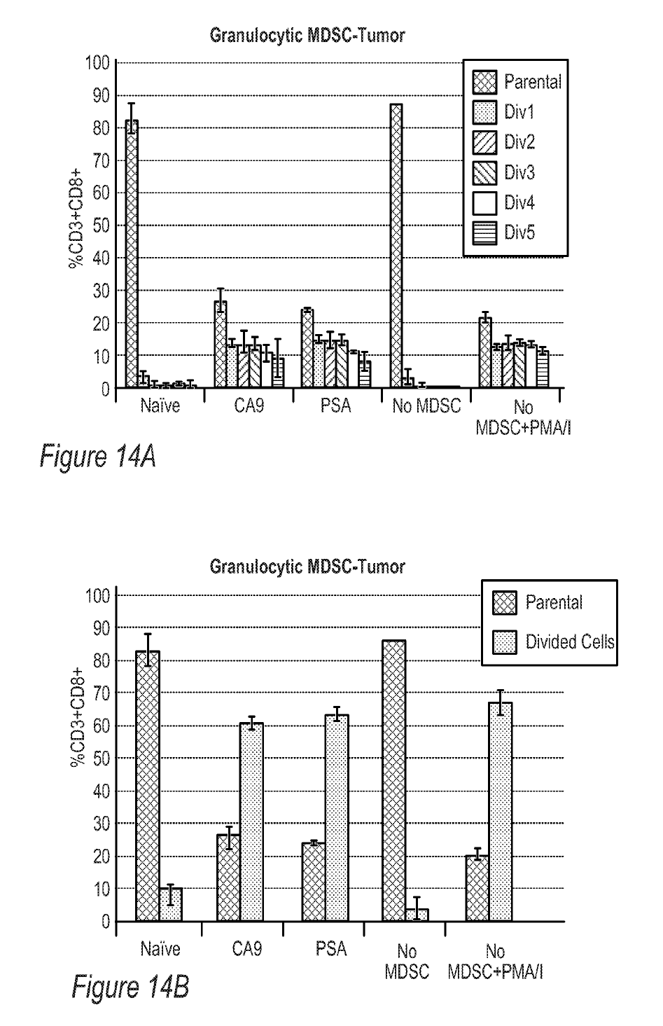

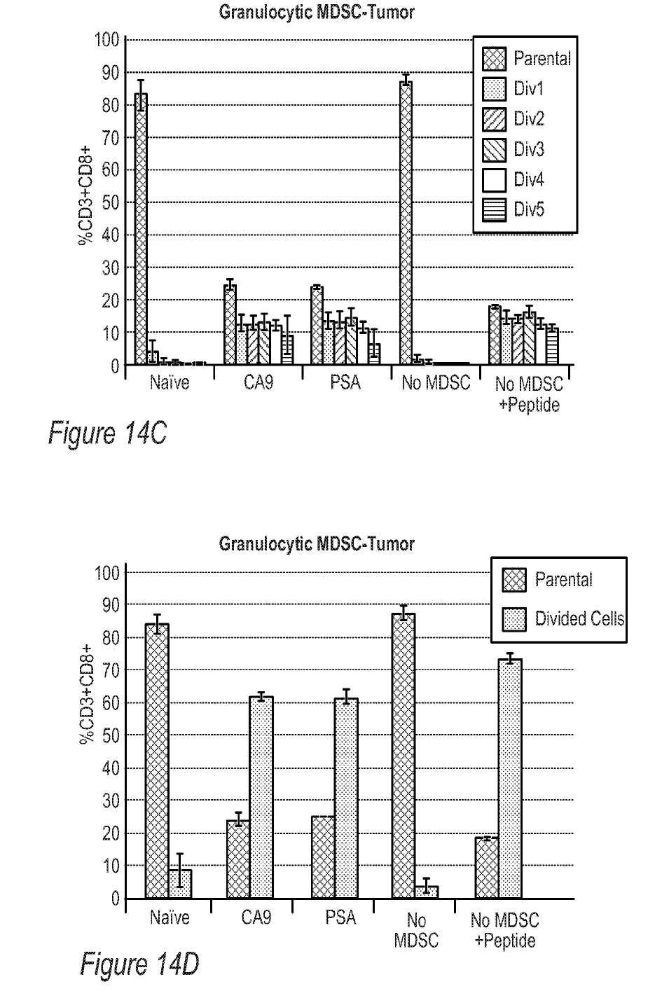

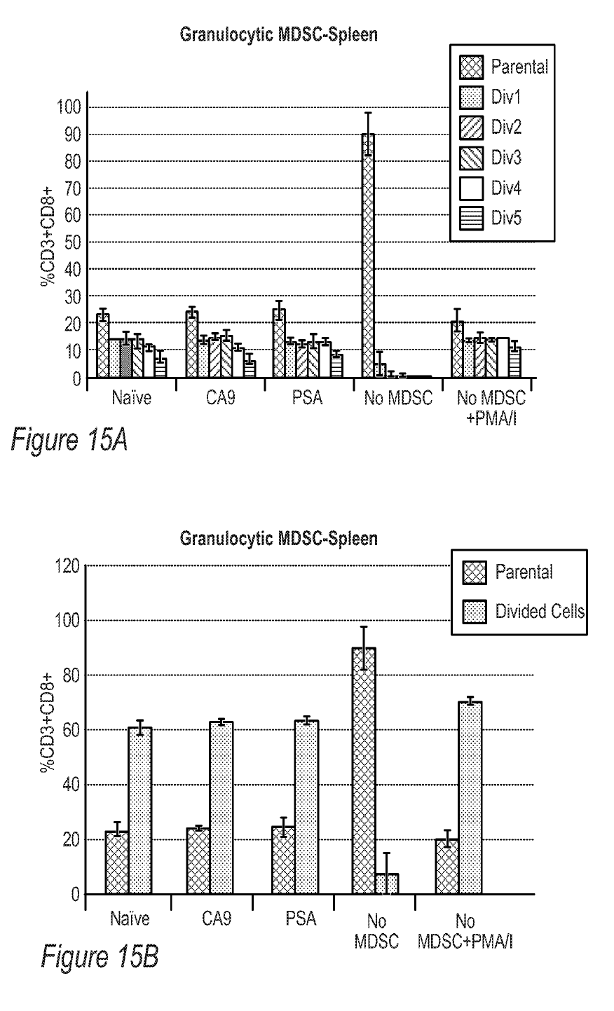

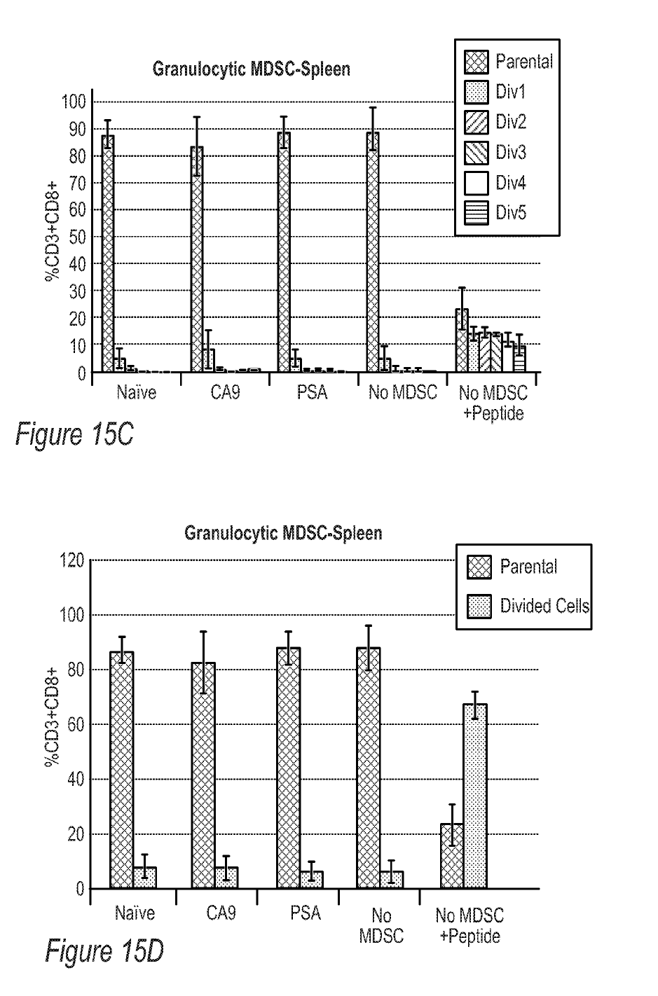

Isolated splenocytes and tumor-infiltrating lymphocytes (TILs) extracted from tumor bearing mice in the above experiment were pooled and stained for CD3, and CD8 to elucidate the effect of immunization with Lm-LLO-E7, Lm-LLO-PSA and Lm-LLO-CA9, Lm-LLO-Her2 (FIG. 12-14) on the presence of MDSCs and Tregs (both splenic and tumoral MDSCs and Tregs) in the tumor. Each column represents the % of T cell population at a particular cell division stage and is subgrouped under a particular treatment group (naïve, peptide-CA9 or PSA-treated, no MDSC/Treg, and no MDSC+PMA/ionomycin) (see FIGS. 12-14).

Blood from tumor-bearing mice was analyzed for the percentages of Tregs and MDSCs present. There is a decrease in both MDSC and Tregs in the blood of mice after Lm vaccination.

### Example 10: MDSCs from TPSA23 Tumors but not Spleen are Less Suppressive after Listeria Vaccination

Suppressor assays were carried out using monocytic and granulocytic MDSCs isolated from TPSA23 tumors with non-specifically activated naïve murine cells, and specifically activated cells (PSA, CA9, PMA/ionomycyn). Results demonstrated that the MDSCs isolated from tumors from the Lm vaccinated groups have a diminished capacity to suppress the division of activated T cells as compared to MDSC from the tumors of naïve mice. (see Lm-LLO-PSA and Lm-LLO-treated Groups in FIGS. 12 & 14, right-hand panel in figures represents pooled cell division data from left-hand panel). In addition, T responder cells from untreated mice where no MDSCs were present and where the cells were unstimulated/activated, remained in their parental (resting) state (FIGS. 12 & 14), whereas T cells stimulated with PMA or ionomycin were observed to replicate (FIGS. 12 & 14). Further, it was observed that both, the Gr+Ly6G+ and the GrdimLy6G-MDSCs are less suppressive after treatment with Listerias. This applies to their decreased abilities to suppress both the division of activated PSA-specific T cells and non-specific (PMA/Ionomycin stimulated) T cells.

Moreover, suppressor assays carried out using MDSCs isolated from TPSA23 tumors with non-specifically activated naïve murine cells demonstrated that the MDSCs isolated from tumors from the Lm vaccinated groups have a diminished capacity to suppress the division of activated T cells as compared to MDSC from the tumors of naïve mice (see FIGS. 12 & 14).

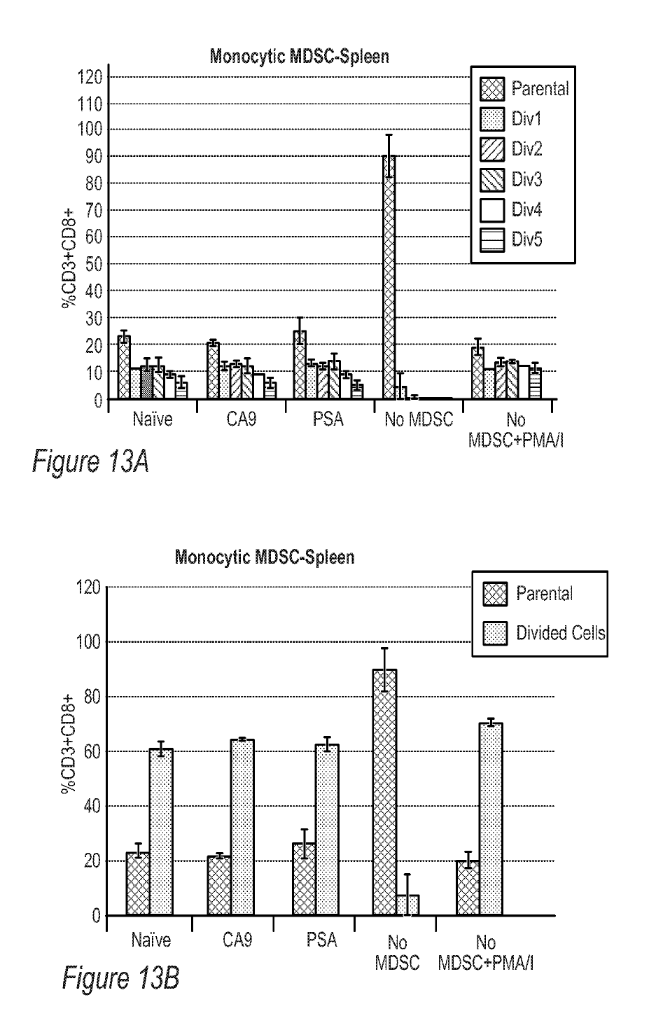

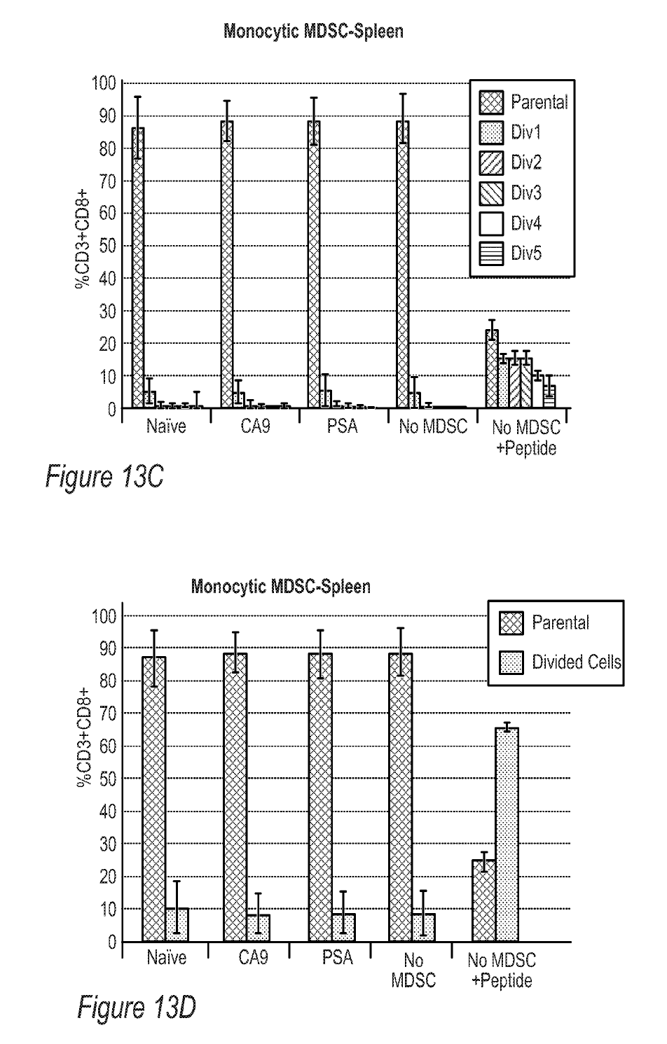

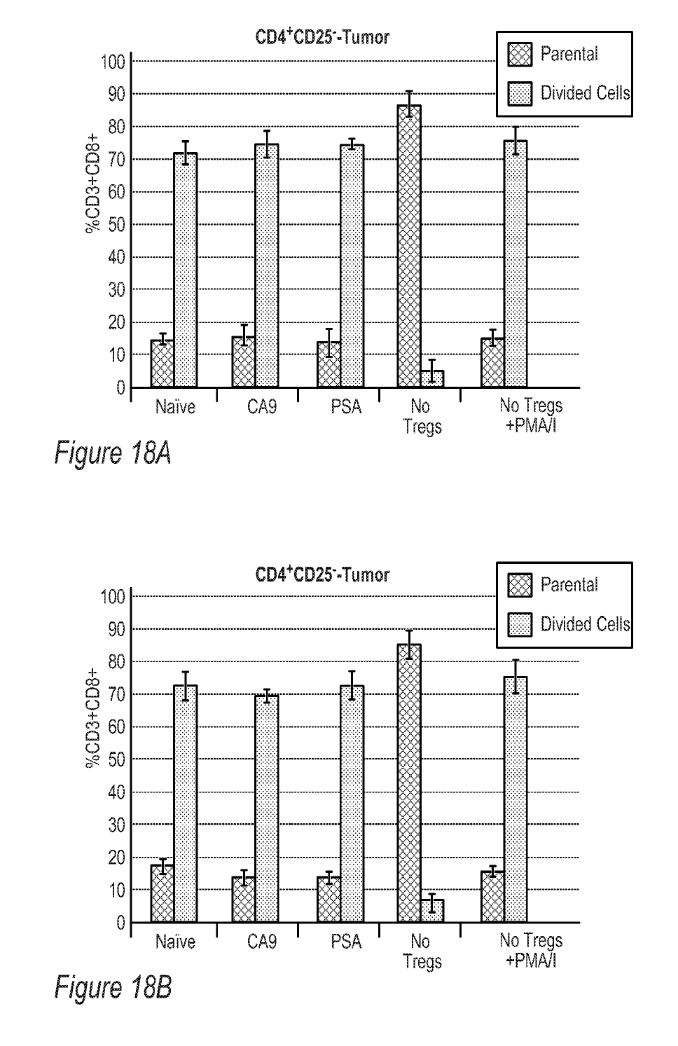

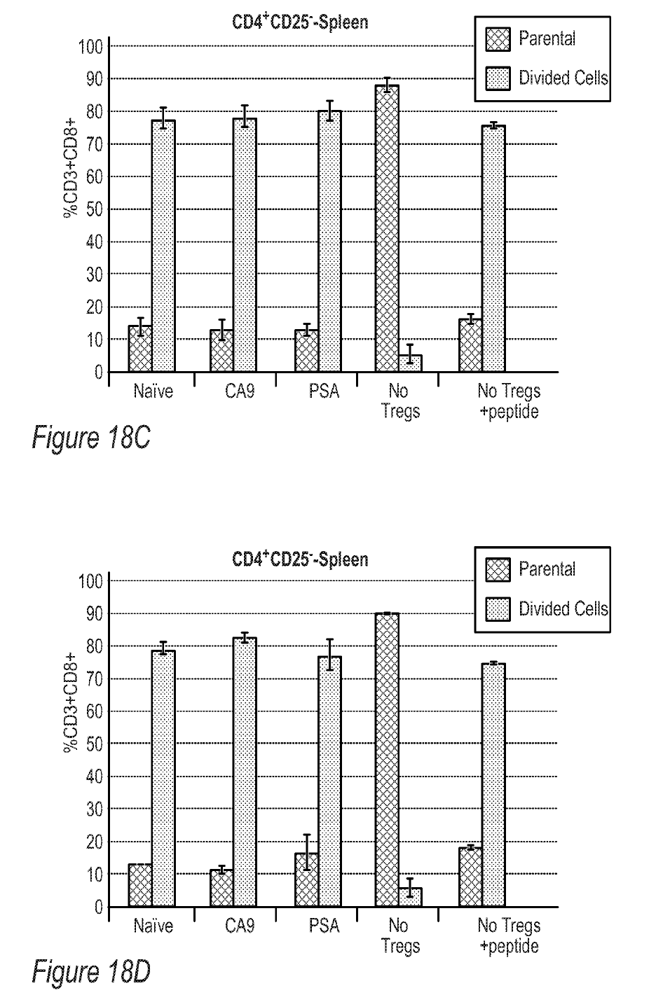

In addition, the observations discussed immediately above relating to FIGS. 12 and 18 were not observed when using splenic MDSCs. In the latter, splenocytes/T cells from the naïve group, the Listeria-treated group (PSA, CA9), and the PMA/ionomycin stimulated group (positive control) all demonstrated the same level of replication (FIGS. 13 & 15). Hence, these results show that Listeria-mediated inhibition of suppressor cells in tumors worked in an antigen-specific and non-specific manner, whereas Listeria has no effect on splenic granulocytic MDSCs as they are only suppressive in an antigen-specific manner.

### Example 11: Tumor T Regulatory Cells' Reduced Suppression

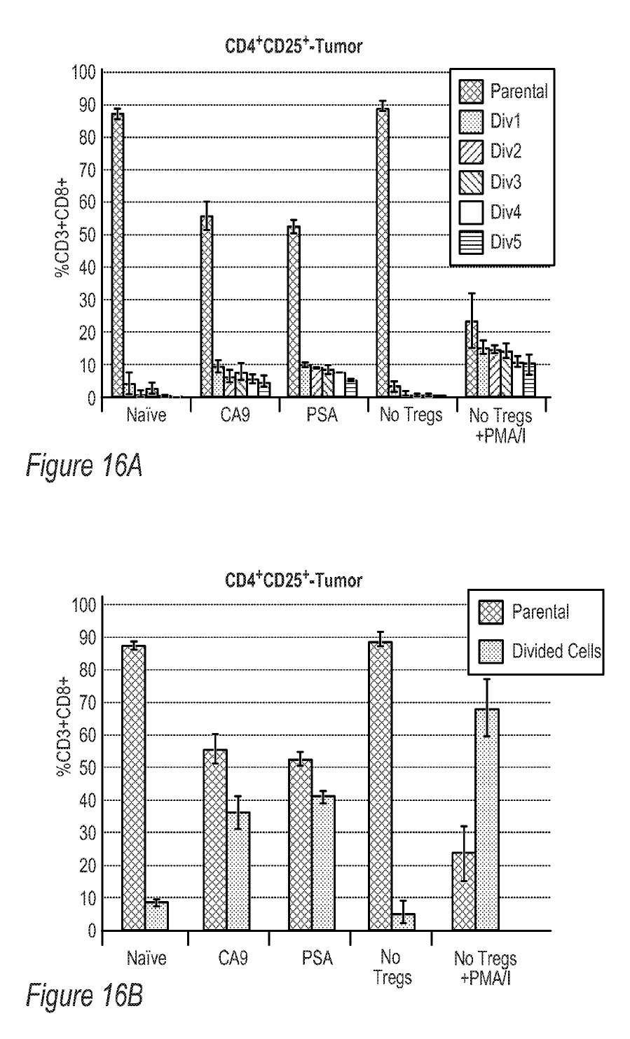

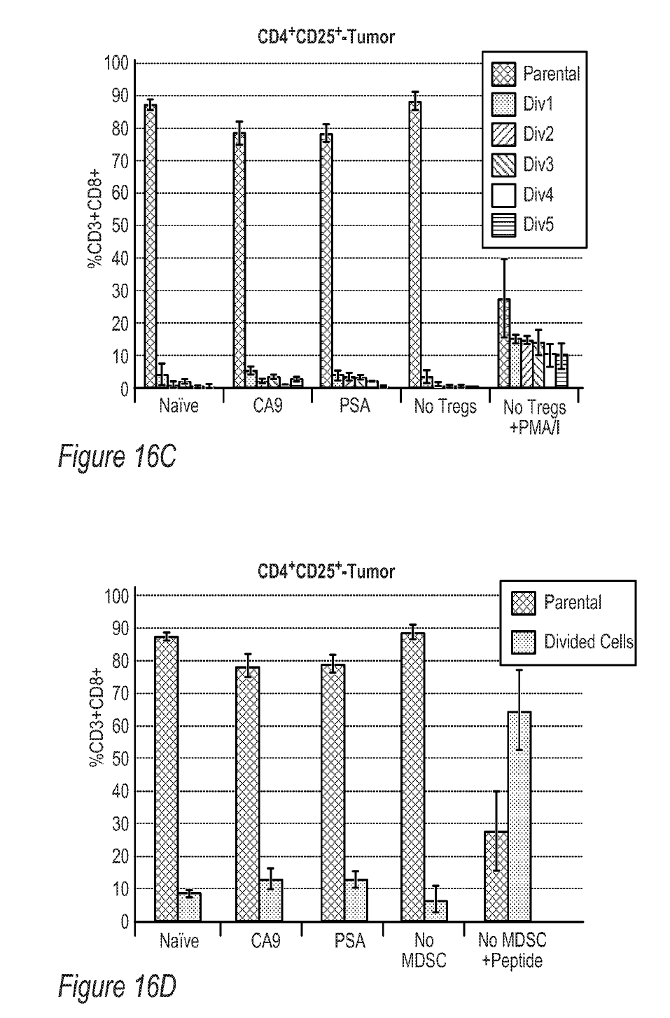

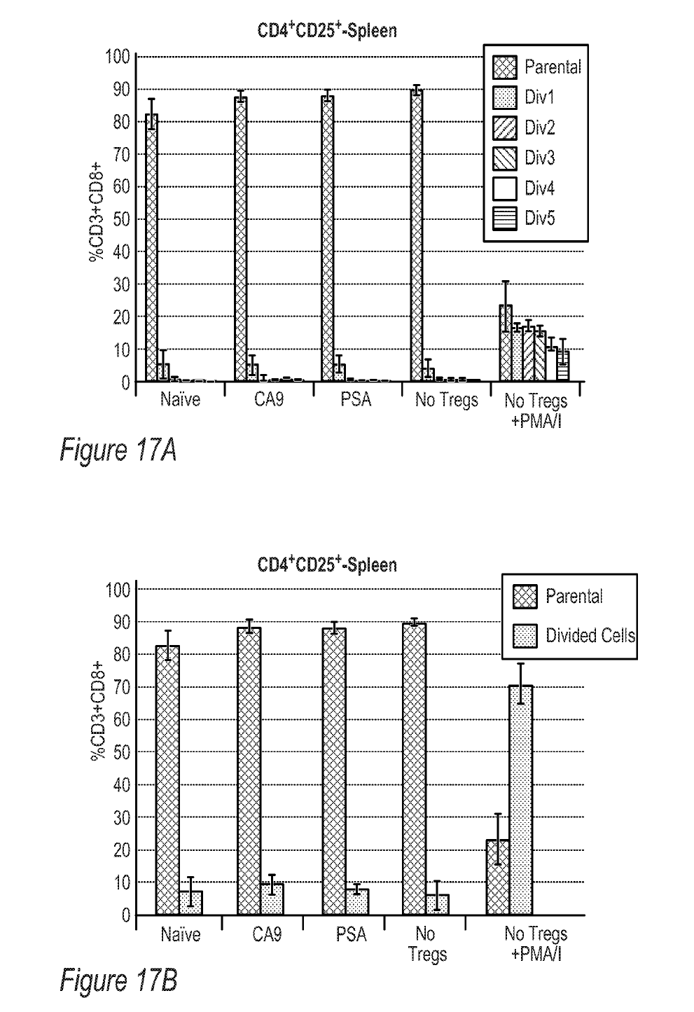

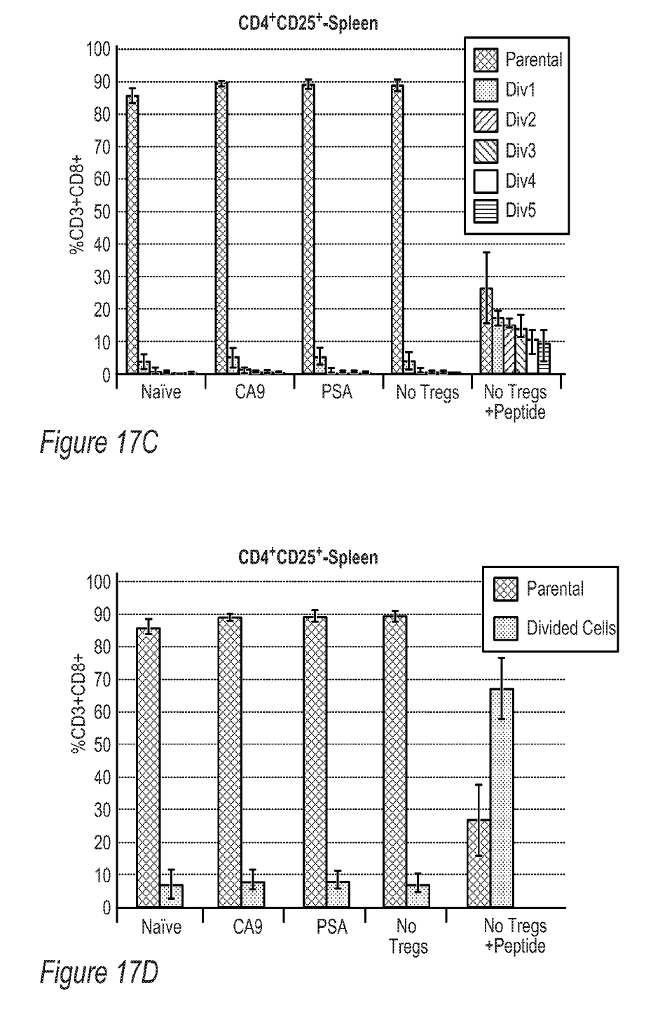

Suppressor assays were carried out using Tregs isolated from TPSA23 tumors after Listeria treatment. It was observed that after treatment with Listeria there is a reduction of the suppressive ability of Tregs from tumors (FIG. 16), however, it was found that splenic Tregs are still suppressive (FIG. 17).

As a control conventional CD4+ T cells were used in place of MDSCs or Tregs and were found not to have an effect on cell division (FIG. 18).

### Example 12: MDSCs and Tregs from 4T1 Tumors but not Spleen are Less Suppressive after Listeria Vaccination

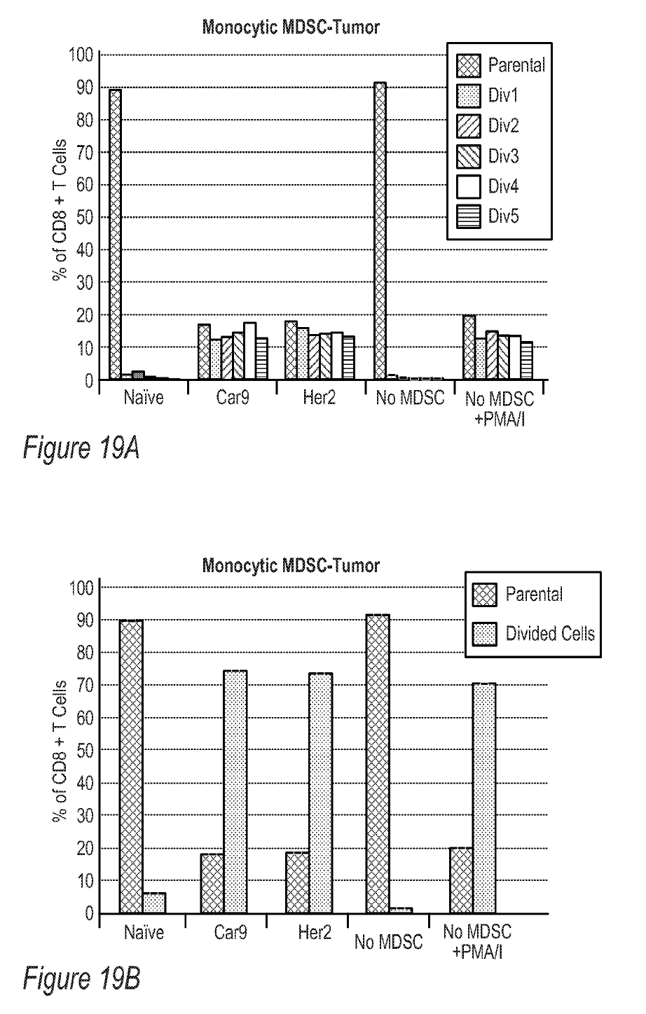

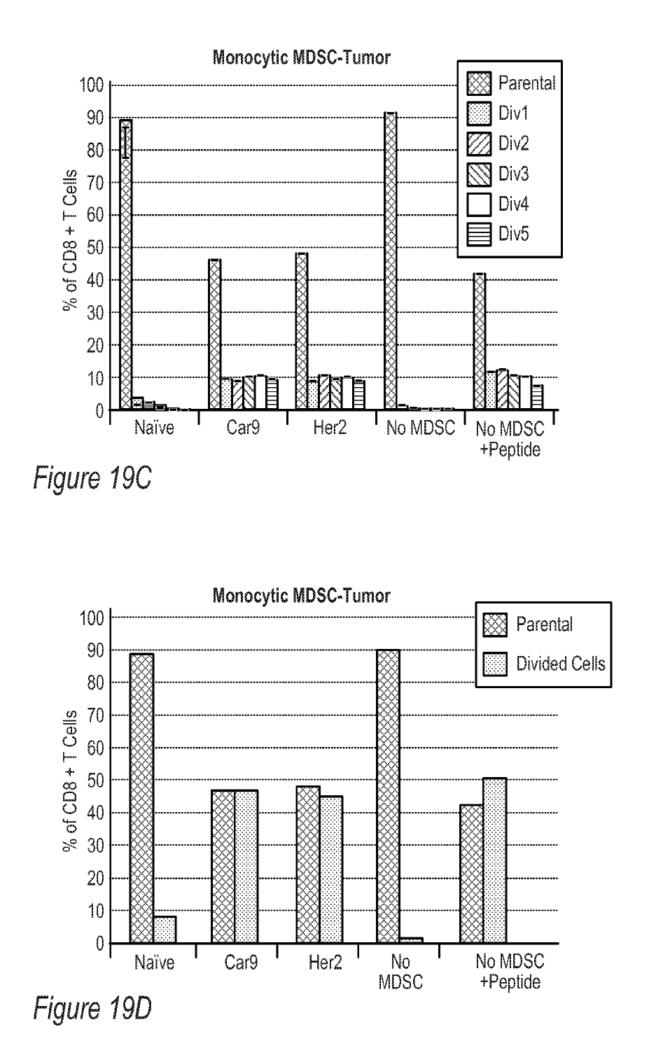

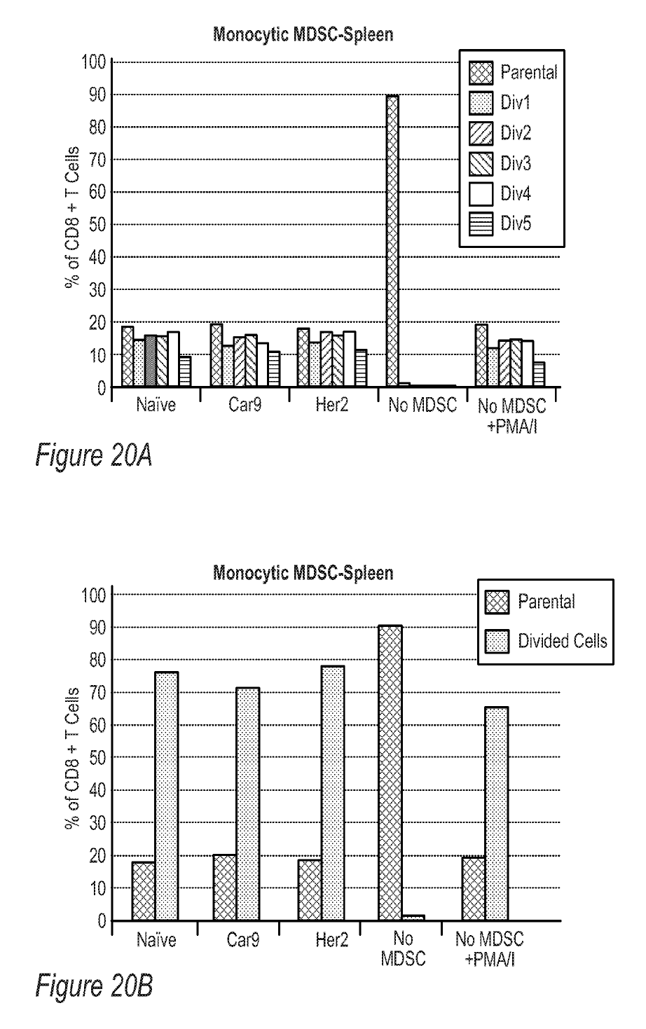

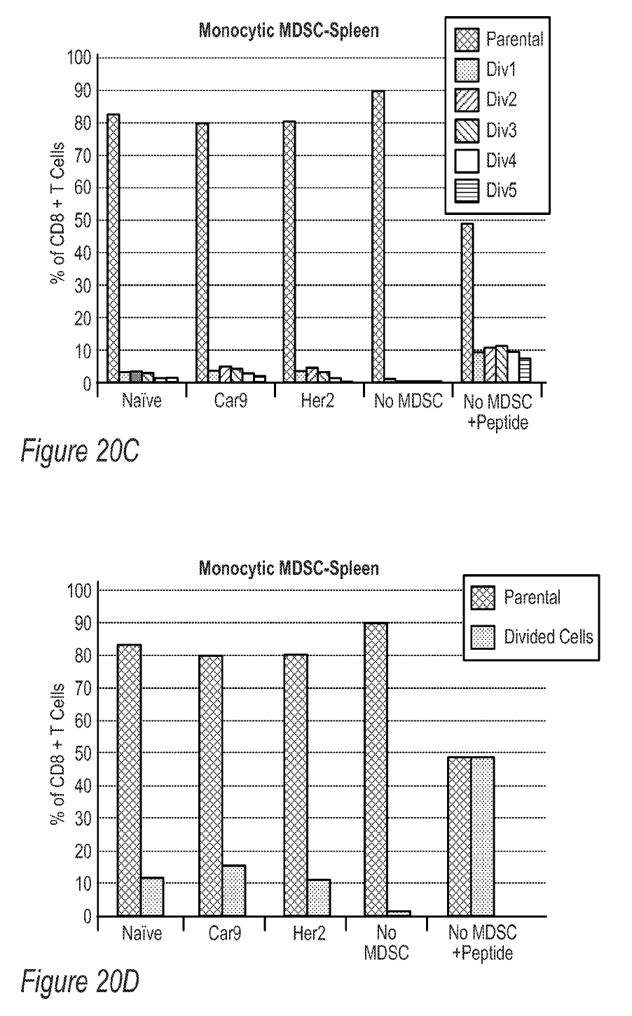

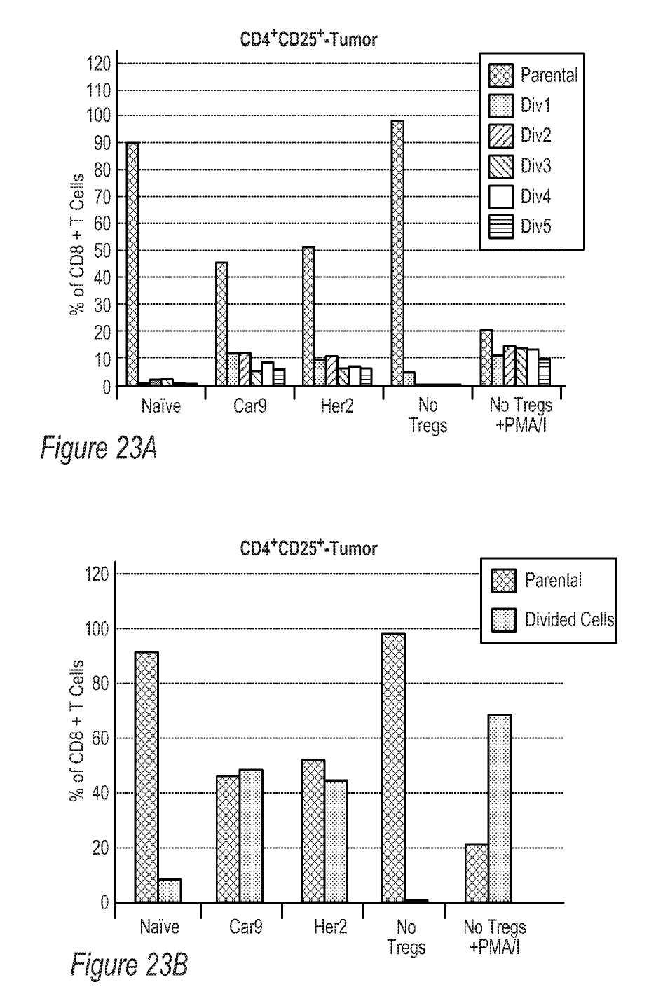

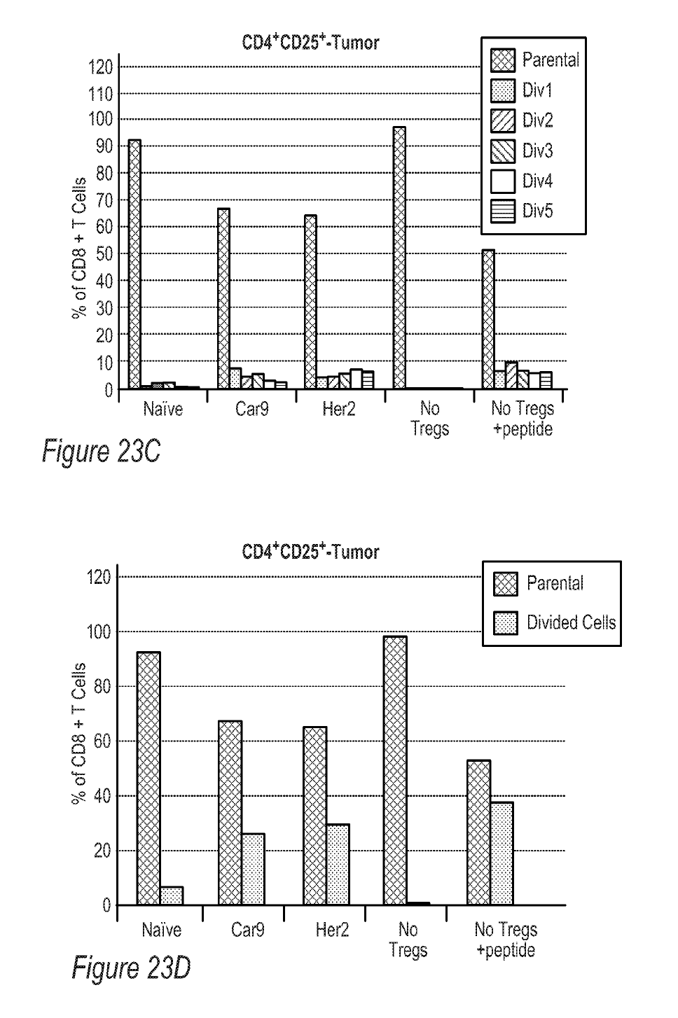

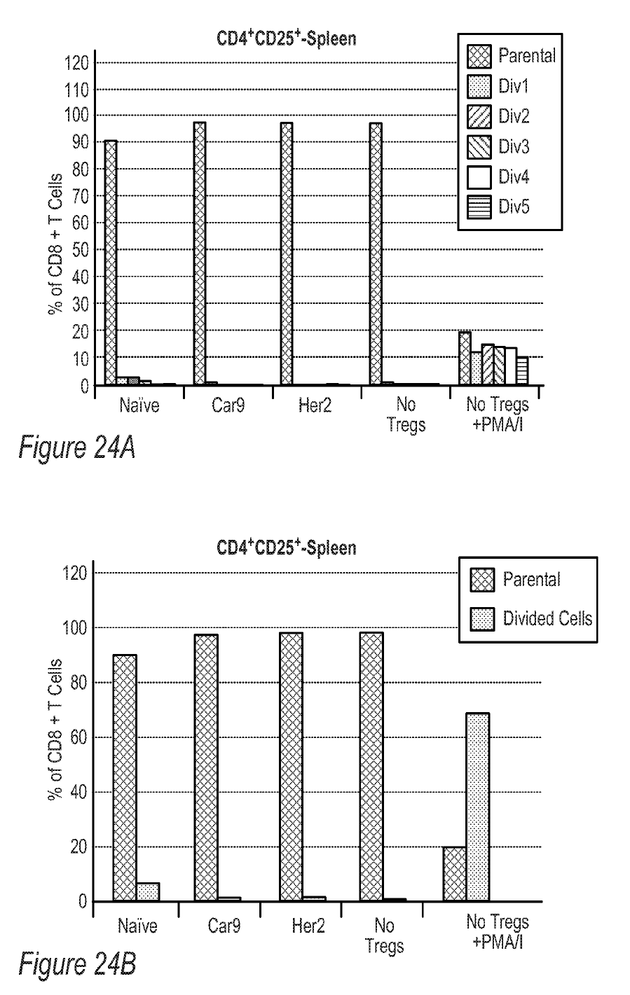

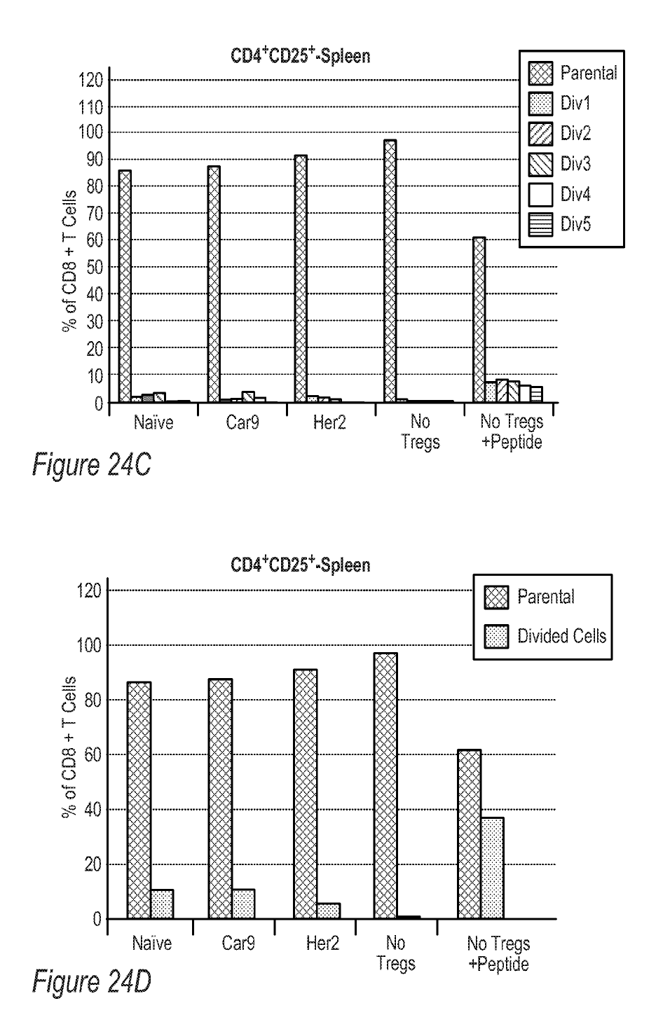

As in the above, the same experiments were carried out using 4T1 tumors and the same observations were made, namely, that MDSCs are less suppressive after Listeria vaccination (FIGS. 19 & 21), that Listeria has no specific effect on splenic monocytic MDSCs (FIGS. 20 & 22), that there is a decrease in the suppressive ability of Tregs from 4T1 tumors after Listeria vaccination (FIG. 23), and that Listeria has no effect on the suppressive ability of splenic Tregs (FIG. 24).

Finally, it was observed that Listeria has no effect on the suppressive ability of splenic Tregs.

### Example 13: Change in the Suppressive Ability of the Granulocity and Monocytic MDSC is Due to the Overexpression of tLLO

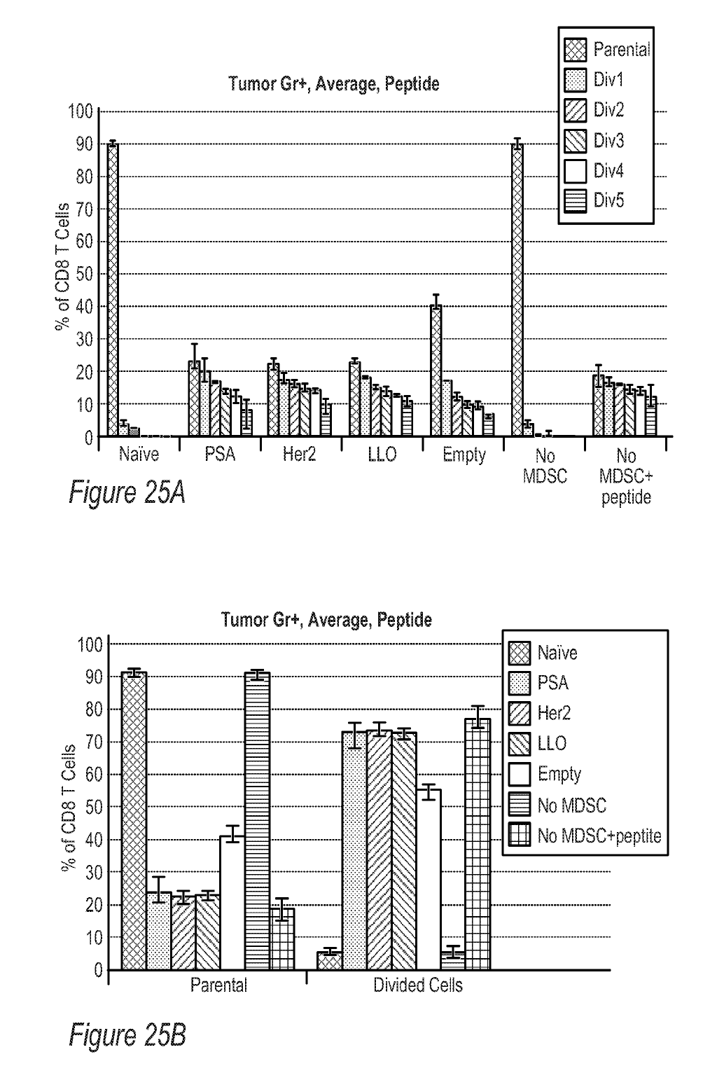

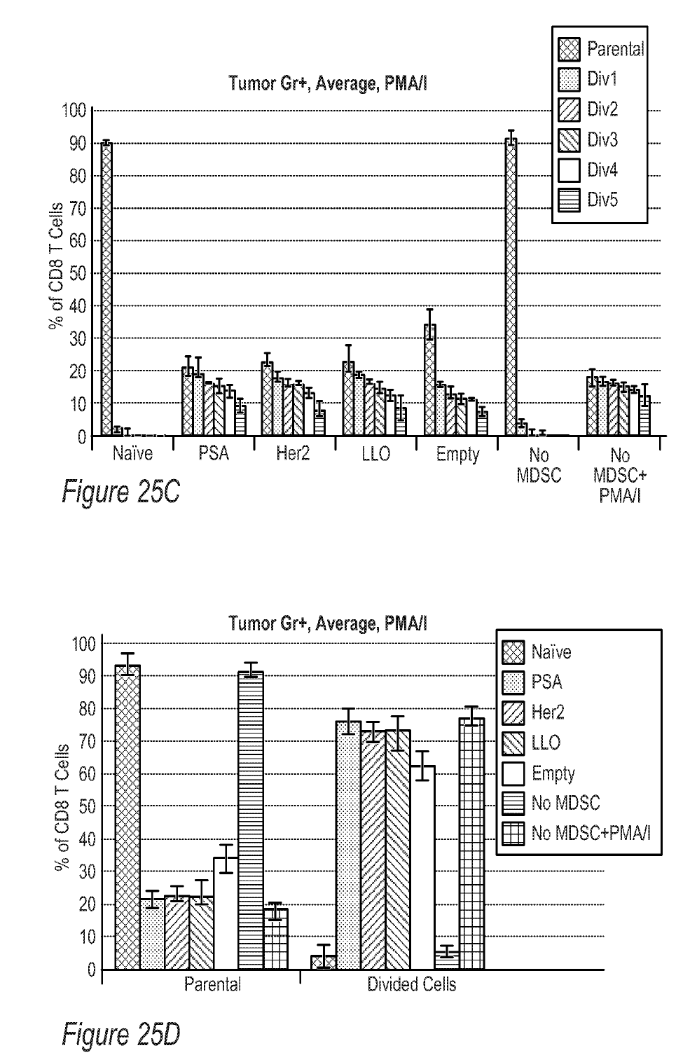

The LLO plasmid shows similar results as the Listerias with either the TAA or an irrelevant antigen (FIG. 25). This means that the change in the suppressive ability of the granulocytic MDSC is due to the overexpression of tLLO and is independent of the partnering fusion antigen. The empty plasmid construct alone also led to a change in the suppressive ability of the MDSC, although not to exactly the same level as any of the vaccines that contain the truncated LLO on the plasmid. The average of the 3 independent experiments show that the difference in suppression between the empty plasmid and the other plasmids with tLLO (with and without a tumor antigen) are significant. Reduction in MDSC suppressive ability was identical regardless of the fact if antigen specific or non-specific stimulated responder T cells were used.

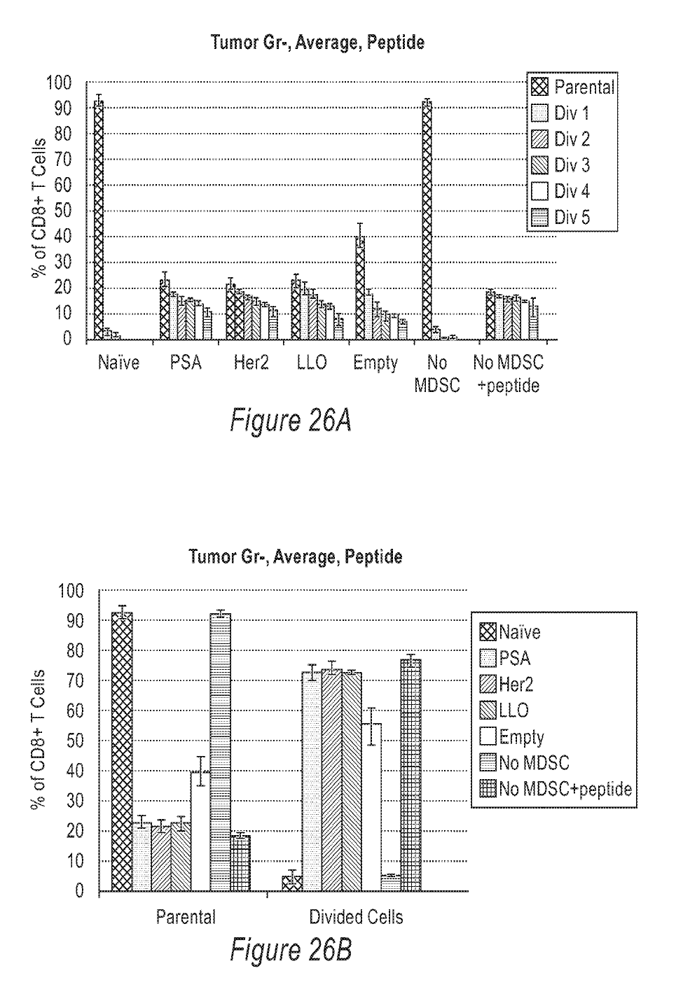

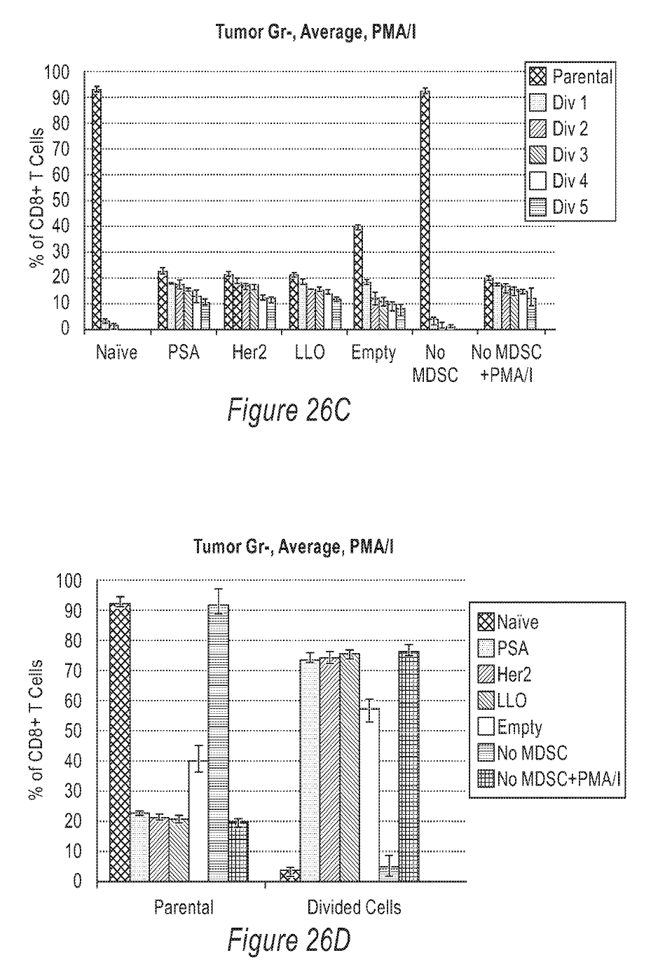

Similar to the granulocytic MDSC, the average of the 3 independent experiments shows that the differences observed in the suppressive ability of the monocytic MDSCs purified from the tumors after vaccination with the Lm-empty plasmid vaccine are significant when compared to the other vaccine constructs (FIG. 26).

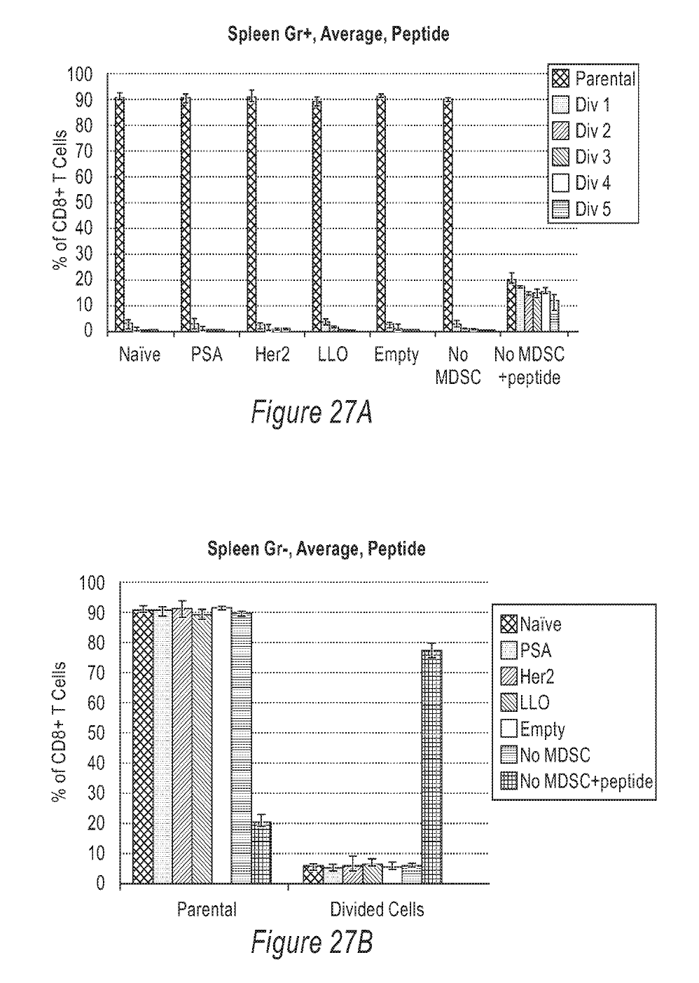

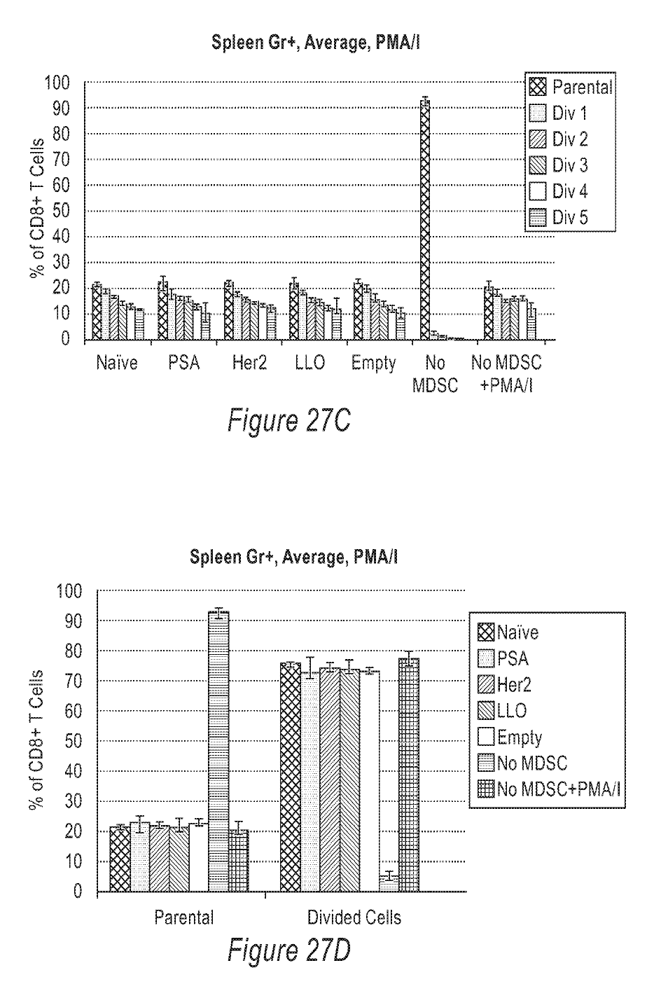

Similar to the above observations, granulocytic MDSC purified from the spleen retain their ability to suppress the division of the antigen-specific responder T cells after Lm vaccination (FIG. 27). However, after non-specific stimulation, activated T cells (with PMA/ionomycin) are still capable of dividing. None of these results are altered with the use of the LLO only or the empty plasmid vaccines showing that the Lm-based vaccines are not affecting the splenic granulocytic MDSC (FIG. 27).

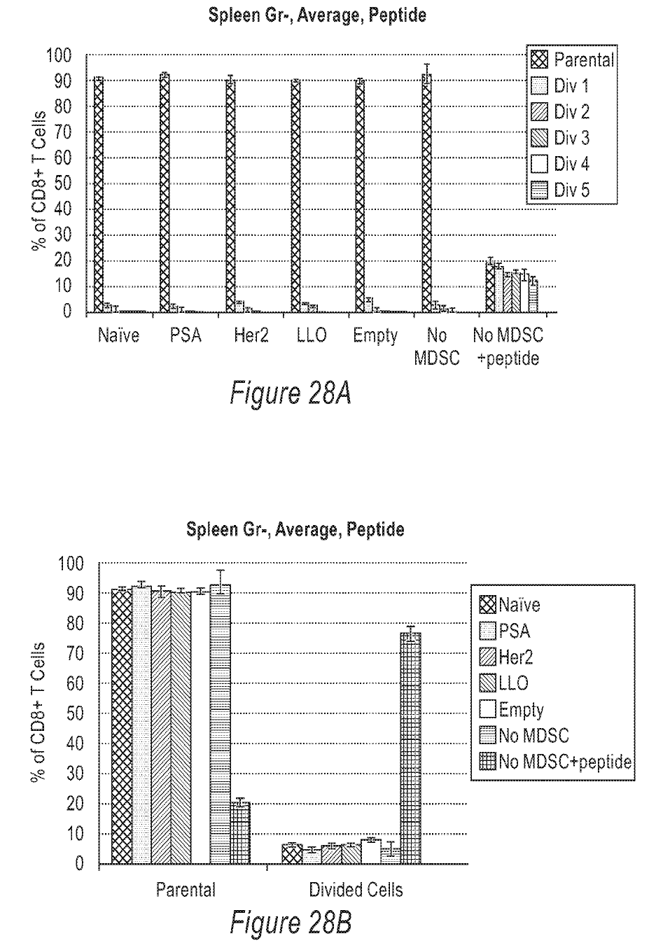

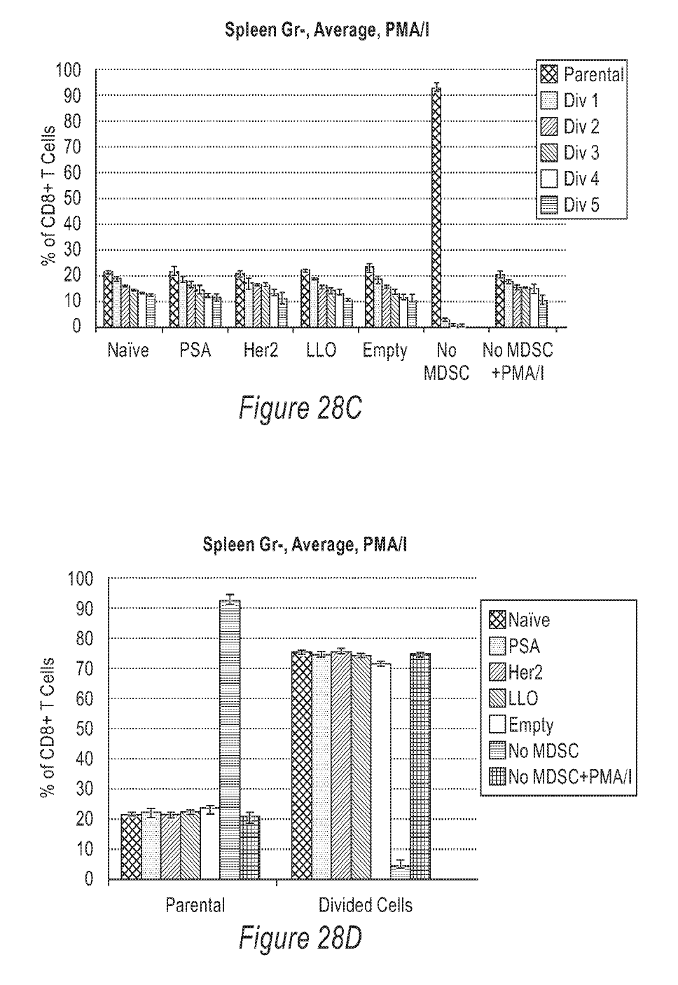

Similarly, monocytic MDSC purified from the spleen retain their ability to suppress the division of the antigen-specific responder T cells after Lm vaccination. However, after non-specific activation (stimulated by PMA/ionomycin), T cells are still capable of dividing. None of these results are altered with the use of the LLO only or the empty plasmid vaccines showing that the Lm vaccines are not affecting the splenic monocytic MDSC (FIG. 28).

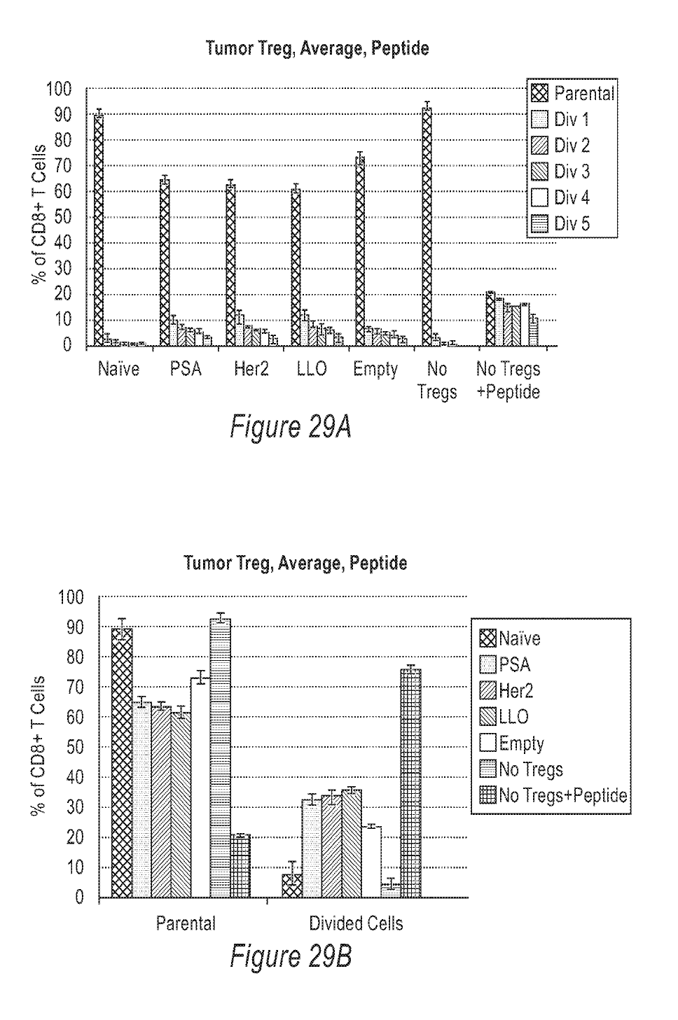

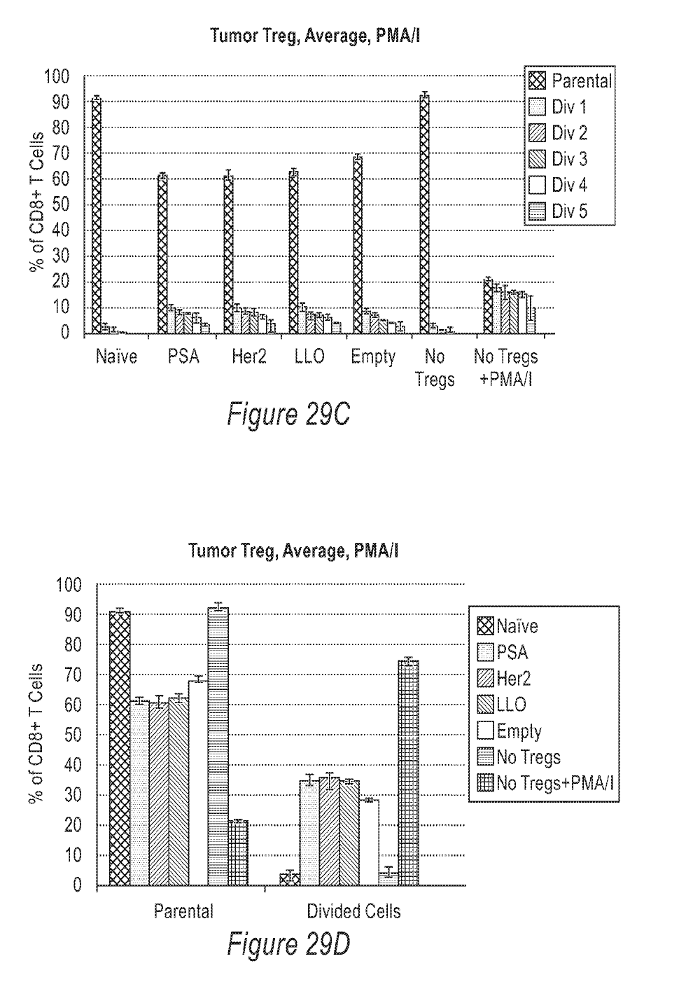

Tregs purified from the tumors of any of the Lm-treated groups have a slightly diminished ability to suppress the division of the responder T cells, regardless of whether the responder cells are antigen specific or non-specifically activated. Especially for the non-specifically activated responder T cells, it looks as though the vaccine with the empty plasmid shows the same results as all the vaccines that contain LLO on the plasmid. Averaging this experiment with the others shows that the differences are not significant (FIG. 29).

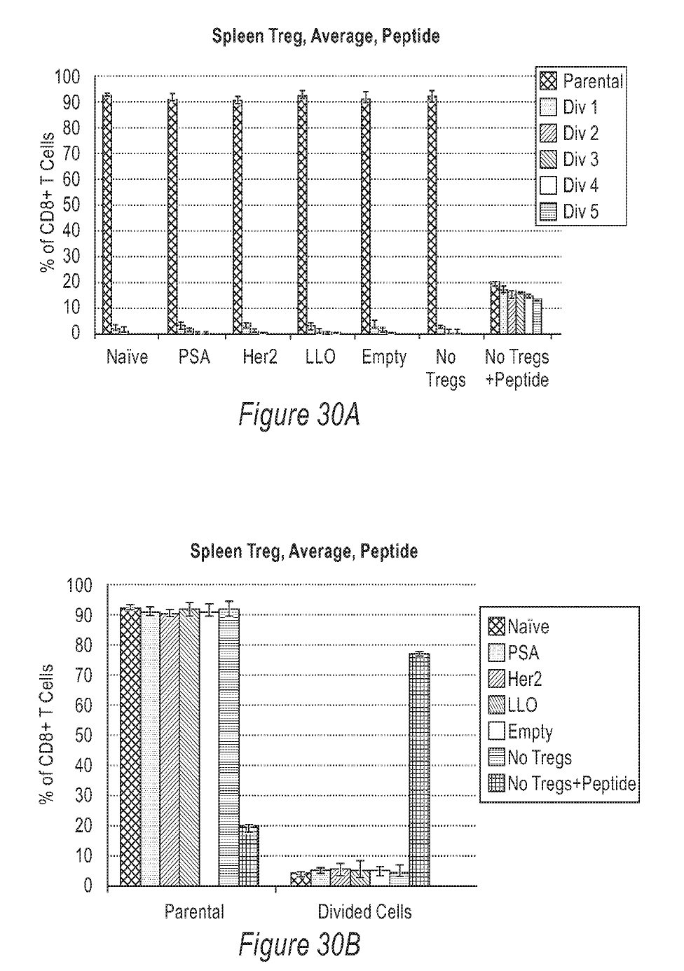

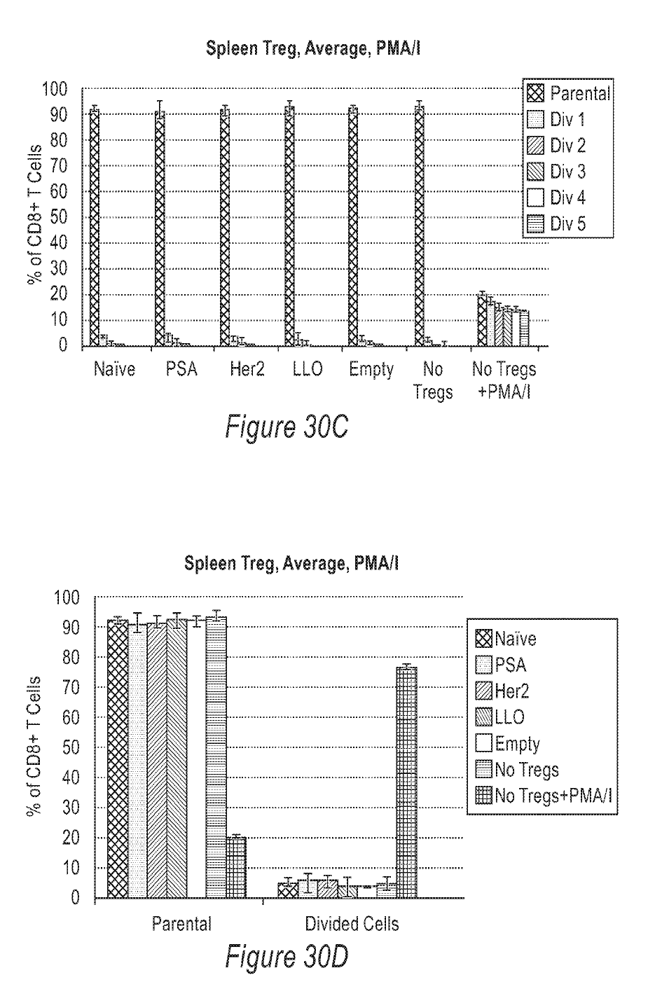

Tregs purified from the spleen are still capable of suppressing the division of both antigen specific and non-specifically activated responder T cells. There is no effect of Lm treatment on the suppressive ability of splenic Tregs (FIG. 30).

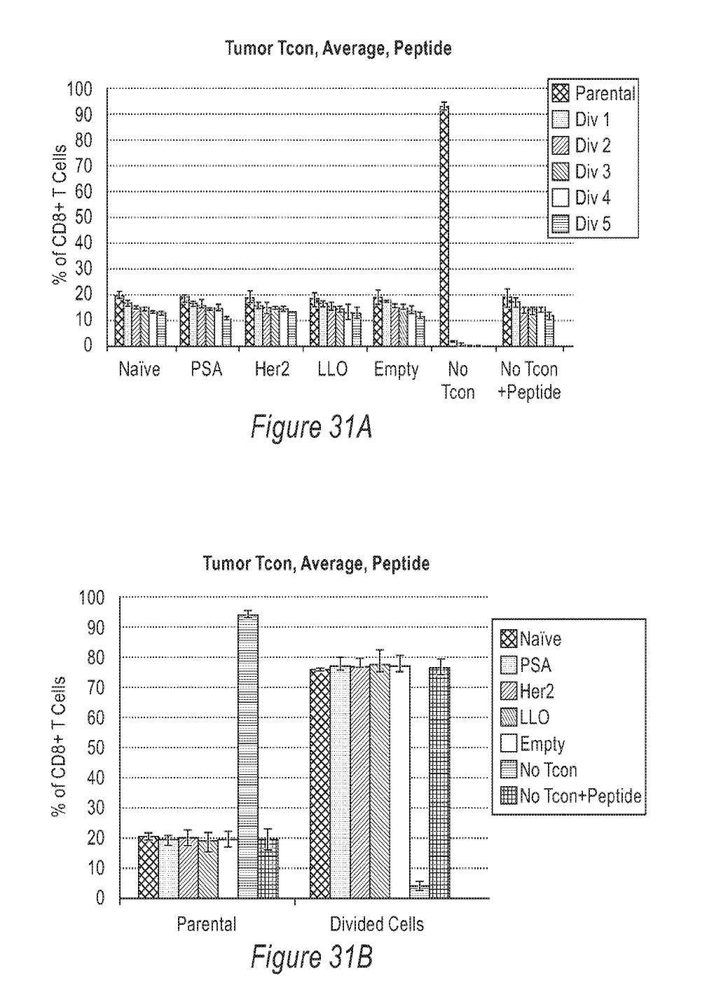

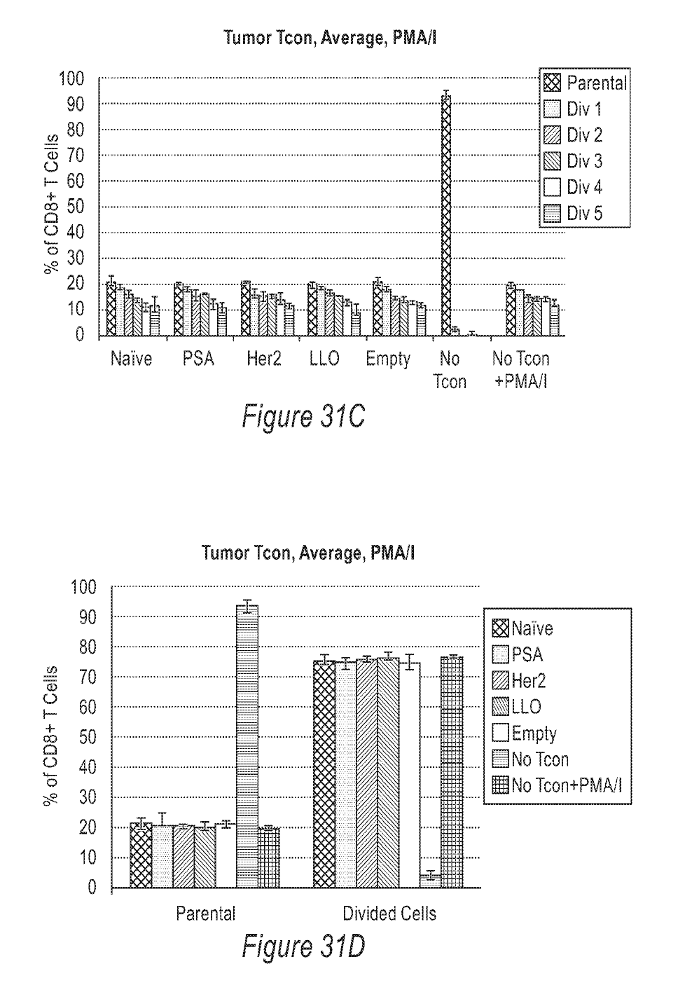

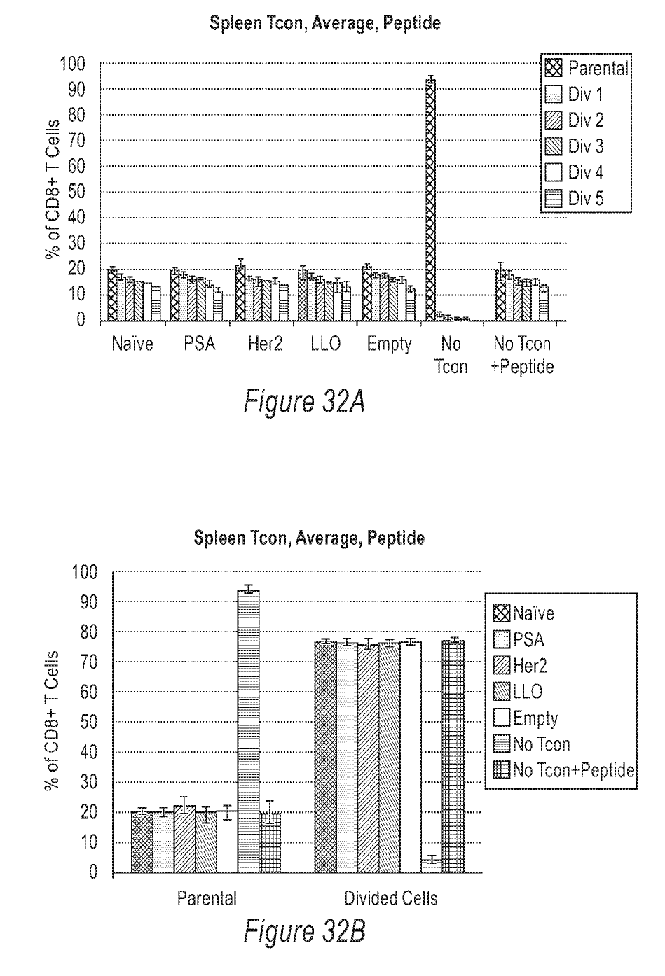

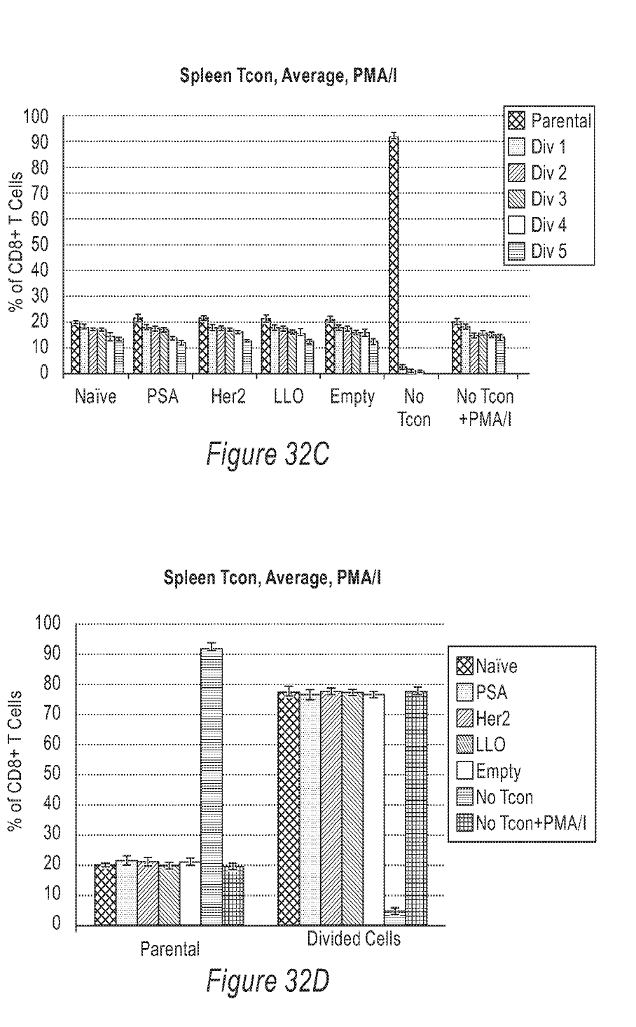

Tcon cells are not capable of suppressing the division of T cells regardless of whether the responder cells are antigens specific or non-specifically activated, which is consistent with the fact that these cells are non-suppressive. Lm has no effect on these cells and there was no difference if the cells were purified from the tumors or the spleen of mice (FIGS. 31-32).

### Materials and Methods for Examples 14-20

Oligonucleotides were synthesized by Invitrogen (Carlsbad, Calif.) and DNA sequencing was done by Genewiz Inc, South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C-RPMI medium. Bioluminescent work was conducted under guidance by the Small Animal Imaging Facility (SAIF) at the University of Pennsylvania (Philadelphia, Pa.).

Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). This plasmid was used as a template to amplify three segments of hHer-2/neu, namely, EC1, EC2, and IC1, by PCR using pfx DNA polymerase (Invitrogen) and the oligos indicated in Table 3.

TABLE 3
Primers for cloning of Human her-2-Chimera

| | | | Amino acid |
|---|---|---|---|
| | | Base pair | region or |
| | DNA sequence | region | junctions |
| Her-2- | TGATCTCGAGACCCACCTGGACATGCTC | 120-510 | 40-170 |
| Chimera (F) | | | |
| HerEC1-ECF2 | CTACCAGGACACGATTTTGTGGAAGAATATCCA | 510/1077 | 170/359 |
| (Junction) | GGAGTTTGCTGGCTGC | | |
| | (SEQ ID NO: 75) | | |
| HerEC1-EC2R | GCAGCCAGCAAACTCCTGGATATT-CTTCCACA | | |
| (Junction) | AAATCGTGTCCTGGTAG | | |
| | (SEQ ID NO: 76) | | |
| HerEC2-IFIF | CTGCCACCAGCTGTGCGCCCGAGGG-CAGCAGA | 1554/2034 | 518/679 |
| (Junction) | AGATCCGGAAGTACACGA | | |
| | (SEQ ID NO: 77) | | |
| HerEC2-ICI4 | TCGTGTACTTCCGGATCTTCTGCTGCCCTCGGG | | |
| (Junction) | C GCACAGCTGGTGGCAG | | |
| | (SEQ ID NO: 78) | | |
| Her-2- | GTGGCCCGGGTCTAGATTAGTCTAAGAGGCAGC | 2034-2424 | 679-808 |
| Chimera (R) | CATAG11G | | |
| | (SEQ ID NO: 79) | | |

The Her-2/neu chimera construct was generated by direct fusion by the SOEing PCR method and each separate hHer-2/neu segment as templates. Primers are shown in Table 4.

TABLE 4

| | | Base pair | Amino acid |
|---|---|---|---|
| | DNA sequence | region | region |
| Her-2-EC1(F) | CCGCCTCGAGGCCGCGAGCACCCAAGTG | 58-979 | 20-326 |
| | (SEQ ID NO: 80) | | |
| Her-2-EC1(R) | CGCGACTAGTTTAATCCTCTGCTGTCACCTC | | |
| | (SEQ ID NO: 81) | | |
| Her-2-EC2(F) | CCGCCTCGAGTACCTTTCTACGGACGTG | 907-1504 | 303-501 |
| | (SEQ ID NO: 82) | | |
| Her-2-EC2(R) | CGCGACTAGTTTACTCTGGCCGGTTGGCAG | | |
| | (SEQ ID NO: 83) | | |
| Her-2-Her-2-IC1(F) | CCGCCTCGAGCAGCAGAAGATCCGGAAGTAC | 2034-3243 | 679-1081 |
| | (SEQ ID NO: 84) | | |
| Her-2-IC1(R) | CGCGACTAGTTTAAGCCCCTTCGGAGGGTG | | |
| | (SEQ ID NO: 85) | | |

Sequence of primers for amplification of different segments human Her2 regions

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 g/ml). In some experiments similar Listeria strains expressing hHer2/neu (Lm-hHer2) fragments were used for comparative purposes. In all studies, an irrelevant Listeria construct (Lm-control) was included to account for the antigen independent effects of Listeria on the immune system. Lm-controls were based on the same Listeria platform as ADXS31-164 (LmddA-ChHer2), but expressed a different antigen such as HPV16-E7 or NY-ESO-1. Expression and secretion of fusion proteins from Listeria were tested. Each construct was passaged twice in vivo.

Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naïve animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm. Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C-RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-γ) using mouse IFN-γ Enzyme-linked immunosorbent assay (ELISA) kit according to manufacturer's recommendations.

Tumor Studies in Her2 Transgenic Animals

Six weeks old FVB/N rat Her2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNA later at −20° C. In order to determine the effect of mutations in the Her2/neu protein on the escape of these tumors, genomic DNA was extracted using a genomic DNA isolation kit, and sequenced.

Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 14

### Generation of L. monocytogenes Strains that Secrete LLO Fragments Fused to Her-2 Fragments: Construction of ADXS31-164

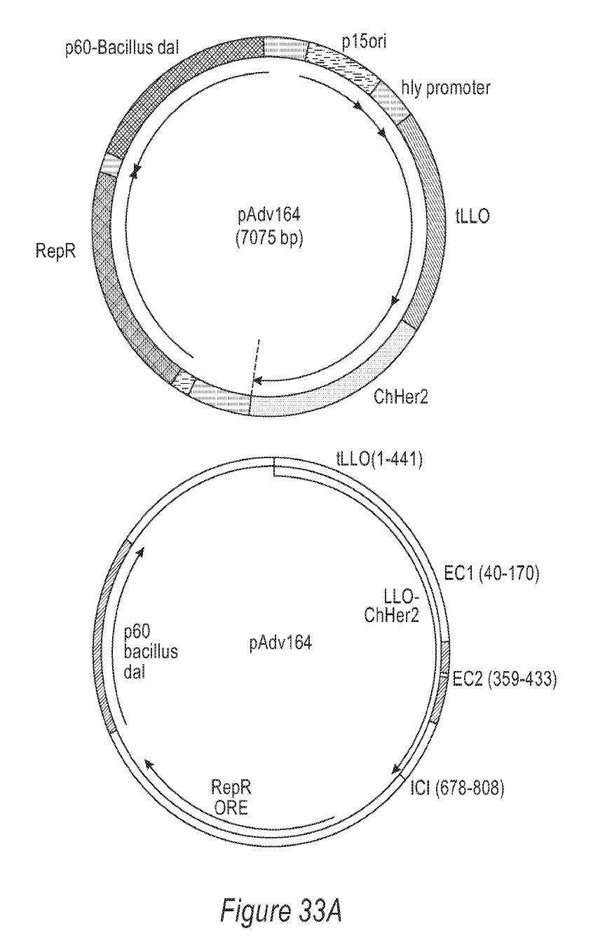

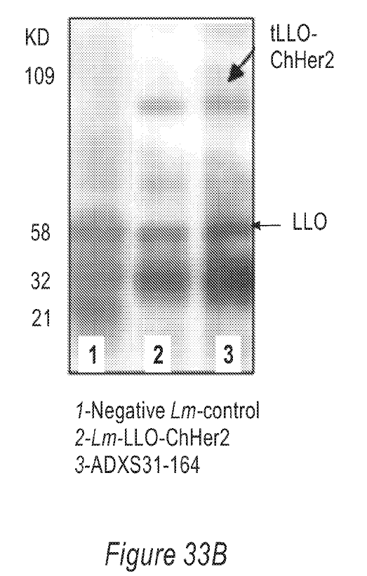

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 33A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria strains. 2) Unlike pAdv138, pAdv164 does not harbor a copy of the prfA gene in the plasmid (see sequence below and FIG. 33A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 33B) as a band of ˜104 KD was detected by an anti-LLO antibody using Western Blot analysis. The Listeria backbone strain expressing only tLLO was used as negative control.

pAdv164 sequence (7075 base pairs) (see FIG. 33):

(SEQ ID NO: 86)
cggagtgtatactggcttactatgttggcactgatgagggtgtcagtgaagtgcttcatgtggcaggagaaaaaaggctgcaccggt
gcgtcagcagaatatgtgatacaggatatattccgcttcctcgctcactgactcgctacgctcggtcgttcgactgcggcgagcgga
aatggcttacgaacggggcggagatttcctggaagatgccaggaagatacttaacagggaagtgagagggccgcggcaaagccgttt
ttccataggctccgcccccctgacaagcatcacgaaatctgacgctcaaatcagtggtggcgaaacccgacaggactataaagatac
caggcgtttccccctggcggctccctcgtgcgctctcctgttcctgcctttcggtttaccggtgtcattccgctgttatggccgcgt
ttgtctcattccacgcctgacactcagttccgggtaggcagttcgctccaagctggactgtatgcacgaaccccccgttcagtccga
ccgctgcgccttatccggtaactatcgtcttgagtccaacccggaaagacatgcaaaagcaccactggcagcagccactggtaattg
atttagaggagttagtcttgaagtcatgcgccggttaaggctaaactgaaaggacaagttttggtgactgcgctcctccaagccagt
tacctcggttcaaagagttggtagctcagagaaccttcgaaaaaccgccctgcaaggcggttttttcgttttcagagcaagagatta
cgcgcagaccaaaacgatctcaagaagatcatcttattaatcagataaaatatttctagccctcctttgattagtatattcctatct
taaagttacttttatgtggaggcattaacatttgttaatgacgtcaaaaggatagcaagactagaataaagctataaagcaagcata
taatattgcgtttcatctttagaagcgaatttcgccaatattataattatcaaaagagaggggtggcaaacggtatttggcattatt
aggttaaaaaatgtagaaggagagtgaaacccatgaaaaaaataatgctagtttttattacacttatattagttagtctaccaattg
cgcaacaaactgaagcaaaggatgcatctgcattcaataaagaaaattcaatttcatccatggcaccaccagcatctccgcctgcaa
gtcctaagacgccaatcgaaaagaaacacgcggatgaaatcgataagtatatacaaggattggattacaataaaaacaatgtattag
tataccacggagatgcagtgacaaatgtgccgccaagaaaaggttacaaagatggaaatgaatatattgttgtggagaaaaagaaga
aatccatcaatcaaaataatgcagacattcaagttgtgaatgcaatttcgagcctaacctatccaggtgctctcgtaaaagcgaatt
cggaattagtagaaaatcaaccagatgttctccctgtaaaacgtgattcattaacactcagcattgatttgccaggtatgactaatc
aagacaataaaatagttgtaaaaaatgccactaaatcaaacgttaacaacgcagtaaatacattagtggaaagatggaatgaaaaat
atgctcaagcttatccaaatgtaagtgcaaaaattgattatgatgacgaaatggcttacagtgaatcacaattaattgcgaaatttg
gtacagcatttaaagctgtaaataatagcttgaatgtaaacttcggcgcaatcagtgaagggaaaatgcaagaagaagtcattagtt
ttaaacaaatttactataacgtgaatgttaatgaacctacaagaccttccagatttttcggcaaagctgttactaaagagcagttgc
aagcgcttggagtgaatgcagaaaatcctcctgcatatatctcaagtgtggcgtatggccgtcaagtttatttgaaattatcaacta
attcccatagtactaaagtaaaagctgcttttgatgctgccgtaagcggaaaatctgtctcaggtgatgtagaactaacaaatatca
tcaaaaattcttccttcaaagccgtaatttacggaggttccgcaaaagatgaagttcaaatcatcgacggcaacctcggagacttac
gcgatattttgaaaaaaggcgctacttttaatcgagaaacaccaggagttcccattgcttatacaacaaacttcctaaaagacaatg
aattagctgttattaaaaacaactcagaatatattgaaacaacttcaaaagcttatacagatggaaaaattaacatcgatcactctg
gaggatacgttgctcaattcaacatttcttgggatgaagtaaattatgatctcgagacccacctggacatgctccgccacctctacc
agggctgccaggtggtgcagggaaacctggaactcacctacctgcccaccaatgccagcctgtccttcctgcaggatatccaggagg
tgcagggctacgtgctcatcgctcacaaccaagtgaggcaggtcccactgcagaggctgcggattgtgcgaggcacccagctctttg
aggacaactatgccctggccgtgctagacaatggagacccgctgaacaataccacccctgtcacaggggcctccccaggaggcctgc
gggagctgcagcttcgaagcctcacagagatcttgaaaggaggggtcttgatccagcggaacccccagctctgctaccaggacacga
ttttgtggaagaatatccaggagtttgctggctgcaagaagatctttgggagcctggcatttctgccggagagctttgatggggacc
cagcctccaacactgccccgctccagccagagcagctccaagtgtttgagactctggaagagatcacaggttacctatacatctcag
catggccggacagcctgcctgacctcagcgtcttccagaacctgcaagtaatccggggacgaattctgcacaatggcgcctactcgc
tgaccctgcaagggctgggcatcagctggctggggctgcgctcactgagggaactgggcagtggactggccctcatccaccataaca
cccacctctgcttcgtgcacacggtgccctgggaccagctctttcggaacccgcaccaagctctgctccacactgccaaccggccag
aggacgagtgtgtgggcgagggcctggcctgccaccagctgtgcgcccgagggcagcagaagatccggaagtacacgatgcggagac
tgctgcaggaaacggagctggtggagccgctgacacctagcggagcgatgcccaaccaggcgcagatgcggatcctgaaagagacgg
agctgaggaaggtgaaggtgcttggatctggcgcttttggcacagtctacaagggcatctggatccctgatggggagaatgtgaaaa
ttccagtggccatcaaagtgttgagggaaaacacatcccccaaagccaacaaagaaatcttagacgaagcatacgtgatggctggtg
tgggctccccatatgtctcccgccttctgggcatctgcctgacatccacggtgcagctggtgacacagcttatgccctatggctgcc
tcttagactaatctagacccgggccactaactcaacgctagtagtggatttaatcccaaatgagccaacagaaccagaaccagaaac
agaacaagtaacattggagttagaaatggaagaagaaaaaagcaatgatttcgtgtgaataatgcacgaaatcattgcttatttttt
taaaaagcgatatactagatataacgaaacaacgaactgaataaagaatacaaaaaaagagccacgaccagttaaagcctgagaaac
tttaactgcgagccttaattgattaccaccaatcaattaaagaagtcgagacccaaaatttggtaaagtatttaattactttattaa
tcagatacttaaatatctgtaaacccattatatcgggtttttgaggggatttcaagtctttaagaagataccaggcaatcaattaag
aaaaacttagttgattgccttttttgttgtgattcaactttgatcgtagcttctaactaattaattttcgtaagaaaggagaacagc
tgaatgaatatcccttttgttgtagaaactgtgcttcatgacggcttgttaaagtacaaatttaaaaatagtaaaattcgctcaatc
actaccaagccaggtaaaagtaaaggggctatttttgcgtatcgctcaaaaaaaagcatgattggcggacgtggcgttgttctgact
tccgaagaagcgattcacgaaaatcaagatacatttacgcattggacaccaaacgtttatcgttatggtacgtatgcagacgaaaac
cgttcatacactaaaggacattctgaaaacaatttaagacaaatcaataccttctttattgattttgatattcacacggaaaaagaa
actatttcagcaagcgatattttaacaacagctattgatttaggttttatgcctacgttaattatcaaatctgataaaggttatcaa
gcagattttgtttttagaaacgccagtctatgtgacttcaaaatcagaatttaaatctgtcaaagcagccaaaataatctcgcaaaa
tatccgagaatattttggaaagtctttgccagttgatctaacgtgcaatcattttgggattgctgtataccaagaacggacaatgta
gaattttttgatcccaattaccgttattctttcaaagaatggcaagattggtctttcaaacaaacagataataagggctttactcgt
tcaagtctaacggttttaagcggtacagaaggcaaaaaacaagtagatgaaccctggtttaatctcttattgcacgaaacgaaattt
tcaggagaaaagggtttagtagggcgcaatagcgttatgtttaccctctctttagcctactttagttcaggctattcaatcgaaacg
tgcgaatataatatgtttgagtttaataatcgattagatcaacccttagaagaaaaagaagtaatcaaaattgttagaagtgcctat
tcagaaaactatcaaggggctaatagggaatacattaccattctttgcaaagcttgggtatcaagtgatttaaccagtaaagattta
tttgtccgtcaagggtggtttaaattcaagaaaaaaagaagcgaacgtcaacgtgttcatttgtcagaatggaaagaagatttaatg
gcttatattagcgaaaaaagcgatgtatacaagccttatttagcgacgaccaaaaaagagattagagaagtgctaggcattcctgaa
cggacattagataaattgctgaaggtactgaaggcgaatcaggaaattttctttaagattaaaccaggaagaaatggtggcattcaa
cttgctagtgttaaatcattgttgctatcgatcattaaattaaaaaaagaagaacgagaaagctatataaaggcgctgacagcttcg
tttaatttagaacgtacatttattcaagaaactctaaacaaattggcagaacgccccaaaacggacccacaactcgatttgtttagc
tacgatacaggctgaaaataaaacccgcactatgccattacatttatatctatgatacgtgtttgtttttcttgctggctagcttaa
ttgcttatatttacctgcaataaaggatttcttacttccattatactcccattttccaaaaacatacggggaacacgggaacttatt
gtacaggccacctcatagttaatggtttcgagccttcctgcaatctcatccatggaaatatattcatccccctgccggcctattaat
gtgacttttgtgcccggcggatattcctgatccagctccaccataaattggtccatgcaaattcggccggcaattttcaggcgtttt
cccttcacaaggatgtcggtccctttcaattttcggagccagccgtccgcatagcctacaggcaccgtcccgatccatgtgtctttt
tccgctgtgtactcggctccgtagctgacgctctcgccttttctgatcagtttgacatgtgacagtgtcgaatgcagggtaaatgcc
ggacgcagctgaaacggtatctcgtccgacatgtcagcagacgggcgaaggccatacatgccgatgccgaatctgactgcattaaaa
aagccttttttcagccggagtccagcggcgctgttcgcgcagtggaccattagattctttaacggcagcggagcaatcagctcttta
aagcgctcaaactgcattaagaaatagcctctttctttttcatccgctgtcgcaaaatgggtaaatacccctttgcactttaaacga
gggttgcggtcaagaattgccatcacgttctgaacttcttcctctgtttttacaccaagtctgttcatccccgtatcgaccttcaga
tgaaaatgaagagaaccttttttcgtgtggcgggctgcctcctgaagccattcaacagaataacctgttaaggtcacgtcatactca
gcagcgattgccacatactccgggggaaccgcgccaagcaccaatataggcgccttcaatccctttttgcgcagtgaaatcgcttca
tccaaaatggccacggccaagcatgaagcacctgcgtcaagagcagcctttgctgtttctgcatcaccatgcccgtaggcgtttgct
ttcacaactgccatcaagtggacatgttcaccgatatgttttttcatattgctgacattttcctttatcgcggacaagtcaatttcc
gcccacgtatctctgtaaaaaggttttgtgctcatggaaaactcctctcttttttcagaaaatcccagtacgtaattaagtatttga
gaattaattttatattgattaatactaagtttacccagttttcacctaaaaacaaatgatgagataatagctccaaaggctaaagag
gactataccaactatttgttaattaa

### Example 15: ADXS31-164 is as Immunogenic as LM-LLO-ChHER2

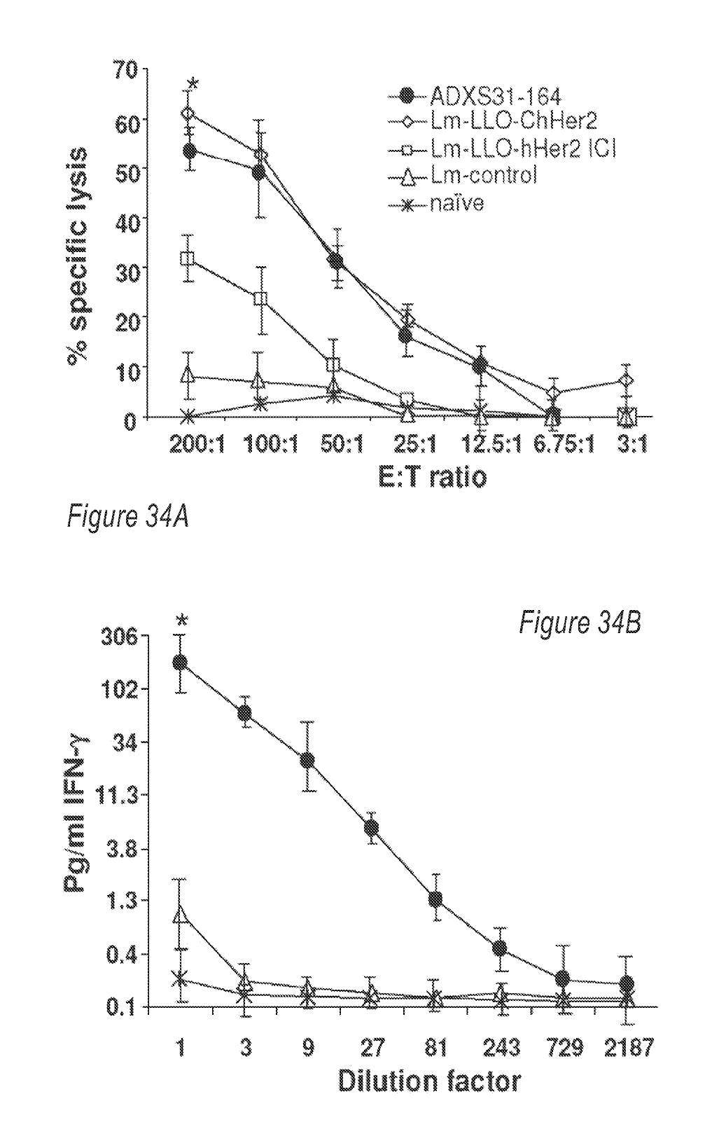

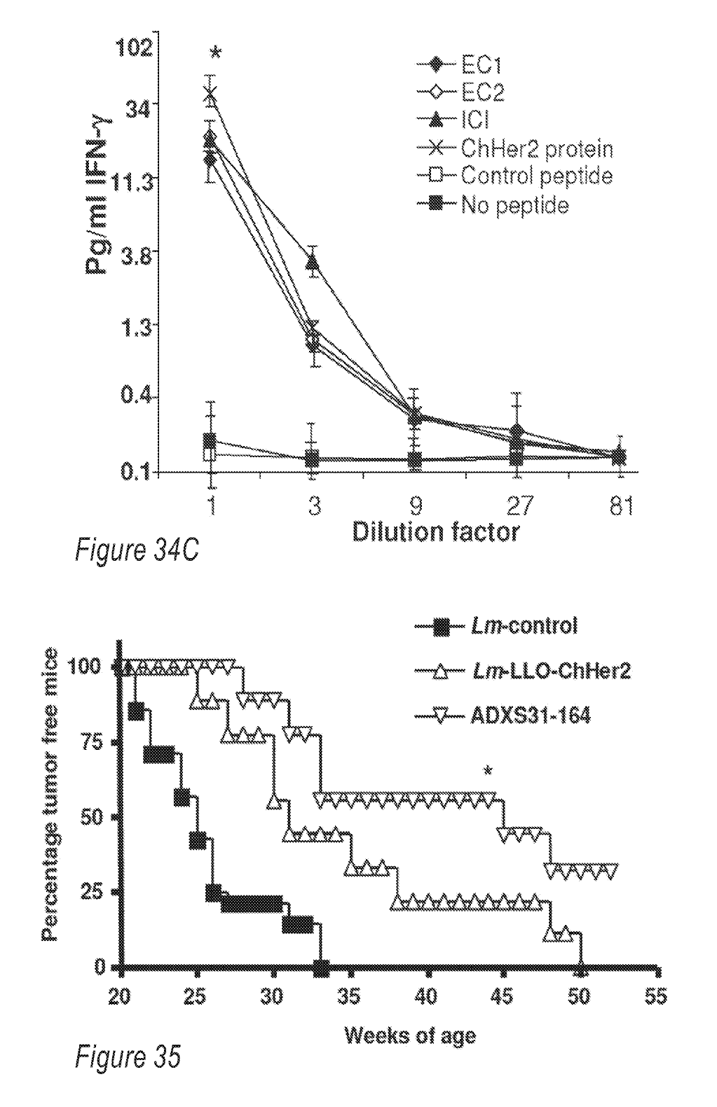

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria (FIG. 34A). ADXS31-164 was also able to stimulate the secretion of IFN-γ by the splenocytes from wild type FVB/N mice (FIG. 34B). This was detected in the culture supernatants of these cells that were co-cultured with mitomycin C treated NT-2 cells, which express high levels of Her2/neu antigen (FIG. 34C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 87 or KIFGSLAFL SEQ ID NO: 88) or intracellular (RLLQETELV SEQ ID NO: 89) domains of the Her2/neu molecule (FIG. 34C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 16: ADXS31-164 was More Efficacious than LM-LLO-ChHER2 in Preventing the Onset of Spontaneous Mammary Tumors

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Listeria-Her2/neu recombinant vaccines caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 35). These results indicate that despite being more attenuated, ADXS31-164 is more efficacious than Lm-LLO-ChHer2 in preventing the onset of spontaneous mammary tumors in Her2/neu transgenic animals.

### Example 17: Mutations in Her2/Neu Gene Upon Immunization with ADXS31-164

Mutations in the MHC class I epitopes of Her2/neu have been considered responsible for tumor escape upon immunization with small fragment vaccines or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control vaccines. Mutations were not observed within the Her-2/neu gene of any vaccinated tumor samples suggesting alternative escape mechanisms (data not shown).

### Example 18: ADXS31-164 Causes a Significant Decrease in Intra-Tumoral T Regulatory Cells

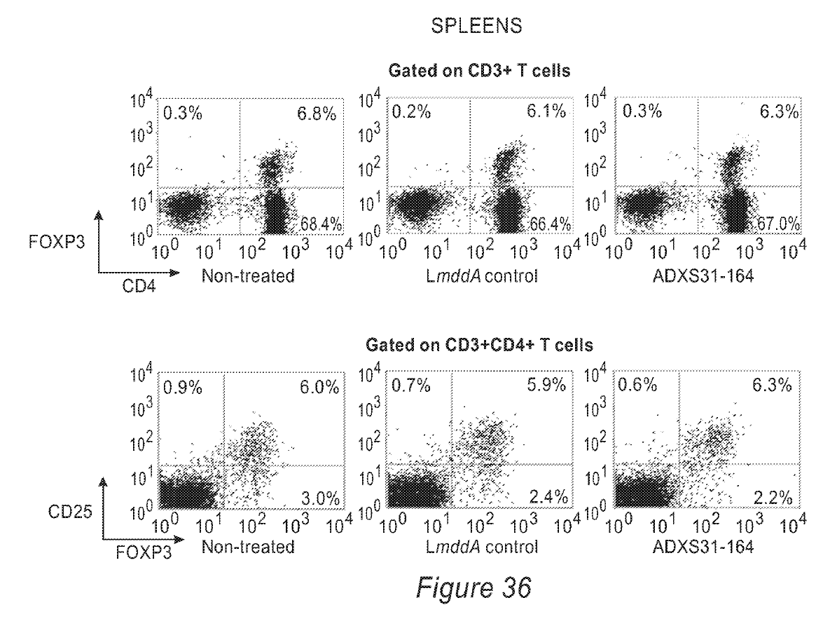

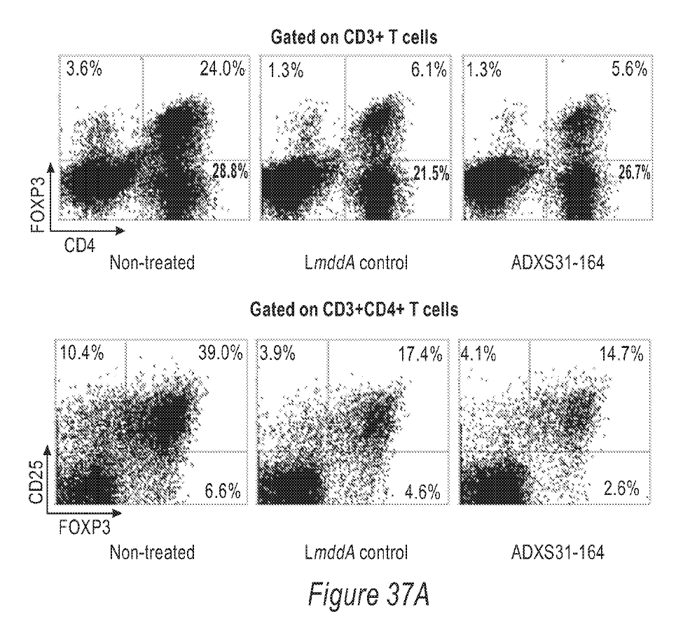

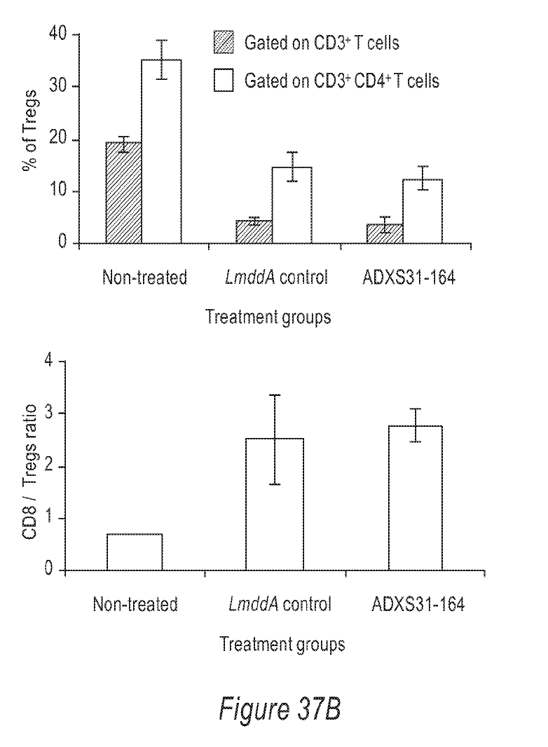

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria or the naïve animals (FIG. 36). In contrast, immunization with the Listerias caused a considerable impact on the presence of Tregs in the tumors (FIG. 37). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 37B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 19: No Escape Mutations were Introduced by Listeria Expressing her-2 Chimera

Tumor samples of the mice immunized with different vaccines such as Lm-LLO-138, LmddA164 and irrelevant vaccine Lm-LLO-NY were harvested. The DNA was purified from these samples and the DNA fragments corresponding to Her-2/neu regions IC1, EC1 and EC2 were amplified and were sequenced to determine if there were any immune escape mutations. The alignment of sequence from each DNA was performed using CLUSTALW. The results of the analysis indicated that there were no mutations in the DNA sequences harvested from tumors. The detailed analysis of these sequences is shown below.

Alignment of EC2 (975-1029 bp of Her-2-neu)
Reference
(SEQ ID NO: 90)
GGTCACAGCTGAGGACGGAACACAGCGTTGTGAGAAATGCAGCAAGCCC
TGTGCT
Lm-LLO-138-2
GGTCACAGCTGAGGACGGAACACAGCGTTGTGAGAAATGCAGCAAGCCC
TGTGCT
Lm-LLO-138-3
GGTCACAGCTGAGGACGGAACACAGCGTTGTGAGAAATGCAGCAAGCCC
TGTGCT
Lm-ddA-164-1
GGTCACAGCTGAGGACGGAACACAGCGTTGTGAGAAATGCAGCAAGCCC
TGTGCT
LmddA164-2
GGTCACAGCTGAGGACGGAACACAGCGTTGTGAGAAATGCAGCAAGCCC
TGTGCT
Lm-ddA-164-3
GGTCACAGCTGAGGACGGAACACAGCGTTGTGAGAAATGCAGCAAGCCC
TGTGCT
LmddA164-4
GGTCACAGCTGAGGACGGAACACAGCGTTGTGAGAAATGCAGCAAGCCC
TGTGCT
Lm-ddA-164-5
GGTCACAGCTGAGGACGGAACACAGCGTTGTGAGAAATGCAGCAAGCCC
TGTGCT
LmddA-164-6
GGTCACAGCTGAGGACGGAACACAGCGTTCTGAGAAATGCAGCAAGCCC
TGTGCT
Reference
(SEQ ID NO: 91)
CGAGTGTGCTATGGTCTGGGCATGGAGCACCTTCGAGGGGCGAGGGCCA
TCACCAGTGAC
Lm-LLO-138-2
CGAGTGTGCTATGGTCTGGGCATGGAGCACCTTCGAGGGGCGAGGGCCA
TCACCAGTGAC
Lm-LLO-138-3
CGAGTGTGCTATGGTCTGGGCATGGAGCACCTTCGAGGGGCGAGGGCCA
TCACCAGTGAC
Lm-ddA-164-1
CGAGTGTGCTATGGTCTGGGCATGGAGCACCTTCGAGGGGCGAGGGCCA
TCACCAGTGAC
LmddA164-2
CGAGTGTGCTATGGTCTGGGCATGGAGCACCTTCGAGGGGCGAGGGCCA
TCACCAGTGAC
Lm-ddA-164-3
CGAGTGTGCTATGGTCTGGGCATGGAGCACCTTCGAGGGGCGAGGGCCA
TCACCAGTGAC
LmddA164-4
CGAGTGTGCTATGGTCTGGGCATGGAGCACCTTCGAGGGGCGAGGGCCA
TCACCAGTGAC
Lm-ddA-164-5
CGAGTGTGCTATGGTCTGGGCATGGAGCACCTTCGAGGGGCGAGGGCCA
TCACCAGTGAC
LmddA-164-6
CGAGTGTGCTATGGTCTGGGCATGGAGCACCTTCGAGGGGCGAGGGCCA
TCACCAGTGAC
Reference
(SEQ ID No: 92)
AATGTCCAGGAGTTTGATGGCTGCAAGAAGATCTTTGGGAGCCTGGCAT
TTTTGCCGGAG
Lm-LLO-138-2
AATGTCCAGGAGTTTGATGGCTGCAAGAAGATCTTTGGGAGCCTGGCAT
TTTTGCCGGAG
Lm-LLO-138-3
AATGTCCAGGAGTTTGATGGCTGCAAGAAGATCTTTGGGAGCCTGGCAT
TTTTGCCGGAG
Lm-ddA-164-1
AATGTCCAGGAGTTTGATGGCTGCAAGAAGATCTTTGGGAGCCTGGCAT
TTTTGCCGGAG
LmddA164-2
AATGTCCAGGAGTTTGATGGCTGCAAGAAGATCTTTGGGAGCCTGGCAT
TTTTGCCGGAG
Lm-ddA-164-3
AATGTCCAGGAGTTTGATGGCTGCAAGAAGATCTTTGGGAGCCTGGCAT
TTTTGCCGGAG
LmddA164-4
AATGTCCAGGAGTTTGATGGCTGCAAGAAGATCTTTGGGAGCCTGGCAT
TTTTGCCGGAG
Lm-ddA-164-5
AATGTCCAGGAGTTTGATGGCTGCAAGAAGATCTTTGGGAGCCTGGCAT
TTTTGCCGGAG
LmddA-164-6
AATGTCCAGGAGTTTGATGGCTGCAAGAAGATCTTTGGGAGCCTGGCAT
TTTTGCCGGAG
Reference
(SEQ ID No: 93)
AGCTTTGATGGGGACCCCTCCTCCGGCATTGCTCCGCTGAGGCCTGAGC
AGCTCCAAGTG
Lm-LLO-138-2
AGCTTTGATGGGGACCCCTCCTCCGGCATTGCTCCGCTGAGGCCTGAGC
AGCTCCAAGTG
Lm-LLO-138-3
AGCTTTGATGGGGACCCCTCCTCCGGCATTGCTCCGCTGAGGCCTGAGC
AGCTCCAAGTG
Lm-ddA-164-1
AGCTTTGATGGGGACCCCTCCTCCGGCATTGCTCCGCTGAGGCCTGAGC
AGCTCCAAGTG
LmddA164-2
AGCTTTGATGGGGACCCCTCCTCCGGCATTGCTCCGCTGAGGCCTGAGC
AGCTCCAAGTG
Lm-ddA-164-3
AGCTTTGATGGGGACCCCTCCTCCGGCATTGCTCCGCTGAGGCCTGAGC
AGCTCCAAGTG
LmddA164-4
AGCTTTGATGGGGACCCCTCCTCCGGCATTGCTCCGCTGAGGCCTGAGC
AGCTCCAAGTG
Lm-ddA-164-5
AGCTTTGATGGGGACCCCTCCTCCGGCATTGCTCCGCTGAGGCCTGAGC
AGCTCCAAGTG
LmddA-164-6
AGCTTTGATGGGGACCCCTCCTCCGGCATTGCTCCGCTGAGGCCTGAGC
AGCTCCAAGTG
Reference
(SEQ ID NO: 94)
TTCGAAACCCTGGAGGAGATCACAGGTTACCTGTACATCTCAGCATGGC
CAGACAGTCTC
Lm-LLO-138-2
TTCGAAACCCTGGAGGAGATCACAGGTTACCTGTACATCTCAGCATGGC
CAGACAGTCTC
Lm-LLO-138-3
TTCGAAACCCTGGAGGAGATCACAGGTTACCTGTACATCTCAGCATGGC
CAGACAGTCTC
Lm-ddA-164-1
TTCGAAACCCTGGAGGAGATCACAGGTTACCTGTACATCTCAGCATGGC
CAGACAGTCTC
LmddA164-2
TTCGAAACCCTGGAGGAGATCACAGGTTACCTGTACATCTCAGCATGGC
CAGACAGTCTC
Lm-ddA-164-3
TTCGAAACCCTGGAGGAGATCACAGGTTACCTGTACATCTCAGCATGGC
CAGACAGTCTC
LmddA164-4
TTCGAAACCCTGGAGGAGATCACAGGTTACCTGTACATCTCAGCATGGC
CAGACAGTCTC
Lm-ddA-164-5
TTCGAAACCCTGGAGGAGATCACAGGTTACCTGTACATCTCAGCATGGC
CANACAGTCTC
LmddA-164-6
TTCGAAACCCTGGAGGAGATCACAGGTTACCTGTACATCTCAGCATGGC
CAGACAGTCT
Reference
(SEQ ID NO: 95)
CGTGACCTCAGTGTCTTCCAGAACCTTCGAATCATTCGGGGACGGATTC
TCCACGATGGC
Lm-LLO-138-2
CGTGACCTCAGTGTCTTCCAGAACCTTCGAATCATTCGGGGACGGATTC
TCCACGATGGC
Lm-LLO-138-3
CGTGACCTCAGTGTCTTCCAGAACCTTCGAATCATTCGGGGACGGATTC
TCCACGATGGC
Lm-ddA-164-1
CGTGACCTCAGTGTCTTCCAGAACCTTCGAATCATTCGGGGACGGATTC
TCCACGATGGC
LmddA164-2
CGTGACCTCAGTGTCTTCCAGAACCTTCGAATCATTCGGGGACGGATTC
TCCACGATGGC
Lm-ddA-164-3
CGTGACCTCAGTGTCTTCCAGAACCTTCGAATCATTCGGGGACGGATTC
TCCACGATGGC
LmddA164-4
CGTGACCTCAGTGTCTTCCAAAACCTTCGAATCATTCGGGGACGGATTC
TCCACGATGGC
Lm-ddA-164-5
CGTGACCTCAGTGTCTTCCAAAACCTTCGAATCATTCGGGGACGGATTC
TCCACGATGGC
LmddA-164-6
CGTGACCTCAGTGTCTTCCAAAACCTTCGAATCATTCGGGGACGGATTC
TCCACGATGGC
Reference
(SEQ ID NO: 96)
GCGTACTCATTGACACTGCAAGGCCTGGGGATCCACTCGCTGGGGCTGC
GCTCACTGCGG
Lm-LLO-138-2
GCGTACTCATTGACACTGCAAGGCCTGGGGATCCACTCGCTGGGGCTGC
GCTCACTGCGG
Lm-LLO-138-3
GCGTACTCATTGACACTGCAAGGCCTGGGGATCCACTCGCTGGGGCTGC
GCTCACTGCGG
Lm-ddA-164-1
GCGTACTCATTGACACTGCAAGGCCTGGGGATCCACTCGCTGGGGCTGC
GCTCACTGCGG
LmddA164-3
GCGTACTCATTGACACTGCAAGGCCTGGGGATCCACTCGCTGGGGCTGC
GCTCACTGCGG
Lm-ddA-164-5
GCGTACTCATTGACACTGCAAGGCCTGGGGATCCACTCGCTGGGGCTGC
GCTCACTGCGG
Lm-ddA-164-6
GCGTACTCATTGACACTGCAAGGCCTGGGGATCCACTCGCTGGGGCTGC
GCTCACTGCGG
Reference
(SEQ ID NO: 97)
GAGCTGGGCAGTGGATTGGCTCTGATTCACCGCAACGCCCATCTCTGCT
TTGTACACACT
Lm-LLO-138-2
GAGCTGGGCAGTGGATTGGCTCTGATTCACCGCAACGCCCATCTCTGCT
TTGTACACACT
Lm-LLO-138-3
GAGCTGGGCAGTGGATTGGCTCTGATTCACCGCAACGCCCATCTCTGCT
TTGTACACACT
Lm-ddA-164-1
GAGCTGGGCAGTGGATTGGCTCTGATTCACCGCAACGCCCATCTCTGCT
TTGTACACACT
LmddA164-3
GAGCTGGGCAGTGGATTGGCTCTGATTCACCGCAACGCCCATCTCTGCT
TTGTACACACT
Lm-ddA-164-5
GAGCTGGGCAGTGGATTGGCTCTGATTCACCGCAACGCCCATCTCTGCT
TTGTACACACT
Lm-ddA-164-6
GAGCTGGGCAGTGGATTGGCTCTGATTCACCGCAACGCCCATCTCTGCT
TTGTACACACT
Reference
(SEQ ID NO: 98)
GTACCTTGGGACCAGCTCTTCCGGAACCCACATCAGGCCCTGCTCCACA
GTGGGAACCGG
Lm-LLO-138-2
GTACCTTGGGACCAGCTCTTCCGGAACCCACATCAGGCCCTGCTCCACA
GTGGGAACCGG
Lm-LLO-138-3
GTACCTTGGGACCAGCTCTTCCGGAACCCACATCAGGCCCTGCTCCACA
GTGGGAACCGG
Lm-ddA-164-1
GTACCTTGGGACCAGCTCTTCCGGAACCCACATCAGGCCCTGCTCCACA
GTGGGAACCGG
LmddA164-3
GTACCTTGGGACCAGCTCTTCCGGAACCCACATCAGGCCCTGCTCCACA
GTGGGAACCGG
Lm-ddA-164-5
GTACCTTGGGACCANCTCTTCCGGAACCCACATCAGGCCCTGCTCCACA
GTGGGAACCGG
Lm-ddA-164-6
GTACCTTGGGACCAGCTCTTCCGGAACCCACATCAGGCCCTGCTCCACA
GTGGGAACCGG
Reference
(SEQ ID NO: 99)
CCGGAAGAGGATTGTGGTCTCGAGGGCTTGGTCTGTAACTCACTGTGTG
CCCACGGGCAC
Lm-LLO-138-2
CCGGAAGAGGATTGTGGTCTCGAGGGCTTGGTCTGTAACTCACTGTGTG
CCCACGGGCAC
Lm-LLO-138-3
CCGGAAGAGGATTGTGGTCTCGAGGGCTTGGTCTGTAACTCACTGTGTG
CCCACGGGCAC
Lm-ddA-164-1
CCGGAAGAGGATTGTGGTCTCGAGGGCTTGGTCTGTAACTCACTGTGTG
CCCACGGGCAC
LmddA164-3
CCGGAAGAGGATTGTGGTCTCGAGGGCTTGGTCTGTAACTCACTGTGTG
CCCACGGGCAC
Lm-ddA-164-6
CCGGAAGAGGATTGTGGTCTCGAGGGCTTGGTCTGTAACTCACTGTGTG
CCCACGGGCAC
Reference
(SEQ ID NO: 100)
TGCTGGGGGCCAGGGCCCACCCAGTGTGTCAACTGCAGTCATTTCCTTC
GGGGCCAGGAG
Lm-LLO-138-2
TGCTGGGGGCCAGGGCCCACCCAGTGTGTCAACTGCAGTCATTTCCTTC
GGGGCCAGGAG
Lm-LLO-138-3
TGCTGGGGGCCAGGGCCCACCCAGTGTGTCAACTGCAGTCATTTCCTTC
GGGGCCAGGAG
Lm-ddA-164-1
TGCTGGGGGCCAGGGCCCACCCAGTGTGTCAACTGCAGTCATTTCCTTC
GGGGCCAGGAG
LmddA164-3
TGCTGGGGGCCAGGGCCCACCCAGTGTGTCAACTGCAGTCATTTCCTTC
GGGGCCAGGAG
Lm-ddA-164-6
TGCTGGGGGCCAGGGCCCACCCA--------------------------
-----------
Alignment of IC1 (2114-3042 bp of Her-2-neu)
Reference
(SEQ ID NO: 101)
CGCCCAGCGGAGCAATGCCCAACCAGGCTCAGATGCGGATCCTAAAAGA
GACGGAGC
Lm-LLO-NY-2
CGCCCAGCGGAGCAATGCCCAACCAGGCTCAGATGCGGATCCTAAAAGA
GACGGAGC
Lm-LLO-138-4
CGCCCAGCGGAGCAATGCCCAACCAGGCTCAGATGCGGATCCTAAAAGA
GACGGAGC
Lm-ddA-164-2
CGCCCAGCGGAGCAATGCCCAACCAGGCTCAGATGCGGATCCTAAAAGA
GACGGAGC
Lm-ddA-164-3
CGCCCAGCGGAGCAATGCCCAACCAGGCTCAGATGCGGATCCTAAAAGA
GACGGAGC
Lm-ddA164-6
CGCCCAGCGGAGCAATGCCCAACCAGGCTCAGATGCGGATCCTAAAAGA
GACGGAGC
Reference
(SEQ ID NO: 102)
TAAGGAAGGTGAAGGTGCTTGGATCAGGAGCTTTTGGCACTGTCTACAA
GGGCATCTGGA
Lm-LLO-NY-1
TAAGGAAGGTGAAGGTGCTTGGATCAGGAGCTTTTGGCACTGTCTACAA
GGGCATCTGGA
Lm-LLO-NY-2
TAAGGAAGGTGAAGGTGCTTGGATCAGGAGCTTTTGGCACTGTCTACAA
GGGCATCTGGA
Lm-LLO-138-1
TAAGGAAGGTGAACGTGCTTGGATCAGGAGCTTTTGGCACTGTCTACAA
GGGCATCTGGA
Lm-LLO-138-2
TAAGGAAGGTGAAGGTGCTTGGATCAGGAGCTTTTGGCACTGTCTACAA
GGGCATCTGGA
Lm-LLO-138-3
TAAGGAAGGTGAAGGTGCTTGGATCAGGAGCTTTTGGCACTGTCTACAA
GGGCATCTGGA
Lm-LLO-138-4
TAAGGAAGGTGAAGGTGCTTGGATCAGGAGCTTTTGGCACTGTCTACAA
GGGCATCTGGA
Lm-ddA-164-1
TAAGGAAGGTGAAGGTGCTTGGATCAGGAGCTTTTGGCACTGTCTACAA
GGGCATCTGGA
Lm-ddA-164-2
TAAGGAAGGTGAAGGTGCTTGGATCAGGAGCTTTTGGCACTGTCTACAA
GGCATCTGGA
Lm-ddA-164-3
TAAGGAAGGTGAAGGTGCTTGGATCAGGAGCTTTTGGCACTGTCTACAA
GGGCATCTGGA
Lm-ddA-164-4
TAAGGAAGGTGAAGGTGCTTGGATCAGGAGCTTTTGGCACTGTCTACAA
GGGCATCTGGA
Lm-ddA-164-5
TAAGGAAGGTGAAGGTGCTTGGATCAGGAGCTTTTGGCACTGTCTACAA
GGGCATCTGGA
Lm-ddA164-6
TAAGGAAGGTGAAGGTGCTTGGATCAGGAGCTTTTGGCACTGTCTACAA
GGGCATCTGGA
Reference
(SEQ ID NO: 103)
TCCCAGATGGGGAGAATGTGAAAATCCCCGTGGCTATCAAGGTGTTGAG
AGAAAACACAT
Lm-LLO-NY-1
TCCCAGATGGGGAGAATGTGAAAATCCCCGTGGCTATCAAGGTGTTGAG
AGAAAACACAT
Lm-LLO-NY-2
TCCCAGATGGGGAGAATGTGAAAATCCCCGTGGCTATCAAGGTGTTGAG
AGAAAACACAT
Lm-LLO-138-1
TCCCAGATGGGGAGAATGTGAAAATCCCCGTGGCTATCAAGGTGTTGAG
AGAAAACACAT
Lm-LLO-138-2
TCCCAGATGGGGAGAATGTGAAAATCCCCGTGGCTATCAAGGTGTTGAG
AGAAAACACAT
Lm-LLO-138-3
TCCCAGATGGGGAGAATGTGAAAATCCCCGTGGCTATCAAGGTGTTGAG
AGAAAACACAT
Lm-LLO-138-4
TCCCAGATGGGGAGAATGTGAAAATCCCCGTGGCTATCAAGGTGTTGAG
AGAAAACACAT
Lm-ddA-164-1
TCCCAGATGGGGAGAATGTGAAAATCCCCGTGGCTATCAAGGTGTTGAG
AGAAAACACAT
Lm-ddA-164-2
TCCCAGATGGGGAGAATGTGAAAATCCCCGTGGCTATCAAGGTGTTGAG
AGAAAACACAT
Lm-ddA-164-3
TCCCAGATGGGGAGAATGTGAAAATCCCCGTGGCTATCAAGGTGTTGAG
AGAAAACACAT
Lm-ddA-164-4
TCCCAGATGGGGAGAATGTGAAAATCCCCGTGGCTATCAAGGTGTTGAG
AGAAAACACAT
Lm-ddA-164-5
TCCCAGATGGGGAGAATGTGAAAATCCCCGTGGCTATCAAGGTGTTGAG
AGAAAACACAT
Lm-ddA164-6
TCCCAGATGGGGAGAATGTGAAAATCCCCGTGGCTATCAAGGTGTTGAG
AGAAAACACAT
Reference
(SEQ ID NO: 104)
CTCCTAAAGCCAACAAAGAAATTCTAGATGAAGCGTATGTGATGGCTGG
TGTGGGTTCTC
Lm-LLO-NY-1
CTCCTAAAGCCAACAAAGAAATTCTAGATGAAGCGTATGTGATGGCTGG
TGTGGGTTCTC
Lm-LLO-NY-2
CTCCTAAAGCCAACAAAGAAATTCTAGATGAAGCGTATGTGATGGCTGG
TGTGGGTTCTC
Lm-LLO-138-1
CTCCTAAAGCCAACAAAGAAATTCTAGATGAAGCGTATGTGATGGCTGG
TGTGGGTTCTC
Lm-LLO-138-2
CTCCTAAAGCCAACAAAGAAATTCTAGATGAAGCGTATGTGATGGCTGG
TGTGGGTTCTC
Lm-LLO-138-3
CTCCTAAAGCCAACAAAGAAATTCTAGATGAAGCGTATGTGATGGCTGG
TGTGGGTTCTC
lm-LLO-138-4
CTCCTAAAGCCAACAAAGAAATTCTAGATGAAGCGTATGTGATGGCTGG
TGTGGGTTCTC
Lm-ddA-164-1
CTCCTAAAGCCAACAAAGAAATTCTAGATGAAGCGTATGTGATGGCTGG
TGTGGGTTCTC
Lm-ddA-164-2
CTCCTAAAGCCAACAAAGAAATTCTAGATGAAGCGTATGTGATGGCTGG
TGTGGGTTCTC
Lm-ddA-164-3
CTCCTAAAGCCAACAAAGAAATTCTAGATGAAGCGTATGTGATGGCTGG
TGTGGGTTCTC
Lm-ddA-164-4
CTCCTAAAGCCAACAAAGAAATTCTAGATGAAGCGTATGTGATGGCTGG
TGTGGGTTCTC
Lm-ddA-164-5
CTCCTAAAGCCAACAAAGAAATTCTAGATGAAGCGTATGTGATGGCTGG
TGTGGGTTCTC
Lm-ddA164-6
CTCCTAAAGCCAACAAAGAAATTCTAGATGAAGCGTATGTGATGGCTGG
TGTGGGTTCTC
Reference
(SEQ ID NO: 105)
CGTATGTGTCCCGCCTCCTGGGCATCTGCCTGACATCCACAGTACAGCT
GGTGACACAGC
Lm-LLO-NY-1
CGTATGTGTCCCGCCTCCTGGGCATCTGCCTGACATCCACAGTACAGCT
GGTGACACAGC
Lm-LLO-NY-2
CGTATGTGTCCCGCCTCCTGGGCATCTGCCTGACATCCACAGTACAGCT
GGTGACACAGC
Lm-LLO-138-1
CGTATGTGTCCCGCCTCCTGGGCATCTGCCTGACATCCACAGTACAGCT
GGTGACACAGC
Lm-LLO-138-2
CGTATGTGTCCCGCCTCCTGGGCATCTGCCTGACATCCACAGTACAGCT
GGTGACACAGC
Lm-LLO-138-3
CGTATGTGTCCCGCCTCCTGGGCATCTGCCTGACATCCACAGTACAGCT
GGTGACACAGC
Lm-LLO-138-4
CGTATGTGTCCCGCCTCCTGGGCATCTGCCTGACATCCACAGTACAGCT
GGTGACACAGC
Lm-ddA-164-1
CGTATGTGTCCCGCCTCCGGGCATCTGCCTGACATCCACAGTACAGCT
GGTGACACAGC
Lm-ddA-164-2
CGTATGTGTCCCGCCTCCTGGGCATCTGCCTGACATCCACAGTACAGCT
GGTGACACAGC
Lm-ddA-164-3
CGTATGTGTCCCGCCTCCTGGGCATCTGCCTGACATCCACAGTACAGCT
GGTGACACAGC
Lm-ddA-164-4
CGTATGTGTCCCGCCTCCTGGGCATCTGCCTGACATCCACAGTACAGCT
GGTGACACAGC
Lm-ddA-164-5
CGTATGTGTCCCGCCTCCTGGGCATCTGCCTGACATCCACAGTACAGCT
GGTGACACAGC
Lm-ddA164-6
CGTATGTGTCCCGCCTCCTGGGCATCTGCCTGACATCCACAGTACAGCT
GGTGACACAGC
Reference
(SEQ ID NO: 106)
TTATGCCCTACGGCTGCCTTCTGGACCATGTCCGAGAACACCGAGGTCG
CCTAGGCTCCC
Lm-LLO-NY-1
TTATGCCCTACGGCTGCCTTCTGGACCATGTCCGAGAACACCGAGGTCG
CCTAGGCTCCC
Lm-LLO-NY-2
TTATGCCCTACGGCTGCCTTCTGGACCATGTCCGAGAACACCGAGGTCG
CCTAGGCTCCC
Lm-LLO-138-1
TTATGCCCTACGGCTGCCTTCTGGACCATGTCCGAGAACACCGAGGTCG
CCTAGGCTCCC
Lm-LLO-138-2
TTATGCCCTACGGCTGCCTTCTGGACCATGTCCGAGAACACCGAGGTCG
CCTAGGCTCCC
Lm-LLO-138-3
TTATGCCCTACGGCTGCCTTCTGGACCATGTCCGAGAACACCGAGGTCG
CCTAGGCTCCC
Lm-LLO-138-4
TTATGCCCTACGGCTGCCTTCTGGACCATGTCCGAGAACACCGAGGTCG
CCTAGGCTCCC
Lm-ddA-164-1
TTATGCCCTACGGCTGCCTTCTGGACCATGTCCGAGAACACCGAGGTCG
CCTAGGCTCCC
Lm-ddA-164-2
TTATGCCCTACGGCTGCCTTCTGGACCATGTCCGAGAACACCGAGGTCG
CCTAGGCTCCC
Lm-ddA-164-3
TTATGCCCTACGGCTGCCTTCTGGACCATGTCCGAGAACACCGAGGTCG
CCTAGGCTCCC
Lm-ddA-164-4
TTATGCCCTACGGCTGCCTTCTGGACCATGTCCGAGAACACCGAGGTCG
CCTAGGCTCCC
Lm-ddA-164-5
TTATGCCCTACGGCTGCCTTCTGGACCATGTCCGAGAACACCGAGGTCG
CCTAGGCTCCC
Lm-ddA164-6
TTATGCCCTACGGCTGCCTTCTGGACCATGTCCGAGAACACCGAGGTCG
CCTAGGCTCCC
Reference
(SEQ ID NO: 107)
AGGACCTGCTCAACTGGTGTGTTCAGATTGCCAAGGGGATGAGCTACCT
GGAGGACGTGC
Lm-LLO-NY-1
AGGACCTGCTCAACTGGTGTGTTCAGATTGCCAAGGGGATGAGCTACCT
GGAGGACGTGC
Lm-LLO-NY-2
AGGACCTGCTCAACTGGTGTGTTCAGATTGCCAAGGGGATGAGCTACCT
GGAGGACGTGC
Lm-LLO-138-1
AGGACCTGCTCAACTGGTGTGTTCAGATTGCCAAGGGGATGAGCTACCT
GGAGGACGTGC
Lm-LLO-138-2
AGGACCTGCTCAACTGGTGTGTTCAGATTGCCAAGGGGATGAGCTACCT
GGAGGACGTGC
Lm-LLO-138-3
AGGACCTGCTCAACTGGTGTGTTCAGATTGCCAAGGGGATGAGCTACCT
GGAGGACGTGC
Lm-LLO-138-4
AGGACCTGCTCAACTGGTGTGTTCAGATTGCCAAGGGGATGAGCTACCT
GGAGGACGTGC
Lm-ddA-164-1
AGGACCTGCTCAACTGGTGTGTTCAGATTGCCAAGGGGATGAGCTACCT
GGAGGACGTGC
Lm-ddA-164-2
AGGACCTGCTCAACTGGTGTGTTCAGATTGCCAAGGGGATGAGCTACCT
GGAGGACGTGC
Lm-ddA-164-3
AGGACCTGCTCAACTGGTGTGTTCAGATTGCCAAGGGGATGAGCTACCT
GGAGGACGTGC
Lm-ddA-164-4
AGGACCTGCTCAACTGGTGTGTTCAGATTGCCAAGGGGATGAGCTACCT
GGAGGACGTGC
Lm-ddA-164-5
AGGACCTGCTCAACTGGTGTGTTCAGATTGCCAAGGGGATGAGCTACCT
GGAGGACGTGC
Lm-ddA164-6
AGGACCTGCTCAACTGGTGTGTTCAGATTGCCAAGGGGATGAGCTACCT
GGAGGACGTGC
Reference
(SEQ ID NO: 108)
GGCTTGTACACAGGGACCTGGCTGCCCGGAATGTGCTAGTCAAGAGTCC
CAACCACGTCA
Lm-LLO-NY-1
GGCTTGTACACAGGGACCTGGCTGCCCGGAATGTGCTAGTCAAGAGTCC
CAACCACGTCA
Lm-LLO-NY-2
GGCTTGTACACAGGGACCTGGCTGCCCGGAATGTGCTAGTCAAGAGTCC
CAACCACGTCA
Lm-LLO-138-1
GGCTTGTACACAGGGACCTGGCTGCCCGGAATGTGCTAGTCAAGAGTCC
CAACCACGTCA
Lm-LLO-138-2
GGCTTGTACACAGGGACCTGGCTGCCCGGAATGTGCTAGTCAAGAGTCC
CAACCACGTCA
Lm-LLO-138-3
GGCTTGTACACAGGGACCTGGCTGCCCGGAATGTGCTAGTCAAGAGTCC
CAACCACGTCA
Lm-LLO-138-4
GGCTTGTACACAGGGACCTGGCTGCCCGGAATGTGCTAGTCAAGAGTCC
CAACCACGTCA
Lm-ddA-164-1
GGCTTGTACACAGGGACCTGGCTGCCCGGAATGTGCTAGTCAAGAGTCC
CAACCACGTCA
Lm-ddA-164-2
GGCTTGTACACAGGGACCTGGCTGCCCGGAATGTGCTAGTCAAGAGTCC
CAACCACGTCA
Lm-ddA-164-4
GGCTTGTACACAGGGACCTGGCTGCCCGGAATGTGCTAGTCAAGAGTCC
CAACCACGTCA
Lm-ddA-164-3
GGCTTGTACACAGGGACCTGGCTGCCCGGAATGTGCTAGTCAAGAGTCC
CAACCACGTCA
Lm-ddA-164-5
GGCTTGTACACAGGGACCTGGCTGCCCGGAATGTGCTAGTCAAGAGTCC
CAACCACGTCA
Lm-ddA164-6
GGCTTGTACACAGGGACCTGGCTGCCCGGAATGTGCTAGTCAAGAGTCC
CAACCACGTCA
Reference
(SEQ ID NO: 109)
AGATTACAGATTTCGGGCTGGCTCGGCTGCTGGACATTGATGAGACAGA
GTACCATGCAG
Lm-LLO-NY-1
AGATTACAGATTTCGGGCTGGCTCGGCTGCTGGACATTGATGAGACAGA
GTACCATGCAG
Lm-LLO-NY-2
AGATTACAGATTTCGGGCTGGCTCGGCTGCTGGACATTGATGAGACAGA
GTACCATGCAG
Lm-LLO-138-1
AGATTACAGATTTCGGGCTGGCTCGGCTGCTGGACATTGATGAGACAGA
GTACCATGCAG
Lm-LLO-138-2
AGATTACAGATTTCGGGCTGGCTCGGCTGCTGGACATTGATGAGACAGA
GTACCATGCAG
Lm-LLO-138-3
AGATTACAGATTTCGGGCTGGCTCGGCTGCTGGACATTGATGAGACAGA
GTACCATGCAG
Lm-LLO-138-4
AGATTACAGATTTCGGGCTGGCTCGGCTGCTGGACATTGATGAGACAGA
GTACCATGCAG
Lm-ddA-164-1
AGATTACAGATTTCGGGCTGGCTCGGCTGCTGGACATTGATGAGACAGA
GTACCATGCAG
Lm-ddA-164-2
AGATTACAGATTTCGGGCTGGCTCGGCTGCTGGACATTGATGAGACAGA
GTACCATGCAG
Lm-ddA-164-3
AGATTACAGATTTCGGGCTGGCTCGGCTGCTGGACATTGATGAGACAGA
GTACCATGCAG
Lm-ddA-164-4
AGATTACAGATTTCGGGCTGGCTCGGCTGCTGGACATTGATGAGACAGA
GTACCATGCAG
Lm-ddA-164-5
AGATTACAGATTTCGGGCTGGCTCGGCTGCTGGACATTGATGAGACAGA
GTACCATGCA
Lm-ddA164-6
AGATTACAGATTTCGGGCTGGCTCGGCTGCTGGACATTGATGAGACAGA
GTACCATGCAG
Reference
(SEQ ID NO: 110)
ATGGGGGCAAGGTGCCCATCAAATGGATGGCATTGGAATCTATTCTCAG
ACGCCGGTTCA
Lm-LLO-NY-1
ATGGGGGCAAGGTGCCCATCAAATGGATGGCATTGGAATCTATTCTCAG
ACGCCGGTTCA
Lm-LLO-NY-2
ATGGGGGCAAGGTGCCCATCAAATGGATGGCATTGGAATCTATTCTCAG
ACGCCGGTTCA
Lm-LLO-138-1
ATGGGGGCAAGGTGCCCATCAAATGGATGGCATTGGAATCTATTCTCAG
ACGCCGGTTCA
Lm-LLO-138-2
ATGGGGGCAAGGTGCCCATCAAATGGATGGCATTGGAATCTATTCTCAG
ACGCCGGTTCA
Lm-LLO-138-3
ATGGGGGCAAGGTGCCCATCAAATGGATGGCATTGGAATCTATTCTCAG
ACGCCGGTTCA
Lm-LLO-138-4
ATGGGGGCAAGGTGCCCATCAAATGGATGGCATTGGAATCTATTCTCAG
ACGCCGGTTCA
Lm-ddA-164-1
ATGGGGGCAAGGTGCCCATCAAATGGATGGCATTGGAATCTATTCTCAG
ACGCCGGTTCA
Lm-ddA-164-2
ATGGGGGCAAGGTGCCCATCAAATGGATGGCATTGGAATCTATTCTCAG
ACGCCGGTTCA
Lm-ddA-164-3
ATGGGGGCAAGGTGCCCATCAAATGGATGGCATTGGAATCTATTCTCAG
ACGCCGGTTCA
Lm-ddA-164-4
ATGGGGGCAAGGTGCCCATCAAATGGATGGCATTGGAATCTATTCTCAG
ACGCCGGTTCA
Lm-ddA-164-5
ATGGGGGCAAGGTGCCCATCAAATGGATGGCATTGGAATCTATTCTCAG
ACGCCGGTTCA
Lm-ddA-164-6
ATGGGGGCAAGGTGCCCATCAAATGGATGGCATTGGAATCTATTCTCAG
ACGCCGGTTCA
Reference
(SEQ ID NO: 111)
CCCATCAGAGTGATGTGTGGAGCTATGGAGTGACTGTGTGGGAGCTGAT
GACTTTTGGGG
Lm-LLO-NY-1
CCCATCAGAGTGATGTGTGGAGCTATGGAGTGACTGTGTGGGAGCTGAT
GACTTTTGGGG
Lm-LLO-NY-2
CCCATCAGAGTGATGTGTGGAGCTATGGAGTGACTGTGTGGGAGCTGAT
GACTTTTGGGG
Lm-LLO-138-1
CCCATCAGAGTGATGTGTGGAGCTATGGAGTGACTGTGTGGGAGCTGAT
GACTTTTGGGG
Lm-LLO-138-2
CCCATCAGAGTGATGTGTGGAGCTATGGAGTGACTGTGTGGGAGCTGAT
GACTTTTGGGG
Lm-LLO-138-3
CCCATCAGAGTGATGTGTGGAGCTATGGAGTGACTGTGTGGGAGCTGAT
GACTTTTGGGG
Lm-LLO-138-4
CCCATCAGAGTGATGTGTGGAGCTATGGAGTGACTGTGTGGGAGCTGAT
GACTTTTGGGG
Lm-ddA-164-1
CCCATCAGAGTGATGTGTGGAGCTATGGAGTGACTGTGTGGGAGCTGAT
GACTTTTGGGG
Lm-ddA-164-2
CCCATCAGAGTGATGTGTGGAGCTATGGAGTGACTGTGTGGGAGCTGAT
GACTTTTGGGG
Lm-ddA-164-3
CCCATCAGAGTGATGTGTGGAGCTATGGAGTGACTGTGTGGGAGCTGAT
GACTTTTGGGG
Lm-ddA-164-4
CCCATCAGAGTGATGTGTGGAGCTATGGAGTGACTGTGTGGGAGCTGAT
GACTTTTGGGG
Lm-ddA-164-5
CCCATCAGAGTGATGTGTGGAGCTATGGAGTGACTGTGTGGGAGCTGAT
GACTTTTGGGG
Lm-ddA164-6
CCCATCAGAGTGATGTGTGGAGCTATGGAGTGACTGTGTGGGAGCTGAT
GACTTTTGGGG
Reference
(SEQ ID NO: 112)
CCAAACCTTACGATGGAATCCCAGCCCGGGAGATCCCTGATTTGCTGGA
GAAGGGAGAA
Lm-LLO-NY-1
CCAAACCTTACGATGGAATCCCAGCCCGGGAGATCCCTGATTTGCTGGA
GAAGGGAGAA
Lm-LLO-NY-2
CCAAACCTTACGATGGAATCCCAGCCCGGGAGATCCCTGATTTGCTGGA
GAAGGGAGAA
Lm-LLO-138-1
CCAAACCTTACGATGGAATCCCAGCCCGGGAGATCCCTGATTTGCTGGA
GAAGGGAGAA
Lm-LLO-138-3
CCAAACCTTACGATGGAATCCCAGCCCGGGAGATCCCTGATTTGCTGGA
GAAGGGAGAA
Lm-LLO-138-4
CCAAACCTTACGATGNAATCCCAGCCCGGGAGATCCCTGATTTGCTGGA
GAAGGGAGAA
Lm-ddA164-6
CCAAACCTTACGATGGAATCCCAGCCCGGGAGATCCCTGATTTGCTGGA
GAAGGGAGAA
Lm-ddA-164-2
CCAAACCTTACGATGGAATCCCAGCCCGGGAGATCCCTGATTTGCTGGA
GAAGGGAGAA
Lm-LLO-138-2
CCAAACCTTACGATGGAATCCCAGCCCGGGAGATCCCTGATTTGCTGGA
GAAGGGAGAA
Lm-ddA-164-3
CCAAACCTTACGATGGAATCCCAGCCCGGGAGATCCCTGATTTGCTGGA
GAAGGGAGAA
Lm-ddA-164-5
CCAAACCTTACGATGGAATCCCAGCCCGGGAGATCCCTGATTTGCTGGA
GAAGGGAGAA
Lm-ddA-164-1
CCAAACCTTACGATGGAATCCCAGCCCGGGAGATCCCTGATTTGCTGGA
GAAGGGAGAA
Lm-ddA-164-4
CCAAACCTTACGATGGAATCCCAGCCCGGGAGATCCCTGATTTGCTGGA
GAAGGGAGAA
Reference
(SEQ ID NO: 113)
CGCCTACCTCAGCCTCCAATCTGCACCATTGATGTCTACATGATTATGG
TCAAATGTT
Lm-LLO-NY-1
CGCCTACCTCAGCCTCCAATCTGCACCATTGATGTCTACATGATTATGG
TCAAATGTT
Lm-LLO-NY-2
CGCCTACCTCAGCCTCCAATCTGCACCATTGATGTCTACATGATTATGG
TCAAATGTT
Lm-LLO-138-1
CGCCTACCTCAGCCTCCAATCTGCACCATTGATGTCTACATGATTATGG
TCAAATGTT
Lm-LLO-138-2
CGCCTACCTCAGCCTCCAATCTGCACCATTGATGTCTACATGATTATGG
TCAAATGTT
Lm-LLO-138-3
CGCCTACCTCAGCCTCCAATCTGCACCATTGATGTCTACATGATTATGG
TCAAATGTT
Lm-LLO-138-4
CGCCTACCTCAGCCTCCAATCTGCACCATTGATGTCTACATGATTATGG
TCAAATGTT
Lm-ddA-164-1
CGCCTACCTCAGCCTCCAATCTGCACCATTGATGTCTACATGATTATGG
TCAAATGTT
Lm-ddA-164-2
CGCCTACCTCAGCCTCCAATCTGCACCATTGATGTCTACATGATTATGG
TCAAATGTT
Lm-ddA-164-3
CGCCTACCTCAGCCTCCAATCTGCACCATTGATGTCTACATGATTATGG
TCAAATGTT
Lm-ddA-164-4
CGCCTACCTCAGCCTCCAATCTGCACCATTGATGTCTACATGATTATGG
TCAAATGTT
Lm-ddA-164-5
CGCCTACCTCAGCCTCCAATCTGCACCATTGATGTCTACATGATTATGG
TCAAATGTT
Lm-ddA164-6
CGCCTACCTCAGCCTCCAATCTGCACCATTGATGTCTACATGATTATGG
TCAAATGTT
Reference
(SEQ ID NO: 114)
GGATGATTGACTCTGAATGTCGCCCGAGATTCCGGGAGTTGGTGTCAGA
ATTTT
Lm-LLO-NY-1
GGATGATTGACTCTGAATGTCGCCCGAGATTCCGGGAGTTGGTGTCAGA
ATTTT
Lm-LLO-NY-2
GGATGATTGACTCTGAATGTCGCCCGAGATTCCGGGAGTTGGTGTCAGA
ATTTT
Lm-LLO-138-2
GGATGATTGACTCTGAATGTCCCCCGAGATTCCGGGAGTTGGTGTCAAA
ATTTT
Lm-LLO-138-3
GGATGATTGACTCTGAATGTCGCCCGAGATTCCGGGAGTTGGTGTCAGA
ATTTT
Lm-LLO-138-4
GGATGATTGACTCTGAATGTCGCCCGAGATTCCGGGAGTTGGTGTCAGA
ATTTT
Lm-ddA-164-1
GGATGATTGACTCTGAATGTCGCCCGAGATTCCGGGAGTTGGTGTCAGA
ATTTT
Lm-ddA-164-2
GGATGATTGACTCTGAATGTCGCCCGAGATTCCGGGAGTTGGTGTCAGA
ATTTT
Lm-ddA-164-3
GGATGATTGACTCTGAATGTCGCCCGAGATTCCGGGAGTTGGTGTCAGA
ATTTT
Lm-ddA-164-5
GGATGATTGACTCTGAATGTCGCCCGAGATTCCGGGAGTTGGTGTCAGA
ATTTT
Lm-ddA-164-4
GGATGATTGACTCTGAATGTCGCCCGAGATTCCGGGAGTTGGTGTCAGA
ATTTT
Lm-ddA164-6
GGATGATTGACTCTGAATGTCGCCCGAGATTCCGGGAGTTGGTGTCAGA
ATTTT
Reference
(SEQ ID NO: 115)
CACGTATGGCGAGGGACCCCCAGCGTTTTGTGGTCATCCAGAACGAGGA
CTT
Lm-LLO-NY-1
CACGTATGGCGAGGGACCCCCAGCGTTTTGTGGTCATCCAGAACGAGGA
CTT
Lm-LLO-NY-2
CACGTATGGCGAGGGACCCCCAGCGTTTTGTGGTCATCCAGAACGAGGA
CTT
Lm-LLO-138-2
CACGTATGGCGAGGGACCCCCAGCGTTTTGTGGTCATCCAGAACGAGGA
CTT
Lm-LLO-138-3
CACGTATGGCGAGGGACCCCCAGCGTTTTGTGGTCATCCAGAACGAGGA
CTT
Lm-LLO-138-4
CACGTATGGCGAGGGACCCCCAGCGTTTTGTGGTCATCCAGAACGAGGA
CTT
Lm-ddA164-1
CACGTATGGCGAGGGACCCCCAGCGTTTTGTGGTCATCCAGAACGAGGA
CTT
Lm-ddA-164-2
CACGTATGGCGAGGGACCCCCAGCGTTTTGTGGTCATCCAGAACGAGGA
CTT
Lm-ddA-164-3
CACGTATGGCGAGGGACCCCCAGCGTTTTGTGGTCATCCAGAACGAGGA
CTT
Lm-ddA-164-5
CACGTATGGCGAGGGACCCCCAGCGTTTTGTGGTCATCCAGAACGAGGA
CTT
Lm-ddA-164-6
CACGTATGGCGAGGGACCCCCAGCGTTTTGTGGTCATCCAGAACGAGGA
CTT
Alignment of EC1 (399-758 bp of Her-2-neu)
Reference
(SEQ ID NO: 116)
CCCAGGCAGAACCCCAGAGGGGCTGCGGGAGCTGCAGCTTCGAAGTCTC
ACAGAGATCCT
Lm-LLO-138-1
CCCAGGCAGAACCCCAGAGGGGCTGCGGGAGCTGCAGCTTCGAAGTCTC
ACAGAGATCCT
Lm-LLO-138-2
CCCAGGCAGAACCCCAGAGGGGCTGCGGGAGCTGCAGCTTCGAAGTCTC
ACAGAGATCCT
Lm-ddA-164-1
CCCAGGCAGAACCCCAGAGGGGCTGCGGGAGCTGCAGCTTCGAAGTCTC
ACAGAGATCCT
LmddA-164-2
CCCAGGCAGAACCCCAGAGGGGCTGCGGGAGCTGCAGCTTCGAAGTCTC
ACAGAGATCCT
Lmdd-164-3
CCCAGGCAGAACCCCAGAGGGGCTGCGGGAGCTGCAGCTTCGAAGTCTC
ACAGAGATCCT
LmddA164-4
CCCAGGCAGAACCCCAGAGGGGCTGCGGGAGCTGCAGCTTCGAAGTCTC
ACAGAGATCCT
Reference
(SEQ ID NO: 117)
GAAGGGAGGAGTTTTGATCCGTGGGAACCCTCAGCTCTGCTACCAGGAC
ATGGTTTTGTG
Lm-LLO-138-1
GAAGGGAGGAGTTTTGATCCGTGGGAACCCTCAGCTCTGCTACCAGGAC
ATGGTTTTGTG
Lm-LLO-138-2
GAAGGGAGGAGTTTTGATCCGTGGGAACCCTCAGCTCTGCTACCAGGAC
ATGGTTTTGTG
Lm-ddA-164-1
GAAGGGAGGAGTTTTGATCCGTGGGAACCCTCAGCTCTGCTACCAGGAC
ATGGTTTTGTG
LmddA-164-2
GAAGGGAGGAGTTTTGATCCGTGGGAACCCTCAGCTCTGCTACCAGGAC
ATGGTTTTGTG
LmddA-164-3
GAAGGGAGGAGTTTTGATCCGTGGGAACCCTCAGCTCTGCTACCAGGAC
ATGGTTTTGTG
LmddA164-4
GAAGGGAGGAGTTTTGATCCGTGGGAACCCTCAGCTCTGCTACCAGGAC
ATGGTTTTGTG
Reference
(SEQ ID NO: 118)
CCGGGCCTGTCCACCTTGTGCCCCCGCCTGCAAAGACAATCACTGTTGG
GGTGAGAGTCC
Lm-LLO-138-1
CCGGGCCTGTCCACCTTGTGCCCCCGCCTGCAAAGACAATCACTGTTGG
GGTGAGAGTCC
Lm-LLO-138-2
CCGGGCCTGTCCACCTTGTGCCCCCGCCTGCAAAGACAATCACTGTTGG
GGTGAGAGTCC
Lm-ddA-164-1
CCGGGCCTGTCCACCTTGTGCCCCCGCCTGCAAAGACAATCACTGTTGG
GGTGAGAGTCC
LmddA-164-2
CCGGGCCTGTCCACCTTGTGCCCCCGCCTGCAAAGACAATCACTGTTGG
GGTGAGAGTCC
LmddA-164-3
CCGGGCCTGTCCACCTTGTGCCCCCGCCTGCAAAGACAATCACTGTTGG
GGTGAGAGTCC
LmddA164-4
CCGGGCCTGTCCACCTTGTGCCCCCGCCTGCAAAGACAATCACTGTTGG
GGTGAGAGTCC
Reference
(SEQ ID NO: 119)
GGAAGACTGTCAGATCTTGACTGGCACCATCTGTACCAGTGGTTGTGCC
CGGTGCAAGGG
Lm-LLO-138-1
GGAAGACTGTCAGATCTTGACTGGCACCATCTGTACCAGTGGTTGTGCC
CGGTGCAAGGG
Lm-LLO-138-2
GGAAGACTGTCAGATCTTGACTGGCACCATCTGTACCAGTGGTTGTGCC
CGGTGCAAGGG
Lm-ddA-164-1
GGAAGACTGTCAGATCTTGACTGGCACCATCTGTACCAGTGGTTGTGCC
CGGTGCAAGGG
LmddA-164-2
GGAAGACTGTCAGATCTTGACTGGCACCATCTGTACCAGTGGTTGTGCC
CGGTGCAAGGG
LmddA-164-3
GGAAGACTGTCAGATCTTGACTGGCACCATCTGTACCAGTGGTTGTGCC
CGGTGCAAGGG
LmddA164-4
GGAAGACTGTCAGATCTTGACTGGCACCATCTGTACCAGTGGTTGTGCC
CGGTGCAAGGG
Reference
(SEQ ID NO: 120)
CCGGCTGCCCACTGACTGCTGCCATGAGCAGTGTGCCGCAGGCTGCACG
GGCCCCAAGCA
Lm-LLO-138-1
CCGGCTGCCCACTGACTGCTGCCATGAGCAGTGTGCCGCAGGCTGCACG
GGCCCCAAGCA
Lm-LLO-138-2
CCGGCTGCCCACTGACTGCTGCCATGAGCAGTGTGCCGCAGGCTGCACG
GGCCCCAAGCA
Lm-ddA-164-1
CCGGCTGCCCACTGACTGCTGCCATGAGCAGTGTGCCGCAGGCTGCACG
GGCCCCAAGCA
LmddA-164-2
CCGGCTGCCCACTGACTGCTGCCATGAGCAGTGTGCCGCAGGCTGCACG
GGCCCCAAGTA
LmddA-164-3
CCGGCTGCCCACTGACTGCTGCCATGAGCAGTGTGCCGCAGGCTGCACG
GGCCCCAAGTAL
mddA164-4
CCGGCTGCCCACTGACTGCTGCCATGAGCAGTGTGCCGCAGGCTGCACG
GGCCCCAAGTA

### Example 20: Peripheral Immunization with ADXS31-164 can Delay the Growth of a Metastatic Breast Cancer Cell Line in the Brain

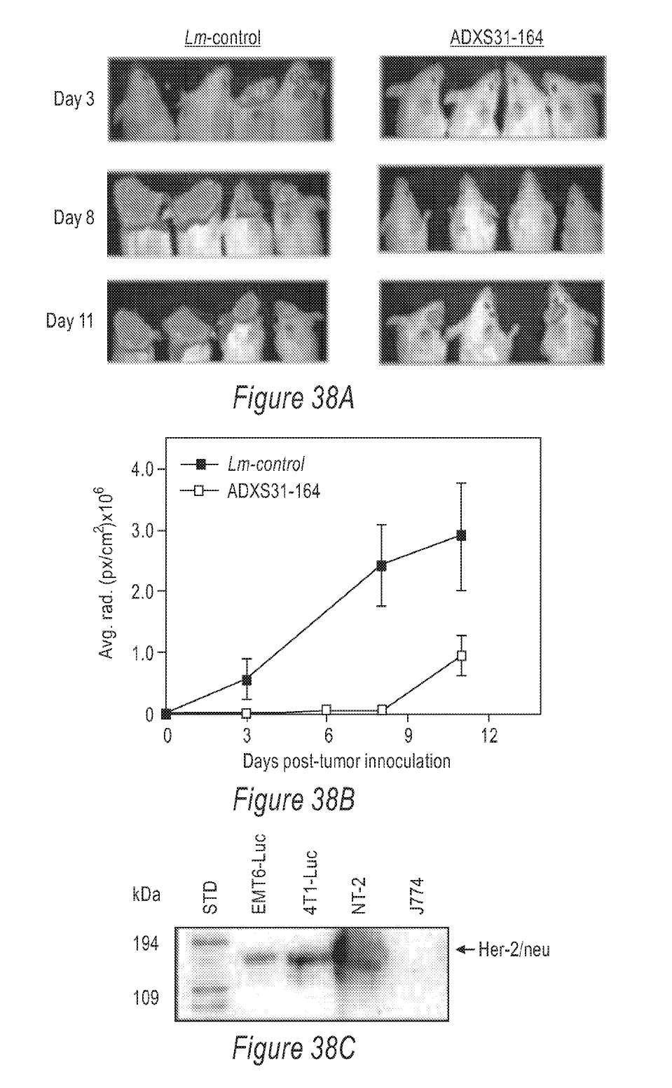

Mice were immunized IP with ADXS31-164 or irrelevant Lm-control vaccines and then implanted intra-cranially with 5,000 EMT6-Luc tumor cells, expressing luciferase and low levels of Her2/neu (FIG. 38C). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 38A and 38B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

While certain features of disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

1. An immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide fused to a heterologous antigen or fragment thereof, said composition further comprising an additional active agent, wherein said additional active agent comprises an attenuated oncolytic virus, a chimeric antigen receptor engineered T cell (CAR T cells), a therapeutic or immunomodulating antibody, a targeting thymidine kinase inhibitor (TKI), or T cell receptor engineered T cells (Receptor engineered T cells), or any combination thereof.

2. (canceled)

3. The composition of claim 1, wherein said attenuated oncolytic virus is selected from the group comprising a vesicular stomatitis virus (VSV), a newcastle disease virus (NDV), a retrovirus, a reovirus, a measles virus, a sinbis virus, an influenza virus, a herpes simplex virus, a vaccinia virus, and an adenovirus.

4. The composition of claim 1, wherein said oncolytic virus expresses a programmed cell death receptor (PD-1) binding agonist or antagonist.

5. The composition of claim 1, wherein said immunomodulating antibody is a PD-1 antagonist selected from the group comprising an antibody or a fragment thereof, a PD-1 antagonist, or a PD-1 partial antagonist, or any combination thereof.

6. The composition of claim 1, wherein said CAR T cells comprise a nucleic acid that encodes an antigen binding domain.

7. The composition of claim 6, wherein said antigen binding domain is an antibody or an antigen-binding fragment thereof and wherein said antigen binding fragment thereof is a Fab or scFv.

8. (canceled)

9. The composition of claim 6, wherein said antigen binding domain binds to a prostate specific antigen (PSA) domain, a human papilloma virus (HPV) antigen domain or a chimeric Her2/neu antigen domain.

10. The composition of claim 1, wherein said antibody recognizes a prostate specific antigen (PSA) epitope, a human papilloma virus (HPV) antigen epitope or a chimeric Her2/neu antigen epitope.

11. The composition of claim 1, wherein said Receptor engineered T cells comprise selective binding specificity to a cell-surface tumor ligand.

12. The composition of claim 11, wherein said ligand comprises a prostate specific antigen (PSA) cell-surface tumor ligand, a human papilloma virus (HPV) cell-surface tumor ligand or a chimeric Her2/neu cell-surface tumor ligand.

13. The composition of claim 1, wherein said thymidine kinase inhibitor (TKI) comprises imatinib mesylate (IM), dasatinib (D), nilotinib (N), bosutinib (B), INNO 406, zelborafinib, gefitinib, erlotinib or sunitinib.

14. The composition of claim 1, wherein said nucleic acid molecule comprising a first open reading frame encoding a fusion polypeptide, is integrated into the Listeria genome.

15. The composition of claim 1, wherein said nucleic acid molecule comprising a first open reading frame encoding a fusion polypeptide, is in a plasmid in said recombinant Listeria strain.

16. The composition of claim 15, wherein said plasmid is stably maintained in said recombinant Listeria strain in the absence of antibiotic selection.

17. The composition of claim 15, wherein said plasmid does not confer antibiotic resistance upon said recombinant Listeria.

18. The composition of claim 1, wherein said heterologous antigen is a tumor-associated antigen.

19. The composition according to claim 18, wherein said tumor-associated antigen is a prostate specific antigen (PSA), a human papilloma virus (HPV) antigen, a chimeric Her2/neu antigen, or an angiogenic antigen.

20. The composition of claim 19, wherein said PSA antigen comprises an amino acid sequence set forth in SEQ ID NO: 26.

21. The composition of claim 19, wherein said HPV antigen comprises an amino acid sequence set forth in SEQ ID NO: 54.

22. The composition of claim 19, wherein said cHER2 antigen comprises an amino acid sequence set forth in SEQ ID NO: 57.

23. (canceled)

24. The composition of claim 19, wherein when said tumor-associated antigen is a prostate specific antigen (PSA), and if present, said antigen binding domain binds to a PSA domain and/or said monoclonal antibody recognizes a PSA epitope.

25. The composition of claim 19, wherein when said tumor associated antigen is a human papilloma virus (HPV) antigen, and if present, said antigen binding domain binds to an HPV antigen and/or said monoclonal antibody recognizes an HPV epitope.

26. The composition of claim 19, wherein when said tumor-associated antigen is a chimeric Her2/neu antigen, and if present, said antigen binding domain binds to a chimeric Her2/neu antigen domain and/or said monoclonal antibody recognizes a Her2/neu antigen.

27. The composition of claim 1, wherein said recombinant Listeria strain is attenuated and wherein said Listeria is Listeria monocytogenes.

28. The composition of claim 27, wherein said attenuated Listeria comprises a mutation, in at least one endogenous gene and wherein said mutation comprises inactivation, truncation, deletion, replacement or disruption.

29. (canceled)

30. The composition of claim 28, wherein said endogenous gene is an actA virulence gene, a prfA virulence gene, a dal gene, an inlB gene, a dat gene or a combination thereof.

31. The composition of claim 30, wherein said endogenous genes comprise the dal/dat and actA genes.

32. The composition of claim 31, wherein said nucleic acid comprising a first open reading frame, further comprises a second open reading frame.

33. The composition of claim 32, wherein said second open reading frame encodes a PrfA protein comprising a D133V mutation, and wherein said PrfA protein complements said mutation, deletion, disruption, inactivation, replacement, or truncation in said prfA gene.

34. The composition of claim 32, wherein said second open reading frame encodes a metabolic enzyme and wherein said metabolic enzyme complements said mutation, deletion, disruption, inactivation, replacement, or truncation in said dal and dat genes.

35. The composition according to claim 34, wherein said metabolic enzyme encoded by said second open reading frame is an alanine racemase enzyme or a D-amino acid transferase enzyme.

36. The composition of claim 1, further comprising an adjuvant.

37. The composition of claim 36, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

38. (canceled)

39. A method of eliciting an enhanced anti-tumor T cell response in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a Truncated LLO, a truncated ActA or a PEST-sequence peptide or a PEST-sequence peptide fused to a heterologous antigen or fragment thereof, wherein:
(a) said composition further comprises an additional active agent, wherein said additional active agent comprises an attenuated oncolytic virus, a chimeric antigen receptor engineered T cell (CAR T cells), a therapeutic or immunomodulating monoclonal antibody, a targeting thymidine kinase inhibitor (TKI), or T cell receptor engineered T cells (Receptor engineered T cells), or any combination thereof;
(b) said method further comprises a step of administering an effective amount of a composition comprising an additional active agent to said subject; or
(c) said method further comprises a step of administering a targeted radiation therapy to said subject; or any combination thereof of (a)-(c).

40.-87. (canceled)
